(19) United States
(12) Patent Application Publication
(10) Pub. No.: US 2024/0150371 A1
(43) Pub. Date: May 9, 2024

(54) Compounds for the Treatment of Kinase-Dependent Disorders

(71) Applicant: Exelixis, Inc., Alameda, CA (US)

(72) Inventors: Lynne Canne Bannen, Novato, CA (US); Minna Bui, Oakland, CA (US); Faming Jiang, Castro Valley, CA (US); Yong Wang, South San Francisco, CA (US); Wei Xu, Danville, CA (US)

(21) Appl. No.: 18/132,124

(22) Filed: Apr. 7, 2023

Related U.S. Application Data
(63) Continuation of application No. 16/964,274, filed on Jul. 23, 2020, now Pat. No. 11,673,897.
(60) Provisional application No. 62/622,702, filed on Jan. 26, 2018.
(60) Provisional application No. 62/622,626, filed on Jan. 26, 2018.

(51) Int. Cl. C07D 491/147; C07D 401/12; C07D 401/14; C07D 413/14; C07D 471/04

(52) U.S. Cl. CPC C07D 491/147; C07D 401/12; C07D 401/14; C07D 413/14; C07D 471/04

(57) ABSTRACT

Disclosed herein are compounds of Formula I′. Compounds of Formula I′ inhibit, regulate and/or modulate kinase receptor, particularly Axl and Mer signal transduction pathways related to the changes in cellular activities as mentioned above, compositions which contain these compounds, and methods of using them to treat kinase-dependent diseases and conditions. The present invention also provides methods for making compounds as mentioned above, and compositions which contain these compounds.

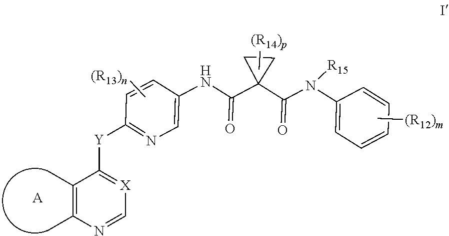

### CROSS-REFERENCE TO RELATED APPLICATIONS

This application is a continuation application based on U.S. application Ser. No. 16/964,274, filed Jul. 23, 2020, which is a United States National Phase of PCT/US2019/015296, filed Jan. 25, 2019, which claims the benefit of priority to U.S. Provisional Application No. 62/622,626, filed Jan. 26, 2018, and to U.S. Provisional Application Ser. No. 62/622,702, filed Jan. 26, 2018, the entire contents of which are incorporated herein.

### FIELD OF THE INVENTION

The invention relates to compounds for modulating protein kinase enzymatic activity for modulating cellular activities such as proliferation, differentiation, programmed cell death, migration, and chemoinvasion. Even more specifically, the invention relates to compounds which inhibit, regulate, and/or modulate Axl and Mer receptor tyrosine kinases, compositions which contain these compounds, methods of using them to treat kinase-dependent diseases and conditions, synthesis of the compounds, and processes for formulating the compounds for pharmaceutical purposes.

### SEQUENCE LISTING

The instant application contains a Sequence Listing which has been submitted electronically in XML format and which is hereby incorporated by reference in its entirety. Said XML copy, created on Jun. 28, 2023, is named “224990-527168_Sequence-listing” and is 72,159 bytes in size.

### BACKGROUND OF THE INVENTION

Human Axl belongs to the TAM subfamily of receptor tyrosine kinases that includes Mer. TAM kinases are characterized by an extracellular ligand binding domain consisting of two immunoglobulin-like domains and two fibronectin type III domains. Axl is overexpressed in a number of tumor cell types and was initially cloned from patients with chronic myelogenous leukemia. When overexpressed, Axl exhibits transforming potential. Axl signaling is believed to cause tumor growth through activation of proliferative and anti-apoptotic signaling pathways. Axl has been associated with cancers such as lung cancer, myeloid leukemia, uterine cancer, ovarian cancer, gliomas, melanoma, thyroid cancer, renal cell carcinoma, osteosarcoma, gastric cancer, prostate cancer, and breast cancer. The over-expression of Axl results in a poor prognosis for patients with the indicated cancers.

Activation of Mer, like Axl, conveys downstream signaling pathways that cause tumor growth and activation. Mer binds ligands such as the soluble protein Gas-6. Gas-6 binding to Mer induces autophosphorylation of Mer on its intracellular domain, resulting in downstream signal activation. Over-expression of Mer in cancer cells leads to increased metastasis most likely by generation of soluble Mer extracellular domain protein as a decoy receptor. Tumor cells secrete a soluble form of the extracellular Mer receptor which reduces the ability of soluble Gas-6 ligand to activate Mer on endothelial cells leading to cancer progression.

Therefore a need exists for compounds that inhibit TAM receptor tyrosine kinases such as Axl and Mer for the treatment of selected cancers.

### SUMMARY OF THE INVENTION

In one aspect, the present invention provides compounds for modulating kinase activity and methods of treating diseases mediated by kinase activity utilizing the compounds of Formula I′.

In one aspect, the invention includes a compound of Formula I′:

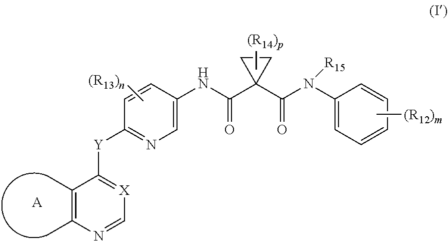

or a pharmaceutically acceptable salt thereof, wherein:
X is N or CH;
Y is selected from O, S, SO, SO₂, NH, and —N(C₁₋₆ alkyl)-;
(i) ring A is

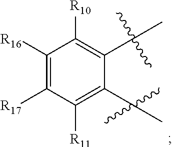

R₁₆ is selected from the group consisting of (C₂-C₆) alkenyl; (C₂-C₆) alkynyl; (C₆-C₁₀) aryl; (C₃-C₁₀) cycloalkyl; 5-14 membered heteroaryl; 4-14 membered heterocycloalkyl; —CN; —NHOH, —C(O)R^{a}; —C(O)NR^{a}R^{a}; —C(O)NHOR^{a}; —C(O)OR^{a}; —C(O)NR^{a}S(O)₂R^{a}; —OC(O)NR^{a}R^{a}; C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —S(O)NR^{a}R^{a}; —S(O)₂NR^{a}C(O)R^{a}; —P(O)R^{a}R^{a}; —P(O)(OR^{a})(OR^{a}); —B(OH)₂; —B(OR^{a})₂; and S(O)₂NR^{a}R^{a}; and
R₁₇ is selected from —H; halo; (C₁-C₆) alkyl; (C₂-C₆) alkenyl; (C₂-C₆) alkynyl; (C₁-C₆) haloalkyl; (C₁-C₆) haloalkoxy; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; (4-14 membered heterocycloalkyl)-(C₁-C₄) alkylene-; —CN; —NO₂; —OR^{a}; —SR^{a}; —NHOR^{a}; —C(O)R^{a}; —C(O)NR^{a}R^{a}; —C(O)NHOR^{a}; —C(O)OR^{a}; —C(O)NR^{a}S(O)₂R^{a}; —OC(O)R^{a}; —OC(O)NR^{a}R^{a}; —NR^{a}W; —NR^{a}R^{a}; —NR^{a}C(O)R^{a}; —NR^{a}C(═NR^{a})R^{a}; —NR^{a}C(O)OR^{a}; —NR^{a}C(O)NR^{a}R^{a}; —C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —NR^{a}C(═NR^{a})NR^{a}R^{a}; —NR^{a}S(O)R^{a}; —NR^{a}S(O)₂R^{a}; —NR^{a}S(O)2NR^{a}R^{a}; —S(O)R^{a}; —S(O)NR^{a}R^{a}; —S(O)₂R^{a}; —S(O)₂NR^{a}C(O)R^{a}; —P(O)R^{a}R^{a}; —P(O)(OR^{a})(OR^{a}); —B(OH)₂; —B(OR^{a})₂; and —S(O)₂NR^{a}R^{a}; wherein the (C₁-C₆) alkyl; (C₂-C₆) alkenyl; (C₂-C₆) alkynyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R₁₆ or R₁₇ are each optionally substituted with 1, 2, 3, 4, or 5 independently selected R^{b} substituents, provided when R₁₆ or R₁₇ is 5-membered heteroaryl or 5-7 membered heterocycloalkyl, then the 5-membered heteroaryl or 5-7 membered heterocycloalkyl does not connect to the fused phenyl ring moiety through a ring nitrogen atom; or
R₁₆ is selected from —H; halo; (C₁-C₆) alkyl; (C₂-C₆) alkenyl; (C₂-C₆) alkynyl; (C₁-C₆) haloalkyl; (C₁-C₆)haloalkoxy; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; (4-14 membered heterocycloalkyl)-(C₁-C₄) alkylene-; —CN; —NO₂; —OR^{a}; —SR^{a}; —NHOR^{a}; —C(O)R^{a}; —C(O)NR^{a}R^{a}; —C(O)NHOR^{a}; —C(O)OR^{a}; —C(O)NR^{a}S(O)₂R^{a}; —OC(O)R^{a}; —OC(O)NR^{a}R^{a}; —NHR^{a}; —NR^{a}R^{a}; —NR^{a}C(O)R^{a}; —NR^{a}C(═NR^{a})R^{a}; —NR^{a}C(O)OR^{a}; —NR^{a}C(O)NR^{a}R^{a}; —C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —NR^{a}C(═NR^{a})NR^{a}R^{a}; —NR^{a}S(O)R^{a}; —NR^{a}S(O)₂R^{a}; —NR^{a}S(O)₂NR^{a}R^{a}; —S(O)R^{a}; —S(O)NR^{a}R^{a}; —S(O)₂R^{a}; —S(O)₂NR^{a}C(O)R^{a}; —P(O)R^{a}R^{a}; —P(O)(OR^{a})(OR^{a}); —B(OH)₂; —B(OR^{a})₂; and —S(O)₂NR^{a}R^{a}; wherein the (C₁-C₆) alkyl; (C₂-C₆) alkenyl; (C₂-C₆) alkynyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R₁₆ is each optionally substituted with 1, 2, 3, 4, or 5 independently selected R^{b} substituents; and
R₁₇ is selected from the group consisting of (C₂-C₆) alkenyl; (C₂-C₆) alkynyl; —CN; —NHOH, —C(O)R^{a}; —C(O)NR^{a}R^{a}; —C(O)NHOR^{a}; —C(O)OR^{a}; —C(O)NR^{a}S(O)₂R^{a}; —OC(O)NR^{a}R^{a}; C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —S(O)NR^{a}R^{a}; —S(O)₂NR^{a}C(O)R^{a}; —P(O)R^{a}R^{a}; —P(O)(OR^{a})(OR^{a}); —B(OH)₂; —B(OR^{a})₂; and S(O)₂NR^{a}R^{a}, provided when R₁₆ or R₁₇ is 5-membered heteroaryl or 5-7 membered heterocycloalkyl, then the 5-membered heteroaryl or 5-7 membered heterocycloalkyl does not connect to the fused phenyl ring moiety through a ring nitrogen atom; or
R₁₆ and R₁₇ taken together with the atoms to which they are attached form a fused C₃0.7 cycloalkyl ring or a fused 4- to 10-membered heterocycloalkyl ring; wherein the fused C₃0.7 cycloalkyl ring and fused 4- to 10-membered heterocycloalkyl ring are each optionally substituted with 1, 2, or 3 independently selected R^{b} substituents; or
(ii) ring A is

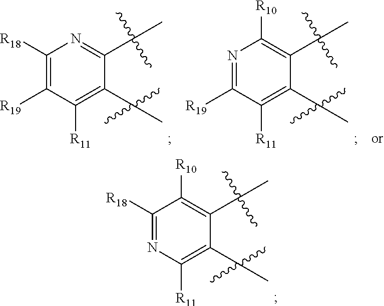

R₁₈ and R₁₉ are each independently selected from —H; halo; (C₁-C₆) alkyl; (C₂-C₆) alkenyl; (C₂-C₆) alkynyl; (C₁-C₆) haloalkyl; (C₁-C₆)haloalkoxy; (C₆-C₁₀) aryl; (C₃-C₁₀) cycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; (4-14 membered heterocycloalkyl)-(C₁-C₄) alkylene-; —CN; —NO₂; —OR^{a}; —SR^{a}; —NHOR^{a}; —C(O)R^{a}; —C(O)NR^{a}R^{a}; —C(O)NHOR^{a}; —C(O)OR^{a}; —C(O)NR^{a}S(O)₂R^{a}; —OC(O)R^{a}; —OC(O)NR^{a}R^{a}; —NHR^{a}; —NR^{a}R^{a}; —NR^{a}C(O)R^{a}; —NR^{a}C(═NR^{a})R^{a}; —NR^{a}C(O)OR^{a}; —NR^{a}C(O)NR^{a}R^{a}; —C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —NR^{a}C(═NR^{a})NR^{a}R^{a}; —NR^{a}S(O)R^{a}; —NR^{a}S(O)₂R^{a}; —NR^{a}S(O)₂NR^{a}R^{a}; —S(O)R^{a}; —S(O)NR^{a}R^{a}; —S(O)₂R^{a}; —S(O)₂NR^{a}C(O)R^{a}; —P(O)R^{a}R^{a}; —P(O)(OR^{a})(OR^{a}); —B(OH)₂; —B(OR^{a})₂; and —S(O)₂NR^{a}R^{a}; wherein the (C₁-C₆) alkyl; (C₂-C₆) alkenyl; (C₂-C₆) alkynyl; (C₆-C₁₀) aryl; (C₃-C₁₀) cycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R₁₈ or R₁₉ are each optionally substituted with 1, 2, 3, 4, or 5 independently selected R^{b} substituents; or
R₁₈ and R₁₉ taken together with the atoms to which they are attached form a fused C₃₋₇ cycloalkyl ring or a fused 4- to 10-membered heterocycloalkyl ring; wherein the fused C₃₋₇ cycloalkyl ring and fused 4- to 10-membered heterocycloalkyl ring are each optionally substituted with 1, 2, or 3 independently selected R^{b} substituents;
R₁₀ and R₁₁ are each independently selected from the group consisting of —H; halo; (C₁-C₆) alkyl; (C₁-C₆) haloalkyl; (C₁-C₆)haloalkoxy; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; (4-14 membered heterocycloalkyl)-(C₁-C₄) alkylene-; —CN; —NO₂; —OR^{a}; —SR^{a}; —NHOR^{a}; —C(O)R^{a}; —C(O)NR^{a}R^{a}; —C(O)OR^{a}; —C(O)NR^{a}S(O)₂R^{a}; —OC(O)R^{a}; —OC(O)NR^{a}R^{a}; —NR^{a}W; —NR^{a}R^{a}; —NR^{a}C(O)R^{a}; —NR^{a}C(═NR^{a})R^{a}; —NR^{a}C(O)OR^{a}; —NR^{a}C(O)NR^{a}R^{a}; —C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —NR^{a}C(═NR^{a})NR^{a}R^{a}; —NR^{a}S(O)R^{a}; —NR^{a}S(O)₂R^{a}; —NR^{a}S(O)₂NR^{a}R^{a}; —S(O)R^{a}; —S(O)NR^{a}R^{a}; —S(O)₂R^{a}; —S(O)₂NR^{a}C(O)R^{a}; —P(O)R^{a}R^{a}; —P(O)(OR^{a})(OR^{a}); —B(OH)₂; —B(OR^{a})₂; and S(O)₂NR^{a}R^{a}; wherein the (C₁-C₆) alkyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R₁ or R₂ are each optionally substituted with 1, 2, 3, 4, or 5 independently selected R^{b} substituents;
each R₁₃ is independently selected from the group consisting of —H; halo; —OH; —CN; optionally substituted (C₁-C₆) alkyl; (C₁-C₆) alkoxy; (C₁-C₆) haloalkoxy; —NH₂; —NH(C₁-C₆)alkyl; —N(C₁-C₆ alkyl)₂; and (C₃-C₆) cycloalkyl; wherein the (C₁-C₆) alkoxy; —NH(C₁-C₆)alkyl; —N(C₁-C₆ alkyl)₂; and (C₃-C₆) cycloalkyl of R₃ are each optionally substituted with 1, 2, or 3 independently selected R^{g} substituents;
each R₁₄ is independently selected from the group consisting of halo; —OH; —NH₂; —CN; (C₁-C₆) alkyl; (C₁-C₆) alkoxy; (C₁-C₆) haloalkyl; (C₁-C₆) haloalkoxy; —COOH; —NH(C₁-C₆)alkyl; —N(C₁-C₆ alkyl)₂; phenyl; phenyl-(C₁-C₂) alkylene; (C₃-C₆) cycloalkyl; (C₃-C₆) cycloalkyl-(C₁-C₄) alkylene-; 4- to 6-membered heterocycloalkyl; (4- to 6-membered heterocycloalkyl)-(C₁-C₄) alkylene-; 5- to 6-membered heteroaryl; (5- to 6-membered heteroaryl)-(C₁-C₄) alkylene-; and —OR^{e}; wherein the (C₁-C₆) alkyl; phenyl; phenyl-(C₁-C₂) alkylene; (C₃-C₆) cycloalkyl; (C₃-C₆) cycloalkyl-(C₁-C₄) alkylene-; 4- to 6-membered heterocycloalkyl; (4- to 6-membered heterocycloalkyl)-(C₁-C₄) alkylene-; 5- to 6-membered heteroaryl; and (5- to 6-membered heteroaryl)-(C₁-C₄) alkylene- of R₁₄ are each optionally substituted with 1, 2, or 3 independently selected R^{g} substituents;
R₁₅ is H;
each R₁₂ is independently selected from the group consisting of —H; halo; —OH; —COOR^{e}; —CONR^{e}R^{e}; —CN; —NH₂; —NH((C₁-C₆) alkyl); —N((C₁-C₆) alkyl)₂; (C₁-C₆) alkyl; (C₁-C₆) alkoxy; (C₁-C₆) haloalkyl; (C₁-C₆) haloalkoxy; —CONR^{a}R^{a}; —NR^{a}COR^{a}; —NR^{a}CONR^{a}R^{a}; —SO₂R; —NR^{a}S(O)₂R^{a}; —NR^{a}S(O)₂NR^{a}R^{a}; (C₃-C₆) cycloalkyl; 4- to 6-membered heterocycloalkyl; phenyl; 5- or 6-membered heteroaryl; (C₃-C₆) cycloalkyl-(C₁-C₄) alkylene-; (4- to 6-membered heterocycloalkyl)-(C₁-C₄) alkylene-; phenyl-(C₁-C₂) alkylene; and (5- or 6-membered heteroaryl)-(C₁-C₄) alkylene-; wherein the (C₁-C₆) alkyl; (C₃-C₆) cycloalkyl; 4- to 6-membered heterocycloalkyl; phenyl; 5- or 6-membered heteroaryl; (C₃-C₆) cycloalkyl-(C₁-C₄) alkylene-; (4-to 6-membered heterocycloalkyl)-(C₁-C₄) alkylene-; phenyl-(C₁-C₂) alkylene; and (5- or 6-membered heteroaryl)-(C₁-C₄) alkylene- of R₁₂ are each optionally substituted with 1, 2, or 3 independently selected R^{f} substituents;
each R^{a} is independently selected from the group consisting of —H; —CN; (C₁-C₆) alkyl; (C₁-C₆) haloalkyl; (C₂-C₆) alkenyl; (C₂-C₆) alkynyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene-; wherein the (C₁-C₆) alkyl; (C₁-C₆) haloalkyl; (C₂-C₆) alkenyl; (C₂-C₆) alkynyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R^{a} are each optionally substituted with 1, 2, 3, 4, or 5 independently selected R^{d} substituents;
each R^{b} is independently selected from the group consisting of halo; (C₁-C₆) alkyl; (C₂-C₆) alkenyl; (C₂-C₆) alkynyl; (C₁-C₆) haloalkyl; (C₁-C₆) haloalkoxy; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; (4-10 membered heterocycloalkyl)-(C₁-C₄) alkylene-; —CN; —OH; —NH₂; —NO₂; —NHOR^{c}; —OR^{c}; —SR^{c}; —C(O)R^{c}; —C(O)NR^{c}R^{c}; —C(O)OR^{c}; —C(O)NR^{c}S(O)₂R^{c}; —OC(O)R^{c}; —OC(O)NR^{c}R^{c}; —C(═NOH)R^{c}; —C(═NOH)NR^{c}; —C(═NCN)NR^{c}R^{c}; —NR^{c}C(═NCN)NR^{c}R^{c}; —C(═NR^{c})NR^{c}R^{c}; —NR^{c}C(═NR^{c})NR^{c}R^{c}; —NHR^{c}; —NR^{c}R^{c}; —NR^{c}C(O)R^{c}; —NR^{c}C(═NR^{c})R^{c}; —NR^{c}C(O)OR^{c}; —NR^{c}C(O)NR^{c}R^{c}; —NR^{c}S(O)R^{c}; —NR^{c}S(O)₂R^{c}; —NR^{c}S(O)₂NR^{c}R^{c}; —S(O)R^{c}; —S(O)NR^{c}R^{c}; —S(O)₂R^{c}; —S(O)₂NR^{c}C(O)R^{c}; —Si(R^{c})₃; —P(O)R^{c}R^{c}; —P(O)(OR^{c})(OR^{c}); —B(OH)₂; —B(OR^{c})₂; and —S(O)₂NR^{c}R^{c}; wherein the (C₁-C₆) alkyl; (C₁-C₆) haloalkyl; (C₁-C₆) haloalkoxy; (C₂-C₆) alkenyl; (C₂-C₆) alkynyl; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalky-(C₁-C₄) alkylene-; (5-10 membered heteroaryl)-(C₁-C₄) alkylene-; and (4-10 membered heterocycloalkyl)-(C₁-C₄) alkylene- of R^{b} are each further optionally substituted with 1, 2, or 3 independently selected R^{d} substituents;
each R^{c} is independently selected from the group consisting of —H; (C₁-C₆) alkyl; (C₁-C₆) haloalkyl; (C₂-C₆) alkenyl; (C₂-C₆) alkynyl; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; and (4-10 membered heterocycloalkyl)-(C₁-C₄) alkylene-; wherein the (C₁-C₆) alkyl; (C₂-C₆) alkenyl; (C₂-C₆) alkynyl; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; and (4-10 membered heterocycloalkyl)-(C₁-C₄) alkylene- of R^{c} are each optionally substituted with 1, 2, 3, 4, or 5 independently selected R^{f} substituents;
each R^{d} is independently selected from the group consisting of (C₁-C₆) alkyl; (C₁-C₆) haloalkyl; halo; (C₆-C₁₀) aryl; 5-10 membered heteroaryl; (C₃-C₁₀) cycloalkyl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; (4-10 membered heterocycloalkyl)-(C₁-C₄) alkylene-; —CN; —NH₂; —NHOR^{e}; —OR^{e}; —SR^{e}; —C(O)R^{e}; —C(O)NR^{e}R^{e}; —C(O)OR^{e}; —OC(O)R^{e}; —OC(O)NR^{e}R^{e}; —NHR^{e}; —NR^{e}R^{e}; —NR^{e}C(O)R^{e}; —NR^{e}C(O)NR^{e}R^{e}; —NR^{e}C(O)OR^{e}; —C(═NR^{e})NR^{e}R^{e}; —NR^{e}C(═NR^{e})NR^{e}R^{e}; —NR^{e}C(═NOH)NR^{e}R^{e}; —NR^{e}C(═NCN)NR^{e}R^{e}; —S(O)R^{e}; —S(O)NR^{e}R^{e}; —S(O)₂R^{e}; —NR^{e}S(O)₂R^{e}; —NR^{e}S(O)₂NR^{e}R^{e}; and —S(O)₂NR^{e}R^{e}; wherein the (C₁-C₆) alkyl; (C₁-C₆) haloalkyl; (C₆-C₁₀) aryl; 5-10 membered heteroaryl; (C₃-C₁₀) cycloalkyl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; and (4-10 membered heterocycloalkyl)-(C₁-C₄) alkylene- of R^{d} are each optionally substituted with 1, 2, or 3 independently selected R^{f} substituents;
each R^{e} is independently selected from the group consisting of —H; (C₁-C₆) alkyl; (C₃-C₆) cycloalkyl; (C₃-C₆) cycloalkyl-(C₁-C₄) alkylene-; (C₆-C₁₀) aryl; (C₆-C₁₀) aryl-(C₁-C₄) alkylene-; 5- or 6-membered heteroaryl; (5- or 6-membered heteroaryl)-(C₁-C₄) alkylene-; 4-7-membered heterocycloalkyl; (4-7-membered heterocycloalkyl)-(C₁-C₄) alkylene-; (C₁-C₆) haloalkyl; (C₁-C₆) haloalkoxy; (C₂-C₄) alkenyl; and (C₂-C₄) alkynyl; wherein the (C₁-C₄) alkyl; (C₃-C₆) cycloalkyl; (C₆. C₁₀) aryl; 5 or 6-membered heteroaryl; 4-7-membered heterocycloalkyl; (C₆-C₁₀) aryl-(C₁-C₄) alkylene-; (5- or 6-membered heteroaryl)-(C₁-C₄) alkylene-; (4-7-membered heterocycloalkyl)-(C₁-C₄) alkylene-; (C₂-C₄) alkenyl; and (C₂-C₄) alkynyl of R^{e} are each optionally substituted with 1, 2, or 3 R^{f} substituents;
or any two R^{a} substituents together with the nitrogen atom to which they are attached form 4-5-, 6-, 7-, 8-, 9-, or 10-membered heterocycloalkyl, each of which is optionally substituted with 1, 2, or 3 independently selected R^{f} substituents;
or any two R^{c} substituents together with the nitrogen atom to which they are attached form 4-5-, 6-, 7-, 8-, 9-, or 10-membered heterocycloalkyl, each of which is optionally substituted with 1, 2, or 3 independently selected R^{f} substituents;
or any two R^{e} substituents together with the nitrogen atom to which they are attached form 4-5-, 6-, 7-, 8-, 9-, or 10-membered heterocycloalkyl, each of which is optionally substituted with 1, 2, or 3 independently selected R^{f} substituents;
each R^{f} is independently selected from the group consisting of halo; —OH; —CN; —COOH; —NH₂; —NH—(C₁-C₆) alkyl; —N((C₁-C₆) alky)₂; (C₁-C₆) alkyl; (C₁-C₆) alkoxy; (C₁-C₆) alkylthio; (C₁-C₆) haloalkyl; (C₁-C₆)haloalkoxy; phenyl; 5-6 membered heteroaryl; 4-6 membered heterocycloalkyl; and (C₃-C₆) cycloalkyl; wherein the (C₁-C₆) alkyl; phenyl; (C₃-C₆) cycloalkyl; 4-6 membered heterocycloalkyl; and 5-6 membered heteroaryl of R^{f} are each optionally substituted with 1, 2, or 3 substituents selected from halo; —OH; —CN; —COOH; —NH₂; (C₁-C₄) alkyl; (C₁-C₄) alkoxy; (C₁-C₄) haloalkyl; (C₁-C₄) haloalkoxy; phenyl; (C₃-C₁₀) cycloalkyl; 5-6 membered heteroaryl; and 4-6 membered heterocycloalkyl;
each R^{g} is independently selected from the group consisting of halo; —OH; —CN; —COOH; —COO—(C₁-C₄) alkyl; —NH₂; —NH—(C₁-C₆) alkyl; —N((C₁-C₆) alky)₂; (C₁-C₆) alkyl; (C₁-C₆) alkoxy; (C₁-C₆) alkylthio; (C₁-C₆)haloalkyl; (C₁-C₆)haloalkoxy; phenyl; 5-6 membered heteroaryl; 4-6 membered heterocycloalkyl; and (C₃-C₆) cycloalkyl;
the ring nitrogen atom on the quinoline moiety in Formula A is optionally oxidized;
the subscript n is an integer of 1, 2, 3, or 4;
the subscript m is an integer of 1, 2, 3, 4, or 5; and
the subscript p is an integer of 0, 1, 2, 3, or 4.

In one embodiment, the compound of Formula I′ is a compound of Formula I:

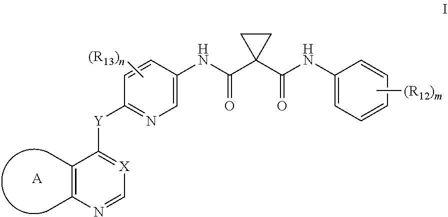

wherein:
X is selected from N and C—H;
Y is O, S, SO, SO₂, NH, or N—(C₁-C₆ alkyl);
R₁₃ is selected from —H, halo, —CN, —C(O)NH₂, and optionally substituted C₁₋₆ alkyl;
R₁₂ is —H or halo;

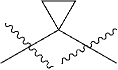 [SMILES: *C1(*)CC1]

is optionally substituted with one, two, three, or four groups independently selected from the group consisting of halo, and C₁-C₆ alkyl, wherein “ [symbol] ” indicate points of attachment;

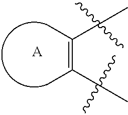

is selected from the group consisting of

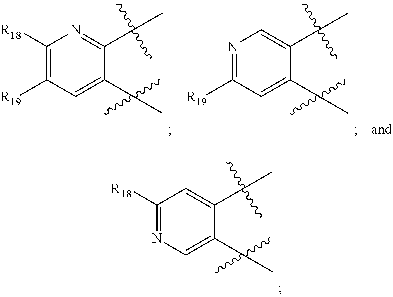

wherein R₁₈ and R₁₉ are selected from the group consisting of H, halo, —CN, optionally substituted C₁-C₆ alkyl, C(O)NR₅R₆, and optionally substituted C₁-C₆ alkoxy; or
when

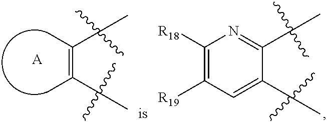

R₁₈ and R₁₉ can be joined together to form a 5 or 6-membered optionally substituted cycloalkyl or optionally substituted heterocycloalkyl;
R₅ and R₆ are selected from the group consisting of H, optionally substituted C₁₋₆ alkyl, or R₅ and R₆ taken together with the nitrogen to which they are attached to form a 5- or 6-membered optionally substituted heterocycle; and
n and m are each independently 1 or 2;
wherein when

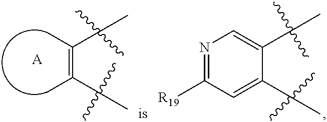

and X is C—H, R₁₉ is not optionally substituted C₁-C₆ alkyl, halo, or optionally substituted C₁-C₆ alkoxy.

In one embodiment, the compound of Formula I′ is a compound of Formula II:

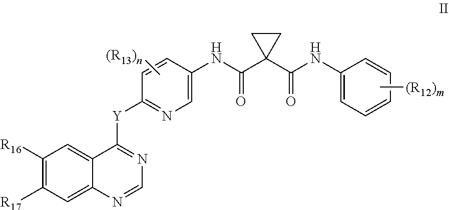

or a pharmaceutically acceptable salt thereof, wherein:
R₁₆ is selected from the group consisting of —CN, optionally substituted 5-6 membered heteroaryl, —COORₐ, and —CO—NR₅R₆;
R₁₇ is selected from H and optionally substituted C₁-C₆ alkoxy;
R₁₃ is selected from the group consisting of —H, halo, —CN, or optionally substituted C₁-C₆ alkyl, or optionally substituted C₁-C₆ alkoxy;
R₁₂ is —H or halo;

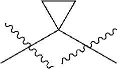 [SMILES: *C1(*)CC1]

is optionally substituted with one, two, three, or four groups independently selected from the group consisting of halo, and C₁-C₆ alkyl, wherein “ [symbol] ” indicate points of attachment;
R₅ and R₆ are each independently H or optionally substituted C₁-C₆ alkyl, optionally substituted C₃-C₆ heterocycloalkyl, optionally substituted C₁-C₆ cycloalkyl;
Y is O, S, SO, SO₂, NH, or N—(C₁-C₆ alkyl); and
n and m are each independently 1 or 2.

In one aspect, the invention includes a pharmaceutical composition comprising a compound described herein, and a pharmaceutically acceptable carrier or excipient.

In another aspect, the invention includes a method of treating a disease, disorder, or syndrome mediated at least in part by modulating in vivo activity of a protein kinase, comprising administering to a subject in need thereof a therapeutically effective amount of a compound described herein or a pharmaceutical composition of claim 40.

### DETAILED DESCRIPTION OF THE INVENTION

### Abbreviations and Definitions

The following abbreviations and terms have the indicated meanings throughout:

| Abbreviation | Meaning |
|---|---|
| Ac | Acetyl |
| anhyd | Anhydrous |
| Aq | Aqueous |
| Ar | Argon |
| Boc | Tert-butoxycarbonyl |
| Br | Broad |
| ° C. | Degrees Celsius |
| c- | Cyclo |
| calcd | Calculated |
| CBZ | CarboBenZoxy = benzyloxycarbonyl |
| d | Doublet |
| dd | Doublet of doublets |
| ddd | Doublet of doublets of doublets |
| dt | Doublet of triplets |
| DCM | Dichloromethane |
| DMF | N,N-Dimethylformamide |
| DMSO | Dimethyl sulfoxide |
| Dppf | 1,1′-bis(diphenylphosphano)ferrocene |
| EA | Elemental Analysis |
| EI | Electron Impact ionization |
| eq or equiv | Equivalent |
| Fmoc | Fluorenylmethyloxycarbonyl |
| g | Gram(s) |
| h or hr | Hour(s) |
| HPLC | High pressure liquid chromatography |
| H₂ | Hydrogen |
| L | Liter(s) |
| LiHMDS | Lithium bis(trimethylsilyl)azide |
| M | Molar or molarity |
| m | Multiplet |
| MHz | Megahertz (frequency) |
| Min | Minute(s) |
| mL | Milliliter(s) |
| Mp | Melting point |
| m/z | Mass to charge ratio |
| μL | Microliter(s) |
| Mol | Mole(s) |
| MS | Mass spectral analysis |
| N₂ | Nitrogen |
| N | Normal or normality |
| nM | Nanomolar |
| NMR | Nuclear magnetic resonance spectroscopy |
| Pd/C | Palladium on carbon |
| Q | Quartet |
| RT | Room temperature |
| s | Singlet |
| soln | Solution |
| S/C | Substrate/catalyst ratio |
| t or tr | Triplet |
| THF | Tetrahydrofuran |
| TLC | Thin layer chromatography |
| v/v | Volume to volume |

The symbol “-” means a single bond, and “=” means a double bond.

As used herein, the singular forms “a,” “an,” and “the” include plural reference unless the context clearly dictates otherwise.

When a variable is defined generically, with a number of possible substituents, each individual radical can be defined with or without the bond. For example, if R^{z} can be hydrogen, this can be indicated as “—H” or “H” in the definition of R^{z}.

When chemical structures are depicted or described, unless explicitly stated otherwise, all carbons are assumed to have hydrogen substitution to conform to a valence of four. For example, in the structure on the left-hand side of the schematic below, there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

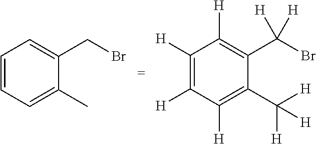

If a group “R” is depicted as “floating” on a ring system, as for example in the formula:

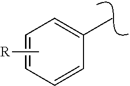

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the formulae:

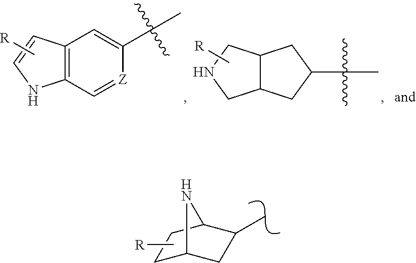

then, unless otherwise defined, a substituent “R” may reside on any atom of the fused ring system, assuming replacement of a depicted hydrogen (for example the —NH— in the formula above), implied hydrogen (for example, in the formula above, where the hydrogens are not shown but understood to be present), or expressly defined hydrogen (for example, where in the formula above, “Z” equals ═CH—) from one of the ring atoms, so long as a stable structure is formed. In the example depicted, the “R” group may reside on either the 5-membered or the 6-membered ring of the fused ring system. When a group “R” is depicted as existing on a ring system containing saturated carbons, for example in the formula:

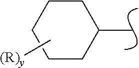

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group, there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

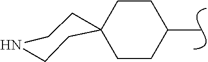

“Halogen” or “halo” refers to fluorine, chlorine, bromine, or iodine.

The term “Cₙ₋ₘ” or “Cₙ-Cₘ” indicates a range which includes the endpoints, wherein n and m are integers and indicate the number of carbons. Examples include C₁₄, C₁-C₄, C₁₋₆, C₁-C₆, and the like.

“Alkyl” refers to a branched or straight hydrocarbon chain of one to eight carbon atoms, such as methyl, ethyl, n-propyl, isopropyl, n-butyl, isobutyl, sec-butyl, t-butyl, pentyl, hexyl, and heptyl. (C₁-C₆)alkyl is preferred. The term “Cₙ₋ₘ alkyl” or (Cₙ-Cₘ) alkyl, refers to an alkyl group having n to m carbon atoms. When optionally substituted, one or more hydrogen atoms of the alkyl group (e.g., from 1 to 4, from 1 to 2, or 1) may be replaced with a moiety as described below under “Optional Substitution.” In some aspects, the alkyl group is unsubstituted or not optionally substituted.

“Alkylene” refers to an optionally substituted bivalent saturated aliphatic radical having from 1 to 10 carbon atoms, 1 to 8 carbon atoms, 1 to 6 carbon atoms, 1 to 4 carbon atoms, or 1 to 2 carbon atoms. When optionally substituted, one or more hydrogen atoms of the alkylene group (e.g., from 1 to 4, from 1 to 2, or 1) may be replaced with a moiety as described below under “Optional Substitution.” In some aspects, the alkylene group is unsubstituted or not optionally substituted. The term “Cn-m alkylene” refers to an alkylene group having n to m carbon atoms. Examples of alkylene groups include, but are not limited to, methylene, ethan-1,2-diyl, propan-1,3-diyl, propan-1,2-diyl, butan-1,4-diyl, butan-1,3-diyl, butan-1,2-diyl, 2-methyl-propan-1,3-diyl, and the like.

The term “alkenyl” refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” or (Cₙ-Cₘ) alkenyl refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl, and the like.

The term “alkynyl” refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” or (Cₙ-Cₘ) alkynyl refers to an alkynyl group having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

“Alkoxy” refers to a moiety of the formula —OR′, wherein R′ is an (C₁-C₆)alkyl moiety as defined herein. The term “Cₙ₋ₘ alkoxy” or (Cₙ-Cₘ) alkoxy refers to an alkoxy group, the alkyl group of which has n to m carbons. Examples of alkoxy moieties include, but are not limited to, methoxy, ethoxy, isopropoxy, and the like.

An alkoxy group can be unsubstituted or optionally substituted. When optionally substituted, one or more hydrogen atoms of the alkoxy group (e.g., from 1 to 4, from 1 to 2, or 1) may be replaced with a moiety as described below under “Optional Substitution,” with the proviso that no hydrogen atom alpha to the ether oxygen is replaced by a hydroxy, amino, or thio group. In some aspects, the alkoxy group is unsubstituted or not optionally substituted.

“Alkoxycarbonyl” refers to a group —C(O)—R′ wherein R′ is (C₁-C₆)alkoxy as defined herein.

The term “amino” refers to a group of formula —NH₂.

The term “carbamyl” refers to a group of formula —C(O)NH₂.

The term “carbonyl”, employed alone or in combination with other terms, refers to a —C(═O)— group, which also may be written as C(O).

The term “cyano” or “nitrile” refers to a group of formula —C—N, which also may be written as —CN or CN.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “sulfide” refers to a sulfur atom as a divalent substituent, forming a thiocarbonyl group (C═S) when attached to carbon.

The term “heteroatom” used herein is meant to include boron, phosphorus, sulfur, oxygen, and nitrogen.

The term “haloalkyl” as used herein refers to an alkyl group in which one or more of the hydrogen atoms has been replaced by a halogen atom. The term “Cₙ₋ₘ haloalkyl” or (Cₙ-Cₘ) haloalkyl refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F5, CHF₂, CCl₃, CHCl₂, C₂Cl₅, and the like. In some embodiments, the haloalkyl group is a fluoroalkyl group.

The term “haloalkoxy,” employed alone or in combination with other terms, refers to a group of formula —O-haloalkyl, wherein the haloalkyl group is as defined above. The term “Cₙ₋ₘ haloalkoxy” or (Cₙ-Cₘ) haloalkoxy refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

“Aryl” means a monovalent six- to fourteen-membered, mono- or bi-carbocyclic ring (e.g., having two fused rings), wherein the monocyclic ring is aromatic and at least one of the rings in the bicyclic ring is aromatic. The term “Cₙ₋ₘ aryl” or “(Cₙ-Cₘ) aryl” refers to an aryl group having from n to m ring carbon atoms. In some embodiments, aryl groups have from 6 to about 10 carbon atoms. In some embodiments aryl groups have 6 carbon atoms. In some embodiments aryl groups have 10 carbon atoms. Unless stated otherwise, the valency of the group may be located on any atom of any ring within the radical, valency rules permitting. Representative examples include phenyl, naphthyl, and indanyl, and the like.

An aryl group can be unsubstituted or optionally substituted. When optionally substituted, one or more hydrogen atoms of the aryl group (e.g., from 1 to 5, from 1 to 2, or 1) may be replaced with a moiety as described below under “Optional Substitution.” In some aspects, the alkoxy group is unsubstituted or not optionally substituted.

“Arylene” means a divalent six- to fourteen-membered, mono- or bi-carbocyclic ring, wherein the monocyclic ring is aromatic and at least one of the rings in the bicyclic ring is aromatic. Representative examples include phenylene, naphthylene, and indanylene, and the like.

“Cycloalkyl” refers to a non-aromatic hydrocarbon ring system (monocyclic, bicyclic, or polycyclic), including cyclized alkyl and alkenyl groups. The term “Cₙ₋ₘ cycloalkyl” or “(Cₙ-Cₘ) cycloalkyl” refers to a cycloalkyl that has n to m ring member carbon atoms. Cycloalkyl groups can include mono- or polycyclic (e.g., having 2, 3, or 4 fused rings) groups and spirocycles. Cycloalkyl groups can have 3, 4, 5, 6, 7, 8, 9, 10, 11, 12, 13, or 14 ring-forming carbons (C₃₋₁₄). In some embodiments, the cycloalkyl group has 3 to 14 members, 3 to 10 members, 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl, or cyclohexyl. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl. In some embodiments, cycloalkyl includes a single saturated carbocyclic ring of three to eight ring carbons, such as cyclopropyl, cyclobutyl, cyclopentyl, and cyclohexyl. Cycloalkyl may optionally be substituted with one or more substituents, such as one, two, or three substituents. In some embodiments, the cycloalkyl substituent is selected from the group consisting of (C₁-C₆)alkyl, hydroxy, (C₁-C₆)alkoxy, halo(C₁-C₆)alkyl, halo(C₁-C₆)alkoxy, halo, amino, mono- and di(C₁-C₆)alkylamino, hetero(C₁-C₆)alkyl, acyl, aryl, and heteroaryl.

A cycloalkyl group can be unsubstituted or optionally substituted. When optionally substituted, one or more hydrogen atoms of the cycloalkyl group (e.g., from 1 to 4, from 1 to 2, or 1) may be replaced with a moiety as described below under “Optional Substitution.” In some aspects, a substituted cycloalkyl group can incorporate an exo- or endocyclic alkene (e.g., cyclohex-2-en-1-yl). In some aspects, a cycloalkyl group is unsubstituted or not optionally substituted.

“Cycloalkyloxycarbonyl” means a group —C(O)—OR′ wherein R′ is (C₃-C₆)cycloalkyl as defined herein.

“Phenyloxycarbonyl” refers to a group —C(O)—Ophenyl.

“Heteroaryl” means a monocyclic, fused bicyclic, or fused tricyclic, monovalent radical of 5 to 14 ring atoms containing one or more, preferably one, two, three, or four ring heteroatoms independently selected from —O—, —S(O)ₙ— (n is 0, 1, or 2), —N—, and —N(R′)—, and the remaining ring atoms being carbon, wherein the ring comprising a monocyclic radical is aromatic and wherein at least one of the fused rings comprising a bicyclic or tricyclic radical is aromatic. One or two ring carbon atoms of any nonaromatic rings comprising a bicyclic or tricyclic radical may be replaced by a —C(O)—, —C(S)—, or —C(═NH)— group. R′ is hydrogen, alkyl, hydroxy, alkoxy, acyl, or alkylsulfonyl. Unless stated otherwise, the valency may be located on any atom of any ring of the heteroaryl group, valency rules permitting. In particular, when the point of valency is located on the nitrogen, an additional nitrogen substituent is not present. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, thiazolyl, isothiazolyl, thiadiazolyl, benzothiazolyl, benzothienyl, and the derivatives thereof, and N-oxide or a protected derivative thereof.

A five-membered heteroaryl ring is a heteroaryl group having five ring atoms wherein one or more (e.g., 1, 2, 3, or 4) ring atoms are independently selected from N, O, and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered heteroaryl ring is a heteroaryl group having six ring atoms wherein one or more (e.g., 1, 2, 3, or 4) ring atoms are independently selected from N, O, and S. Exemplary six-membered ring heteroaryls are pyridyl, pyrazinyl, pyrimidinyl, triazinyl, and pyridazinyl.

“Heteroarylene” means a monocyclic, fused bicyclic, or fused tricyclic, divalent radical of 5 to 14 ring atoms containing one or more, preferably one, two, three, or four ring heteroatoms independently selected from —O—, —S(O)ₙ— (n is 0, 1, or 2), —N—, and —N(R¹⁹)—, and the remaining ring atoms being carbon, wherein the ring comprising a monocyclic radical is aromatic and wherein at least one of the fused rings comprising a bicyclic or tricyclic radical is aromatic. One or two ring carbon atoms of any nonaromatic rings comprising a bicyclic or tricyclic radical may be replaced by a —C(O)—, —C(S)—, or —C(═NH)— group. R¹⁹ is hydrogen, alkyl, or alkenyl. Unless stated otherwise, the valencies may be located on any atom of any ring of the heteroarylene group, valency rules permitting. In particular, when the point of valency is located on the nitrogen, an additional nitrogen substituent is not present. More specifically, the term heteroaryl includes, but is not limited to, thien-diyl, benzo[d]isoxazol-diyl, benzo[d]isothiazol-diyl, 1H-indazol-diyl (optionally substituted at the N1 position with R¹⁹), benzo[d]oxazol-diyl, benzo[d]thiazol-diyl, 1H-benzo[d]imidazol-diyl (optionally substituted at the N1 position with R¹⁹), 1H-benzo[d][1,2,3]triazol-diyl (optionally substituted at the N1 position with R¹⁹) imidazo[1,2-a]pyridin-diyl, cinnolin-diyl, quinolin-diyl, pyridin-diyl, 1-oxido-pyridin-diyl, [1,2,4]triazolo[4,3-a]pyridin-diyl, and 2,3-dihydroimidazo[1,2-a]pyridin-diyl, and the like.

As used herein, “heterocycloalkyl” or “heterocyclo” refer to a non-aromatic ring or ring system, which may optionally contain one or more alkenylene groups as part of the ring structure, which has at least one heteroatom ring member independently selected from boron, nitrogen, sulfur, oxygen, and phosphorus, and which has 4-14 ring members, 4-10 ring members, 4-7 ring members, or 4-6 ring members. Included within the term “heterocycloalkyl” are monocyclic 4-, 5-, 6-, and 7-membered heterocycloalkyl groups. Heterocycloalkyl groups can include mono- or bicyclic or polycyclic (e.g., having two or three fused or bridged rings) ring systems or spirorcycles. In some embodiments, the heterocycloalkyl group is a monocyclic group having 1, 2, or 3 heteroatoms independently selected from nitrogen, sulfur, and oxygen. Ring-forming carbon atoms and heteroatoms of a heterocycloalkyl group can be optionally oxidized to form an oxo or sulfido group or other oxidized linkage (e.g., C(O), S(O), C(S), S(O)₂, N-oxide, and the like.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, and the like. A heterocycloalkyl group containing a fused aromatic ring can be attached through any ring-forming atom, including a ring-forming atom of the fused aromatic ring. Examples of heterocycloalkyl groups include azetidinyl, azepanyl, dihydrobenzofuranyl, dihydrofuranyl, dihydropyranyl, morpholino, 3-oxa-9-azaspiro[5.5]undecanyl, 1-oxa-8-azaspiro[4.5]decanyl, piperidinyl, piperazinyl, oxopiperazinyl, pyranyl, pyrrolidinyl, quinuclidinyl, tetrahydrofuranyl, tetrahydropyranyl, 1,2,3,4-tetrahydroquinolinyl, tropanyl, 4,5,6,7-tetrahydrothiazolo[5,4-c]pyridinyl, and thiomorpholino.

“Heterocycloalkyl” or “heterocyclo,” can be unsubstituted or optionally substituted. When optionally substituted, one or more hydrogen atoms of the group (e.g., from 1 to 4, from 1 to 2, or 1) may be replaced with a moiety independently selected from fluoro, hydroxy, alkoxy, amino, alkylamino, acylamino, thio, and alkylthio. In some aspects, a substituted heterocycyl group can incorporate an exo- or endocyclic alkene (e.g., cyclohex-2-en-1-yl). In some aspects, the heterocycyl group is unsubstituted or not optionally substituted.

Optional Substitution

A group is optionally substituted herein unless expressly provided otherwise. The term “optionally substituted” refers to being substituted or unsubstituted. In certain embodiments, alkyl, alkenyl, alkynyl, carbocycloalkyl, heterocyclyoalkyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” “substituted” or “unsubstituted” cyclyoalkyl, “substituted” or “unsubstituted” heterocycloalkyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen (halo), —CN, —NO₂, —N₃, —SO₂H, —SO₃H, —OH, —OR^{a}, —ON(R^{bb})₂, —N(R^{bb})₂, —N(R^{bb})₃+X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR′, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂—OP(═O)(NR^{bb})₂, —NR^{bb}P(═O)(OR^{cc})₂, —NR^{bb}P(═O)(NR^{bb})₂, —OP(R^{cc})₂, —OP(R^{cc})₃, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, (C₃-C₁₀) carbocycloalkyl, 3-14 membered heterocycloalkyl, (C₆-C₁₄) aryl, and 5-14 membered heteroaryl, wherein each alkyl, alkenyl, alkynyl, cycloalkyl, heterocycloalkyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, —NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from (C₁-C₁₀) alkyl, (C₁-C₁₀) perhaloalkyl, (C₂-C₁₀) alkenyl, (C₂-C₁₀) alkynyl, (C₃-C₁₀) cycloalkyl, 3-14 membered heterocycloalkyl, (C₆-C₁₄) aryl, and 5-14 membered heteroaryl, or two R^{aa} groups are joined to form a 3-14 membered heterocycloalkyl or 5-14 membered heteroaryl ring, wherein each alkyl, alkenyl, alkynyl, cycloalkyl, heterocycloalkyl, aryl, and heteroaryl is independently substituted with 0, 1, 2, 3, 4, or 5 R^{dd} groups;
each instance of R^{bb} is, independently, selected from hydrogen, (C₁-C₁₀) perhaloalkyl, (C₂-C₁₀) alkenyl, (C₂-C₁₀) alkynyl, (C₃-C₁₀) cycloalkyl, C₆₋₁₄ aryl, and 5-14 membered heteroaryl, or two R^{bb} groups are joined to form a 3-14 membered heterocycloalkyl or 5-14 membered heteroaryl ring, wherein each alkyl, alkenyl, alkynyl, cycloalkyl, heterocycloalkyl, aryl, and heteroaryl is independently substituted with 0, 1, 2, 3, 4, or 5 R^{dd} groups;
each instance of R^{cc} is, independently, selected from hydrogen, (C₁-C₁₀) alkyl, (C₁-C₁₀) perhaloalkyl, (C₂-C₁₀) alkenyl, (C₂-C₁₀) alkynyl, (C₃-C₁₀) cycloalkyl, 3-14 membered heterocycloalkyl, (C₆-C₁₄) aryl, and 5-14 membered heteroaryl, or two R^{cc} groups are joined to form a 3-14 membered heterocycloalkyl or 5-14 membered heteroaryl ring, wherein each alkyl, alkenyl, alkynyl, cycloalkyl, heterocycloalkyl, aryl, and heteroaryl is independently substituted with 0, 1, 2, 3, 4, or 5 R^{dd} groups;
each instance of R^{dd} is, independently, selected from halogen, —CN, —NO₂, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{cc}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{E})₂, —C(═O)SR^{ee}, —C(═S)SR^{ee}, —SC(═S)SR^{ee}, —P(═O)₂R^{ee} —P(═O)(R^{ee})₂—OP(═O)(R^{ee})₂, —OP(═O)(OR^{ee})₂, (C₁-C₁₀) alkyl, (C₁-C₁₀) perhaloalkyl, (C₂-C₁₀) alkenyl, (C₂-C₁₀) alkynyl, (C₃-C₁₀) cycloalkyl, 3-10 membered heterocycloalkyl, (C₆-C₁₀) aryl, 5-10 membered heteroaryl, wherein each alkyl, alkenyl, alkynyl, cycloalkyl, heterocycloalkyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from (C₁-C₆) alkyl, (C₁-C₆) perhaloalkyl, (C₂-C₆) alkenyl, (C₂-C₆) alkynyl, (C₃-C₁₀) cycloalkyl, (C₆-C₁₀) aryl, 3-10 membered heterocycloalkyl, and 3-10 membered heteroaryl, wherein each alkyl, alkenyl, alkynyl, heteroalkyl, heteroalkenyl, heteroalkynyl, cycloalkyl, heterocycloalkyl, aryl, and heteroaryl is independently substituted with 0, 1, 2, 3, 4, or 5 R^{gg} groups;
each instance of R^{ff} is, independently, selected from hydrogen, (C₁-C₆) alkyl, (C₁-C₆) perhaloalkyl, (C₂-C₆) alkenyl, (C₂-C₆) alkynyl, (C₃-C₁₀) cycloalkyl, (C₆-C₁₀) aryl, and 5-10 membered heteroaryl, or two R groups are joined to form a 3-10 membered heterocycloalkyl or 5-10 membered heteroaryl ring, wherein each alkyl, alkenyl, alkynyl, cycloalkyl, heterocycloalkyl, aryl, and heteroaryl is independently substituted with 0, 1, 2, 3, 4, or 5 R^{gg} groups; and
each instance of R^{gg} is, independently, halogen, —CN, —NO₂, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃X⁻, —NH(C₁₋₆ alkyl)₂⁺X⁻, —NH₂(C₁₋₆ alkyl)⁺X⁻, —NH₃X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, (C₁-C₆) alkyl, (C₁-C₆) perhaloalkyl, (C₂-C₆) alkenyl, (C₂-C₆) alkynyl, (C₃-C₁₀) cycloalkyl, (C₆-C₁₀) aryl, 3-10 membered heterocycloalkyl, 5-10 membered heteroaryl; or two geminal R^{gg} substituents can be joined to form ═O or ═S; wherein X⁻ is a counterion.

As noted previously, nitrogen atoms can be substituted or unsubstituted as valency permits and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{c})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, (C₁-C₁₀) alkyl, (C₁-C₁₀) perhaloalkyl, (C₂-C₁₀) alkenyl, (C₂-C₁₀) alkynyl, (C₃-C₁₀) cycloalkyl, 3-14 membered heterocycloalkyl, (C₆-C1₄) aryl, and 5-14 membered heteroaryl, or two R^{cc} groups attached to an N atom are joined to form a 3-14 membered heterocycloalkyl or 5-14 membered heteroaryl ring, wherein each alkyl, alkenyl, alkynyl, cycloalkyl, heterocycloalkyl, aryl, and heteroaryl is independently substituted with 0, 1, 2, 3, 4, or 5 R^{dd} groups, and wherein R^{a}, R^{bb}, R^{cc} and R^{dd} are as defined above.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, (C₁-C₁₀) alkyl (e.g., aralkyl, heteroaralkyl), (C₂-C₁₀) alkenyl, (C₂-C₁₀) alkynyl, (C₃-C₁₀) cycloalkyl, 3-14 membered heterocycloalkyl, (C₆-C₁₄) aryl, and 5-14 membered heteroaryl groups, wherein each alkyl, alkenyl, alkynyl, cycloalkyl, heterocycloalkyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{a}, —N(R^{bb})₂, —C(═O)SR^{aa}, —C(═O)R′, —CO₂R^{a}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{a}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo), —OR^{aa} (when the O atom is attached to a carbonyl group, wherein R^{LG} is as defined herein), —O(C═O)R^{LG}, or —O(SO)₂R^{LG} (e.g., tosyl, mesyl, besyl), wherein R^{LG} is optionally substituted alkyl, optionally substituted aryl, or optionally substituted heteroaryl. In certain embodiments, the leaving group is a halogen.

The terms for which definitions are given above are specifically exemplified in the Examples.

“Yield” for each of the reactions described herein is expressed as a percentage of the theoretical yield.

“Patient” for the purposes of the present invention includes humans and any other animals, particularly mammals, and other organisms. Thus the methods are applicable to both human therapy and veterinary applications. In a preferred embodiment the patient is a mammal, and in a most preferred embodiment the patient is human. Examples of the preferred mammals include mice, rats, other rodents, rabbits, dogs, cats, swine, cattle, sheep, horses, and primates.

“Kinase-dependent diseases or conditions” refer to pathologic conditions that depend on the activity of one or more kinases. Kinases either directly or indirectly participate in the signal transduction pathways of a variety of cellular activities including proliferation, adhesion, migration, differentiation, and invasion. Diseases associated with kinase activities include tumor growth, the pathologic neovascularization that supports solid tumor growth, and associated with other diseases where excessive local vascularization is involved such as ocular diseases (diabetic retinopathy, age-related macular degeneration, and the like) and inflammation (psoriasis, rheumatoid arthritis, and the like).

“Therapeutically effective amount” is an amount of a compound of the invention that, when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

“Cancer” refers to cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Head and neck: squamous cell carcinomas of the head and neck, laryngeal and hypopharyngeal cancer, nasal cavity and paranasal sinus cancer, nasopharyngeal cancer, salivary gland cancer, oral and orppharyngeal cancer; Lung: bronchogenic carcinoma (squamous cell, undifferentiated small cell, undifferentiated large cell, adenocarcinoma, non-small cell lung cancer), alveolar (bronchiolar) carcinoma, bronchial adenoma, sarcoma, lymphoma, chondromatous hamartoma, mesothelioma; Colon: colorectal cancer, adenocarcinoma, gastrointestinal stromal tumors, lymphoma, carcinoids, Turcot Syndrome; Gastrointestinal: gastric cancer, gastroesophageal junction adenocarcinoma, esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Breast: metastatic breast cancer, ductal carcinoma in situ, invasive ductal carcinoma, tubular carcinoma, medullary carcinoma, mucinous carcinoma, lobular carcinoma in situ, triple negative breast cancer; Genitourinary tract: kidney (adenocarcinoma, Wilm's tumor [nephroblastoma], lymphoma, leukemia, renal cell carcinoma), bladder and urethra (squamous cell carcinoma, transitional cell carcinoma, adenocarcinoma, urothelial carcinoma), prostate (adenocarcinoma, sarcoma, castrate resistant prostate cancer), testis (seminoma, teratoma, embryonal carcinoma, teratocarcinoma, choriocarcinoma, sarcoma, interstitial cell carcinoma, fibroma, fibroadenoma, adenomatoid tumors, lipoma), clear cell carcinoma, papillary carcinoma; Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma, and giant cell tumors; Thyroid: medullary thyroid cancer, differentiated thyroid cancer, papillary thyroid cancer, follicular thyroid cancer, hurthle cell cancer, and anaplastic thyroid cancer; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial cancer), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

The term compound as used herein is meant to include all stereoisomers, geometric isomers, tautomers, and isotopes of the structures depicted. The term is also meant to refer to compounds of the inventions, regardless of how they are prepared, e.g., synthetically, through biological process (e.g., metabolism or enzyme conversion), or a combination thereof.

Compounds of the invention can also include all isotopes of atoms occurring in the intermediates or final compounds. Isotopes include those atoms having the same atomic number but different mass numbers. For example, isotopes of hydrogen include tritium and deuterium.

Any one of the process steps or sequences disclosed and/or claimed herein can be performed under an inert gas atmosphere, more particularly under argon or nitrogen. In addition, the methods of the present invention may be carried out as semi-continuous or continuous processes, more preferably as continuous processes.

Moreover, many of the process steps and sequences that are described herein can be telescoped.

In general, the nomenclature used in this Application is based on naming conventions adopted by the International Union of Pure and Applied Chemistry (IUPAC). Chemical structures shown herein were prepared using CHEMDRAW®. Any open valency appearing on a carbon, oxygen, or nitrogen atom in the structures herein indicates the presence of a hydrogen atom.

### Embodiments of the Invention

In one aspect, the invention includes a compound of Formula I′:

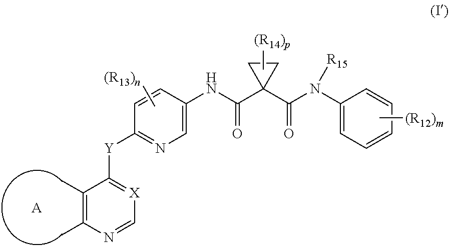

or a pharmaceutically acceptable salt thereof, wherein:
X is N or CH;
Y is selected from O, S, SO, SO₂, NH, and —N(C₁₋₆ alkyl)-;
(i) ring A is

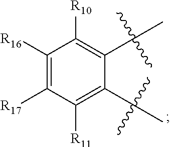

R₁₆ is selected from the group consisting of (C₂-C₆) alkenyl; (C₂-C₆) alkynyl; (C₆-C₁₀) aryl; (C₃-C₁₀) cycloalkyl; 5-14 membered heteroaryl; 4-14 membered heterocycloalkyl; —CN; —NHOH, —C(O)R^{a}; —C(O)NR^{a}R^{a}; —C(O)NHOR^{a}; —C(O)OR^{a}; —C(O)NR^{a}S(O)₂R^{a}; —OC(O)NR^{a}R^{a}; C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —S(O)NR^{a}R^{a}; —S(O)₂NR^{a}C(O)R^{a}; —P(O)R^{a}R^{a}; —P(O)(OR^{a})(OR^{a}); —B(OH)₂; —B(OR^{a})₂; and S(O)₂NR^{a}R^{a}; and
R₁₇ is selected from —H; halo; (C₁-C₆) alkyl; (C₂-C₆) alkenyl; (C₂-C₆) alkynyl; (C₁-C₆) haloalkyl; (C₁-C₆)haloalkoxy; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; (4-14 membered heterocycloalkyl)-(C₁-C₄) alkylene-; —CN; —NO₂; —OR^{a}; —SR^{a}; —NHOR^{a}; —C(O)R^{a}; —C(O)NR^{a}R^{a}; —C(O)NHOR^{a}; —C(O)OR^{a}; —C(O)NR^{a}S(O)₂R^{a}; —OC(O)R^{a}; —OC(O)NR^{a}R^{a}; —NR^{a}W; —NR^{a}R^{a}; —NR^{a}C(O)R^{a}; —NR^{a}C(═NR^{a})R^{a}; —NR^{a}C(O)OR^{a}; —NR^{a}C(O)NR^{a}R^{a}; —C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —NR^{a}C(═NR^{a})NR^{a}R^{a}; —NR^{a}S(O)R^{a}; —NR^{a}S(O)₂R^{a}; —NR^{a}S(O)₂NR^{a}R^{a}; —S(O)R^{a}; —S(O)NR^{a}R^{a}; —S(O)₂R^{a}; —S(O)₂NR^{a}C(O)R^{a}; —P(O)R^{a}R^{a}; —P(O)(OR^{a})(OR^{a}); —B(OH)₂; —B(OR^{a})₂; and —S(O)₂NR^{a}R^{a}; wherein the (C₁-C₆) alkyl; (C₂-C₆) alkenyl; (C₂-C₆) alkynyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R₁₆ or R₁₇ are each optionally substituted with 1, 2, 3, 4, or 5 independently selected R^{b} substituents, provided when R₁₆ or R₁₇ is 5-membered heteroaryl or 5-7 membered heterocycloalkyl, then the 5-membered heteroaryl or 5-7 membered heterocycloalkyl does not connect to the fused phenyl ring moiety through a ring nitrogen atom; or
R₁₆ is selected from —H; halo; (C₁-C₆) alkyl; (C₂-C₆) alkenyl; (C₂-C₆) alkynyl; (C₁-C₆) haloalkyl; (C₁-C₆)haloalkoxy; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; (4-14 membered heterocycloalkyl)-(C₁-C₄) alkylene-; —CN; —NO₂; —OR^{a}; —SR^{a}; —NHOR^{a}; —C(O)R^{a}; —C(O)NR^{a}R^{a}; —C(O)NHOR^{a}; —C(O)OR^{a}; —C(O)NR^{a}S(O)₂R^{a}; —OC(O)R^{a}; —OC(O)NR^{a}R^{a}; —NR^{a}W; —NR^{a}R^{a}; —NR^{a}C(O)R^{a}; —NR^{a}C(═NR^{a})R^{a}; —NR^{a}C(O)OR^{a}; —NR^{a}C(O)NR^{a}R^{a}; —C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —NR^{a}C(═NR^{a})NR^{a}R^{a}; —NR^{a}S(O)R^{a}; —NR^{a}S(O)₂R^{a}; —NR^{a}S(O)₂NR^{a}R^{a}; —S(O)R^{a}; —S(O)NR^{a}R^{a}; —S(O)₂R^{a}; —S(O)₂NR^{a}C(O)R^{a}; —P(O)R^{a}R^{a}; —P(O)(OR^{a})(OR^{a}); —B(OH)₂; —B(OR^{a})₂; and —S(O)₂NR^{a}R^{a}; wherein the (C₁-C₆) alkyl; (C₂-C₆) alkenyl; (C₂-C₆) alkynyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R₁₆ is each optionally substituted with 1, 2, 3, 4, or 5 independently selected R^{b} substituents; and
R₁₇ is selected from the group consisting of (C₂-C₆) alkenyl; (C₂-C₆) alkynyl; —CN; —NHOH, —C(O)R^{a}; —C(O)NR^{a}R^{a}; —C(O)NHOR^{a}; —C(O)OR^{a}; —C(O)NR^{a}S(O)₂R^{a}; —OC(O)NR^{a}R^{a}; C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —S(O)NR^{a}R^{a}; —S(O)₂NR^{a}C(O)R^{a}; —P(O)R^{a}R^{a}; —P(O)(OR^{a})(OR^{a}); —B(OH)₂; —B(OR^{a})₂; and S(O)₂NR^{a}R^{a}, provided when R₁₆ or R₁₇ is 5-membered heteroaryl or 5-7 membered heterocycloalkyl, then the 5-membered heteroaryl or 5-7 membered heterocycloalkyl does not connect to the fused phenyl ring moiety through a ring nitrogen atom; or
R₁₆ and R₁₇ taken together with the atoms to which they are attached form a fused C₃₋₇ cycloalkyl ring or a fused 4- to 10-membered heterocycloalkyl ring; wherein the fused C₃₋₇ cycloalkyl ring and fused 4- to 10-membered heterocycloalkyl ring are each optionally substituted with 1, 2, or 3 independently selected R^{b} substituents; or
(ii) ring A is

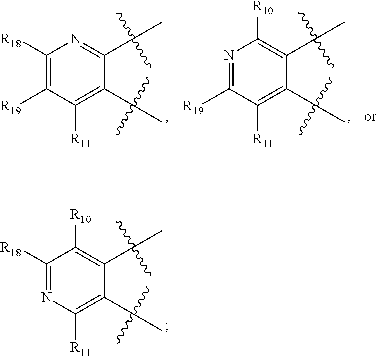

R₁₈ and R₁₉ are each independently selected from —H; halo; (C₁-C₆) alkyl; (C₂-C₆) alkenyl; (C₂-C₆) alkynyl; (C₁-C₆) haloalkyl; (C₁-C₆)haloalkoxy; (C₆-C₁₀) aryl; (C₃-C₁₀) cycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; (4-14 membered heterocycloalkyl)-(C₁-C₄) alkylene-; —CN; —NO₂; —OR^{a}; —SR^{a}; —NHOR^{a}; —C(O)R^{a}; —C(O)NR^{a}R^{a}; —C(O)NHOR^{a}; —C(O)OR^{a}; —C(O)NR^{a}S(O)₂R^{a}; —OC(O)R^{a}; —OC(O)NR^{a}R^{a}; —NHR^{a}; —NR^{a}R^{a}; —NR^{a}C(O)R^{a}; —NR^{a}C(═NR^{a})R^{a}; —NR^{a}C(O)OR^{a}; —NR^{a}C(O)NR^{a}R^{a}; —C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —NR^{a}C(═NR^{a})NR^{a}R^{a}; —NR^{a}S(O)R^{a}; —NR^{a}S(O)₂R^{a}; —NR^{a}S(O)₂NR^{a}R^{a}; —S(O)R^{a}; —S(O)NR^{a}R^{a}; —S(O)₂R^{a}; —S(O)₂NR^{a}C(O)R^{a}; —P(O)R^{a}R^{a}; —P(O)(OR^{a})(OR^{a}); —B(OH)₂; —B(OR^{a})₂; and —S(O)₂NR^{a}R^{a}; wherein the (C₁-C₆) alkyl; (C₂-C₆) alkenyl; (C₂-C₆) alkynyl; (C₆-C₁₀) aryl; (C₃-C₁₀) cycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R₁₈ or R₁₉ are each optionally substituted with 1, 2, 3, 4, or 5 independently selected R^{b} substituents; or
R₁₈ and R₁₉ taken together with the atoms to which they are attached form a fused C₃₋₇ cycloalkyl ring or a fused 4- to 10-membered heterocycloalkyl ring; wherein the fused C₃₋₇ cycloalkyl ring and fused 4- to 10-membered heterocycloalkyl ring are each optionally substituted with 1, 2, or 3 independently selected R^{b} substituents;
R₁₀ and R₁₁ are each independently selected from the group consisting of —H; halo; (C₁-C₆) alkyl; (C₁-C₆) haloalkyl; (C₁-C₆)haloalkoxy; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; (4-14 membered heterocycloalkyl)-(C₁-C₄) alkylene-; —CN; —NO₂; —OR^{a}; —SR^{a}; —NHOR^{a}; —C(O)R^{a}; —C(O)NR^{a}R^{a}; —C(O)OR^{a}; —C(O)NR^{a}S(O)₂R^{a}; —OC(O)R^{a}; —OC(O)NR^{a}R^{a}; —NR^{a}W; —NR^{a}R^{a}; —NR^{a}C(O)R^{a}; —NR^{a}C(═NR^{a})R^{a}; —NR^{a}C(O)OR^{a}; —NR^{a}C(O)NR^{a}R^{a}; —C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —NR^{a}C(═NR^{a})NR^{a}R^{a}; —NR^{a}S(O)R^{a}; —NR^{a}S(O)₂R^{a}; —NR^{a}S(O)₂NR^{a}R^{a}; —S(O)R^{a}; —S(O)NR^{a}R^{a}; —S(O)₂R^{a}; —S(O)₂NR^{a}C(O)R^{a}; —P(O)R^{a}R^{a}; —P(O)(OR^{a})(OR^{a}); —B(OH)₂; —B(OR^{a})₂; and S(O)₂NR^{a}R^{a}; wherein the (C₁-C₆) alkyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R₁ or R₂ are each optionally substituted with 1, 2, 3, 4, or 5 independently selected R^{b} substituents;
each R₁₃ is independently selected from the group consisting of —H; halo; —OH; —CN; optionally substituted (C₁-C₆) alkyl; (C₁-C₆) alkoxy; (C₁-C₆) haloalkoxy; —NH₂; —NH(C₁-C₆)alkyl; —N(C₁-C₆ alkyl)₂; and (C₃-C₆) cycloalkyl; wherein the (C₁-C₆) alkoxy; —NH(C₁-C₆)alkyl; —N(C₁-C₆ alkyl)₂; and (C₃-C₆) cycloalkyl of R₃ are each optionally substituted with 1, 2, or 3 independently selected R^{g} substituents;
each R₁₄ is independently selected from the group consisting of halo; —OH; —NH₂; —CN; (C₁-C₆) alkyl; (C₁-C₆) alkoxy; (C₁-C₆) haloalkyl; (C₁-C₆) haloalkoxy; —COOH; —NH(C₁-C₆)alkyl; —N(C₁-C₆ alkyl)₂; phenyl; phenyl-(C₁-C₂) alkylene; (C₃-C₆) cycloalkyl; (C₃-C₆) cycloalkyl-(C₁-C₄) alkylene-; 4- to 6-membered heterocycloalkyl; (4- to 6-membered heterocycloalkyl)-(C₁-C₄) alkylene-; 5- to 6-membered heteroaryl; (5- to 6-membered heteroaryl)-(C₁-C₄) alkylene-; and —OR^{e}; wherein the (C₁-C₆) alkyl; phenyl; phenyl-(C₁-C₂) alkylene; (C₃-C₆) cycloalkyl; (C₃-C₆) cycloalkyl-(C₁-C₄) alkylene-; 4- to 6-membered heterocycloalkyl; (4- to 6-membered heterocycloalkyl)-(C₁-C₄) alkylene-; 5- to 6-membered heteroaryl; and (5- to 6-membered heteroaryl)-(C₁-C₄) alkylene- of R₁₄ are each optionally substituted with 1, 2, or 3 independently selected R^{g} substituents;
R₁₅ is H;
each R₁₂ is independently selected from the group consisting of —H; halo; —OH; —COOR^{e}; —CONR^{e}R^{e}; —CN; —NH₂; —NH((C₁-C₆) alkyl); —N((C₁-C₆) alkyl)₂; (C₁-C₆) alkyl; (C₁-C₆) alkoxy; (C₁-C₆) haloalkyl; (C₁-C₆) haloalkoxy; —CONR^{a}R^{a}; —NR^{a}COR^{a}; —NR^{a}CONR^{a}R^{a}; —SO₂R; —NR^{a}S(O)₂R^{a}; —NR^{a}S(O)₂NR^{a}R^{a}; (C₃-C₆) cycloalkyl; 4- to 6-membered heterocycloalkyl; phenyl; 5- or 6-membered heteroaryl; (C₃-C₆) cycloalkyl-(C₁-C₄) alkylene-; (4- to 6-membered heterocycloalkyl)-(C₁-C₄) alkylene-; phenyl-(C₁-C₂) alkylene; and (5- or 6-membered heteroaryl)-(C₁-C₄) alkylene-; wherein the (C₁-C₆) alkyl; (C₃-C₆) cycloalkyl; 4- to 6-membered heterocycloalkyl; phenyl; 5- or 6-membered heteroaryl; (C₃-C₆) cycloalkyl-(C₁-C₄) alkylene-; (4-to 6-membered heterocycloalkyl)-(C₁-C₄) alkylene-; phenyl-(C₁-C₂) alkylene; and (5- or 6-membered heteroaryl)-(C₁-C₄) alkylene- of R₁₂ are each optionally substituted with 1, 2, or 3 independently selected R^{f} substituents;
each R^{a} is independently selected from the group consisting of —H; —CN; (C₁-C₆) alkyl; (C₁-C₆) haloalkyl; (C₂-C₆) alkenyl; (C₂-C₆) alkynyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene-; wherein the (C₁-C₆) alkyl; (C₁-C₆) haloalkyl; (C₂-C₆) alkenyl; (C₂-C₆) alkynyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R^{a} are each optionally substituted with 1, 2, 3, 4, or 5 independently selected R^{d} substituents;
each R^{b} is independently selected from the group consisting of halo; (C₁-C₆) alkyl; (C₂-C₆) alkenyl; (C₂-C₆) alkynyl; (C₁-C₆) haloalkyl; (C₁-C₆) haloalkoxy; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; (4-10 membered heterocycloalkyl)-(C₁-C₄) alkylene-; —CN; —OH; —NH₂; —NO₂; —NHOR^{c}; —OR^{c}; —SR^{c}; —C(O)R^{c}; —C(O)NR^{c}R^{c}; —C(O)OR^{c}; —C(O)NR^{c}S(O)₂R^{c}; —OC(O)R^{c}; —OC(O)NR^{c}R^{c}; —C(═NOH)R^{c}; —C(═NOH)NR^{c}; —C(═NCN)NR^{c}R^{c}; —NR^{c}C(═NCN)NR^{c}R^{c}; —C(═NR^{c})NR^{c}R^{c}; —NR^{c}C(═NR^{c})NR^{c}R^{c}; —NHR^{c}; —NR^{c}R^{c}; —NR^{c}C(O)R^{c}; —NR^{c}C(═NR^{c})R^{c}; —NR^{c}C(O)OR^{c}; —NR^{c}C(O)NR^{c}R^{c}; —NR^{c}S(O)R^{c}; —NR^{c}S(O)₂R^{c}; —NR^{c}S(O)₂NR^{c}R^{c}; —S(O)R^{c}; —S(O)NR^{c}R^{c}; —S(O)₂R^{c}; —S(O)₂NR^{c}C(O)R^{c}; —Si(R^{c})₃; —P(O)R^{c}R^{c}; —P(O)(OR^{c})(OR^{c}); —B(OH)₂; —B(OR^{c})₂; and —S(O)₂NR^{c}R^{c}; wherein the (C₁-C₆) alkyl; (C₁-C₆) haloalkyl; (C₁-C₆) haloalkoxy; (C₂-C₆) alkenyl; (C₂-C₆) alkynyl; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalky-(C₁-C₄) alkylene-; (5-10 membered heteroaryl)-(C₁-C₄) alkylene-; and (4-10 membered heterocycloalkyl)-(C₁-C₄) alkylene- of R^{b} are each further optionally substituted with 1, 2, or 3 independently selected R^{d} substituents;
each R^{c} is independently selected from the group consisting of —H; (C₁-C₆) alkyl; (C₁-C₆) haloalkyl; (C₂-C₆) alkenyl; (C₂-C₆) alkynyl; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; and (4-10 membered heterocycloalkyl)-(C₁-C₄) alkylene-; wherein the (C₁-C₆) alkyl; (C₂-C₆) alkenyl; (C₂-C₆) alkynyl; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; and (4-10 membered heterocycloalkyl)-(C₁-C₄) alkylene- of R^{c} are each optionally substituted with 1, 2, 3, 4, or 5 independently selected R^{f} substituents;
each R^{d} is independently selected from the group consisting of (C₁-C₆) alkyl; (C₁-C₆) haloalkyl; halo; (C₆-C₁₀) aryl; 5-10 membered heteroaryl; (C₃-C₁₀) cycloalkyl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; (4-10 membered heterocycloalkyl)-(C₁-C₄) alkylene-; —CN; —NH₂; —NHOR^{c}; —OR^{c}; —SR^{c}; —C(O)R^{c}; —C(O)NR^{e}R^{e}; —C(O)OR^{c}; —OC(O)R^{c}; —OC(O)NR^{c}R^{e}; —NHR^{c}; —NR^{c}R^{c}; —NR^{c}C(O)R^{c}; —NR^{e}C(O)NR^{c}R^{c}; —NR^{c}C(O)OR^{e}; —C(═NR^{c})NR^{c}R^{c}; —NR^{e}C(═NR^{c})NR^{c}R^{c}; —NR^{c}C(═NOH)NR^{c}R^{c}; —NR^{c}C(═NCN)NR^{c}R^{e}; —S(O)R^{e}; —S(O)NR^{c}R^{c}; —S(O)₂R^{e}; —NR^{c}S(O)₂R^{e}; —NR^{c}S(O)₂NR^{e}R^{e}; and —S(O)₂NR^{e}R^{e}; wherein the (C₁-C₆) alkyl; (C₁-C₆) haloalkyl; (C₆-C₁₀) aryl; 5-10 membered heteroaryl; (C₃-C₁₀) cycloalkyl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; and (4-10 membered heterocycloalkyl)-(C₁-C₄) alkylene- of R^{d} are each optionally substituted with 1, 2, or 3 independently selected R^{f} substituents;
each R^{c} is independently selected from the group consisting of —H; (C₁-C₆) alkyl; (C₃-C₆) cycloalkyl; (C₃-C₆) cycloalkyl-(C₁-C₄) alkylene-; (C₆-C₁₀) aryl; (C₆-C₁₀) aryl-(C₁-C₄) alkylene-; 5- or 6-membered heteroaryl; (5- or 6-membered heteroaryl)-(C₁-C4) alkylene-; 4-7-membered heterocycloalkyl; (4-7-membered heterocycloalkyl)-(C₁-C₄) alkylene-; (C₁-C₆) haloalkyl; (C₁-C₆) haloalkoxy; (C₂-C₄) alkenyl; and (C₂-C₄) alkynyl; wherein the (C₁-C₄) alkyl; (C₃-C₆) cycloalkyl; (C₆-C₁₀) aryl; 5 or 6-membered heteroaryl; 4-7-membered heterocycloalkyl; (C₆-C₁₀) aryl-(C₁-C₄) alkylene-; (5- or 6-membered heteroaryl)-(C₁-C₄) alkylene-; (4-7-membered heterocycloalkyl)-(C₁-C₄) alkylene-; (C₂-C₄) alkenyl; and (C₂-C₄) alkynyl of R^{c} are each optionally substituted with 1, 2, or 3 R^{f} substituents;
or any two R^{a} substituents together with the nitrogen atom to which they are attached form 4-5-, 6-, 7-, 8-, 9-, or 10-membered heterocycloalkyl, each of which is optionally substituted with 1, 2, or 3 independently selected R^{f} substituents;
or any two R^{c} substituents together with the nitrogen atom to which they are attached form 4-5-, 6-, 7-, 8-, 9-, or 10-membered heterocycloalkyl, each of which is optionally substituted with 1, 2, or 3 independently selected R^{f} substituents;
or any two R^{e} substituents together with the nitrogen atom to which they are attached form 4-5-, 6-, 7-, 8-, 9-, or 10-membered heterocycloalkyl, each of which is optionally substituted with 1, 2, or 3 independently selected R^{f} substituents;
each R^{f} is independently selected from the group consisting of halo; —OH; —CN; —COOH; —NH₂; —NH—(C₁-C₆) alkyl; —N((C₁-C₆) alky)₂; (C₁-C₆) alkyl; (C₁-C₆) alkoxy; (C₁-C₆) alkylthio; (C₁-C₆) haloalkyl; (C₁-C₆)haloalkoxy; phenyl; 5-6 membered heteroaryl; 4-6 membered heterocycloalkyl; and (C₃-C₆) cycloalkyl; wherein the (C₁-C₆) alkyl; phenyl; (C₃-C₆) cycloalkyl; 4-6 membered heterocycloalkyl; and 5-6 membered heteroaryl of R^{f} are each optionally substituted with 1, 2, or 3 substituents selected from halo; —OH; —CN; —COOH; —NH₂; (C₁-C₄) alkyl; (C₁-C₄) alkoxy; (C₁-C₄) haloalkyl; (C₁-C₄) haloalkoxy; phenyl; (C₃-C₁₀) cycloalkyl; 5-6 membered heteroaryl; and 4-6 membered heterocycloalkyl;
each R^{g} is independently selected from the group consisting of halo; —OH; —CN; —COOH; —COO—(C₁-C₄) alkyl; —NH₂; —NH—(C₁-C₆) alkyl; —N((C₁-C₆) alky)₂; (C₁-C₆) alkyl; (C₁-C₆) alkoxy; (C₁-C₆) alkylthio; (C₁-C₆)haloalkyl; (C₁-C₆)haloalkoxy; phenyl; 5-6 membered heteroaryl; 4-6 membered heterocycloalkyl; and (C₃-C₆) cycloalkyl;
the ring nitrogen atom on the quinoline moiety in Formula A is optionally oxidized;
the subscript n is an integer of 1, 2, 3, or 4;
the subscript m is an integer of 1, 2, 3, 4, or 5; and
the subscript p is an integer of 0, 1, 2, 3, or 4.

In some embodiments of this aspect, when X is C—H, Ring A must be

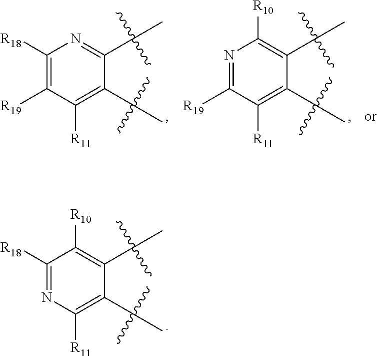

In one embodiment of this aspect, R₁₆ is selected from —H, halo, (C₁-C₆) alkyl, (C₆-C₁₀) aryl, (C₃-C₁₀) cycloalkyl, 5-14 membered heteroaryl, 4-14 membered heterocycloalkyl, —CN, —NO₂, —OR^{a}, —C(O)R^{a}, —C(O)NR^{a}R^{a}, —C(O)OR^{a}, —NHR^{a}, —NR^{a}R^{a}, and —NR^{a}C(O)R^{a}, wherein the (C₁-C₆) alkyl, (C₆-C₁₀) aryl, (C₃-C₁₀) cycloalkyl, 5-14 membered heteroaryl, or 4-14 membered heterocycloalkyl, of R₁₆ is each optionally substituted with 1, 2, 3, 4, or 5 independently selected R^{b} substituents.

In another embodiment of this aspect, R₁₆ is selected from —H, halo, (C₁-C₆) alkyl, phenyl, (C₃-C₁₀) cycloalkyl, 5-14 membered heteroaryl, 4-14 membered heterocycloalkyl, —CN, —NO₂, (C₁-C₆) alkoxy, —COOH, —COO((C₁-C₆) alkyl), —C(O)((C₁-C₆) alkyl), —C(O)NH₂, —C(O)NH—(C₁-C₆) alkyl, —C(O)N((C₁-C₆) alky)₂, —NH₂, —NH—(C₁-C₆) alkyl, or —N((C₁-C₆) alky)₂, wherein each R₁₆ is optionally and independently substituted with halo, —OH, —CN, —COOH, —NH₂, —NH—(C₁-C₆) alkyl, —N((C₁-C₆) alky)₂, (C₁-C₆) alkyl, (C₁-C₆) alkoxy, (C₁-C₆) alkylthio, (C₁-C₆) haloalkyl, (C₁-C₆)haloalkoxy, phenyl, 5-6 membered heteroaryl, 4-6 membered heterocycloalkyl, and (C₃-C₆) cycloalkyl, wherein the (C₁-C₆) alkyl, phenyl, (C₃-C₆) cycloalkyl, 4-6 membered heterocycloalkyl, and 5-6 membered heteroaryl substituents are each optionally further substituted with halo, —OH, —CN, —COOH, —NH₂, (C₁-C₄) alkyl, (C₁-C₄) alkoxy, (C₁-C₄) haloalkyl, (C₁-C₄) haloalkoxy, phenyl, (C₃-C₁₀) cycloalkyl, 5-6 membered heteroaryl, and 4-6 membered heterocycloalkyl.

In another embodiment of this aspect, R₁₆ is selected from —H, halo, (C₁-C₆) alkyl, 5-14 membered heteroaryl, 4-14 membered heterocycloalkyl, —CN, (C₁-C₆) alkoxy, —COOH, —COO((C₁-C₆) alkyl), —C(O)((C₁-C₆) alkyl), —C(O)NH₂, —C(O)NH—(C₁-C₆) alkyl, and —C(O)N((C₁-C₆) alky)₂, wherein each R₁₆ is optionally and independently substituted with (C₁-C₆) alkyl, (C₁-C₆) alkoxy, phenyl, 5-6 membered heteroaryl, wherein the (C₁-C₆) alkyl, phenyl, and 5-6 membered heteroaryl substituents are each optionally further substituted with halo, (C₁-C₄) alkyl, and (C₁-C₄) alkoxy.

In a further embodiment, R₁₆ is selected from H, 5 or 6 membered heteroaryl, —COOH, —COO((C₁-C₆) alkyl), —C(O)NH₂, and —C(O)NH—(C₁-C₆) alkyl, wherein each R₁₆ is optionally and independently substituted with (C₁-C₆) alkyl, which is optionally further substituted with (C₁-C₄) alkoxy.

In still a further embodiment, R₁₆ is selected from (C₁-C₄) alkoxy-(C₁-C₆) alkyl-(5 or 6 membered heteroaryl), (C₁-C₆) alkyl-(5 or 6 membered heteroaryl), 5 or 6 membered heteroaryl), —COOH, —COO((C₁-C₆) alkyl), —C(O)NH₂, and —C(O)NH—(C₁-C₆) alkyl.

In yet a further embodiment, R₁₆ is selected from (C₁-C₄) alkoxy-(C₁-C₆) alkyl-(5 or 6 membered heteroaryl), (C₁-C₆) alkyl-(5 or 6 membered heteroaryl), 5 or 6 membered heteroaryl), —COOH, —COO((C₁-C₆) alkyl), —C(O)NH₂, and —C(O)NH—(C₁-C₆) alkyl.

In still a further embodiment, R₁₆ is selected from —COOH, —COOCH₃, C(O)NH₂, C(O)NHCH₃, C(O)NHCH₂CH₃,

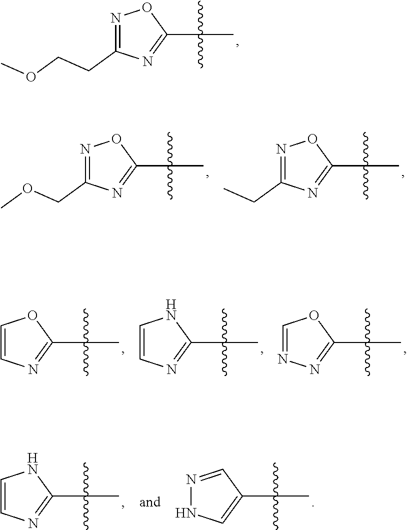

In one embodiment, R₁₇ is selected from —H, halo, (C₁-C₆) alkyl, (C₆-C₁₀) aryl, (C₃-C₁₀) cycloalkyl, (5-14 membered heteroaryl), (4-14 membered heterocycloalkyl), —CN, —NO₂, —OR^{a}, C(O)R^{a}, —C(O)NR^{a}R^{a}, —C(O)OR^{a}, —NHR^{a}, and —NR^{a}R^{a}, wherein the (C₁-C₆) alkyl, (C₆-C₁₀) aryl, (C₃-C₁₀) cycloalkyl, (5-14 membered heteroaryl), or (4-14 membered heterocycloalkyl), of R₁₇ is each optionally substituted with 1, 2, 3, 4, or 5 independently selected R^{b} substituents, provided when R₁₆ or R₁₇ is 5-membered heteroaryl or 5-7 membered heterocycloalkyl, then the 5-membered heteroaryl or 5-7 membered heterocycloalkyl does not connect to the fused phenyl ring moiety through a ring nitrogen atom.

In another embodiment, R₁₇ is selected from —H, halo, (C₁-C₆) alkyl, 5-14 membered heteroaryl, 4-14 membered heterocycloalkyl, —CN, (C₁-C₆) alkoxy, —COOH, —COO((C₁-C₆) alkyl), —C(O)((C₁-C₆) alkyl), —C(O)NH₂, —C(O)NH—(C₁-C₆) alkyl, and —C(O)N((C₁-C₆) alky)₂, wherein each R₁₆ is optionally and independently substituted with (C₁-C₆) alkyl, (C₁-C₆) alkoxy, phenyl, 5-6 membered heteroaryl, OH, CN, NO₂, or halo, wherein the (C₁-C₆) alkyl, phenyl, and 5-6 membered heteroaryl substituents are each optionally further substituted with halo, (C₁-C₄) alkyl, and (C₁-C₄) alkoxy.

In a further embodiment, R₁₇ is selected from H, (C₁-C₆) alkoxy, —COOH, —COO((C₁-C₆) alkyl), —C(O)NH₂, and —C(O)NH—(C₁-C₆) alkyl, wherein each R₁₆ is optionally and independently substituted with (C₁-C₆) alkoxy, (C₁-C₆) alkyl, or OH, which is optionally further substituted with (C₁-C₄) alkoxy.

In a further embodiment, R₁₇ is selected from H, (C₁-C₆) alkoxy, or hydroxyl-(C₁-C₆) alkoxy. In yet a further embodiment, R₁₇ is selected from methoxy,

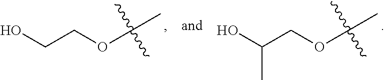

In one embodiment, R₁₆ and R₁₇ taken together with the atoms to which they are attached form a fused C₃₋₇ cycloalkyl ring or a fused 4- to 10-membered heterocycloalkyl ring.

In another embodiment of this aspect, R₁₈ and R₁₉ are each independently selected from —H, halo, (C₁-C₆) alkyl, (C₆-C₁₀) aryl, (C₃-C₁₀) cycloalkyl, —CN, —NO₂, —OR^{a}, —C(O)R^{a}, —C(O)NR^{a}R^{a}, —C(O)OR^{a}, —NHR^{a}, —NR^{a}R^{a}, —NR^{a}C(O)R^{a}, wherein the (C₁-C₆) alkyl, or (C₆-C₁₀) aryl, (C₃-C₁₀) cycloalkyl of R₁₈ or R₁₉ are each optionally substituted with 1, 2, 3, 4, or 5 independently selected R^{b} substituents.

In another embodiment, R₁₈ and R₁₉ are each independently selected from —H, OH, (C₁-C₆) alkyl, phenyl, (C₃-C₁₀) cycloalkyl, (C₁-C₆) alkoxy, —COOH, —COO((C₁-C₆) alkyl), —C(O)((C₁-C₆) alkyl), —C(O)NH₂, —C(O)NH—(C₁-C₆) alkyl, and —C(O)N((C₁-C₆) alky)₂, wherein each R₁₈ or R₁₉ is optionally and independently substituted with one or more (C₁-C₆) alkyl, (C₁-C₆) alkoxy, phenyl, 5-6 membered heteroaryl, 5-6 membered heterocycloalkyl, OH, CN, NO₂, —C(O)NH₂, —C(O)NH—(C₁-C₆) alkyl, and —C(O)N((C₁-C₆) alky)₂, —NH₂, —NH—(C₁-C₆) alkyl, or —N((C₁-C₆) alky)₂, wherein the (C₁-C₆) alkyl, phenyl, and 5-6 membered heteroaryl substituents are each optionally further substituted with halo, (C₁-C₄) alkyl, or (C₁-C₄) alkoxy.

In a further embodiment, R₁₈ and R₁₉ are each independently selected from OH, (C₁-C₆) alkoxy, (C₁-C₆) alkoxy-(C₁-C₆) alkoxy, 5 or 6 membered heterocycloalkyl-(C₁-C₆) alkoxy, (C₁-C₆) alkoxy-phenyl-(C₁-C₆) alkoxy, carbamoyl-(C₁-C₆) alkoxy, dialkylamino-(C₁-C₆) alkoxy, and dihydroxy-(C₁-C₆) alkoxy.

In still a further embodiment, R₁₈ and R₁₉ are each independently selected from OH, methoxy, 2-methoxyethoxy, 3-morpholinopropoxy, 2-morpholinoethoxy, methoxyphenylmethoxy, carbamoylmethoxy, 3-dimethylaminopropoxy, 2,3-dihydroxypropoxy.

In another embodiment, R₁₈ and R₁₉ taken together with the atoms to which they are attached form a fused C₃₋₇ cycloalkyl ring or a fused 4- to 10-membered heterocycloalkyl ring. In a further embodiment, R₁₈ and R₁₉ taken together with the atoms to which they are attached form a fused 5 to 6-membered heterocycloalkyl ring. In still a further embodiment, R₁₈ and R₁₉ taken together with the atoms to which they are attached form the moiety

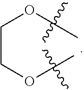 [SMILES: *OCCO*]

In one embodiment of this aspect, R₁₀ and R₁₁ are each independently selected from the group consisting of —H, halo, (C₁-C₆) alkyl, —CN, —NO₂, (C₁-C₆) alkoxy, phenyl, and 5-6 membered heteroaryl. In another embodiment, R₁₀ and R₁₁ are each independently selected from the group consisting of —H, halo, and (C₁-C₆) alkyl. In a further embodiment, R₁₀ and R₁₁ are each H.

In one embodiment of this aspect, each R₁₃ is independently selected from the group consisting of —H, halo, —OH, —CN, (C₁-C₆) alkyl, (C₁-C₆) haloalkyl, (C₁-C₆) alkoxy, —C(O)NH₂, —C(O)NH—(C₁-C₆) alkyl, —C(O)N((C₁-C₆) alky)₂, —NH₂, —NH(C₁-C₆)alkyl, and —N(C₁-C₆ alkyl)₂. In a further embodiment, each R₁₃ is independently selected from the group consisting of —H, halo, —CN, (C₁-C₆) alkyl, (C₁-C₆) alkoxy, —C(O)NH₂, and (C₁-C₆) haloalkyl. In still a further embodiment, each R₁₃ is independently selected from the group consisting of —H, F, Cl, Br, methyl, methoxy, —CN, trifluoromethyl, and —C(O)NH₂.

In one embodiment of this aspect, each R₁₄ is independently selected from the group consisting of H, halo, and (C₁-C₆) alkyl. In a further embodiment, each R₁₄ is H.

In one embodiment of this aspect, each R₁₂ is independently selected from the group consisting of —H, halo, —OH, (C₁-C₆) alkyl, (C₁-C₆) alkoxy, or (C₁-C₆) haloalkyl. In a further embodiment, each R₁₂ is independently selected from the group consisting of —H, halo, and (C₁-C₆) alkyl. In still a further embodiment, each R₁₂ is halo. In still a further embodiment, m is 1, and R₁₂ is F. In yet a further embodiment, m is 1, and R₁₂ is F in the para position of the phenyl ring to which it is attached.

In one embodiment of this aspect, each R^{a} is independently selected from the group consisting of —H, (C₁-C₆) alkyl, (C₁-C₆) alkoxy, (C₁-C₆) haloalkyl, (C₆-C₁₀) aryl, (C₃-C₁₀) cycloalkyl, 5-14 membered heteroaryl, and 4-14 membered heterocycloalkyl, wherein the (C₁-C₆) alkyl, (C₁-C₆) haloalkyl, (C₆-C₁₀) aryl, (C₃-C₁₀) cycloalkyl, 5-14 membered heteroaryl, 4-14 membered heterocycloalkyl, of R^{a} are each optionally substituted with 1, 2, 3, 4, or 5 independently selected R^{d} substituents.

In another embodiment, each R^{a} is independently selected from the group consisting of —H, (C₁-C₆) alkyl, (C₁-C₆) alkoxy-(C₁-C₆) alkyl, hydroxy-(C₁-C₆) alkyl, 5-6 membered heterocyclo-(C₁-C₆) alkyl, phenyl-(C₁-C₆) alkyl, (C₁-C₆) alkoxy-phenyl-(C₁-C₆) alkyl, amino-(C₁-C₆) alkyl, di(C₁-C₆) alkylamino-(C₁-C₆) alkyl, hydroxyl-(C₁-C₆) alkyl, dihydroxy-(C₁-C₆) alkyl, and carbamoyl-(C₁-C₆) alkyl.

In one embodiment of this aspect, each R^{b} is independently selected from the group consisting of halo, (C₁-C₆) alkyl, (C₆-C₁₀) aryl, (C₃-C₁₀) cycloalkyl, 5-10 membered heteroaryl, 4-10 membered heterocycloalkyl, —CN, —OH, —NH₂, —NO₂, —C(O)NH₂, —C(O)NH—(C₁-C₆) alkyl, —C(O)N((C₁-C₆) alky)₂, —NH₂, —NH(C₁-C₆)alkyl, and —N(C₁-C₆ alkyl)₂, wherein the (C₁-C₆) alkyl, (C₆-C₁₀) aryl, (C₃-C₁₀) cycloalkyl, 5-10 membered heteroaryl, and 4-10 membered heterocycloalkyl, of R^{b} are each further optionally substituted with 1, 2, or 3 independently selected R^{d} substituents.

In another embodiment, each R^{b} is independently selected from the group consisting of (C₁-C₆) alkyl, —C(O)NH₂, —C(O)NH—(C₁-C₆) alkyl, —C(O)N((C₁-C₆) alky)₂, —NH(C₁-C₆)alkyl, and —N(C₁-C₆ alkyl)₂, wherein the (C₁-C₆) alkyl, of R^{b} is further optionally substituted with 1, 2, or 3 independently selected R^{d} substituents. In a further embodiment, each R^{b} is (C₁-C₆) alkyl, optionally substituted with 1, 2, or 3 independently selected R^{d} substituents.

In one embodiment of this aspect, each R^{c} is (C₁-C₆) alkyl.

In one embodiment of this aspect, each R^{d} is independently selected from the group consisting of (C₁-C₆) alkyl, (C₆-C₁₀) aryl, 5-10 membered heteroaryl, (C₃-C₁₀) cycloalkyl, 4-10 membered heterocycloalkyl, —CN, —NH₂, —OR^{e}, —SR^{e}, —C(O)R^{e}, —C(O)NR^{e}R^{e}, —C(O)OR^{e}, —NHR^{e} and —NR^{e}R^{e}, wherein the (C₁-C₆) alkyl, (C₆-C₁₀) aryl, 5-10 membered heteroaryl, (C₃-C₁₀) cycloalkyl, and 4-10 membered heterocycloalkyl, of R^{d} are each optionally substituted with 1, 2, or 3 independently selected R^{f} substituents.

In another embodiment, each R^{d} is independently selected from the group consisting of (C₁-C₆) alkyl, phenyl, 5-6 membered heterocycloalkyl, (C₁-C₆) alkoxy, and carbamoyl, optionally substituted with 1, 2, or 3 independently selected R^{f} substituents.

In one embodiment of this aspect, each R^{e} is independently selected from the group consisting of —H and (C₁-C₆) alkyl.

In one embodiment of this aspect, each R^{f} is independently selected from the group consisting of halo, —OH, —CN, —COOH, —NH₂, —NH—(C₁-C₆) alkyl, —N((C₁-C₆) alky)₂, (C₁-C₆) alkyl, (C₁-C₆) alkoxy, phenyl, wherein the (C₁-C₆) alkyl, and phenyl, of R^{f} are each optionally substituted with 1, 2, or 3 substituents selected from halo, —OH, —CN, —COOH, —NH₂, (C₁-C₄) alkyl, (C₁-C₄) alkoxy, (C₁-C₄) haloalkyl, (C₁-C₄) haloalkoxy, phenyl, (C₃-C₁₀) cycloalkyl, 5-6 membered heteroaryl, and 4-6 membered heterocycloalkyl.

In another embodiment, each R^{f} is independently selected from the group consisting of halo, (C₁-C₆) alkyl, and (C₁-C₆) alkoxy. In a further embodiment, R^{f} is (C₁-C₆) alkyl.

In one embodiment of this aspect, each R^{g} is independently selected from the group consisting of halo, —OH, —CN, —COOH, —COO—(C₁-C₄) alkyl, —NH₂, —NH—(C₁-C₆) alkyl, (C₁-C₆) alkyl, (C₁-C₆) alkoxy, and (C₁-C₆) alkoxy-(C₁-C₆) alkyl. In a further embodiment, each R^{g} is independently selected from the group consisting of halo and (C₁-C₆) alkoxy-(C₁-C₆) alkyl.

In one embodiment, the subscript n is 1 or 2. In a further embodiment, the subscript n is 1. In another further embodiment, the subscript n is 2.

In one embodiment, the subscript m is 1.

In one embodiment, the subscript p is 0 or 1. In a further embodiment, the subscript p is 0. In another further embodiment, the subscript p is 1.

In one embodiment, the compound of Formula I′ is a compound of Formula I′a:

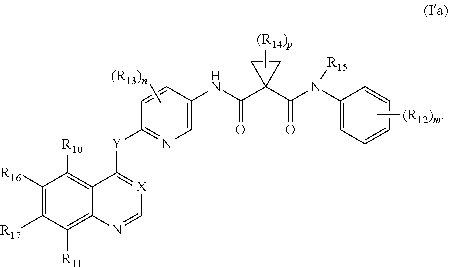

In one embodiment, the compound of Formula I′ is a compound of Formula I′b, I′c or I′d:

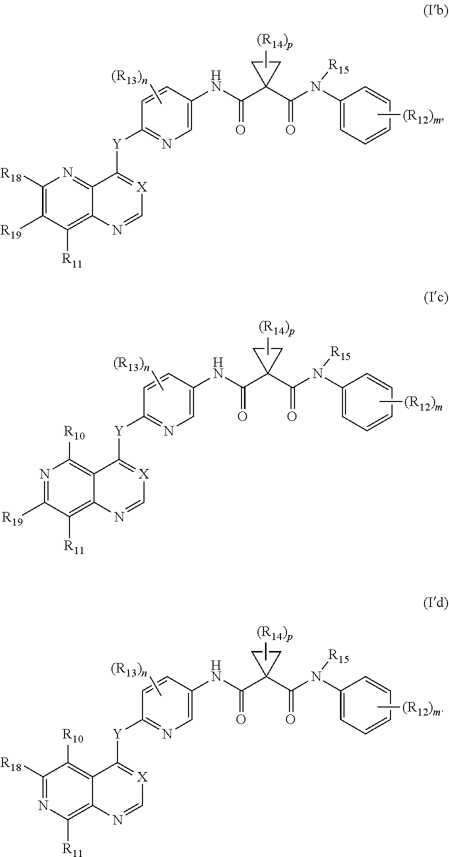

In one embodiment, the compound of Formula I′ is a compound of Formula (I′a-1):

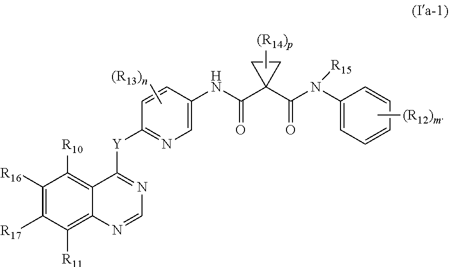

In one embodiment, the compound of Formula I′ is a compound of Formula (I′b-1):

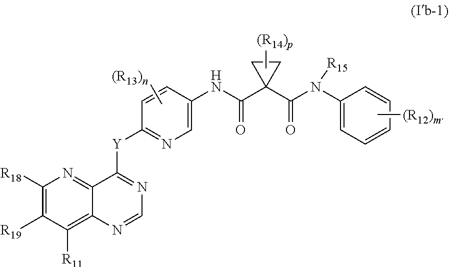

In one embodiment, the compound of Formula I′ is a compound of Formula (I′b-2):

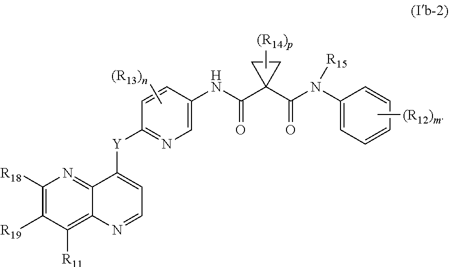

In one embodiment, the compound of Formula I′ is a compound of Formula (I′c-1):

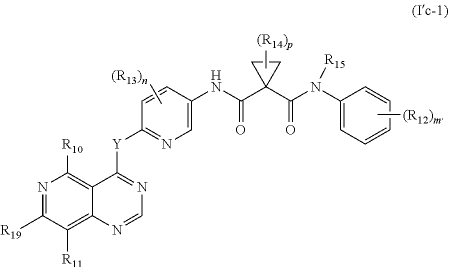

In one embodiment, the compound of Formula I′ is a compound of Formula (I′c-2):

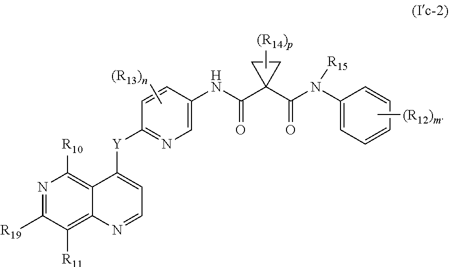

In one embodiment, the compound of Formula I′ is a compound of Formula (I′d-1):

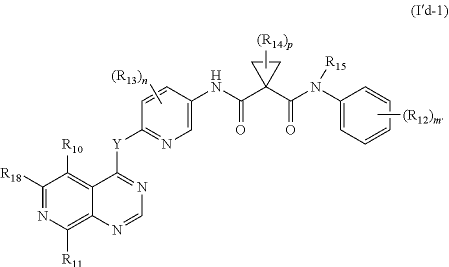

In one embodiment, the compound of Formula I′ is a compound of Formula (I′d-2):

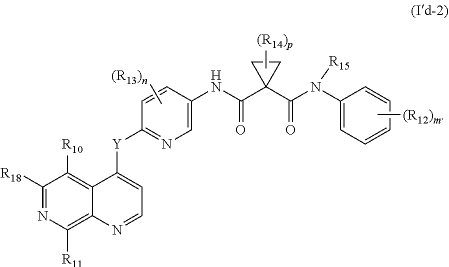

In one embodiment, R₁₆ is selected from —H, (C₁-C₆) alkyl, (C₂-C₆) alkenyl, (C₂-C₆) alkynyl, —C(═NO—(C₁-C₆) alkyl)R^{a}; halo, —CN, OR^{a}, —C(O)OR^{a}; —C(O)NR^{a}R^{a}, —C(O)NHOR^{a}, —S(O)₂NR^{a}R^{a}, phenyl, 5- to 6-membered heteroaryl, (C₃-C₆) cycloalkyl, and 4- to 6-membered heterocycloalkyl, wherein the (C₁-C₆) alkyl; (C₂-C₆) alkenyl, (C₂-C₆) alkynyl, phenyl, 5- to 6-membered heteroaryl, (C₃-C₆) cycloalkyl, and 4- to 6-membered heterocycloalkyl of R₁₆ are each optionally substituted with 1, 2, or 3 R^{g} substituents.

In one embodiment, R₁₇ selected from —H, (C₁-C₆) alkyl; (C₂-C₆) alkenyl, (C₂-C₆) alkynyl, —C(═NO—(C₁-C₆) alkyl)R^{a}; halo, —CN, OR^{a}, —C(O)OR^{a}; —C(O)NR^{a}R^{a}, —C(O)NHOR^{a}, —S(O)₂NR^{a}R^{a}, phenyl, 5- to 6-membered heteroaryl, (C₃-C₆) cycloalkyl, and 4- to 6-membered heterocycloalkyl, wherein the (C₁-C₆) alkyl, (C₂-C₆) alkenyl, (C₂-C₆) alkynyl, phenyl, 5- to 6-membered heteroaryl, (C₃-C₆) cycloalkyl, and 4- to 6-membered heterocycloalkyl of R₁₆ are each optionally substituted with 1, 2, or 3 R^{g} substituents.

In one embodiment, R₁₈ and R₁₉ are each independently selected from —H, (C₁-C₆) alkyl; (C₂-C₆) alkenyl, (C₂-C₆) alkynyl, —C(═NO—(C₁-C₆) alkyl)R^{a}; halo, —CN, OR^{a}, —C(O)OR^{a}; C(O)NR^{a}R^{a}, —C(O)NHOR^{a}, —S(O)₂NR^{a}R^{a}, phenyl, 5- to 6-membered heteroaryl, (C₃-C₆) cycloalkyl, and 4- to 6-membered heterocycloalkyl, wherein the (C₁-C₆) alkyl, (C₂-C₆) alkenyl, (C₂-C₆) alkynyl, phenyl, 5- to 6-membered heteroaryl, (C₃-C₆) cycloalkyl, and 4- to 6-membered heterocycloalkyl of R₁₆ are each optionally substituted with 1, 2, or 3 R^{b} substituents.

In another embodiment, R₁₆ is selected from H, halo, NH₂, NH(C₁₋₆ alkyl), N(C₁₋₆ alkyl), methoxy, methyl, CN, 3-morphlinopropoxy, 2-methoxyethoxy, (oxetan-3-yloxy)carbamoyl, cyclopropylcarbamoyl, carbamoyl, 2-(pyrrolidin-1-yl)ethylcarbamoyl, 1-(t-butoxycarbonylpyrrolidin-2-yl)methylcarbamoyl, 1-(pyrrolidin-2-yl)methylcarbamoyl, 2-methoxyethylamino; azetidin-1-yl; dimethylcarbamoyl, methylamino; 3-morpholinopropoxy; 2-methoxyethoxy; 2-hydroxyethoxy; propoxy; 2-hydroxypropoxy; methoxycarbonyl; carboxy; methylcarbamoyl; 2-oxazolyl; pyrazol-3-yl; pyrazol-4-yl; 4-isoxazolyl; 3,5-dimethylisoxazol-4-yl; 1-methyl-pyrazol-4-yl; 2-methyl-pyrazol-3-yl; 2-ethyl-pyrazol-3-yl; 2-(2-hydroxyethyl)-pyrazol-3-yl; 2-(2,2,2-trifluoroethyl)-pyrazol-3-yl; 2-(2-fluoroethyl)-pyrazol-3-yl; 2-(2,2-difluoroethyl)-pyrazol-3-yl; 2-trifluoromethyl-pyrazol-3-yl; 2-difluoromethyl-pyrazol-3-yl; 1-methyl-imidazol-4-yl; 1-methyl-imidazol-2-yl; 1H-imidazol-2-yl; (2-hydroxyethoxy)carbamoyl; (2,2-dihydroxyethoxy)carbamoyl; (oxetan-3-yloxy)carbamoyl; methoxycarbamoyl; 2-trimethylsilylethynyl; ethynyl; 1,3,4-oxadiazol-3-yl; 1H-1,2,3-triazol-5-yl; sulfamoyl; acetyl, ethyl carbamoyl, and —C(═NOCH₃)CH₃.

In another embodiment, R₁₈ and R₁₉ are each independently selected from H, halo, NH₂, NH(C₁₋₆ alkyl), N(C₁₋₆ alkyl), methoxy, methyl, CN, 3-morphlinopropoxy, 2-methoxyethoxy, (oxetan-3-yloxy)carbamoyl, cyclopropylcarbamoyl, carbamoyl, 2-(pyrrolidin-1-yl)ethylcarbamoyl, 1-(t-butoxycarbonylpyrrolidin-2-yl)methylcarbamoyl, 1-(pyrrolidin-2-yl)methylcarbamoyl, 2-methoxyethylamino; azetidin-1-yl; dimethylcarbamoyl, methylamino; 3-morpholinopropoxy; 2-methoxyethoxy; 2-hydroxyethoxy; propoxy; 2-hydroxypropoxy; methoxycarbonyl; carboxy; methylcarbamoyl; 2-oxazolyl; pyrazol-3-yl; pyrazol-4-yl; 4-isoxazolyl; 3,5-dimethylisoxazol-4-yl; 1-methyl-pyrazol-4-yl; 2-methyl-pyrazol-3-yl; 2-ethyl-pyrazol-3-yl; 2-(2-hydroxyethyl)-pyrazol-3-yl; 2-(2,2,2-trifluoroethyl)-pyrazol-3-yl; 2-(2-fluoroethyl)-pyrazol-3-yl; 2-(2,2-difluoroethyl)-pyrazol-3-yl; 2-trifluoromethyl-pyrazol-3-yl; 2-difluoromethyl-pyrazol-3-yl; 1-methyl-imidazol-4-yl; 1-methyl-imidazol-2-yl; 1H-imidazol-2-yl; (2-hydroxyethoxy)carbamoyl; (2,2-dihydroxyethoxy)carbamoyl; (oxetan-3-yloxy)carbamoyl; methoxycarbamoyl; 2-trimethylsilylethynyl; ethynyl; 1,3,4-oxadiazol-3-yl; 1H-1,2,3-triazol-5-yl; sulfamoyl; acetyl, —OH, 2-morpholinoethoxy, carbamoylmethyloxy, —OCH₂C(O)NH₂, 3-dimethylaminopropyloxy, 2,3-dihydroxypropoxy, and —C(═NOCH₃)CH₃.

In some embodiments:
1) R₁₆ is R^{a}NHC(O)— and R₁₇ is H or —OR^{a}; or
2) R₁₆ is 5- or 6-membered heteroaryl optionally substituted with 1, 2 or 3 independently selected R^{b} substituents and R₁₇ is H; or
3) R₁₆ is 5- or 6-membered heteroaryl optionally substituted with 1, 2 or 3 independently selected R^{b} substituents and R₁₇ is —OR^{a}; or
4) R₁₆ is H or —OR^{a} and R₁₇ is 5- or 6-membered heteroaryl optionally substituted with 1, 2 or 3 independently selected R^{b} substituents.

In some embodiments:
1) R₁₈ and R₁₉ are each independently H, halo, CN, R^{a}NHC(O)—, —OR^{a} or 5- or 6-membered heteroaryl optionally substituted with 1-3 independently selected R^{b} substituents;
2) R₁₈ is H and R₁₉ is —OR^{a}; or
3) R₁₉ is H and R₁₈ is —OR^{a}; or
4) R₁₈ and R₁₉ are each independently —OR^{a}; or
5) R₁₈ is 5- or 6-membered heteroaryl optionally substituted with 1-3 independently selected R^{b} substituents and R₁₉ is H or —OR^{a}; or
6) R₁₈ is H or —OR^{a} and R₁₉ is 5- or 6-membered heteroaryl optionally substituted with 1-3 independently selected R^{b} substituents; or
7) R₁₈ is R^{a}NHC(O)— and R₁₉ is H or —OR^{a}; or
8) R₁₉ is R^{a}NHC(O)— and R₁₈ is H or —OR.

In another embodiment, R₁₀ and R₁₁ are each H.

In one embodiment, R₁₃ is H, F, Cl, Br, CH₃, CH₃O, CN, —C(O)NH₂, or CF₃ and the subscript n is 1 or 2.

In one embodiment,

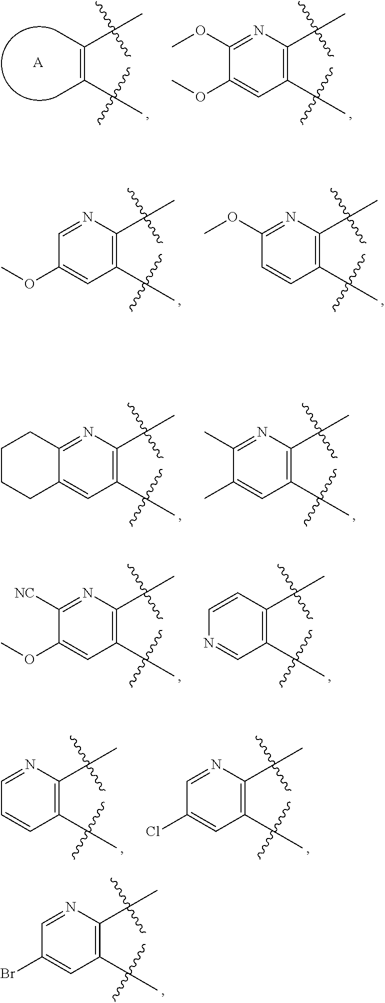

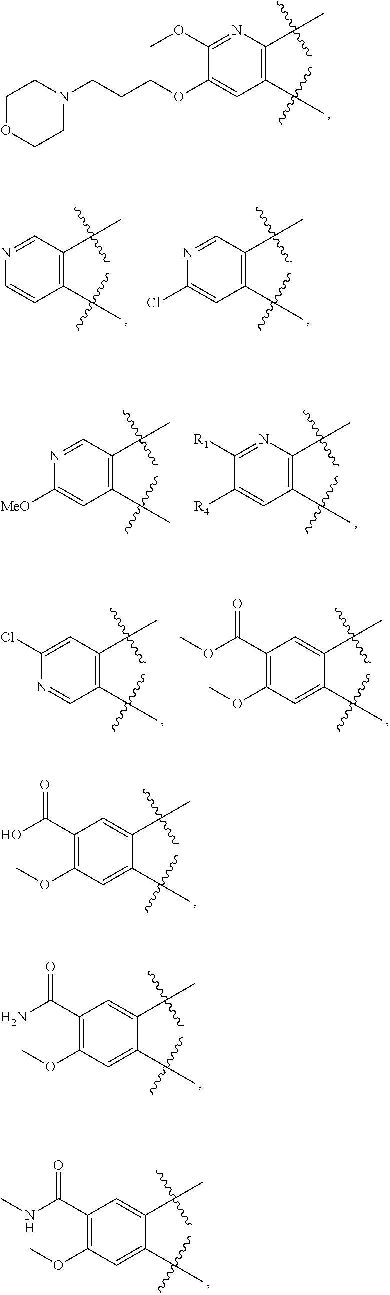

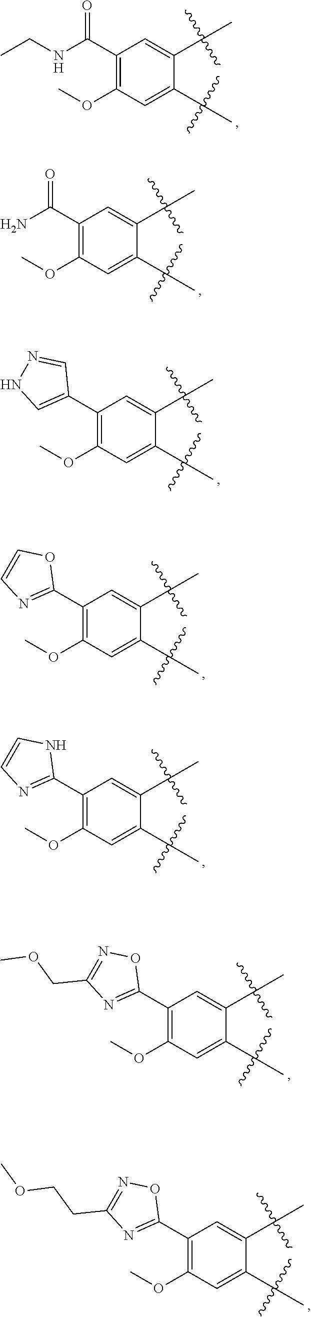

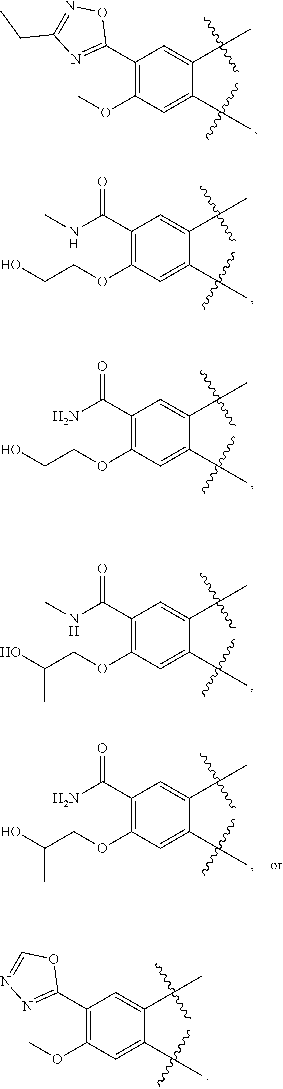 [SMILES: *c1cc(OC)c(-c2nc(CC)no2)cc1* ; *c1cc(OCCO)c(C(=O)NC)cc1* ; *c1cc(OCCO)c(C(N)=O)cc1* ; *c1cc(OCC(C)O)c(C(=O)NC)cc1* ; *c1cc(OCC(C)O)c(C(N)=O)cc1* ; *c1cc(OC)c(-c2nnco2)cc1*]

In one embodiment, the compound of Formula 1′, or a pharmaceutically acceptable salt thereof, is selected from the compounds listed in Table 1, or a pharmaceutically acceptable salt thereof.

TABLE 1
Compounds of Formula I′

| # | Structure | IUPAC Name |
|---|---|---|
| 10 | 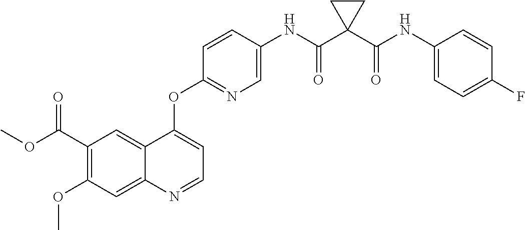 [SMILES: COC(=O)c1cc2c(Oc3ccc(NC(=O)C4(C(=O)Nc5ccc(F)cc5)CC4)cn3)ccnc2cc1OC] | methyl 4-[5-[[1-[(4- fluorophenyl)carbamoyl]cyclo- propanecarbonyl]amino]pyridin-2- yl]oxy-7-methoxyquinoline-6- carboxylate |
| 11 | 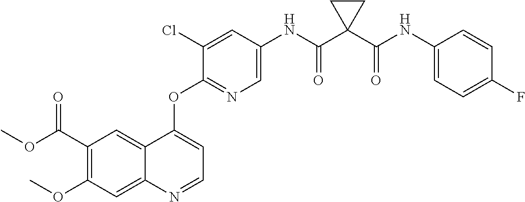 [SMILES: COC(=O)c1cc2c(Oc3ncc(NC(=O)C4(C(=O)Nc5ccc(F)cc5)CC4)cc3Cl)ccnc2cc1OC] | methyl 4-[3-chloro-5-[[1-[(4- fluorophenyl)carbamoyl]cyclo- propanecarbonyl]amino]pyridin-2- yl]oxy-7-methoxyquinoline-6- carboxylate |
| 13 | 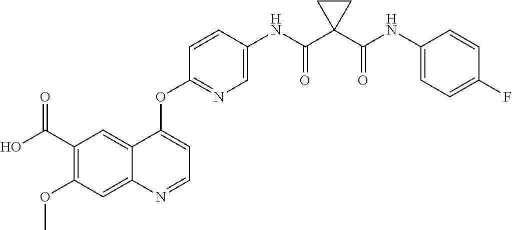 [SMILES: COc1cc2nccc(Oc3ccc(NC(=O)C4(C(=O)Nc5ccc(F)cc5)CC4)cn3)c2cc1C(=O)O] | 4-[5-[[l-[(4- fluorophenyl)carbamoyl]cyclo- propanecarbonyl]amino]pyridin-2- yl]oxy-7-methoxyquinoline-6- carboxylic acid |
| 14 | 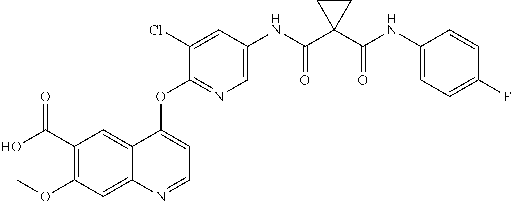 [SMILES: COc1cc2nccc(Oc3ncc(NC(=O)C4(C(=O)Nc5ccc(F)cc5)CC4)cc3Cl)c2cc1C(=O)O] | 4-[3-chloro-5-[[1-[(4- fluorophenyl)carbamoyl]cyclo- propanecarbonyl]amino]pyridin-2- yl]oxy-7-methoxyquinoline-6- carboxylic acid |
| 15 | 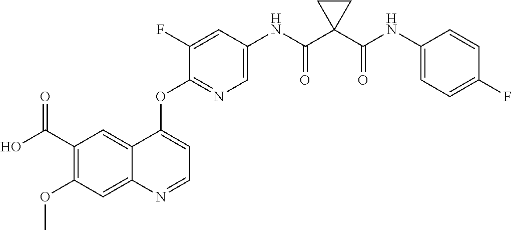 [SMILES: COc1cc2nccc(Oc3ncc(NC(=O)C4(C(=O)Nc5ccc(F)cc5)CC4)cc3F)c2cc1C(=O)O] | 4-[3-fluoro-5-[[1-[(4- fluorophenyl)carbamoyl]cyclo- propanecarbonyl]amino]pyridin-2- yl]oxy-7-methoxyquinoline-6- carboxylic acid |
| 16 | 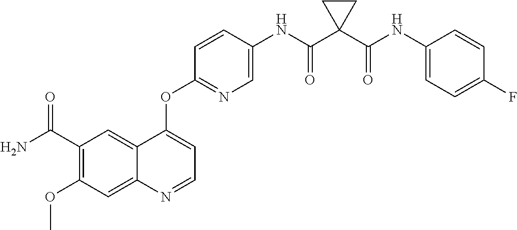 [SMILES: COc1cc2nccc(Oc3ccc(NC(=O)C4(C(=O)Nc5ccc(F)cc5)CC4)cn3)c2cc1C(N)=O] | 1-N′-[6-(6-carbamoyl-7- methoxyquinolin-4-yl)oxypyridin- 3-yl]-1-N-(4- fluorophenyl)cyclopropane-1,1- dicarboxamide |
| 17 | 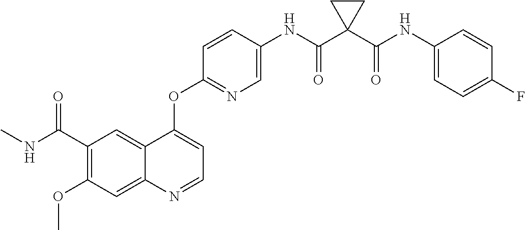 [SMILES: CNC(=O)c1cc2c(Oc3ccc(NC(=O)C4(C(=O)Nc5ccc(F)cc5)CC4)cn3)ccnc2cc1OC] | 1-N-(4-fluorophenyl)-1-N′-[6-[7- methoxy-6- (methylcarbamoyl)quinolin-4- yl]oxypyridin-3-yl]cyclopropane- 1,1-dicarboxamide |
| 18 | 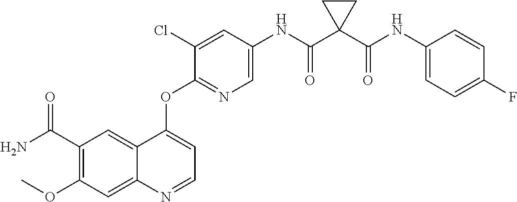 [SMILES: COc1cc2nccc(Oc3ncc(NC(=O)C4(C(=O)Nc5ccc(F)cc5)CC4)cc3Cl)c2cc1C(N)=O] | 1-N′-[6-(6-carbamoyl-7- methoxyquinolin-4-yl)oxy-5- chloropyridin-3-yl]-1-N-(4- fluorophenyl)cyclopropane-1,1- dicarboxamide |
| 19 | 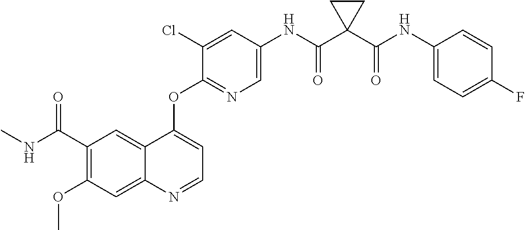 [SMILES: CNC(=O)c1cc2c(Oc3ncc(NC(=O)C4(C(=O)Nc5ccc(F)cc5)CC4)cc3Cl)ccnc2cc1OC] | l-N′-[5-chloro-6-[7-methoxy-6- (methylcarbamoyl)quinolin-4- yl]oxypyridin-3-yl]-1-N-(4- fluorophenyl)cyclopropane-1,1- dicarboxamide |
| 20 | 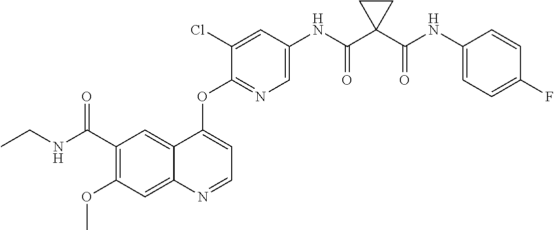 [SMILES: CCNC(=O)c1cc2c(Oc3ncc(NC(=O)C4(C(=O)Nc5ccc(F)cc5)CC4)cc3Cl)ccnc2cc1OC] | 1-N′-[5-chloro-6-[6- (ethylcarbamoyl)-7- methoxyquinolin-4-yl]oxypyridin- 3-yl]-1-N-(4- fluorophenyl)cyclopropane-1,1- dicarboxamide |
| 21 | 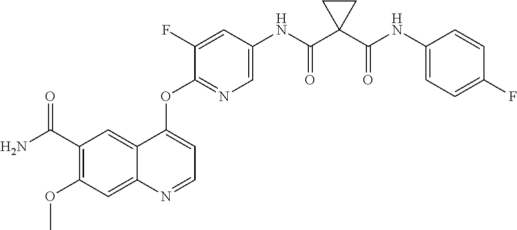 [SMILES: COc1cc2nccc(Oc3ncc(NC(=O)C4(C(=O)Nc5ccc(F)cc5)CC4)cc3F)c2cc1C(N)=O] | 1-N′-[6-(6-carbamoyl-7- methoxyquinolin-4-yl)oxy-5- fluoropyridin-3-yl]-l-N-(4- fluorophenyl)cyclopropane-1,1- dicarboxamide |
| 22 | 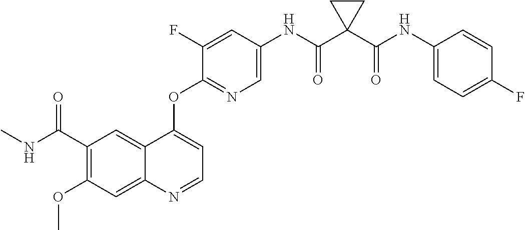 [SMILES: CNC(=O)c1cc2c(Oc3ncc(NC(=O)C4(C(=O)Nc5ccc(F)cc5)CC4)cc3F)ccnc2cc1OC] | 1-N′-[5-fluoro-6-[7-methoxy-6- (methylcarbamoyl)quinolin-4- yl]oxypyridin-3-yl]-1-N-(4- fluorophenyl)cyclopropane-1,1- dicarboxamide |
| 23 | 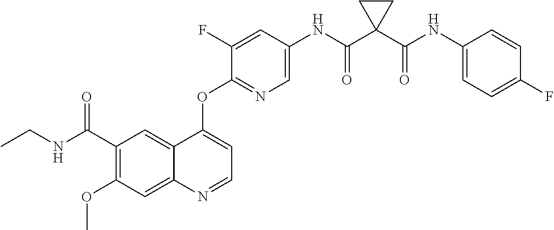 [SMILES: CCNC(=O)c1cc2c(Oc3ncc(NC(=O)C4(C(=O)Nc5ccc(F)cc5)CC4)cc3F)ccnc2cc1OC] | 1-N′-[6-[6-(ethylcarbamoyl)-7- methoxyquinolin-4-yl]oxy-5- fluoropyridin-3-yl]-1-N-(4- fluorophenyl)cyclopropane-1,1- dicarboxamide |
| 25 | 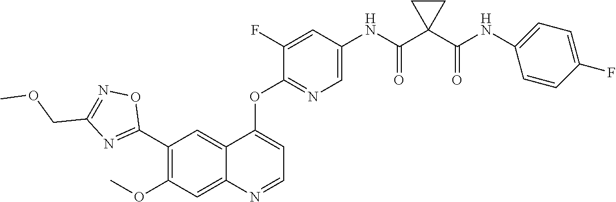 [SMILES: COCc1noc(-c2cc3c(Oc4ncc(NC(=O)C5(C(=O)Nc6ccc(F)cc6)CC5)cc4F)ccnc3cc2OC)n1] | l-N′-[5-fluoro-6-[7-methoxy-6-[3 (methoxymethyl)-1,2,4-oxadiazol- 5-yl]quinolin-4-yl]oxypyridin-3- yl]-1-N-(4- fluorophenyl)cyclopropane-1,1- dicarboxamide |
| 26 | 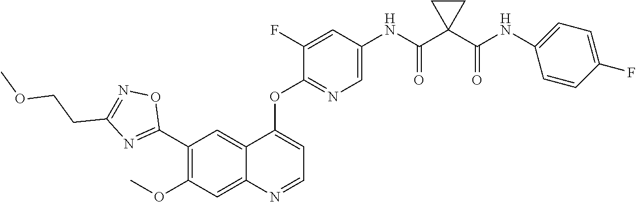 [SMILES: COCCc1noc(-c2cc3c(Oc4ncc(NC(=O)C5(C(=O)Nc6ccc(F)cc6)CC5)cc4F)ccnc3cc2OC)n1] | l-N′-[5-fluoro-6-[7-methoxy-6-[3- (2-methoxyethyl)-1,2,4-oxadiazol- 5-yl]quinolin-4-yl]oxypyridin-3- yl]-1-N-(4- fluorophenyl)cyclopropane-1,1- dicarboxamide |
| 27 | 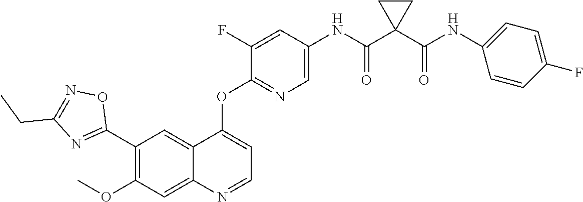 [SMILES: CCc1noc(-c2cc3c(Oc4ncc(NC(=O)C5(C(=O)Nc6ccc(F)cc6)CC5)cc4F)ccnc3cc2OC)n1] | 1-N′-[6-[6-(3-ethyl-1,2,4- oxadiazol-5-yl)-7- methoxyquinolin-4-yl]oxy-5- fluoropyridin-3-yl]-1-N-(4- fluorophenyl)cyclopropane-1,1- dicarboxamide |
| 37 | 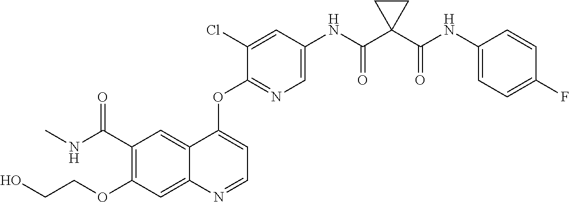 [SMILES: CNC(=O)c1cc2c(Oc3ncc(NC(=O)C4(C(=O)Nc5ccc(F)cc5)CC4)cc3Cl)ccnc2cc1OCCO] | l-N′-[5-chloro-6-[7-(2- hydroxyethoxy)-6- (methylcarbamoyl)quinolin-4- yl]oxypyridin-3-yl]-1-N-(4- fluorophenyl)cyclopropane-1,1- dicarboxamide |
| 38 | 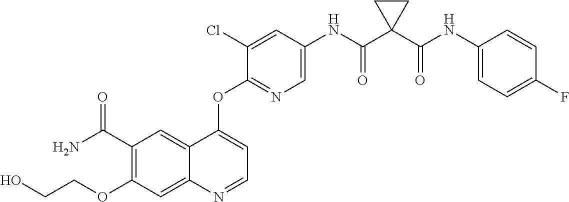 [SMILES: NC(=O)c1cc2c(Oc3ncc(NC(=O)C4(C(=O)Nc5ccc(F)cc5)CC4)cc3Cl)ccnc2cc1OCCO] | 1-N′-[6-[6-carbamoyl-7-(2- hydroxyethoxy)quinolin-4-yl]oxy- 5-chloropyridin-3-yl]-1-N-(4- fluorophenyl)cyclopropane-1,1- dicarboxamide |
| 39 | 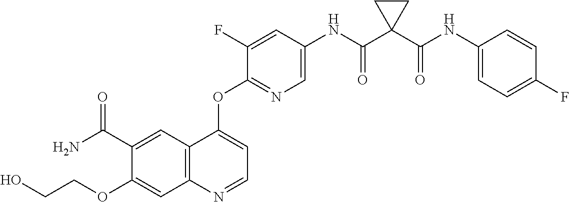 [SMILES: NC(=O)c1cc2c(Oc3ncc(NC(=O)C4(C(=O)Nc5ccc(F)cc5)CC4)cc3F)ccnc2cc1OCCO] | 1-N′-[6-[6-carbamoyl-7-(2- hydroxyethoxy)quinolin-4-yl]oxy- 5-fluoropyridin-3-yl]-1-N-(4- fluorophenyl)cyclopropane-1,1- dicarboxamide |
| 44 | 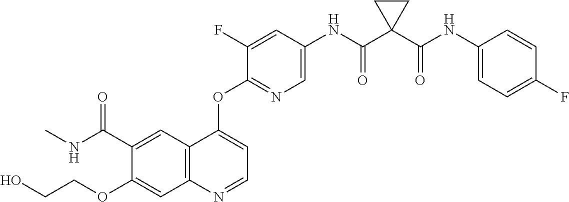 [SMILES: CNC(=O)c1cc2c(Oc3ncc(NC(=O)C4(C(=O)Nc5ccc(F)cc5)CC4)cc3F)ccnc2cc1OCCO] | 1-N′-[5-fluoro-6-[7-(2- hydroxyethoxy)-6- (methylcarbamoyl)quinolin-4- yl]oxypyridin-3-yl]-1-N-(4- fluorophenyl)cyclopropane-1,1- dicarboxamide |
| 50 | 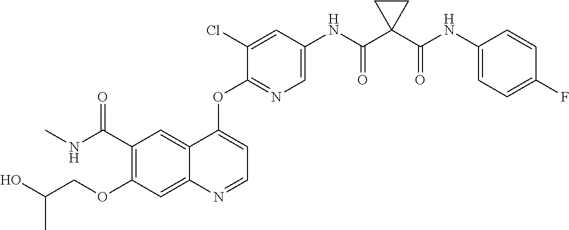 [SMILES: CNC(=O)c1cc2c(Oc3ncc(NC(=O)C4(C(=O)Nc5ccc(F)cc5)CC4)cc3Cl)ccnc2cc1OCC(C)O] | 1-N′-[5-chloro-6-[7-(2- hydroxypropoxy)-6- (methylcarbamoyl)quinolin-4- yl]oxypyridin-3-yl]-1-N-(4- fluorophenyl)cyclopropane-1,1- dicarboxamide |
| 51 | 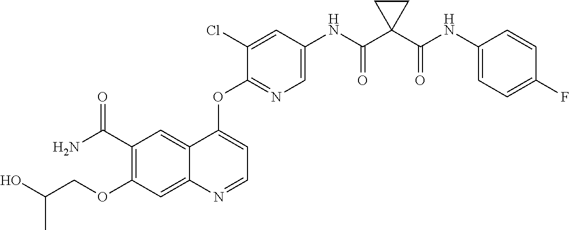 [SMILES: CC(O)COc1cc2nccc(Oc3ncc(NC(=O)C4(C(=O)Nc5ccc(F)cc5)CC4)cc3Cl)c2cc1C(N)=O] | 1-N′-[6-[6-carbamoyl-7-(2- hydroxypropoxy)quinolin-4- yl]oxy-5-chloropyridin-3-yl]-l-N (4-fluorophenyl)cyclopropane-1,1 dicarboxamide |
| 60 | 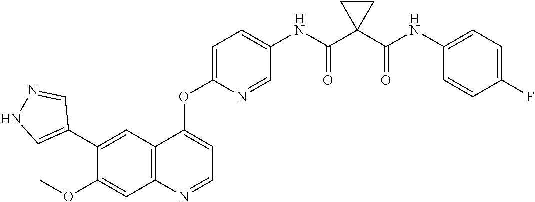 [SMILES: COc1cc2nccc(Oc3ccc(NC(=O)C4(C(=O)Nc5ccc(F)cc5)CC4)cn3)c2cc1-c1cn[nH]c1] | 1-N-(4-fluorophenyl)-1-N′-[6-[7- methoxy-6-(1H-pyrazol-4- yl)quinolin-4-yl]oxypyridin-3- yl]cyclopropane-1,1- dicarboxamide |
| 64 | 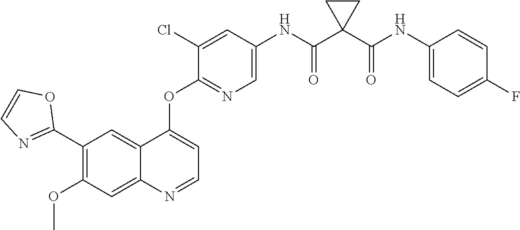 [SMILES: COc1cc2nccc(Oc3ncc(NC(=O)C4(C(=O)Nc5ccc(F)cc5)CC4)cc3Cl)c2cc1-c1ncco1] | l-N′-[5-chloro-6-[7-methoxy-6 (1,3-oxazol-2-yl)quinolin-4- yl]oxypyridin-3-yl]-1-N-(4- fluorophenyl)cyclopropane-1,1 dicarboxamide |
| 67 | 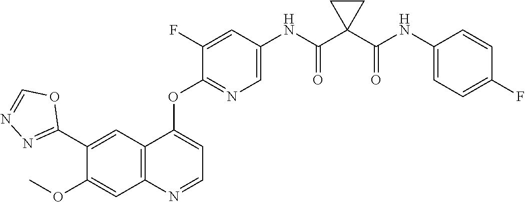 [SMILES: COc1cc2nccc(Oc3ncc(NC(=O)C4(C(=O)Nc5ccc(F)cc5)CC4)cc3F)c2cc1-c1nnco1] | 1-N′-[5-fluoro-6-[7-methoxy-6- (1,3,4-oxadiazol-2-yl)quinolin-4- yl]oxypyridin-3-yl]-1-N-(4- fluorophenyl)cyclopropane-1,1- dicarboxamide |
| 68 | 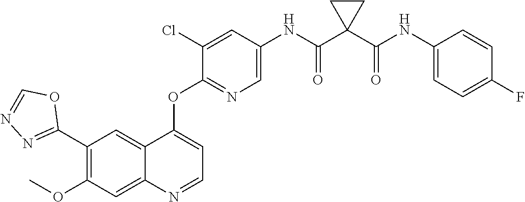 [SMILES: COc1cc2nccc(Oc3ncc(NC(=O)C4(C(=O)Nc5ccc(F)cc5)CC4)cc3Cl)c2cc1-c1nnco1] | 1-N′-[5-chloro-6-[7-methoxy-6- (1,3,4-oxadiazol-2-yl)quinolin-4- yl]oxypyridin-3-yl]-l-N-(4- fluorophenyl)cyclopropane-1,1- dicarboxamide |
| 71 | 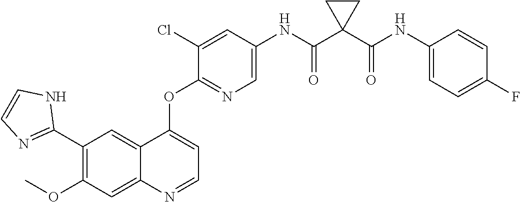 [SMILES: COc1cc2nccc(Oc3ncc(NC(=O)C4(C(=O)Nc5ccc(F)cc5)CC4)cc3Cl)c2cc1-c1ncc[nH]1] | 1-N′-[5-chloro-6-[6-(1H-imidazol- 2-yl)-7-methoxyquinolin-4- yl]oxypyridin-3-yl]-l-N-(4- fluorophenyl)cyclopropane-1,1- dicarboxamide |
| 72 | 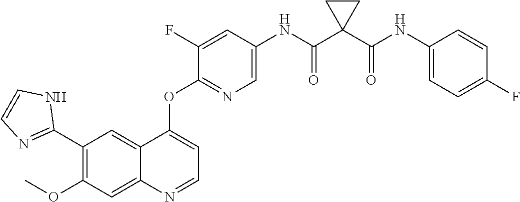 [SMILES: COc1cc2nccc(Oc3ncc(NC(=O)C4(C(=O)Nc5ccc(F)cc5)CC4)cc3F)c2cc1-c1ncc[nH]1] | 1-N′-[5-fluoro-6-[6-(1H-imidazol- 2-yl)-7-methoxyquinolin-4- yl]oxypyridin-3-yl]-1-N-(4- fluorophenyl)cyclopropane-1,1- dicarboxamide |
| 80 | 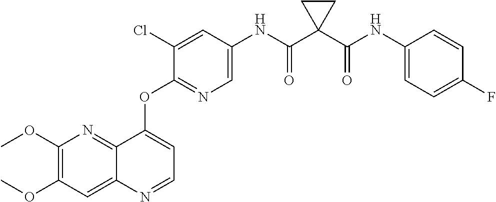 [SMILES: COc1cc2nccc(Oc3ncc(NC(=O)C4(C(=O)Nc5ccc(F)cc5)CC4)cc3Cl)c2nc1OC] | 1-N′-[5-chloro-6-[(6,7-dimethoxy- 1,5-naphthyridin-4-yl)oxy]pyridin- 3-yl]-1-N-(4- fluorophenyl)cyclopropane-1,1- dicarboxamide |
| 81 | 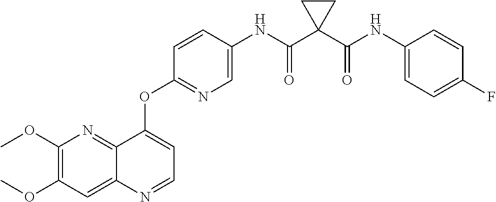 [SMILES: COc1cc2nccc(Oc3ccc(NC(=O)C4(C(=O)Nc5ccc(F)cc5)CC4)cn3)c2nc1OC] | 1-N′-[6-[(6,7-dimethoxy-1,5- naphthyridin-4-yl)oxy]pyridin-3- yl]-1-N-(4- fluorophenyl)cyclopropane-1,1- dicarboxamide |
| 82 | 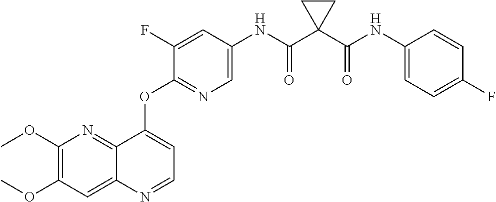 [SMILES: COc1cc2nccc(Oc3ncc(NC(=O)C4(C(=O)Nc5ccc(F)cc5)CC4)cc3F)c2nc1OC] | l-N′-[6-[(6,7-dimethoxy-1,5- naphthyridin-4-yl)oxy]-5- fluoropyridin-3-yl]-1-N-(4- fluorophenyl)cyclopropane-1,1- dicarboxamide |
| 83 | 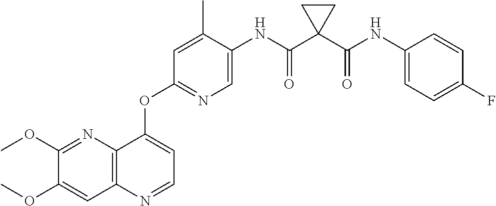 [SMILES: COc1cc2nccc(Oc3cc(C)c(NC(=O)C4(C(=O)Nc5ccc(F)cc5)CC4)cn3)c2nc1OC] | 1-N′-[6-[(6,7-dimethoxy-1,5- naphthyridin-4-yl)oxy]-4- methylpyridin-3-yl]-1-N-(4- fluorophenyl)cyclopropane-1,1- dicarboxamide |
| 84 | 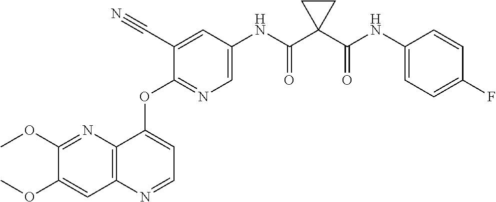 [SMILES: COc1cc2nccc(Oc3ncc(NC(=O)C4(C(=O)Nc5ccc(F)cc5)CC4)cc3C#N)c2nc1OC] | 1-N′-[5-cyano-6-[(6,7-dimethoxy- 1,5-naphthyridin-4-yl)oxy]pyridin- 3-yl]-1-N-(4- fluorophenyl)cyclopropane-1,1- dicarboxamide |
| 85 | 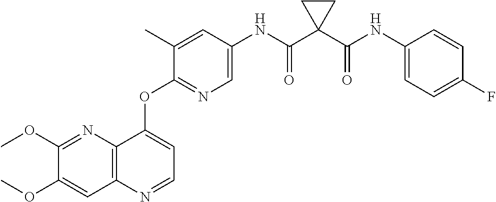 [SMILES: COc1cc2nccc(Oc3ncc(NC(=O)C4(C(=O)Nc5ccc(F)cc5)CC4)cc3C)c2nc1OC] | 1-N′-[6-[(6,7-dimethoxy-1,5- naphthyridin-4-yl)oxy]-5- methylpyridin-3-yl]-1-N-(4- fluorophenyl)cyclopropane-1,1- dicarboxamide |
| 86 | 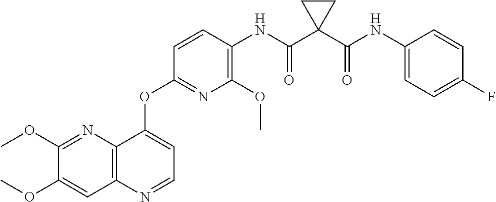 [SMILES: COc1cc2nccc(Oc3ccc(NC(=O)C4(C(=O)Nc5ccc(F)cc5)CC4)c(OC)n3)c2nc1OC] | 1-N′-[6-[(6,7-dimethoxy-1,5- naphthyridin-4-yl)oxy]-2- methoxypyridin-3-yl]-1-N-(4- fluorophenyl)cyclopropane-1,1- dicarboxamide |
| 87 | 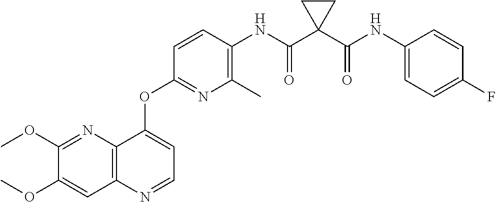 [SMILES: COc1cc2nccc(Oc3ccc(NC(=O)C4(C(=O)Nc5ccc(F)cc5)CC4)c(C)n3)c2nc1OC] | 1-N′-[6-[(6,7-dimethoxy-1,5- naphthyridin-4-yl)oxy]-2- methylpyridin-3-yl]-1-N-(4- fluorophenyl)cyclopropane-1,1- dicarboxamide |
| 88 | 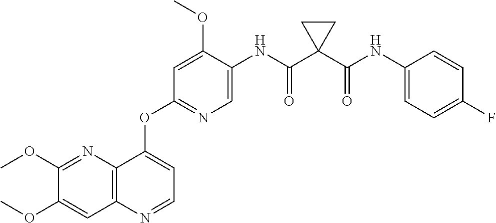 [SMILES: COc1cc(Oc2ccnc3cc(OC)c(OC)nc23)ncc1NC(=O)C1(C(=O)Nc2ccc(F)cc2)CC1] | 1-N′-[6-[(6,7-dimethoxy-1,5- naphthyridin-4-yl)oxy]-4- methoxypyridin-3-yl]-1-N-(4- fluorophenyl)cyclopropane-1,1- dicarboxamide |
| 89 | 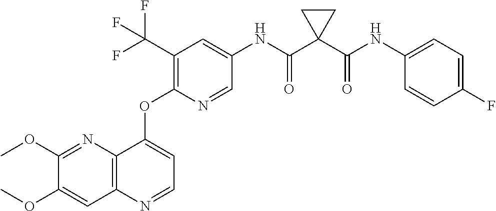 [SMILES: COc1cc2nccc(Oc3ncc(NC(=O)C4(C(=O)Nc5ccc(F)cc5)CC4)cc3C(F)(F)F)c2nc1OC] | 1-N′-[6-[(6,7-dimethoxy-1,5- naphthyridin-4-yl)oxy]-5- (trifluoromethyl)pyridin-3-yl]-1- N-(4-fluorophenyl)cyclopropane- 1,1-dicarboxamide |
| 90 | 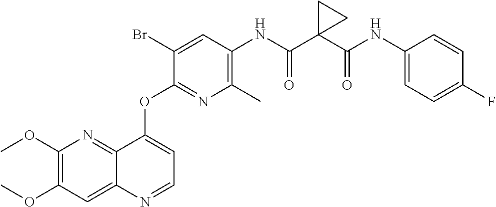 [SMILES: COc1cc2nccc(Oc3nc(C)c(NC(=O)C4(C(=O)Nc5ccc(F)cc5)CC4)cc3Br)c2nc1OC] | 1-N′-[5-bromo-6-[(6,7-dimethoxy- l,5-naphthyridin-4-yl)oxy]-2- methylpyridin-3-yl]-1 -N-(4- fluorophenyl)cyclopropane-1,1- dicarboxamide |
| 91 | 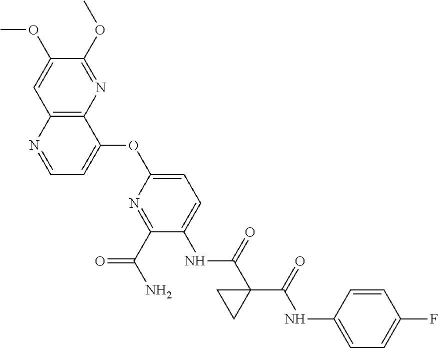 [SMILES: COc1cc2nccc(Oc3ccc(NC(=O)C4(C(=O)Nc5ccc(F)cc5)CC4)c(C(N)=O)n3)c2nc1OC] | 1-N′-[2-carbamoyl-6-[(6,7- dimethoxy-1,5-naphthyridin-4- yl)oxy]pyridin-3-yl]-1-N-(4- fluorophenyl)cyclopropane-1,1- dicarboxamide |
| 92 | 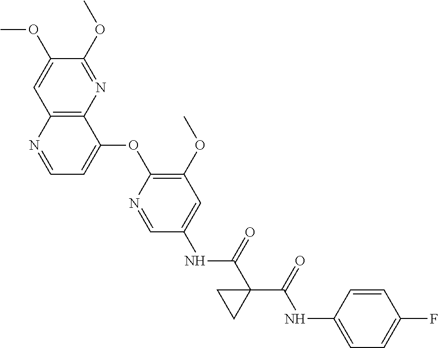 [SMILES: COc1cc2nccc(Oc3ncc(NC(=O)C4(C(=O)Nc5ccc(F)cc5)CC4)cc3OC)c2nc1OC] | 1-N′-[6-[(6,7-dimethoxy-1,5- naphthyridin-4-yl)oxy]-5- methoxypyridin-3-yl]-1-N-(4- fluorophenyl)cyclopropane-1,1- dicarboxamide |
| 96 | 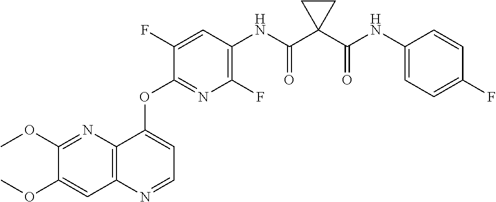 [SMILES: COc1cc2nccc(Oc3nc(F)c(NC(=O)C4(C(=O)Nc5ccc(F)cc5)CC4)cc3F)c2nc1OC] | 1-N′-[6-[(6,7-dimethoxy-1,5- naphthyridin-4-yl)oxy]-2,5- difluoropyridin-3-yl]-1-N-(4- fluorophenyl)cyclopropane-1,1- dicarboxamide |
| 103 | 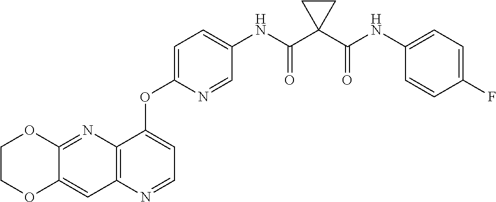 [SMILES: O=C(Nc1ccc(F)cc1)C1(C(=O)Nc2ccc(Oc3ccnc4cc5c(nc34)OCCO5)nc2)CC1] | 1-N′-[6-(2,3-dihydro- [1,4]dioxino[2,3- b][1,5]naphthyridin-6- yloxy)pyridin-3-yl]-1-N-(4- fluorophenyl)cyclopropane-1,1- dicarboxamide |
| 104 | 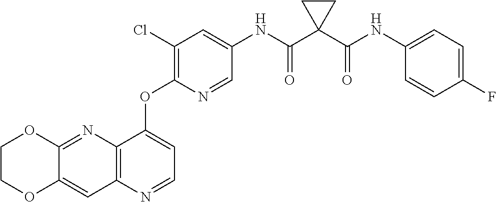 [SMILES: O=C(Nc1ccc(F)cc1)C1(C(=O)Nc2cnc(Oc3ccnc4cc5c(nc34)OCCO5)c(Cl)c2)CC1] | 1-N′-[5-chloro-6-(2,3-dihydro- [1,4]dioxino[2,3- b][1,5]naphthyridin-6- yloxy)pyridin-3-yl]-1-N-(4- fluorophenyl)cyclopropane-1,1- dicarboxamide |
| 105 | 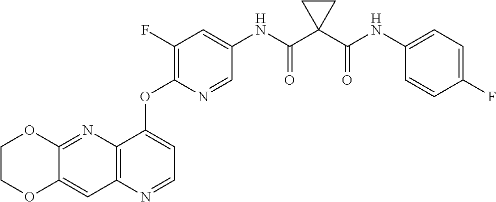 [SMILES: O=C(Nc1ccc(F)cc1)C1(C(=O)Nc2cnc(Oc3ccnc4cc5c(nc34)OCCO5)c(F)c2)CC1] | 1-N′-[6-(2,3-dihydro- [1,4]dioxino[2,3- b][1,5]naphthyridin-6-yloxy)-5 fluoropyridin-3-yl]-1-N-(4- fluorophenyl)cyclopropane-1,1- dicarboxamide |
| 114 | 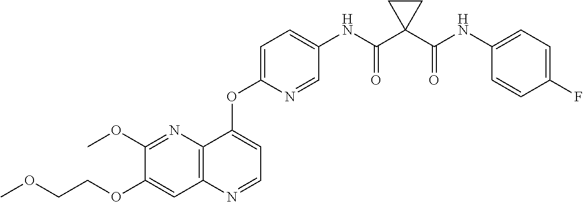 [SMILES: COCCOc1cc2nccc(Oc3ccc(NC(=O)C4(C(=O)Nc5ccc(F)cc5)CC4)cn3)c2nc1OC] | 1-N-(4-fluorophenyl)-1-N′-[6-[[6- methoxy-7-(2-methoxyethoxy)- 1,5-naphthyridin-4-yl]oxy]pyridin- 3-yl]cyclopropane-1,1- dicarboxamide |
| 115 | 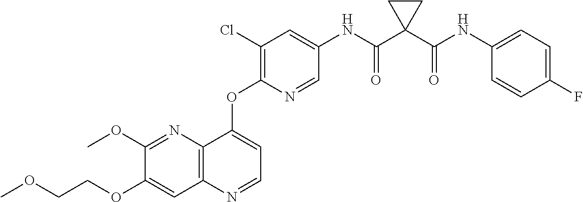 [SMILES: COCCOc1cc2nccc(Oc3ncc(NC(=O)C4(C(=O)Nc5ccc(F)cc5)CC4)cc3Cl)c2nc1OC] | 1-N′-[5-chloro-6-[[6-methoxy-7- (2-methoxyethoxy)-1,5- naphthyridin-4-yl]oxy]pyridin-3- yl]-1-N-(4- fluorophenyl)cyclopropane-1,1- dicarboxamide |
| 116 | 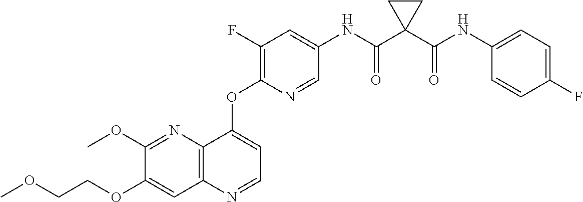 [SMILES: COCCOc1cc2nccc(Oc3ncc(NC(=O)C4(C(=O)Nc5ccc(F)cc5)CC4)cc3F)c2nc1OC] | 1-N′-[5-fluoro-6-[[6-methoxy-7- (2-methoxyethoxy)-1,5- naphthyridin-4-yl]oxy]pyridin-3- yl]-1-N-(4- fluorophenyl)cyclopropane-1,1- dicarboxamide |
| 120 | 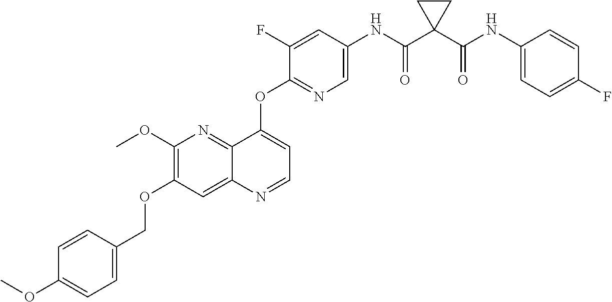 [SMILES: COc1ccc(COc2cc3nccc(Oc4ncc(NC(=O)C5(C(=O)Nc6ccc(F)cc6)CC5)cc4F)c3nc2OC)cc1] | l-N′-[5-fluoro-6-[[6-methoxy-7- [(4-methoxyphenyl)methoxy]-1,5- naphthyridin-4-yl]oxy]pyridin-3- yl]-1-N-(4- fluorophenyl)cyclopropane-1,1- dicarboxamide |
| 121 | 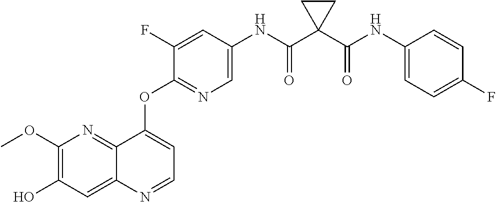 [SMILES: COc1nc2c(Oc3ncc(NC(=O)C4(C(=O)Nc5ccc(F)cc5)CC4)cc3F)ccnc2cc1O] | 1-N′-[5-fluoro-6-[(7-hydroxy-6 methoxy-1,5-naphthyridin-4- yl)oxy]pyridin-3-yl]-1-N-(4- fluorophenyl)cyclopropane-1,1 dicarboxamide |
| 122 | 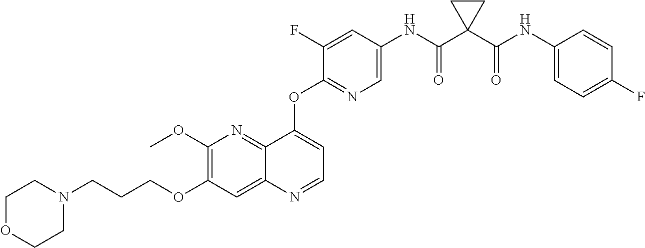 [SMILES: COc1nc2c(Oc3ncc(NC(=O)C4(C(=O)Nc5ccc(F)cc5)CC4)cc3F)ccnc2cc1OCCCN1CCOCC1] | 1-N′-[5-fluoro-6-[[6-methoxy-7- (3-morpholin-4-ylpropoxy)-1,5- naphthyridin-4-yl]oxy]pyridin-3- yl]-1-N-(4- fluorophenyl)cyclopropane-1,1- dicarboxamide |
| 123 | 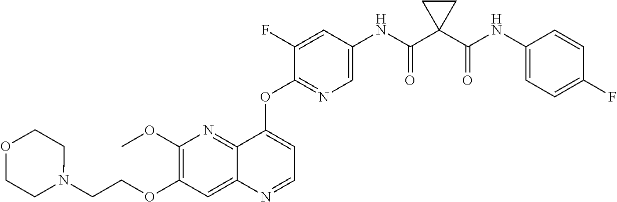 [SMILES: COc1nc2c(Oc3ncc(NC(=O)C4(C(=O)Nc5ccc(F)cc5)CC4)cc3F)ccnc2cc1OCCN1CCOCC1] | 1-N′-[5-fluoro-6-[[6-methoxy-7- (2-morpholin-4-ylethoxy)-1,5- naphthyridin-4-yl]oxy]pyridin-3- yl]-1-N-(4- fluorophenyl)cyclopropane-1,1- dicarboxamide |
| 124 | 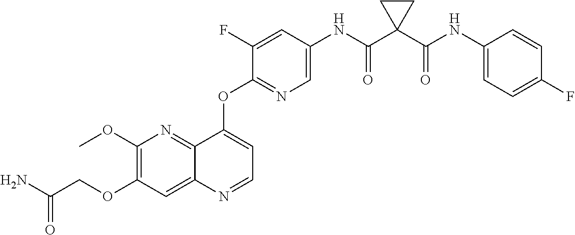 [SMILES: COc1nc2c(Oc3ncc(NC(=O)C4(C(=O)Nc5ccc(F)cc5)CC4)cc3F)ccnc2cc1OCC(N)=O] | 1-N′-[6-[[7-(2-amino-2- oxoethoxy)-6-methoxy-1,5- naphthyridin-4-yl]oxy]-5- fluoropyridin-3-yl]-1-N-(4- fluorophenyl)cyclopropane-1,1- dicarboxamide |
| 125 | 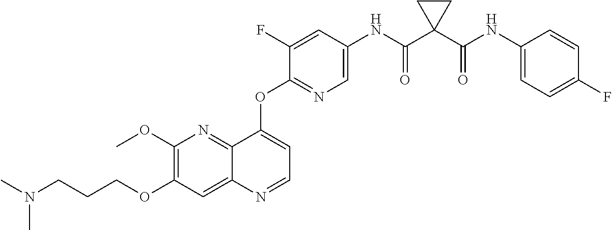 [SMILES: COc1nc2c(Oc3ncc(NC(=O)C4(C(=O)Nc5ccc(F)cc5)CC4)cc3F)ccnc2cc1OCCCN(C)C] | 1-N′-[6-[[7-[3- (dimethylamino)propoxy]-6- methoxy-1,5-naphthyridin-4- yl]oxy]-5-fluoropyridin-3-yl]-1-N (4-fluorophenyl)cyclopropane-1,1- dicarboxamide |
| 127 | 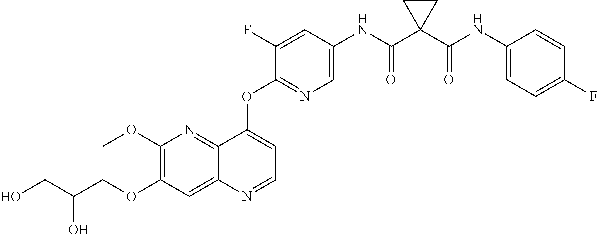 [SMILES: COc1nc2c(Oc3ncc(NC(=O)C4(C(=O)Nc5ccc(F)cc5)CC4)cc3F)ccnc2cc1OCC(O)CO] | 1-N′-[6-[[7-(2,3- dihydroxypropoxy)-6-methoxy- 1,5-naphthyridin-4-yl]oxy]-5- fluoropyridin-3-yl]-1-N-(4- fluorophenyl)cyclopropane-1,1- dicarboxamide |
| 134 | 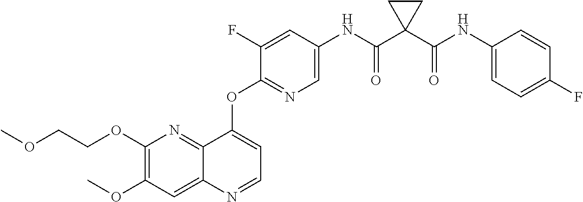 [SMILES: COCCOc1nc2c(Oc3ncc(NC(=O)C4(C(=O)Nc5ccc(F)cc5)CC4)cc3F)ccnc2cc1OC] | 1-N′-[5-fluoro-6-[[7-methoxy-6- (2-methoxyethoxy)-1,5- naphthyridin-4-yl]oxy]pyridin-3- yl]-1-N-(4- fluorophenyl)cyclopropane-1,1- dicarboxamide |
| 135 | 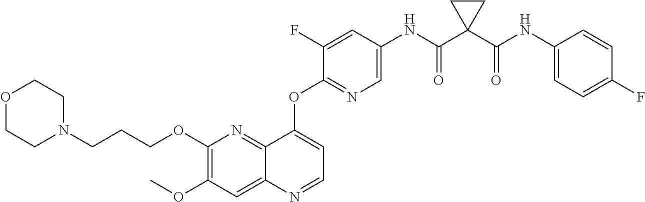 [SMILES: COc1cc2nccc(Oc3ncc(NC(=O)C4(C(=O)Nc5ccc(F)cc5)CC4)cc3F)c2nc1OCCCN1CCOCC1] | 1-N′-[5-fluoro-6-[[7-methoxy-6- (3-morpholin-4-ylpropoxy)-1,5- naphthyridin-4-yl]oxy]pyridin-3- yl]-1-N-(4- fluorophenyl)cyclopropane-1,1- dicarboxamide |
| 145 | 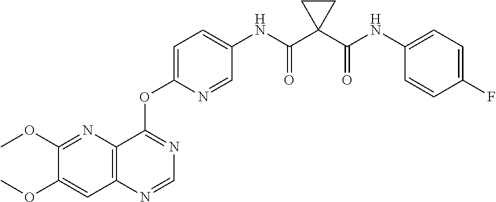 [SMILES: COc1cc2ncnc(Oc3ccc(NC(=O)C4(C(=O)Nc5ccc(F)cc5)CC4)cn3)c2nc1OC] | 1-N′-[6-(6,7-dimethoxypyrido[3,2- d]pyrimidin-4-yl)oxypyridin-3-yl]- 1-N-(4- fluorophenyl)cyclopropane-1,1- dicarboxamide |
| 155 | 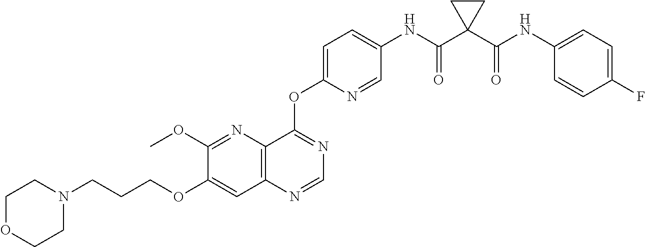 [SMILES: COc1nc2c(Oc3ccc(NC(=O)C4(C(=O)Nc5ccc(F)cc5)CC4)cn3)ncnc2cc1OCCCN1CCOCC1] | 1-N-(4-fluorophenyl)-1-N′-[6-[6- methoxy-7-(3-morpholin-4- ylpropoxy)pyrido[3,2-d]pyrimidin- 4-yl]oxypyridin-3- yl]cyclopropane-1,1- dicarboxamide |
| 156 | 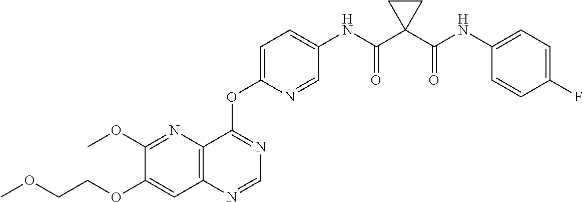 [SMILES: COCCOc1cc2ncnc(Oc3ccc(NC(=O)C4(C(=O)Nc5ccc(F)cc5)CC4)cn3)c2nc1OC] | 1-N-(4-fluorophenyl)-1-N′-[6-[6- methoxy-7-(2- methoxyethoxy)pyrido[3,2- d]pyrimidin-4-yl]oxypyridin-3- yl]cyclopropane-1,1- dicarboxamide |
| 159 | 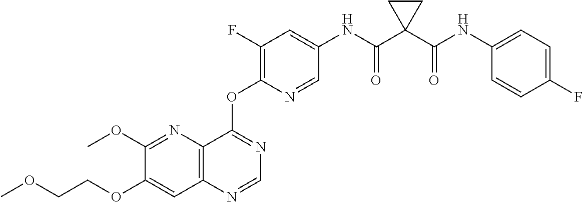 [SMILES: COCCOc1cc2ncnc(Oc3ncc(NC(=O)C4(C(=O)Nc5ccc(F)cc5)CC4)cc3F)c2nc1OC] | 1-N′-[5-fluoro-6-[6-methoxy-7-(2- methoxyethoxy)pyrido[3,2- d]pyrimidin-4-yl]oxypyridin-3-yl]- 1-N-(4- fluorophenyl)cyclopropane-1,1- dicarboxamide |

In one embodiment, the compound of Formula I′ is a compound of Formula I:

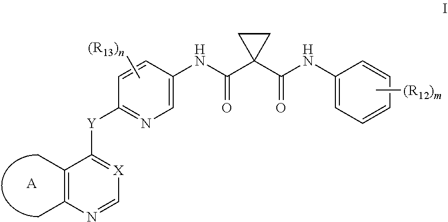

wherein:
X is selected from N and C—H;
Y is O, S, SO, SO₂, NH, or N—(C₁-C₆ alkyl);
R₁₃ is selected from —H, halo, —CN, —C(O)NH₂, and optionally substituted C₁₋₆ alkyl;
R₁₂ is —H or halo;

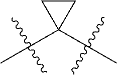 [SMILES: *C1(*)CC1]

is optionally substituted with one, two, three, or four groups independently selected from the group consisting of halo, and C₁-C₆ alkyl, wherein “ [symbol] ” indicate points of attachment;

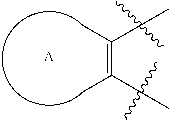

is selected from the group consisting of

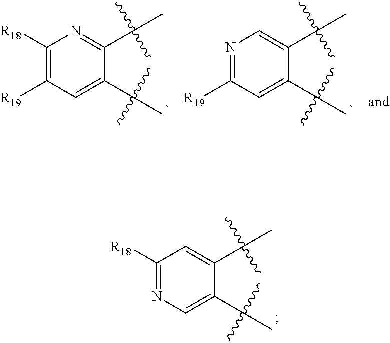

wherein R₁₈ and R₁₉ are selected from the group consisting of H, halo, —CN, optionally substituted C₁-C₆ alkyl, C(O)NR₅R₆, and optionally substituted C₁-C₆ alkoxy; or
when

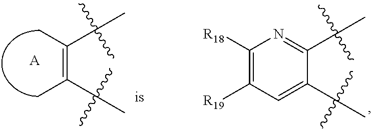

is R₁₈ and R₁₉ can be joined together to form a 5 or 6-membered optionally substituted cycloalkyl or optionally substituted heterocycloalkyl;
R₅ and R₆ are selected from the group consisting of H, optionally substituted C₁₋₆ alkyl, or R₅ and R₆ taken together with the nitrogen to which they are attached to form a 5- or 6-membered optionally substituted heterocycle; and
n and m are each independently 1 or 2;
wherein when

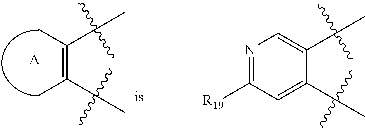

and X is C—H, R₁₉ is not optionally substituted C₁-C₆ alkyl, halo, or optionally substituted C₁-C₆ alkoxy.

In one embodiment, R₁₉ is selected from the group consisting of optionally substituted C₁-C₆ alkoxy and —CN.

In one embodiment,

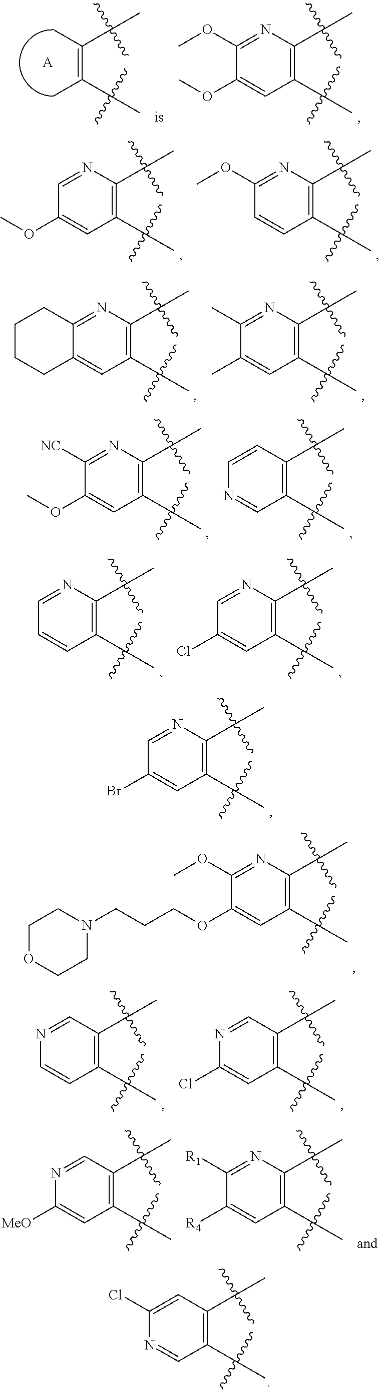

In another embodiment, X is N.

In another embodiment, R₁₃ is H.

In one embodiment, the compound of Formula I, or a pharmaceutically acceptable salt thereof, is selected from the compounds listed in Table 2, or a pharmaceutically acceptable salt thereof.

TABLE 2
Compounds of Formula I

| # | Structure | IUPAC Name |
|---|---|---|
| 80 | 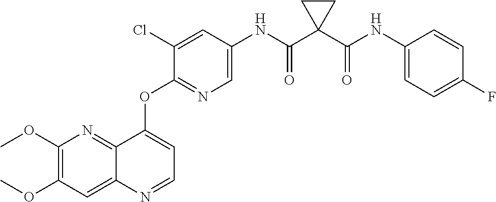 [SMILES: COc1cc2nccc(Oc3ncc(NC(=O)C4(C(=O)Nc5ccc(F)cc5)CC4)cc3Cl)c2nc1OC] | 1-N′-[5-chloro-6-[(6,7-dimethoxy- 1,5-naphthyridin-4-yl)oxy]pyridin- 3-yl]-1-N-(4- fluorophenyl)cyclopropane-1,1- dicarboxamide |
| 81 | 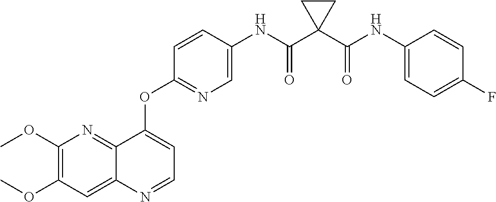 [SMILES: COc1cc2nccc(Oc3ccc(NC(=O)C4(C(=O)Nc5ccc(F)cc5)CC4)cn3)c2nc1OC] | 1-N′-[6-[(6,7-dimethoxy-1,5- naphthyridin-4-yl)oxy]pyridin-3- yl]-1-N-(4- fluorophenyl)cyclopropane-1,1- dicarboxamide |
| 82 | 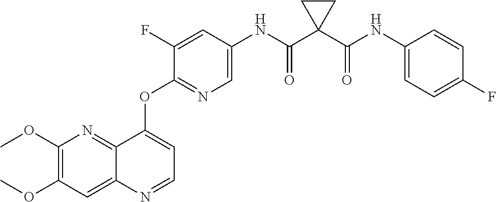 [SMILES: COc1cc2nccc(Oc3ncc(NC(=O)C4(C(=O)Nc5ccc(F)cc5)CC4)cc3F)c2nc1OC] | 1-N′-[6-[(6,7-dimethoxy-1,5- naphthyridin-4-yl)oxy]-5- fluoropyridin-3-yl]-1-N-(4- fluorophenyl)cyclopropane-1,1- dicarboxamide |
| 83 | 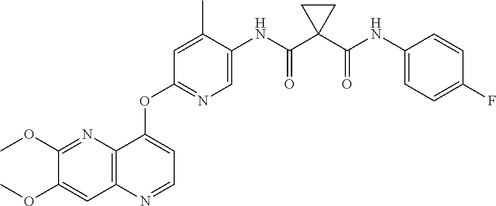 [SMILES: COc1cc2nccc(Oc3cc(C)c(NC(=O)C4(C(=O)Nc5ccc(F)cc5)CC4)cn3)c2nc1OC] | 1-N′-[6-[(6,7-dimethoxy-1,5- naphthyridin-4-yl)oxy]-4- methylpyridin-3-yl]-1-N-(4- fluorophenyl)cyclopropane-1,1- dicarboxamide |
| 84 | 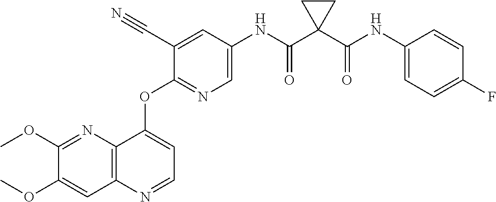 [SMILES: COc1cc2nccc(Oc3ncc(NC(=O)C4(C(=O)Nc5ccc(F)cc5)CC4)cc3C#N)c2nc1OC] | 1-N′-[5-cyano-6-[(6,7-dimethoxy- 1,5-naphthyridin-4-yl)oxy]pyridin- 3-yl]-1-N-(4- fluorophenyl)cyclopropane-1,1- dicarboxamide |
| 85 | 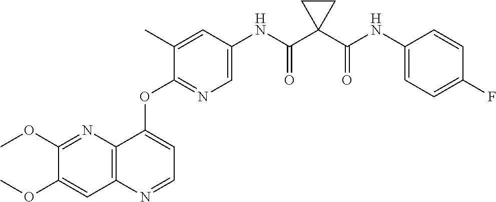 [SMILES: COc1cc2nccc(Oc3ncc(NC(=O)C4(C(=O)Nc5ccc(F)cc5)CC4)cc3C)c2nc1OC] | 1-N′-[6-[(6,7-dimethoxy-1,5- naphthyridin-4-yl)oxy]-5- methylpyridin-3-yl]-1-N-(4- fluorophenyl)cyclopropane-1,1 dicarboxamide |
| 86 | 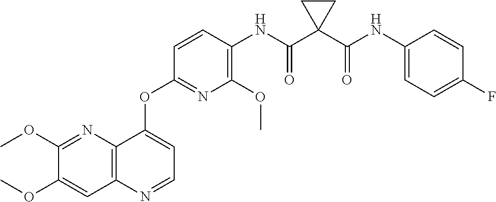 [SMILES: COc1cc2nccc(Oc3ccc(NC(=O)C4(C(=O)Nc5ccc(F)cc5)CC4)c(OC)n3)c2nc1OC] | 1-N′-[6-[(6,7-dimethoxy-1,5- naphthyridin-4-yl)oxy]-2- methoxypyridin-3-yl]-1-N-(4- fluorophenyl)cyclopropane-1,1- dicarboxamide |
| 87 | 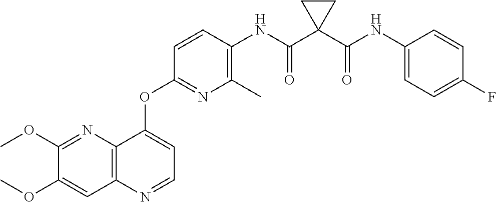 [SMILES: COc1cc2nccc(Oc3ccc(NC(=O)C4(C(=O)Nc5ccc(F)cc5)CC4)c(C)n3)c2nc1OC] | 1-N'-[6-[(6,7-dimethoxy-1,5- naphthyridin-4-yl)oxy]-2- methylpyridin-3-yl]-1-N-(4- fluorophenyl)cyclopropane-1,1- dicarboxamide |
| 88 | 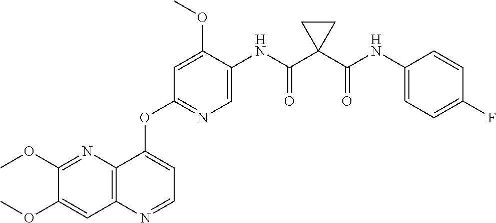 [SMILES: COc1cc(Oc2ccnc3cc(OC)c(OC)nc23)ncc1NC(=O)C1(C(=O)Nc2ccc(F)cc2)CC1] | 1 -N'-[6-[(6,7-dimethoxy-1,5- naphthyridin-4-yl)oxy]-4- methoxypyridin-3-yl]-1-N-(4- fluorophenyl)cyclopropane-1,1- dicarboxamide |
| 89 | 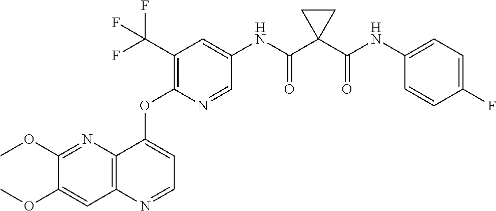 [SMILES: COc1cc2nccc(Oc3ncc(NC(=O)C4(C(=O)Nc5ccc(F)cc5)CC4)cc3C(F)(F)F)c2nc1OC] | 1-N′-[6-[(6,7-dimethoxy-1,5- naphthyridin-4-yl)oxy]-5- (trifluoromethyl)pyridin-3-yl]-1- N-(4-fluorophenyl)cyclopropane- 1,1-dicarboxamide |
| 90 | 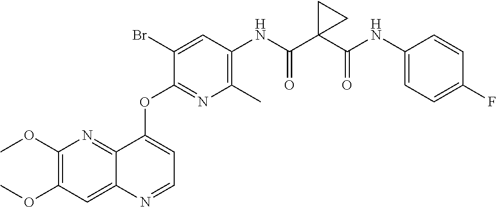 [SMILES: COc1cc2nccc(Oc3nc(C)c(NC(=O)C4(C(=O)Nc5ccc(F)cc5)CC4)cc3Br)c2nc1OC] | 1-N′-[5-bromo-6-[(6,7-dimethoxy- 1,5-naphthyridin-4-yl)oxy]-2- methylpyridin-3-yl]-1-N-(4- fluorophenyl)cyclopropane-1,1- dicarboxamide |
| 91 | 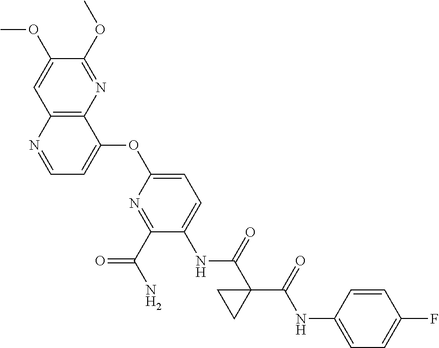 [SMILES: COc1cc2nccc(Oc3ccc(NC(=O)C4(C(=O)Nc5ccc(F)cc5)CC4)c(C(N)=O)n3)c2nc1OC] | 1-N′-[2-carbamoyl-6-[(6,7- dimethoxy-1,5-naphthyridin-4- yl)oxy]pyridin-3-yl]-1-N-(4- fluorophenyl)cyclopropane-1,1- dicarboxamide |
| 92 | 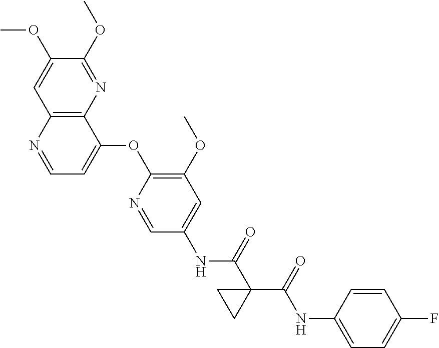 [SMILES: COc1cc2nccc(Oc3ncc(NC(=O)C4(C(=O)Nc5ccc(F)cc5)CC4)cc3OC)c2nc1OC] | 1-N′-[6-[(6,7-dimethoxy-1,5- naphthyridin-4-yl)oxy]-5- methoxypyridin-3-yl]-1-N-(4- fluorophenyl)cyclopropane-1,1- dicarboxamide |
| 96 | 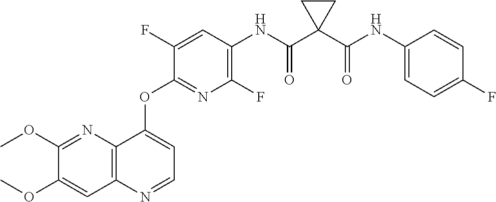 [SMILES: COc1cc2nccc(Oc3nc(F)c(NC(=O)C4(C(=O)Nc5ccc(F)cc5)CC4)cc3F)c2nc1OC] | 1-N′-[6-[(6,7-dimethoxy-1,5- naphthyridin-4-yl)oxy]-2,5- difluoropyridin-3-yl]-1-N-(4- fluorophenyl)cyclopropane-1,1- dicarboxamide |
| 103 | 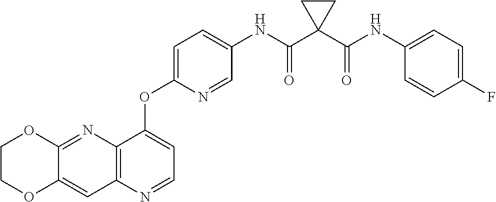 [SMILES: O=C(Nc1ccc(F)cc1)C1(C(=O)Nc2ccc(Oc3ccnc4cc5c(nc34)OCCO5)nc2)CC1] | 1-N′-[6-(2,3-dihydro- [1,4]dioxino[2,3- b][1,5]naphthyridin-6- yloxy)pyridin-3-yl]-1-N-(4- fIuorophenyl)cyclopropane-1,1- dicarboxamide |
| 104 | 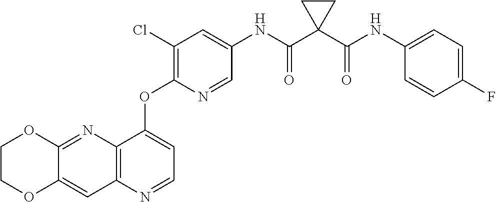 [SMILES: O=C(Nc1ccc(F)cc1)C1(C(=O)Nc2cnc(Oc3ccnc4cc5c(nc34)OCCO5)c(Cl)c2)CC1] | 1-N′-[5-chloro-6-(2,3-dihydro- [1,4]dioxino[2,3- b][1,5]naphthyridin-6- yloxy)pyridin-3-yl]-1-N-(4- fluorophenyl)cyclopropane-1,1- dicarboxamide |
| 105 | 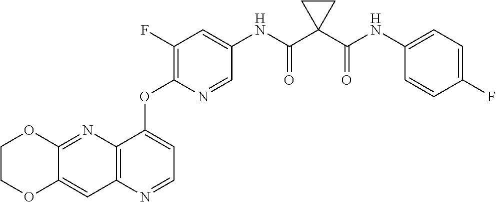 [SMILES: O=C(Nc1ccc(F)cc1)C1(C(=O)Nc2cnc(Oc3ccnc4cc5c(nc34)OCCO5)c(F)c2)CC1] | 1-N′-[6-(2,3-dihydro- [1,4]dioxino[2,3- b][1,5]naphthyridin-6-yloxy)-5 fluoropyridin-3-yl]-1-N-(4- fluorophenyl)cyclopropane-1,1 dicarboxamide |
| 114 | 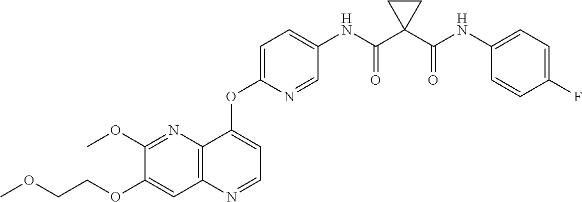 [SMILES: COCCOc1cc2nccc(Oc3ccc(NC(=O)C4(C(=O)Nc5ccc(F)cc5)CC4)cn3)c2nc1OC] | 1-N-(4-fluorophenyl)-1-N′-[6-[[6- methoxy-7-(2-methoxyethoxy)- 1,5-naphthyridin-4-yl]oxy]pyridin- 3-yl]cyclopropane-1,1- dicarboxamide |
| 115 | 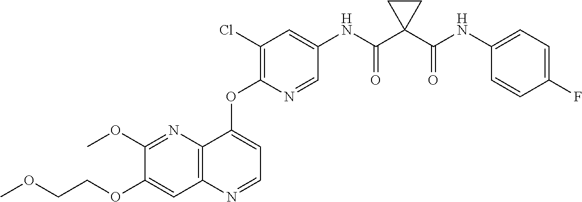 [SMILES: COCCOc1cc2nccc(Oc3ncc(NC(=O)C4(C(=O)Nc5ccc(F)cc5)CC4)cc3Cl)c2nc1OC] | 1-N′-[5-chloro-6-[[6-methoxy-7- (2-methoxyethoxy)-1,5- naphthyridin-4-yl]oxy]pyridin-3 - yl]-1-N-(4- fluorophenyl)cyclopropane-1,1- dicarboxamide |
| 116 | 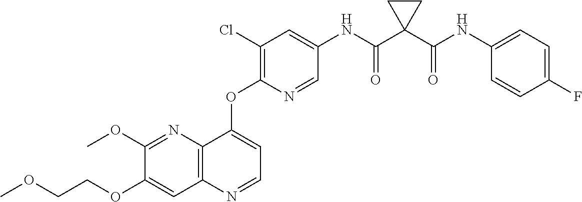 [SMILES: COCCOc1cc2nccc(Oc3ncc(NC(=O)C4(C(=O)Nc5ccc(F)cc5)CC4)cc3Cl)c2nc1OC] | 1-N′-[5-fluoro-6-[[6-methoxy-7- (2-methoxyethoxy)-1,5- naphthyridin-4-yl]oxy]pyridin-3- yl]-1-N-(4- fluorophenyl)cyclopropane-1,1- dicarboxamide |
| 120 | 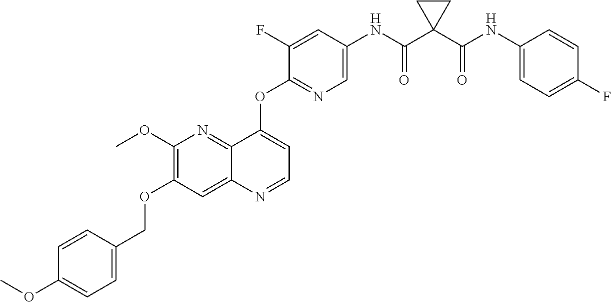 [SMILES: COc1ccc(COc2cc3nccc(Oc4ncc(NC(=O)C5(C(=O)Nc6ccc(F)cc6)CC5)cc4F)c3nc2OC)cc1] | 1-N′-[5-fluoro-6-[[6-methoxy-7- [(4-methoxyphenyl)methoxy]-1,5- naphthyridin-4-yl]oxy]pyridin-3- yl]-1-N-(4- fluorophenyl)cyclopropane-1,1- dicarboxamide |
| 121 | 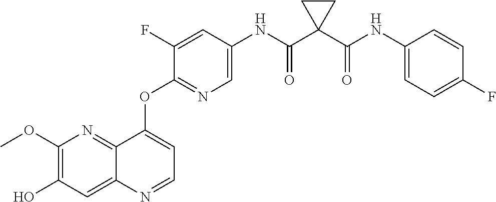 [SMILES: COc1nc2c(Oc3ncc(NC(=O)C4(C(=O)Nc5ccc(F)cc5)CC4)cc3F)ccnc2cc1O] | 1-N′-[5-fluoro-6-[(7-hydroxy-6- methoxy-1,5-naphthyridin-4- yl)oxy]pyridin-3-yl]-1-N-(4- fluorophenyl)cyclopropane-1,1- dicarboxamide |
| 122 | 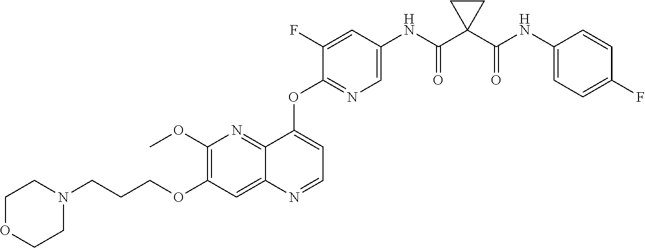 [SMILES: COc1nc2c(Oc3ncc(NC(=O)C4(C(=O)Nc5ccc(F)cc5)CC4)cc3F)ccnc2cc1OCCCN1CCOCC1] | 1-N′-[5-fluoro-6-[[6-methoxy-7- (3-morpholin-4-ylpropoxy)-1,5- naphthyridin-4-yl]oxy]pyridin-3- yl]-1-N-(4- fluorophenyl)cyclopropane-1,1- dicarboxamide |
| 123 | 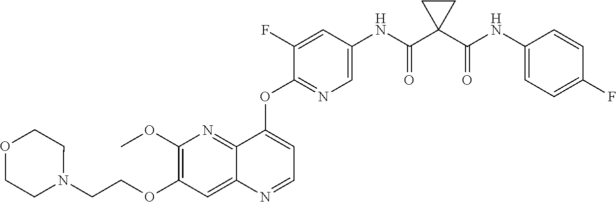 [SMILES: COc1nc2c(Oc3ncc(NC(=O)C4(C(=O)Nc5ccc(F)cc5)CC4)cc3F)ccnc2cc1OCCN1CCOCC1] | 1-N′-[5-fluoro-6-[[6-methoxy-7- (2-morpholin-4-ylethoxy)-1,5- naphthyridin-4-yl]oxy]pyridin-3- yl]-1-N-(4- fluorophenyl)cyclopropane-1,1- dicarboxamide |
| 124 | 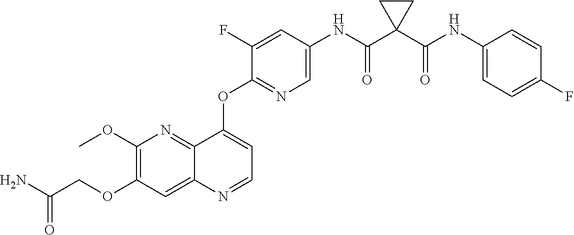 [SMILES: COc1nc2c(Oc3ncc(NC(=O)C4(C(=O)Nc5ccc(F)cc5)CC4)cc3F)ccnc2cc1OCC(N)=O] | 1-N′-[6-[[7-(2-amino-2- oxoethoxy)-6-methoxy-1,5- naphthyridin-4-yl]oxy]-5- fluoropyridin-3-yl]-1-N-(4- fluorophenyl)cyclopropane-1,1- dicarboxamide |
| 125 | 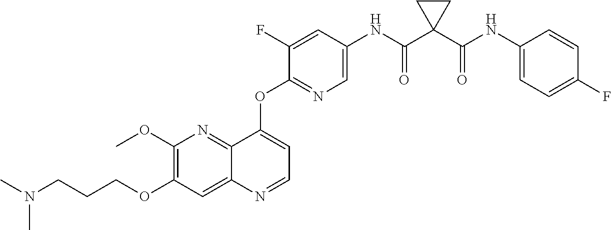 [SMILES: COc1nc2c(Oc3ncc(NC(=O)C4(C(=O)Nc5ccc(F)cc5)CC4)cc3F)ccnc2cc1OCCCN(C)C] | 1-N′-[6-[[7-[3- (dimethylamino)propoxy]-6- methoxy-1,5-naphthyridin-4- yl]oxy]-5-fluoropyridin-3-yl]-1-N- (4-fluorophenyl)cyclopropane-1,1- dicarboxamide |
| 127 | 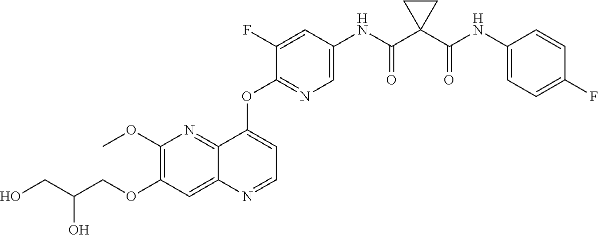 [SMILES: COc1nc2c(Oc3ncc(NC(=O)C4(C(=O)Nc5ccc(F)cc5)CC4)cc3F)ccnc2cc1OCC(O)CO] | 1-N′-[6-[[7-(2,3- dihydroxypropoxy)-6-methoxy- 1,5-naphthyridin-4-yl]oxy]-5- fluoropyridin-3-yl]-l-N-(4- fluorophenyl)cyclopropane-1,1- dicarboxamide |
| 134 | 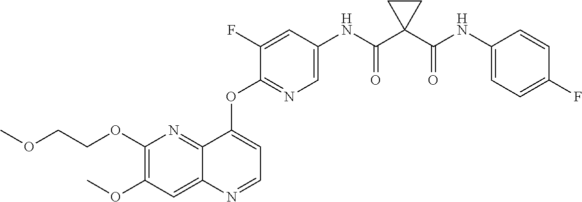 [SMILES: COCCOc1nc2c(Oc3ncc(NC(=O)C4(C(=O)Nc5ccc(F)cc5)CC4)cc3F)ccnc2cc1OC] | 1-N′-[5-fluoro-6-[[7-methoxy-6- (2-methoxyethoxy)-l ,5- naphthyridin-4-yl]oxy]pyridin-3- yl]-1-N-(4- fluorophenyl)cyclopropane-1,1- dicarboxamide |
| 135 | 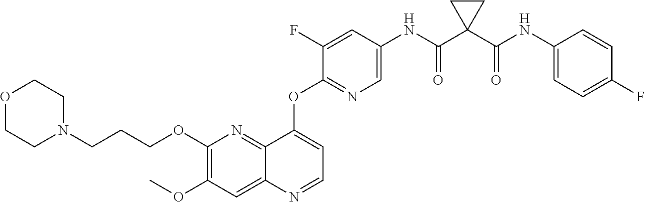 [SMILES: COc1cc2nccc(Oc3ncc(NC(=O)C4(C(=O)Nc5ccc(F)cc5)CC4)cc3F)c2nc1OCCCN1CCOCC1] | 1-N′-[5-fluoro-6-[[7-methoxy-6- (3-morpholin-4-ylpropoxy)-1,5- naphthyridin-4-yl]oxy]pyridin-3- yl]-1-N-(4- fluorophenyl)cyclopropane-1,1- dicarboxamide |
| 145 | 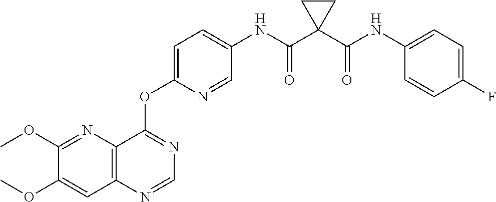 [SMILES: COc1cc2ncnc(Oc3ccc(NC(=O)C4(C(=O)Nc5ccc(F)cc5)CC4)cn3)c2nc1OC] | 1-N′-[6-(6,7-dimethoxypyrido[3,2- d]pyrimidin-4-yl)oxypyridin-3-yl]- 1-N-(4- fluorophenyl)cyclopropane-1,1- dicarboxamide |
| 155 | 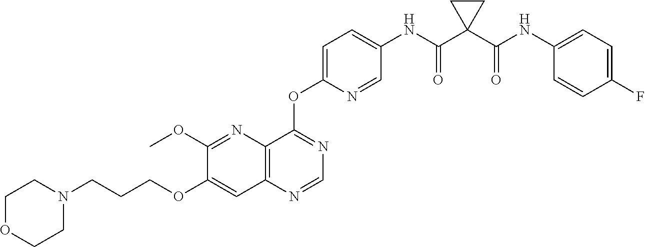 [SMILES: COc1nc2c(Oc3ccc(NC(=O)C4(C(=O)Nc5ccc(F)cc5)CC4)cn3)ncnc2cc1OCCCN1CCOCC1] | 1-N-(4-fluorophenyl)-l-N′-[6-[6- methoxy-7-(3-morpholin-4- ylpropoxy)pyrido[3,2-d]pyrimidin- 4-yl]oxypyridin-3- yl]cydopropane-1,1- dicarboxamide |
| 156 | 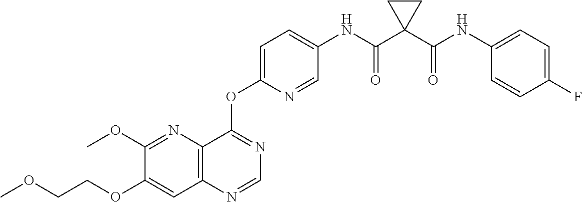 [SMILES: COCCOc1cc2ncnc(Oc3ccc(NC(=O)C4(C(=O)Nc5ccc(F)cc5)CC4)cn3)c2nc1OC] | 1-N-(4-fluorophenyl)-1-N′-[6-[6- methoxy-7-(2- methoxyethoxy)pyrido[3,2- d]pyrimidin-4-yl]oxypyridin-3- yl]cyclopropane-1,1- dicarboxamide |
| 159 | 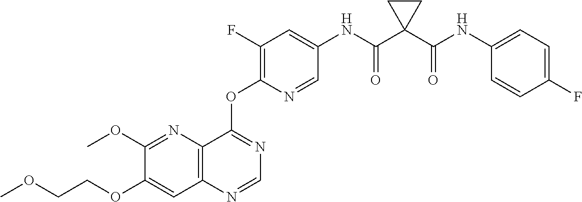 [SMILES: COCCOc1cc2ncnc(Oc3ncc(NC(=O)C4(C(=O)Nc5ccc(F)cc5)CC4)cc3F)c2nc1OC] | 1-N′-[5-fluoro-6-[6-methoxy-7-(2- methoxyethoxy)pyrido[3,2- d]pyrimidin-4-yl]oxypyridin-3-yl]- 1-N-(4- fluorophenyl)cyclopropane-1,1- dicarboxamide |

In one embodiment, the compound of Formula I′ is a compound of Formula II:

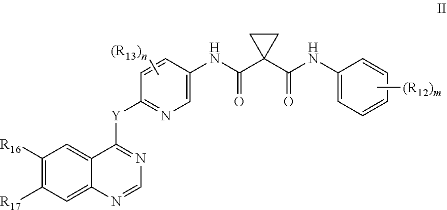

or a pharmaceutically acceptable salt thereof, wherein:
R₁₆ is selected from the group consisting of —CN, optionally substituted 5-6 membered heteroaryl, —COORₐ, and —CO—NR₅R₆;
R₁₇ is selected from H and optionally substituted C₁-C₆ alkoxy;
R₁₃ is selected from the group consisting of —H, halo, —CN, or optionally substituted C₁-C₆ alkyl, or optionally substituted C₁-C₆ alkoxy;
R₁₂ is —H or halo;

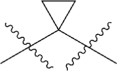 [SMILES: *C1(*)CC1]

is optionally substituted with one, two, three, or four groups independently selected from the group consisting of halo, and C₁-C₆ alkyl, wherein “-” indicate points of attachment;
R₅ and R₆ are each independently H or optionally substituted C₁-C₆ alkyl, optionally substituted C₃-C₆ heterocycloalkyl, optionally substituted C₁-C₆ cycloalkyl;
Y is O, S, SO, SO₂, NH, or N—(C₁-C₆ alkyl); and
n and m are each independently 1 or 2.

In one embodiment, R₁₇ is methoxy.

In another embodiment,

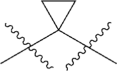 [SMILES: *C1(*)CC1]

is not substituted.

In one embodiment, R₁₂ is halo.

In another embodiment, R₁₂ is para fluoro.

In one embodiment, R₁₆ is —CN or —CO—NR₅R₆.

In a further embodiment, R₁₆ is —CO—NH₂.

In one embodiment, R₁₈ and R₁₉, together with the atoms to which they are attached, are joined together to form a 5- or 6-membered optionally substituted heterocycloalkyl.

In another embodiment, Y is O.

In one embodiment,

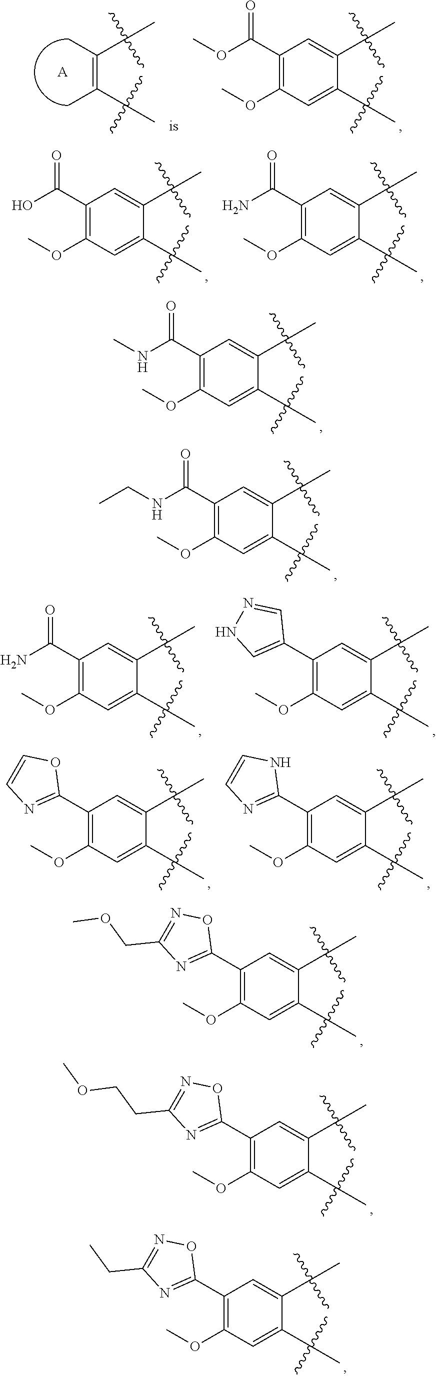

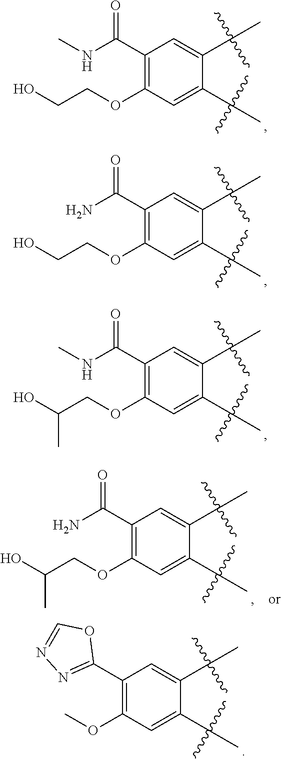

In one embodiment, the compound of Formula II, or a pharmaceutically acceptable salt thereof, is selected from the compounds listed in Table 3, or a pharmaceutically acceptable salt thereof.

TABLE 3
Comopounds of Formula II

| # | Structure | IUPAC Name |
|---|---|---|
| 10 | 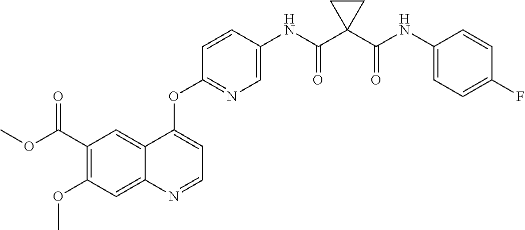 [SMILES: COC(=O)c1cc2c(Oc3ccc(NC(=O)C4(C(=O)Nc5ccc(F)cc5)CC4)cn3)ccnc2cc1OC] | methyl 4-[5-[[1-[(4- fluorophenyl)carbamoyl]cyclo- propanecarbonyl]amino]pyridin-2- yl]oxy-7-methoxyquinoline-6- carboxylate |
| 11 | 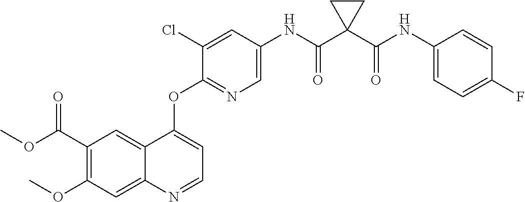 [SMILES: COC(=O)c1cc2c(Oc3ncc(NC(=O)C4(C(=O)Nc5ccc(F)cc5)CC4)cc3Cl)ccnc2cc1OC] | methyl 4-[3-chloro-5-[[1-[(4- fluorophenyl)carbamoyl]cyclo- propanecarbonyl]amino]pyridin-2- yl]oxy-7-methoxyquinoline-6- carboxylate |
| 13 | 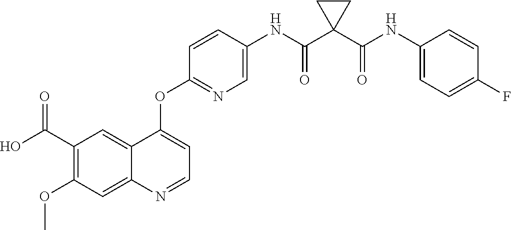 [SMILES: COc1cc2nccc(Oc3ccc(NC(=O)C4(C(=O)Nc5ccc(F)cc5)CC4)cn3)c2cc1C(=O)O] | 4-[5-[[1-[(4- fluorophenyl)carbamoyl]cyclo- propanecarbonyl]amino]pyridin-2- yl]oxy-7-methoxyquinoline-6- carboxylic acid |
| 14 | 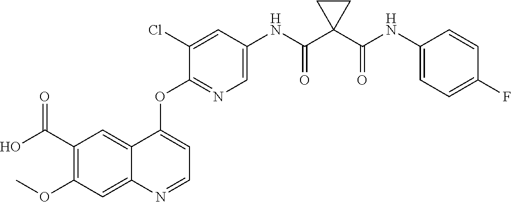 [SMILES: COc1cc2nccc(Oc3ncc(NC(=O)C4(C(=O)Nc5ccc(F)cc5)CC4)cc3Cl)c2cc1C(=O)O] | 4-[3-chloro-5-[[1-[(4- fluorophenyl)carbamoyl]cyclo- propanecarbonyl]amino]pyridin-2- yl]oxy-7-methoxyquinoline-6- carboxylic acid |
| 15 | 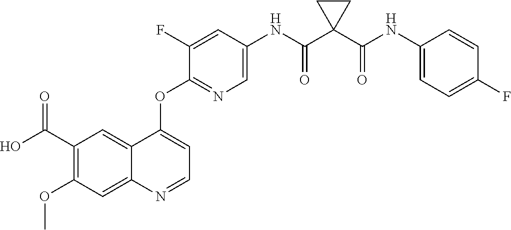 [SMILES: COc1cc2nccc(Oc3ncc(NC(=O)C4(C(=O)Nc5ccc(F)cc5)CC4)cc3F)c2cc1C(=O)O] | 4-[3-fluoro-5-[[1-[(4- fluorophenyl)carbamoyl]cyclo- propanecarbonyl]amino]pyridin-2- yl]oxy-7-methoxyquinoline-6- carboxylic acid |
| 16 | 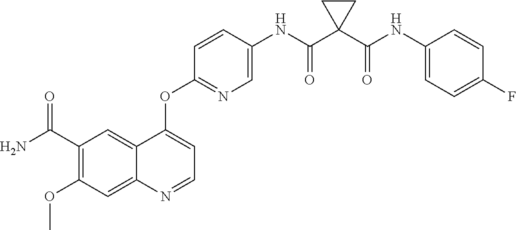 [SMILES: COc1cc2nccc(Oc3ccc(NC(=O)C4(C(=O)Nc5ccc(F)cc5)CC4)cn3)c2cc1C(N)=O] | 1-N′-[6-(6-carbamoyl-7- methoxyquinolin-4-yl)oxypyridin- 3-yl]-1-N-(4- fluorophenyl)cyclopropane-1,1- dicarboxamide |
| 17 | 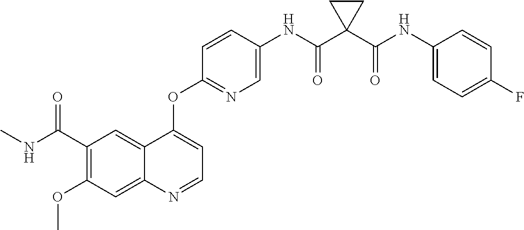 [SMILES: CNC(=O)c1cc2c(Oc3ccc(NC(=O)C4(C(=O)Nc5ccc(F)cc5)CC4)cn3)ccnc2cc1OC] | 1-N-(4-fluorophenyl)-1-N′-[6-[7- methoxy-6- (methylcarbamoyl)quinolin-4- yl]oxypyridin-3-yl]cyclopropane- 1,1-dicarboxamide |
| 18 | 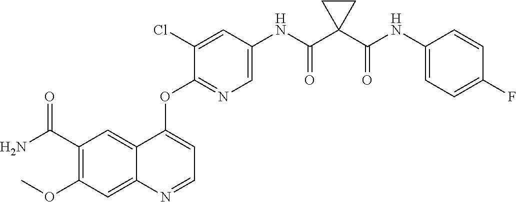 [SMILES: COc1cc2nccc(Oc3ncc(NC(=O)C4(C(=O)Nc5ccc(F)cc5)CC4)cc3Cl)c2cc1C(N)=O] | 1-N′-[6-(6-carbamoyl-7- methoxyquinolin-4-yl)oxy-5- chloropyridin-3-yl]-1-N-(4- fluorophenyl)cyclopropane-1,1- dicarboxamide |
| 19 | 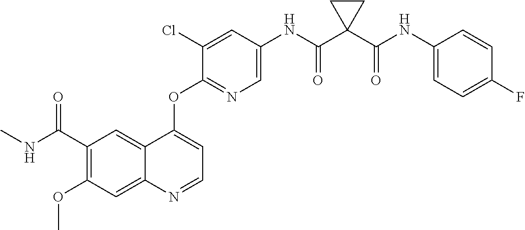 [SMILES: CNC(=O)c1cc2c(Oc3ncc(NC(=O)C4(C(=O)Nc5ccc(F)cc5)CC4)cc3Cl)ccnc2cc1OC] | 1-N′-[5-chloro-6-[7-methoxy-6- (methylcarbamoyl)quinolin-4- yl]oxypyridin-3-yl]-1-N-(4- fluorophenyl)cyclopropane-1,1- dicarboxamide |
| 20 | 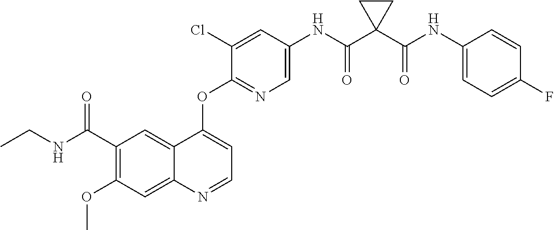 [SMILES: CCNC(=O)c1cc2c(Oc3ncc(NC(=O)C4(C(=O)Nc5ccc(F)cc5)CC4)cc3Cl)ccnc2cc1OC] | 1-N′-[5-chloro-6-[6- (ethylcarbamoyl)-7- methoxyquinolin-4-yl]oxypyridin- 3-yl]-1-N-(4- fluorophenyl)cyclopropane-1,1- dicarboxamide |
| 21 | 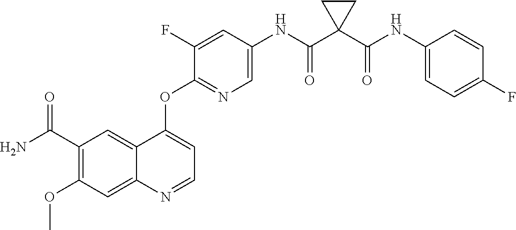 [SMILES: COc1cc2nccc(Oc3ncc(NC(=O)C4(C(=O)Nc5ccc(F)cc5)CC4)cc3F)c2cc1C(N)=O] | 1-N′-[6-(6-carbamoyl-7- methoxyquinolin-4-yl)oxy-5- fluoropyridin-3-yl]-1-N-(4- fluorophenyl)cyclopropane-1,1- dicarboxamide |
| 22 | 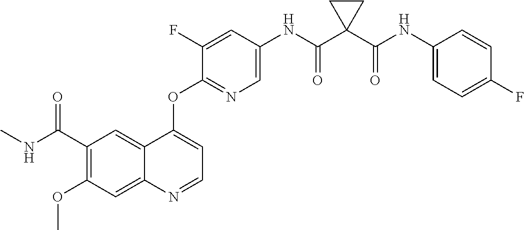 [SMILES: CNC(=O)c1cc2c(Oc3ncc(NC(=O)C4(C(=O)Nc5ccc(F)cc5)CC4)cc3F)ccnc2cc1OC] | 1-N′-[5-fluoro-6-[7-methoxy-6- (methylcarbamoyl)quinolin-4- yl]oxypyridin-3-yl]-1-N-(4- fluorophenyl)cyclopropane-1,1- dicarboxamide |
| 23 | 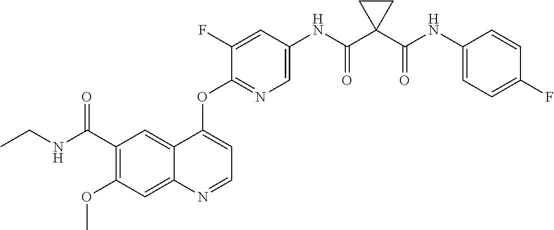 [SMILES: CCNC(=O)c1cc2c(Oc3ncc(NC(=O)C4(C(=O)Nc5ccc(F)cc5)CC4)cc3F)ccnc2cc1OC] | 1-N′-[6-[6-(ethylcarbamoyl)-7- methoxyquinolin-4-yl]oxy-5- fluoropyridin-3-yl]-1-N-(4- fluorophenyl)cyclopropane-1,1- dicarboxamide |
| 25 | 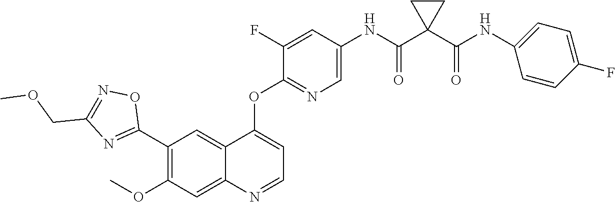 [SMILES: COCc1noc(-c2cc3c(Oc4ncc(NC(=O)C5(C(=O)Nc6ccc(F)cc6)CC5)cc4F)ccnc3cc2OC)n1] | 1-N′-[5-fluoro-6-[7-methoxy-6-[3- (methoxymethyl)-1,2,4-oxadiazol- 5-yl]quinolin-4-yl]oxypyridin-3- yl]-1-N-(4- fluorophenyl)cyclopropane-1,1- dicarboxamide |
| 26 | 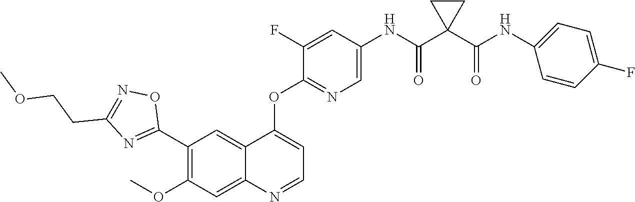 [SMILES: COCCc1noc(-c2cc3c(Oc4ncc(NC(=O)C5(C(=O)Nc6ccc(F)cc6)CC5)cc4F)ccnc3cc2OC)n1] | 1-N′-[5-fluoro-6-[7-methoxy-6-[3- (2-methoxyethyl)-1,2,4-oxadiazol 5-yl]quinolin-4-yl]oxypyridin-3- yl]-1-N-(4- fluorophenyl)cyclopropane-1,1- dicarboxamide |
| 27 | 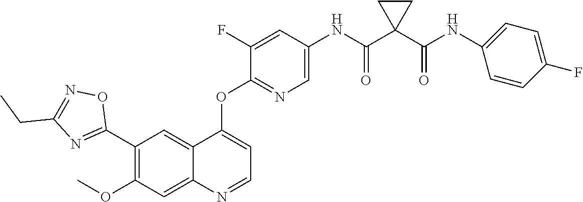 [SMILES: CCc1noc(-c2cc3c(Oc4ncc(NC(=O)C5(C(=O)Nc6ccc(F)cc6)CC5)cc4F)ccnc3cc2OC)n1] | 1-N′-[6-[6-(3-ethyl-1,2,4- oxadiazol-5-yl)-7- methoxyquinolin-4-yl]oxy-5- fluoropyridin-3-yl]-1-N-(4- fluorophenyl)cyclopropane-1,1- dicarboxamide |
| 37 | 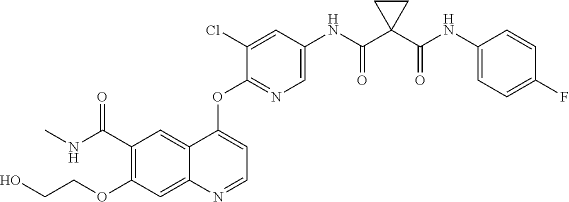 [SMILES: CNC(=O)c1cc2c(Oc3ncc(NC(=O)C4(C(=O)Nc5ccc(F)cc5)CC4)cc3Cl)ccnc2cc1OCCO] | 1-N′-[5-chloro-6-[7-(2- hydroxyethoxy)-6- (methylcarbamoyl)quinolin-4- yl]oxypyridin-3-yl]-1-N-(4- fluorophenyl)cyclopropane-1,1- dicarboxamide |
| 38 | 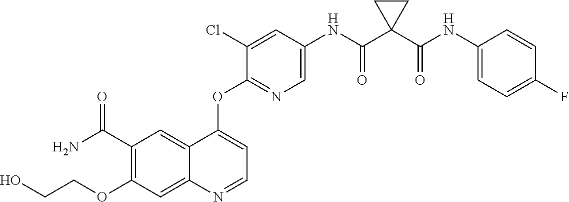 [SMILES: NC(=O)c1cc2c(Oc3ncc(NC(=O)C4(C(=O)Nc5ccc(F)cc5)CC4)cc3Cl)ccnc2cc1OCCO] | 1-N′-[6-[6-carbamoyl-7-(2- hydroxyethoxy)quinolin-4-yl]oxy- 5-chloropyridin-3-yl]-1-N-(4- fluorophenyl)cyclopropane-1,1- dicarboxamide |
| 39 | 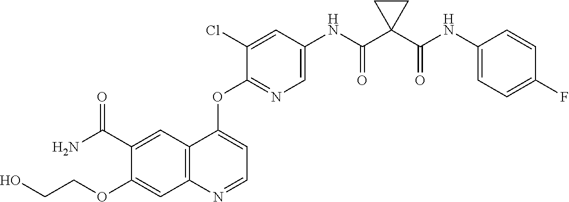 [SMILES: NC(=O)c1cc2c(Oc3ncc(NC(=O)C4(C(=O)Nc5ccc(F)cc5)CC4)cc3Cl)ccnc2cc1OCCO] | 1-N′-[6-[6-carbamoyl-7-(2- hydroxyethoxy)quinolin-4-yl]oxy- 5-fluoropyridin-3-yl]-1-N-(4- fluorophenyl)cyclopropane-1,1- dicarboxamide |
| 44 | 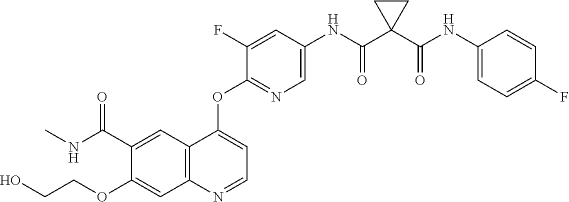 [SMILES: CNC(=O)c1cc2c(Oc3ncc(NC(=O)C4(C(=O)Nc5ccc(F)cc5)CC4)cc3F)ccnc2cc1OCCO] | 1-N′-[5-fluoro-6-[7-(2- hydroxyethoxy)-6- (methylcarbamoyl)quinolin-4- yl]oxypyridin-3-yl]-1-N-(4- fluorophenyl)cyclopropane-1,1- dicarboxamide |
| 50 | 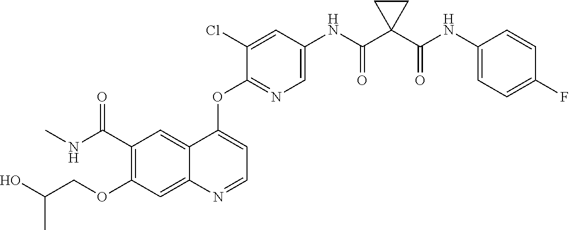 [SMILES: CNC(=O)c1cc2c(Oc3ncc(NC(=O)C4(C(=O)Nc5ccc(F)cc5)CC4)cc3Cl)ccnc2cc1OCC(C)O] | 1-N′-[5-chloro-6-[7-(2- hydroxypropoxy)-6- (methylcarbamoyl)quinolin-4- yl]oxypyridin-3-yl]-1-N-(4- fluorophenyl)cyclopropane-1,1- dicarboxamide |
| 51 | 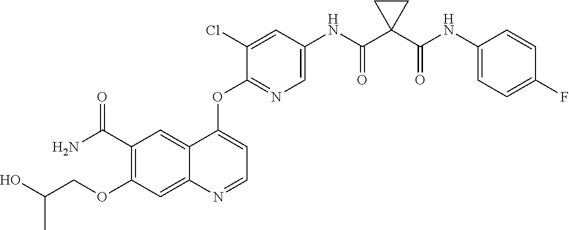 [SMILES: CC(O)COc1cc2nccc(Oc3ncc(NC(=O)C4(C(=O)Nc5ccc(F)cc5)CC4)cc3Cl)c2cc1C(N)=O] | 1-N′-[6-[6-carbamoyl-7-(2- hydroxypropoxy)quinolin-4- yl]oxy-5-chloropyridin-3-yl]-1-N- (4-fluorophenyl)cyclopropane-1,1- dicarboxamide |
| 60 | 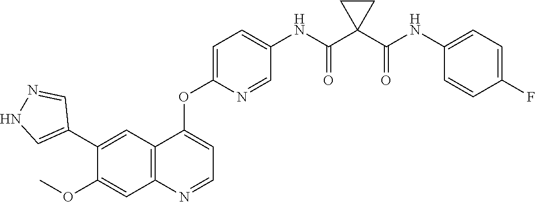 [SMILES: COc1cc2nccc(Oc3ccc(NC(=O)C4(C(=O)Nc5ccc(F)cc5)CC4)cn3)c2cc1-c1cn[nH]c1] | 1-N-(4-fluorophenyl)-1-N′-[6-[7- methoxy-6-(1H-pyrazol-4- yl)quinolin-4-yl]oxypyridin-3- yl]cyclopropane-1,1- dicarboxamide |
| 64 | 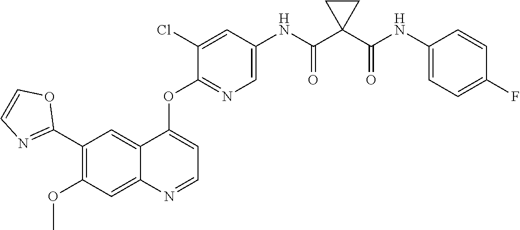 [SMILES: COc1cc2nccc(Oc3ncc(NC(=O)C4(C(=O)Nc5ccc(F)cc5)CC4)cc3Cl)c2cc1-c1ncco1] | 1-N-[5-chloro-6-[7-methoxy-6- (1,3-oxazol-2-yl)quinolin-4- yl]oxypyridin-3-yl]-1-N-(4- fluorophenyl)cyclopropane-1,1- dicarboxamide |
| 67 | 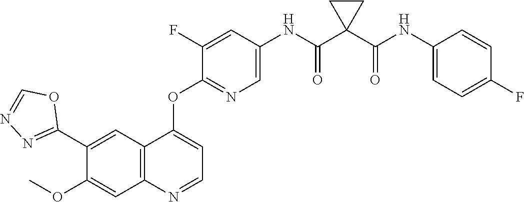 [SMILES: COc1cc2nccc(Oc3ncc(NC(=O)C4(C(=O)Nc5ccc(F)cc5)CC4)cc3F)c2cc1-c1nnco1] | 1-N′-[5-fluoro-6-[7-methoxy-6- (1,3,4-oxadiazol-2-yl)quinolin-4- yl]oxypyridin-3-yl]-1-N-(4- fluorophenyl)cyclopropane-1,1- dicarboxamide |
| 68 | 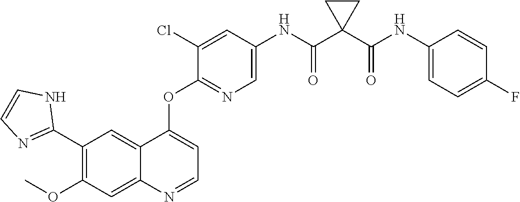 [SMILES: COc1cc2nccc(Oc3ncc(NC(=O)C4(C(=O)Nc5ccc(F)cc5)CC4)cc3Cl)c2cc1-c1ncc[nH]1] | 1-N′-[5-chloro-6-[7-methoxy-6- (1,3,4-oxadiazol-2-yl)quinolin-4- yl]oxypyridin-3-yl]-1-N-(4- fluorophenyl)cyclopropane-1,1- dicarboxamide |
| 71 | 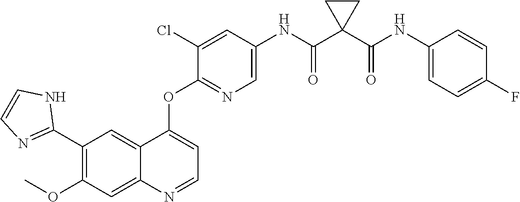 [SMILES: COc1cc2nccc(Oc3ncc(NC(=O)C4(C(=O)Nc5ccc(F)cc5)CC4)cc3Cl)c2cc1-c1ncc[nH]1] | 1-N′-[5-chloro-6-[6-(1H-imidazol- 2-yl)-7-methoxyquinolin-4- yl]oxypyridin-3-yl]-1-N-(4- fluorophenyl)cyclopropane-1,1- dicarboxamide |
| 72 | 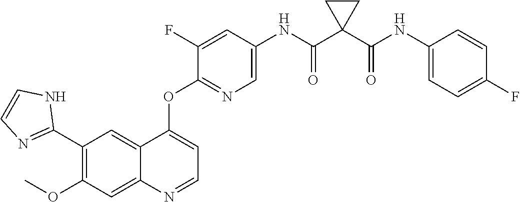 [SMILES: COc1cc2nccc(Oc3ncc(NC(=O)C4(C(=O)Nc5ccc(F)cc5)CC4)cc3F)c2cc1-c1ncc[nH]1] | 1-N′-[5-fluoro-6-[6-(1H-imidazol- 2-yl)-7-methoxyquinolin-4- yl]oxypyridin-3-yl]-1-N-(4- fluorophenyl)cyclopropane-1,1- dicarboxamide |

In one aspect, the invention includes a pharmaceutical composition comprising a compound described herein, and a pharmaceutically acceptable carrier or excipient.

In another aspect, the invention includes a method of treating a disease, disorder, or syndrome mediated at least in part by modulating in vivo activity of a protein kinase, comprising administering to a subject in need thereof a therapeutically effective amount of a compound described herein or a pharmaceutical composition of claim 40.

General Administration

Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as, for example, tablets, suppositories, pills, soft elastic and hard gelatin capsules, powders, solutions, suspensions, aerosols, and the like, preferably in unit dosage forms suitable for simple administration of precise dosages.

The compositions will include a conventional pharmaceutical carrier or excipient and a compound of the invention as the/an active agent, and, in addition, may include other medicinal agents, pharmaceutical agents, carriers, adjuvants, and the like. Compositions of the invention may be used in combination with anticancer or other agents that are generally administered to a patient being treated for cancer. Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate, and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, and the like.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions, and by the use of surfactants.

One preferable route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate, and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, and the like, a compound(s) of the invention, or a pharmaceutically acceptable salt thereof, and optional pharmaceutical adjuvants in a carrier, such as, for example, water, saline, aqueous dextrose, glycerol, ethanol, and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, and dimethylformamide; oils, in particular, cottonseed oil, groundnut oil, corn germ oil, olive oil, castor oil, and sesame oil, glycerol, tetrahydrofurfuryl alcohol, polyethyleneglycols, and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol, and sorbitan esters, microcrystalline cellulose, aluminum metahydroxide, bentonite, agar-agar, and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol, or a suppository wax, which are solid at ordinary temperatures but liquid at body temperature and therefore melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts, are administered in a therapeutically effective amount which will vary depending upon a variety of factors including the activity of the specific compound employed, the metabolic stability, and length of action of the compound, the age, body weight, general health, sex, diet, mode, and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

Combination Therapy

A compound as disclosed herein can be administered as a single therapy or in combination (“co-administered”) with one or more additional therapies for the treatment of a disease or disorder, for instance a disease or disorder associated with hyper-proliferation such as cancer. Therapies that may be used in combination with a compound disclosed herein include: (i) surgery; (ii) radiotherapy (for example, gamma radiation, neutron beam radiotherapy, electron beam radiotherapy, proton therapy, brachytherapy, and systemic radioactive isotopes); (iii) endocrine therapy; (iv) adjuvant therapy, immunotherapy, CAR T-cell therapy; and (v) other chemotherapeutic agents.

The term “co-administered” (“co-administering”) refers to either simultaneous administration, or any manner of separate sequential administration, of a compound of Formula I′ or a salt thereof, and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Typically, any agent that has activity against a disease or condition being treated may be co-administered. Examples of such agents for cancer treatment can be found, for instance, at https://www.cancer.gov/about-cancer/treatment/drugs (last visited Jan. 22, 2019) and in publically available sources such as Cancer Principles and Practice of Oncology by V. T. Devita and S. Hellman (editors), 11^{th} edition (2018), Lippincott Williams & Wilkins Publishers. A person of ordinary skill in the art would be able to discern which combinations of agents would be useful based on the particular characteristics of the drugs and the disease involved.

In one embodiment, the treatment method includes the co-administration of a compound as disclosed herein or a pharmaceutically acceptable salt thereof and at least one immunotherapy. Immunotherapy (also called biological response modifier therapy, biologic therapy, biotherapy, immune therapy, or biological therapy) is treatment that uses parts of the immune system to fight disease. Immunotherapy can help the immune system recognize cancer cells, or enhance a response against cancer cells. Immunotherapies include active and passive immunotherapies. Active immunotherapies stimulate the body's own immune system while passive immunotherapies generally use immune system components created outside of the body.

Examples of active immunotherapies include, but are not limited to vaccines including cancer vaccines, tumor cell vaccines (autologous or allogeneic), dendritic cell vaccines, antigen vaccines, anti-idiotype vaccines, DNA vaccines, viral vaccines, or Tumor-Infiltrating Lymphocyte (TIL) Vaccine with Interleukin-2 (IL-2) or Lymphokine-Activated Killer (LAK) Cell Therapy.

Examples of passive immunotherapies include but are not limited to monoclonal antibodies and targeted therapies containing toxins. Monoclonal antibodies include naked antibodies and conjugated monoclonal antibodies (also called tagged, labeled, or loaded antibodies). Naked monoclonal antibodies do not have a drug or radioactive material attached whereas conjugated monoclonal antibodies are joined to, for example, a chemotherapy drug (chemolabeled), a radioactive particle (radiolabeled), or a toxin (immunotoxin). Examples of these naked monoclonal antibody drugs include, but are not limited to Rituximab (Rituxan), an antibody against the CD20 antigen used to treat, for example, B cell non-Hodgkin lymphoma; Trastuzumab (Herceptin), an antibody against the HER2 protein used to treat, for example, advanced breast cancer; Alemtuzumab (Campath), an antibody against the CD52 antigen used to treat, for example, B cell chronic lymphocytic leukemia (B-CLL); Cetuximab (Erbitux), an antibody against the EGFR protein used, for example, in combination with irinotecan to treat, for example, advanced colorectal cancer and head and neck cancers; and Bevacizumab (Avastin) which is an antiangiogenesis therapy that works against the VEGF protein and is used, for example, in combination with chemotherapy to treat, for example, metastatic colorectal cancer. Examples of the conjugated monoclonal antibodies include, but are not limited to Radiolabeled antibody Ibritumomab tiuxetan (Zevalin) which delivers radioactivity directly to cancerous B lymphocytes and is used to treat, for example, B cell non-Hodgkin lymphoma; radiolabeled antibody Tositumomab (Bexxar) which is used to treat, for example, certain types of non-Hodgkin lymphoma; and immunotoxin Gemtuzumab ozogamicin (Mylotarg) which contains calicheamicin and is used to treat, for example, acute myelogenous leukemia (AML). BL22 is a conjugated monoclonal antibody for treating, for example, hairy cell leukemia, immunotoxins for treating, for example, leukemias, lymphomas, and brain tumors, and radiolabeled antibodies such as OncoScint for example, for colorectal and ovarian cancers and ProstaScint for example, for prostate cancers.

Further examples of therapeutic antibodies that can be used include, but are not limited to, HERCEPTIN™™ (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3epitope) IgG antibody (ImClone System); IMC-C225 which is a chimeric anti-EGFR IgG antibody (ImClone System); VITAXIN™ which is a humanized anti-alpha V beta 3 integrin antibody (Applied Molecular Evolution/Medlmmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatized anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C₅) antibody (Alexion Pharm); D2E7 is a humanized anti-TNF-alpha antibody (CAT/BASF); CDP870 is a humanized anti-TNF-alpha. Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-alpha. IgG4 antibody (Celltech); LDP-02 is a humanized anti-alpha4 beta7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-beta₂ antibody (Cambridge Ab Tech). Others are provided in later paragraphs.

Immunotherapies that can be used in combination with a compound as disclosed herein include adjuvant immunotherapies. Examples include cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guerin (BCG); Keyhole limpet hemocyanin (KLH); Incomplete Freund's adjuvant (IFA); QS-21; DETOX; Levamisole; and Dinitrophenyl (DNP), and combinations thereof, such as, for example, combinations of, interleukins, for example, IL-2 with other cytokines, such as IFN-alpha.

In various embodiments, an immunological therapy or an immunological therapeutic agent can include, one or more of the following: an adoptive cell transfer, an angiogenesis inhibitor, Bacillus Calmette-Guerin therapy, biochemotherapy, a cancer vaccine, a chimeric antigen receptor (CAR) T-cell therapy, a cytokine therapy, gene therapy, an immune checkpoint modulator, an immunoconjugate, a radioconjugate, an oncolytic virus therapy, or a targeted drug therapy. The function or at least one of the functions of the immunological therapy or immunological therapeutic agent, collectively referred to herein as an “immunotherapeutic agent”.

The present disclosure provides a method for preventing, treating, reducing, inhibiting or controlling a neoplasia, a tumor or a cancer in a subject in need thereof, involving administering a therapeutically effective amount of a combination comprising a compound of Formula I′ and an immunotherapeutic agent. In one non-limiting embodiment, the method comprises administering a therapeutically effective amount of a combination comprising a compound of Formula I′ in combination with an immunotherapeutic agent. In various embodiments, the combination provides a cooperative effect, an additive effect, or a synergistic effect in reducing the number of cancer cells when treated with the combination as compared to each treatment alone. In some embodiments, administration of a therapeutically effective amount of a combination comprising a compound of Formula I′ and an immunotherapeutic agent, results in synergistic anti-tumor activity and/or antitumor activity that is more potent than the additive effect of administration of a compound of Formula I′ or immunotherapeutic agent alone.

Human cancers harbor numerous genetic and epigenetic alterations, generating neoantigens potentially recognizable by the immune system (Sjoblom et al. (2006) Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

The present disclosure provides a combination of a compound of Formula I′ and an immunotherapeutic agent. These exemplified combinations can be used to treat a subject with a cancer. In various embodiments, immunotherapeutic agents that find utility in the present compositions, formulations, and methods can include one or more agents or therapies, including: an adoptive cell transfer, an angiogenesis inhibitor, Bacillus Calmette-Guerin therapy, biochemotherapy, a cancer vaccine, a chimeric antigen receptor (CAR) T-cell therapy, a cytokine therapy, gene therapy, an immune checkpoint modulator, for example an immune checkpoint inhibitor, an immunoconjugate, a radioconjugate, an oncolytic virus therapy, or a targeted drug therapy.

In certain embodiments of the present disclosure, a therapeutically effective combination comprises a compound of Formula I′ and an immunotherapeutic agent. In various related embodiments, the compound of Formula I′ enhances the activity of the immunotherapeutic agent.

In certain embodiments of each of the aforementioned aspects, as well as other aspects and embodiments described elsewhere herein, the immunotherapeutic agent enhances the activity of the compound of Formula I′.

In certain embodiments of each of the aforementioned aspects, as well as other aspects and embodiments described elsewhere herein, the compound of Formula I′ and the immunotherapeutic agent act synergistically. In various embodiments described herein, an exemplary immunotherapeutic agent is an immune cell (e.g. T-cell, dendritic cell, a natural killer cell and the like.) modulator chosen from an agonist or an activator of a costimulatory molecule, wherein the modulator is a monoclonal antibody, a bispecific antibody comprising one or more immune checkpoint antigen binding moieties, a trispecific antibody, or an immune cell-engaging multivalent antibody/fusion protein/construct known in the art). In some embodiments, the immunotherapeutic agent can be an antibody that modulates a costimulatory molecule, bind to an antigen on the surface of an immune cell, or a cancer cell. In each of these different embodiments, the antibody modulator can be a monoclonal antibody, a polyclonal antibody, a bispecific antibody, a trispecific or multispecific format antibody, a fusion protein, or a fragment thereof, for example, a Diabody, a Single-chain (sc)-diabody (scFv)2, a Miniantibody, a Minibody, a Barnase-barstar, a scFv-Fc, a sc(Fab)2, a Trimeric antibody construct, a Triabody antibody construct, a Trimerbody antibody construct, a Tribody antibody constuct, a Collabody antibody construct, a (scFv-TNFa)3, or a F(ab)3/DNL antibody construct.

In certain embodiments of each of the aforementioned aspects, as well as other aspects and embodiments described elsewhere herein, the immunotherapeutic agent is an agent that modulates immune responses, for example, a checkpoint inhibitor or a checkpoint agonist. In some embodiments, the immunotherapeutic agent is an agent that enhances anti-tumor immune responses. In some embodiments, the immunotherapeutic agent is an agent that increases cell-mediated immunity. In some embodiments, the immunotherapeutic agent is an agent that increases T-cell activity. In some embodiments, the immunotherapeutic agent is an agent that increases cytolytic T-cell (CTL) activity. In some embodiments, the immunotherapeutic agent is an antibody modulator that targets PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, and/or BTNL2 among others known in the art. In some embodiments, the immunotherapeutic agent is an agent that increases natural killer (NK) cell activity. In some embodiments, the immunotherapeutic agent is an agent that inhibits suppression of an immune response. In some embodiments, the immunotherapeutic agent is an agent that inhibits suppressor cells or suppressor cell activity. In some embodiments, the immunotherapeutic agent is an agent or therapy that inhibits Treg activity. In some embodiments, the immunotherapeutic agent is an agent that inhibits the activity of inhibitory immune checkpoint receptors. In some embodiments, the combination of the present disclosure comprises a compound of Formula I′ and an immunotherapeutic agent, wherein the immunotherapeutic agent includes a T cell modulator chosen from an agonist or an activator of a costimulatory molecule. In one embodiment, the agonist of the costimulatory molecule is chosen from an agonist (e.g., an agonistic antibody or antigen-binding fragment thereof, or a soluble fusion) of GITR, OX40, ICOS, SLAM (e.g., SLAMF7), HVEM, LIGHT, CD2, CD27, CD28, CDS, ICAM-1, LFA-1 (CD11a/CD18), ICOS (CD278), 4-1BB (CD137), CD30, CD40, BAFFR, CD7, NKG2C, NKp80, CD160, B7-H3, or CD83 ligand. In other embodiments, the effector cell combination includes a bispecific T cell engager (e.g., a bispecific antibody molecule that binds to CD3 and a tumor antigen (e.g., EGFR, PSCA, PSMA, EpCAM, HER2 among others).

In some embodiments, the immunotherapeutic agent is a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a modulator of SIRP-alpha activity, a modulator of TIGIT activity, a modulator of VSIG8 activity, a modulator of BTLA activity, a modulator of SIGLEC7 activity, a modulator of SIGLEC9 activity, a modulator of ICOS activity, a modulator of B7H3 activity, a modulator of B7H4 activity, a modulator of FAS activity, a modulator of BTNL2 activity, a cytokine, a chemokine, an interferon, an interleukin, a lymphokine, a member of the tumor necrosis factor (TNF) family, or an immunostimulatory oligonucleotide. In some embodiments, the immunotherapeutic agent is an immune checkpoint modulator (e.g., an immune checkpoint inhibitor e.g. an inhibitor of PD-1 activity, a modulator of PD-L1 activity, a modulator of PD-L2 activity, a modulator of CTLA-4, or a CD40 agonist (e.g., an anti-CD40 antibody molecule), (xi) an OX40 agonist (e.g., an anti-OX40 antibody molecule), or (xii) a CD27 agonist (e.g., an anti-CD27 antibody molecule). In one embodiment, the immunomodulator is an inhibitor of PD-1, PD-L1, PD-L2, CTLA-4, TIM-3, LAG-3, CEACAM (e.g., CEACAM-1, -3 and/or -5), VISTA, BTLA, TIGIT, LAIR1, CD160, 2B4 and/or TGF beta. In one embodiment, the inhibitor of an immune checkpoint molecule inhibits PD-1, PD-L1, LAG-3, TIM-3, CEACAM (e.g., CEACAM-1, -3 and/or -5), CTLA-4, or any combination thereof.

Inhibition of an inhibitory molecule can be performed at the DNA, RNA or protein level. In embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig), or an antibody or antigen-binding fragment thereof, for example, a monoclonal antibody, a bispecific antibody comprising one or more immune checkpoint antigen binding moieties, a trispecific antibody, or an immune cell-engaging multivalent antibody/fusion protein/construct known in the art that binds to the inhibitory molecule; e.g., an antibody or fragment thereof (also referred to herein as “an antibody molecule”) that binds to PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, or a combination thereof.

In some embodiments, where the combination comprises a compound of Formula I′ and an immunotherapeutic agent, wherein the immunotherapeutic agent is a monoclonal antibody or a bispecific antibody. For example, the monoclonal or bispecific antibody may specifically bind a member of the c-Met pathway and/or an immune checkpoint modulator (e.g., the bispecific antibody binds to both a hepatocyte growth factor receptor (HGFR) and an immune checkpoint modulator described herein, such as an antibody that binds PD-1, PD-L1, PD-L2, or CTLA-4, LAG-3, OX40, 41BB, LIGHT, CD40, GITR, TGF-beta, TIM-3, SIRP-alpha, TIGIT, VSIG8, BTLA, SIGLEC7, SIGLEC9, ICOS, B7H3, B7H4, FAS, BTNL2 or CD27). In particular embodiments, the bispecific antibody specifically binds a human HGFR protein and one of PD-1, PD-L1, and CTLA-4.

In some embodiments, the immunotherapeutic agent is a cytokine, for example, a chemokine, an interferon, an interleukin, lymphokine, or a member of the tumor necrosis factor family. In some embodiments, the cytokine is IL-2, IL15, or interferon-gamma.

In some embodiments of any of the above aspects or those described elsewhere herein, the cancer is selected from the group consisting of lung cancer, pancreatic cancer, breast cancer, colon cancer, colorectal cancer, melanoma, gastrointestinal cancer, gastric cancer, renal cancer, ovarian cancer, liver cancer, endometrial cancer, kidney cancer, prostate cancer, thyroid cancer, neuroblastoma, glioma, glioblastoma, glioblastoma multiforme, cervical cancer, stomach cancer, bladder cancer, head and neck cancer, and hepatoma.

In some embodiments of any of the above aspects or those described elsewhere herein, the subject's cancer or tumor does not respond to immune checkpoint inhibition (e.g., to any immune checkpoint inhibitor described herein, such as a PD-1 antagonist or PD-L1 antagonist) or the subject's cancer or tumor has progressed following an initial response to immune checkpoint inhibition (e.g., to any immune checkpoint inhibitor described herein, such as a PD-1 antagonist or PD-L1 antagonist).

In some embodiments of any of the above aspects or those described elsewhere herein, the subject is a human.

A checkpoint inhibitor can be any molecule, agent, treatment and/or method of inhibiting an immune checkpoint, and/or promoting an inhibitor of an immune checkpoint, e.g., by promoting an intrinsic immune checkpoint inhibitor; inhibiting a transcription factor involved in the expression of an immune checkpoint; and/or by acting in concert with some additional extrinsic factor. For example, a checkpoint inhibitor could include a treatment that inhibits transcription factors involved the expression of immune checkpoint genes, or promotes the expression of transcription factors for tumor-suppressor genes, e.g., BACH2 (Luan et al., (2016). Transcription Factors and Checkpoint Inhibitor Expression with Age: Markers of Immunosenescence. Blood, 128(22), 5983). Moreover, a checkpoint inhibitor can inhibit the transcription of immune checkpoint genes; the modification and/or processing of immune checkpoint mRNA; the translation of immune checkpoint proteins; and/or molecules involved in immunity or the immune checkpoint pathway, e.g., PD-1 transcription factors such as HIF-1, STAT3, NF-κB, and AP-1, or the activation of common oncogenic pathways such as JAK/STAT, RAS/ERK, or PI3K/AKT/mTOR (Zerdes et al., Genetic, transcriptional and post-translational regulation of the programmed death protein ligand 1 in cancer: biology and clinical correlations, Oncogenevolume 37, pages 4639-4661 (2018), the disclosure of which is incorporated herein by reference in its entirety).

Checkpoint inhibitors can include treatments, molecules, agents, and/or methods that regulate immune checkpoints at the transcriptional level, e.g., using the RNA-interference pathway co-suppression, and/or post-transcriptional gene silencing (PTGS) (e.g., microRNAs, miRNA; silencing-RNA, small-interfering-RNA, or short-interfering-RNA (siRNA). Transcriptional regulation of checkpoint molecules has been shown to involve mir-16, which has been shown to target the 3′UTR of the checkpoint mRNAs CD80, CD274 (PD-L1) and CD40 (Leibowitz et al., Post-transcriptional regulation of immune checkpoint genes by mir-16 in melanoma, Annals of Oncology (2017) 28; v428-v448). Mir-33a has also been shown to be involved in regulating the expression of PD-1 in cases of lung adenocarcinoma (Boldini et al., Role of microRNA-33a in regulating the expression of PD-1 in lung adenocarcinoma, Cancer Cell Int. 2017; 17: 105, the disclosure of which is incorporated herein by reference in its entirety).

T-cell-specific aptamer-siRNA chimeras have been suggested as a highly specific method of inhibiting molecules in the immune checkpoint pathway (Hossain et al., The aptamer-siRNA conjugates: reprogramming T cells for cancer therapy, Ther. Deliv. 2015 January; 6(1): 1-4, the disclosure of which is incorporated herein by reference in its entirety).

Alternatively, members of the immune checkpoint pathway can be inhibited using treatments that affect associated pathways, e.g., metabolism. For example, oversupplying the glycolytic intermediate pyruvate in mitochondria from CAD macrophages promoted expression of PD-L1 via induction of the bone morphogenetic protein 4/phosphorylated SMAD1/5/IFN regulatory factor 1 (BMP4/p-SMAD1/5/IRF1) signaling pathway. Accordingly, implementing treatments that modulate the metabolic pathway can result in subsequent modulation of the immunoinhibitory PD-1/PD-L1 checkpoint pathway (Watanabe et al., Pyruvate controls the checkpoint inhibitor PD-L1 and suppresses T cell immunity, J Clin Invest. 2017 Jun. 30; 127(7): 2725-2738).

Checkpoint immunity can be regulated via oncolytic viruses that selectively replicate within tumor cells and induce acute immune responses in the tumor-micro-environment, i.e., by acting as genetic vectors that carry specific agents (e.g., antibodies, miRNA, siRNA, and the like.) to cancer cells and effecting their oncolysis and secretion of cytokines and chemokines to synergize with immune checkpoint inhibition (Shi et al., Cancer Immunotherapy: A Focus on the Regulation of Immune Checkpoints, Int J Mol Sci. 2018 May; 19(5): 1389). Currently, there are clinical trials underway that utilize the following viruses as checkpoint inhibitors: poliovirus, measles virus, adenoviruses, poxviruses, herpes simplex virus (HSV), coxsackieviruses, reovirus, Newcastle disease virus (NDV), T-VEC (a herpes virus encoded with GM-CSF (granulocyte-macrophage colony stimulating factor)), and H101 (Shi et al., supra).

Checkpoint inhibitors can operate at the translational level of checkpoint immunity. The translation of mRNA into protein represents a key event in the regulation of gene expression, thus inhibition of immune checkpoint translation is a method in which the immune checkpoint pathway can be inhibited.

Inhibition of the immune checkpoint pathway can occur at any stage of the immune checkpoint translational process. For example, drugs, molecules, agents, treatments, and/or methods can inhibit the initiation process (whereby the 40S ribosomal subunit is recruited to the 5′ end of the mRNA and scans the 5′UTR of the mRNA toward its 3′ end. Inhibition can occur by targeting the anticodon of the initiator methionyl-transfer RNA (tRNA) (Met-tRNAi), its base-pairing with the start codon, or the recruitment of the 60S subunit to begin elongation and sequential addition of amino acids in the translation of immune-checkpoint-specific genes. Alternatively, a checkpoint inhibitor can inhibit checkpoints at the translational level by preventing the formation of the ternary complex (TC), i.e., eukaryotic initiation factor (eIF)₂ (or one or more of its α, β, and γ subunits); GTP; and Met-tRNAi.

Checkpoint inhibition can occur via destabilization of eIF2α by precluding its phosphorylation via protein kinase R (PKR), PERK, GCN2, or HRI, or by precluding TCs from associating with the 40S ribosome and/or other initiation factors, thus preventing the preinitiation complex (PIC) from forming; inhibiting the eIF4F complex and/or its cap-binding protein eIF4E, the scaffolding protein eIF4G, or eIF4A helicase. Methods discussing the translational control of cancer are discussed in Truitt et al., New frontiers in translational control of the cancer genome, Nat Rev Cancer. 2016 Apr. 26; 16(5): 288-304, the disclosure of which is incorporated herein by reference in its entirety.

Checkpoint inhibitors can also include treatments, molecules, agents, and/or methods that regulate immune checkpoints at the cellular and/or protein level, e.g., by inhibiting an immune checkpoint receptor. Inhibition of checkpoints can occur via the use of antibodies, antibody fragments, antigen-binding fragments, small-molecules, and/or other drugs, agents, treatments, and/or methods.

Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response (‘block’ the immune response) against tumor tissues. In contrast to the majority of anti-cancer agents, checkpoint inhibitors do not target tumor cells directly, but rather target lymphocyte receptors or their ligands in order to enhance the endogenous antitumor activity of the immune system. (Pardoll, 2012, Nature Reviews Cancer 12:252-264).

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of antibody (Ab), ipilimumab (YERVOY®), that binds to and inhibits CTLA-4 for the treatment of patients with advanced melanoma (Hodi et al. (2010) N Engl J Med 363:711-23) and the development of antibodies such as nivolumab and pembrolizumab (formerly lambrolizumab; USAN Council Statement (2013) Pembrolizumab: Statement on a nonproprietary name adopted by the USAN Council (ZZ-165), Nov. 27, 2013) that bind specifically to the Programmed Death-1 (PD-1) receptor and block the inhibitory PD-1/PD-1 ligand pathway (Topalian et al. (2012a) N Engl J Med 366:2443-54; Topalian et al. (2012b) Curr Opin Immunol 24:207-12; Topalian et al. (2014) J Clin Oncol 32(10):1020-30; Hamid et al. (2013) N Engl J Med 369:134-144; Hamid and Carvajal (2013) Expert Opin Biol Ther 13(6):847-61; McDermott and Atkins (2013) Cancer Med 2(5):662-73).

PD-1 is a key immune checkpoint receptor expressed by activated T and B cells and mediates immunosuppression. Nivolumab (formerly designated 5C₄, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al. (2014) In vitro characterization of the anti-PD-1 antibody nivolumab, BMS-936558, and in vivo toxicology in non-human primates. Nivolumab has been approved for the treatment of patients with unresectable or metastatic melanoma and disease progression following ipilimumab and, if BRAF V600 mutation positive, a BRAF inhibitor and for the treatment of squamous non-small cell lung cancer.

Recent data suggest a secondary mechanism of anti-CTLA-4 antibodies, which could occur within the tumor itself. CTLA-4 has been found to be expressed in tumors at higher levels on regulatory T-cells (also referred to herein as “Treg cells”) as compared with intra-tumoral effector T-cells (also referred to herein as “Teff cells”), resulting in the hypothesis of anti-CTLA-4 preferentially impacting the Treg cell. “Therapeutic use of anti-CTLA-4 antibodies”, Christian U. Blank and Alexander Enk, International Immunology, Vol. 27, No. 1, pp. 3-10. A recent study of a PD-1 and CTLA-4 combination show that the combination blockade of the CTLA-4 and PD-1 pathways also cooperates to increase the ratio of Teff cells to both regulatory T-cells and MDSCs, thereby reducing suppression and promoting inflammation in the tumor microenvironment. “Combination of CTLA-4 and PD-1 blockade expands infiltrating T-cells and reduces regulatory T and myeloid cells within B16 melanoma tumors”, Curran et al., PNAS|Mar. 2, 2010; vol. 107 (no. 9); pp. 4275-4280, the disclosure of which is incorporated herein by reference in its entirety. The combination of a checkpoint inhibitor and another therapeutic agent(s) may enhance or prolong anti-tumor response of the checkpoint inhibitor and/or effects of the therapeutic agent. In this regard, WO 2015/069770 discloses a combination treatment based on activating the adaptive immune response, in particular the combination of CTLA-4 and PD-1 inhibitors, for the treatment of cancer. The disclosure of WO 2015/069770 is incorporated by reference in its entirety in the disclosure of this application.

One mechanism by which the checkpoint blockade anti-CTLA-4 antibodies mediate anti-tumor effect is by decreasing regulatory T-cells. Due to the distinct mechanism of action of anti-CTLA-4 antibodies, they can successfully combine with the anti-PD1 checkpoint blockade antibodies which work to release the suppressive signaling conferred to effector T-cells. Dual blockade with these antibodies combine to improve anti-tumor response both preclinically (Proc Natl Acad Sci USA 2010, 107, 4275-4280) and in the clinic (N Engl J Med 2013, 369, 122-133; N Engl J Med 2015, 372, 2006-2017).

CTLA-4 attenuates the early activation of naïve and memory T cells through interactions with its ligands B7-1 (CD80) and B7-2 (CD86) (FIG. 1A). PD-1 is an receptor expressed on the surface of activated mature T cells, activated NK cells, B cells, monocytes and multiple normal tissues and plays a crucial role in the maintenance of peripheral tolerance [20-21] (FIG. 1A). In contrast to CTLA-4, PD-1 acts via interactions with its ligands PD-L1 (also known as B7-H1 or CD274) and is involved mainly in T cell activity modulation in peripheral tissues as well as providing a major immune resistance mechanism within the tumor microenvironment.

In some embodiments, the immunotherapeutic agent is a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, or an immunostimulatory oligonucleotide. In some embodiments, the immune checkpoint modulator, i.e. is an inhibitor or antagonist, or is an activator or agonist, for example, a CD28 modulator, a 4-1BB modulator, an OX40 modulator, a CD27 modulator, a CD80 modulator, a CD86 modulator, a CD40 modulator, or a GITR modulator, a Lag-3 modulator, a 41BB modulator, a LIGHT modulator, a CD40 modulator, a GITR modulator, a TGF-beta modulator, a TIM-3 modulator, a SIRP-alpha modulator, a TIGIT modulator, a VSIG8 modulator, a BTLA modulator, a SIGLEC7 modulator, a SIGLEC9 modulator, a ICOS modulator, a B7H3 modulator, a B7H4 modulator, a FAS modulator, and/or a BTNL2 modulator. In some embodiments, the immunotherapeutic agent is an immune checkpoint modulator as described above (e.g., an immune checkpoint modulator antibody, which can be in the form of a monoclonal antibody, a bispecific antibody comprising one or more immune checkpoint antigen binding moieties, a trispecific antibody, or an immune cell-engaging multivalent antibody/fusion protein/construct known in the art).

Combination treatments with immune checkpoint inhibitor immunotherapeutic agent may include antibodies that specifically target immune system checkpoints such as CTLA4, PD1 and PD-L1 are one of the most promising new avenues of immunotherapy for cancer and other diseases. Additional checkpoint targets, such as TIM-3, LAG-3, various B-7 ligands, CHK 1 and CHK2 kinases, BTLA, A2aR, and others, are also under investigation. Currently, three checkpoint inhibitors have received rapid approval from the U.S. Food and Drug Administration for cancer treatment, including ipilimumab (Yervoy®), a CTLA-4 inhibitor, and pembrolizumab (Keytruda®) and nivolumab (Opdivo®), both PD-1 inhibitors. In addition, several checkpoint inhibitor agents are in clinical trials.

Programmed Cell Death Protein 1, (PD-1 or CD279), a 55-kD type 1 transmembrane protein, is a member of the CD28 family of T cell co-stimulatory receptors that include immunoglobulin superfamily member CD28, CTLA-4, inducible co-stimulator (ICOS), and BTLA. PD-1 is highly expressed on activated T cells and B cells. PD-1 expression can also be detected on memory T-cell subsets with variable levels of expression. Two ligands specific for PD-1 have been identified: programmed death-ligand 1 (PD-L1, also known as B7-H1 or CD274) and PD-L2 (also known as B7-DC or CD273). PD-L1 and PD-L2 have been shown to down-regulate T cell activation upon binding to PD-1 in both mouse and human systems (Okazaki et al., Int Immunol., 2007; 19: 813-824). The interaction of PD-1 with its ligands, PD-L1 and PD-L2, which are expressed on antigen-presenting cells (APCs) and dendritic cells (DCs), transmits negative regulatory stimuli to down-modulate the activated T cell immune response. Blockade of PD-1 suppresses this negative signal and amplifies T cell responses.

Numerous studies indicate that the cancer microenvironment manipulates the PD-L1-/PD-1 signaling pathway and that induction of PD-L1 expression is associated with inhibition of immune responses against cancer, thus permitting cancer progression and metastasis. The PD-L1/PD-1 signaling pathway is a primary mechanism of cancer immune evasion for several reasons. First, and most importantly, this pathway is involved in negative regulation of immune responses of activated T effector cells, found in the periphery. Second, PD-L1 is up-regulated in cancer microenvironments, while PD-1 is also up-regulated on activated tumor infiltrating T cells, thus possibly potentiating a vicious cycle of inhibition. Third, this pathway is intricately involved in both innate and adaptive immune regulation through bi-directional signaling. These factors make the PD-1/PD-L1 complex a central point through which cancer can manipulate immune responses and promote its own progression.

CTLA-4 (also known as Cytotoxic T-lymphocyte-associated protein 4, CTLA4, CTLA-4, CD152, cluster of differentiation 152; ALPS5, CD, CELIAC3, GRD4, GSE, and IDDM12). CTLA-4 is a -24.6-kDa single-pass type I membrane protein that plays an inhibitory role in T-cell function. CTLA-4 was originally identified by differential screening of a murine cytolytic T cell cDNA library, See Brunet et al., A new member of the immunoglobulin superfamily—CTLA-4, Nature. 1987 Jul. 16-22; 328(6127):267-70. CTLA- has been shown to interact with the b7 family ligands CD80 (also known as Cluster of differentiation 80, and B7-1); and CD86 (also known as Cluster of Differentiation 86 or B7-2). See Linsley et al., CTLA-4 is a second receptor for the B cell activation antigen B7, J Exp Med. 1991 Sep. 1; 174(3):561-9. Sequence comparison between the human CTLA-4 DNA encoding region, and that of CD28, reveals significant homology between both sequences, with the greatest similarity between juxtamembrane and cytoplasmic regions; accordingly, CTLA-4 is implicated in abrogating/reducing T-cell activity, and opposes the activity of CD28. CTLA-4 deficient mice have been shown to exhibit massive lymphoproliferation. Chambers et al., Lymphoproliferation in CTLA-4-deficient mice is mediated by costimulation-dependent activation of CD4+ T cells, Immunity. 1997 December; 7(6):885-95. It has been reported that CTLA-4 blockade augments T-cell responses both in vitro and in vivo, enhances an induced autoimmune disease, and exacerbates antitumor immunity. (See Luhder, J. Exp. Med. 1998; 187:427-432; Walunas et al., Immunity. 1994; 1:405-413; Kearney, J. Immunol. 1995; 155:1032-1036); Leach, Science 1996; 271:1734-1736). CTLA-4 has also been reported as having alternative and/or additional impact on the initial character of the T-cell immune response (Chambers, Curr. Opin. Immunol. 1997; 9:396-404; Bluestone, J. Immunol. 1997; 158:1989-1993; Thompson, Immunity 1997; 7:445-450).

The first immune-checkpoint inhibitor to be tested in a clinical trial was ipilimumab (Yervoy, Bristol-Myers Squibb), an CTLA-4 mAb. CTLA-4 belongs to the immunoglobulin superfamily of receptors, which also includes PD-1, BTLA, TIM-3, and V-domain immunoglobulin suppressor of T cell activation (VISTA). Anti-CTLA-4 mAb is a powerful checkpoint inhibitor which removes “the break” from both naive and antigen-experienced cells. Therapy enhances the antitumor function of CD8+ T cells, increases the ratio of CD8+ T cells to Foxp3+ T regulatory cells, and inhibits the suppressive function of T regulatory cells. The major drawback to anti-CTLA-4 mAb therapy is the generation of autoimmune toxicities due to on-target effects of an over-exuberant immune system which has lost the ability to turn itself down. It has been reported that up to 25% of patients treated with ipilimumab developed serious grade 3-4 adverse events/autoimmune-type side effects including dermatitis, enterocolitis, hepatitis, endocrinopathies (including hypophysitis, thyroiditis, and adrenalitis), arthritis, uveitis, nephritis, and aseptic meningitis. In contrast to the anti-CTLA-4 experience, anti-PD-1 therapy appears to be better-tolerated and induces a relatively lower rate of autoimmune-type side effects.

In some embodiments, the immunotherapeutic agent is an agent that inhibits the activity of PD-1. In some embodiments, the immunotherapeutic agent is an agent that inhibits the activity of PD-L1 and/or PD-L2. In some embodiments, the immunotherapeutic agent is an agent that inhibits the activity of CTLA-4. In some embodiments, the immunotherapeutic agent is an agent that inhibits the activity of CD80 and/or CD86. In some embodiments, the immunotherapeutic agent is an agent that inhibits the activity of TIGIT. In some embodiments, the immunotherapeutic agent is an agent that inhibits the activity of KIR. In some embodiments, the immunotherapeutic agent is an agent that enhances or stimulates the activity of activating immune checkpoint receptors.

In some of the embodiments of the methods described herein, the immunotherapeutic agent is a PD-1 antagonist, a PD-L1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, or an IDO1 antagonist.

In some embodiments, the PD-1 antagonist is an antibody that specifically binds PD-1. In some embodiments, the antibody that binds PD-1 is pembrolizumab (KEYTRUDA®, MK-3475; Merck), pidilizumab (CT-011; Curetech Ltd.), nivolumab (OPDIVO®, BMS-936558, MDX-1106; Bristol Myer Squibb), MEDIO680 (AMP-514; AstraZenenca/MedImmune), REGN2810 (Regeneron Pharmaceuticals), BGB-A317 (BeiGene Ltd.), PDR-001 (Novartis), or STI-A1110 (Sorrento Therapeutics). In some embodiments, the antibody that binds PD-1 is described in PCT Publication WO 2014/179664, for example, an antibody identified as APE2058, APE1922, APE1923, APE1924, APE 1950, or APE1963 (Anaptysbio), or an antibody containing the CDR regions of any of these antibodies. In other embodiments, the PD-1 antagonist is a fusion protein that includes the extracellular domain of PD-L1 or PD-L2, for example, AMP-224 (AstraZeneca/MedImmune). In other embodiments, the PD-1 antagonist is a peptide inhibitor, for example, AUNP-12 (Aurigene).

In some embodiments, the PD-L1 antagonist is an antibody that specifically binds PD-L1. In some embodiments, the antibody that binds PD-L1 is atezolizumab (RG7446, MPDL3280A; Genentech), MEDI4736 (AstraZeneca/MedImmune), BMS-936559 (MDX-1105; Bristol Myers Squibb), avelumab (MSB0010718C; Merck KGaA), KD033 (Kadmon), the antibody portion of KD033, or STI-A1014 (Sorrento Therapeutics). In some embodiments, the antibody that binds PD-L1 is described in PCT Publication WO 2014/055897, for example, Ab-14, Ab-16, Ab-30, Ab-31, Ab-42, Ab-50, Ab-52, or Ab-55, or an antibody that contains the CDR regions of any of these antibodies, the disclosure of which is incorporated herein by reference in its entirety.

In some embodiments, the CTLA-4 antagonist is an antibody that specifically binds CTLA-4. In some embodiments, the antibody that binds CTLA-4 is ipilimumab (YERVOY®; Bristol Myer Squibb) or tremelimumab (CP-675,206; Pfizer). In some embodiments, the CTLA-4 antagonist a CTLA-4 fusion protein or soluble CTLA-4 receptor, for example, KARR-102 (Kahr Medical Ltd.).

In some embodiments, the LAG3 antagonist is an antibody that specifically binds LAG3. In some embodiments, the antibody that binds LAG3 is IMP701 (Prima BioMed), RVIP731 (Prima BioMed/GlaxoSmithKline), BMS-986016 (Bristol Myer Squibb), LAG525 (Novartis), and GSK2831781 (GlaxoSmithKline). In some embodiments, the LAG3 antagonist includes a soluble LAG3 receptor, for example, IMP321 (Prima BioMed).

In some embodiments, the KIR antagonist is an antibody that specifically binds KIR. In some embodiments, the antibody that binds KIR is lirilumab (Bristol Myer Squibb/Innate Pharma).

In some embodiments, the immunotherapeutic agent used in the combinations disclosed herein (e.g., in combination with a compound of Formula I′) is an activator or agonist of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CD28, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, or CD83 ligand.

In some embodiments, the OX40 agonist includes OX40 ligand, or an OX40-binding portion thereof. For example, the OX40 agonist may be MEDI6383 (AstraZeneca). In some embodiments, the OX40 agonist is an antibody that specifically binds OX40. In some embodiments, the antibody that binds OX40 is MEDI6469 (AstraZeneca/Medlmmune), MEDI0562 (AstraZeneca/Medlmmune), or MOXR0916 (RG7888; Genentech). In some embodiments, the OX40 agonist is a vector (e.g., an expression vector or virus, such as an adenovirus) capable of expressing OX40 ligand. In some embodiments the OX40-expressing vector is Delta-24-RGDOX (DNAtrix) or DNX2401 (DNAtrix).

In some embodiments, the 4-1BB (CD137) agonist is a binding molecule, such as an anticalin. In some embodiments, the anticalin is PRS-343 (Pieris AG). In some embodiments, the 4-1BB agonist is an antibody that specifically binds 4-1BB. In some embodiments, antibody that binds 4-1BB is PF-2566 (PF-05082566; Pfizer) or urelumab (BMS-663513; Bristol Myer Squibb).

In some embodiments, the CD27 agonist is an antibody that specifically binds CD27. In some embodiments, the antibody that binds CD27 is varlilumab (CDX-1127; Celldex).

In some embodiments, the GITR agonist comprises GITR ligand or a GITR-binding portion thereof. In some embodiments, the GITR agonist is an antibody that specifically binds GITR. In some embodiments, the antibody that binds GITR is TRX518 (GITR, Inc.), MK-4166 (Merck), or INBRX-110 (Five Prime Therapeutics/Inhibrx).

TIM-3 has been identified as another important inhibitory receptor expressed by exhausted CD8+ T cells. In mouse models of cancer, it has been shown that the most dysfunctional tumor-infiltrating CD8+ T cells actually co-express PD-1 and TIM-3.

LAG-3 is another recently identified inhibitory receptor that acts to limit effector T-cell function and augment the suppressive activity of T regulatory cells. It has recently been revealed that PD-1 and LAG-3 are extensively co-expressed by tumor-infiltrating T cells in mice, and that combined blockade of PD-1 and LAG-3 provokes potent synergistic antitumor immune responses in mouse models of cancer.

PD-1 pathway blockade can be combined with vaccines or other a compound of Formula I′ antibodies for improved therapeutic efficacy (Hirano, F. et al, Cancer Res., 65(3): 1089-1096 (2005); Li, B. et al, Clin. Cancer Res., 15: 1507-1509 (2009); and Curran, M. A. et al, Proc. Natl. Acad. Set, 107(9):4275-4280 (2010)).

In some embodiments, immunotherapeutic agents useful in the compositions and methods described herein may include a monoclonal antibody, a bispecific antibody comprising one or more immune checkpoint antigen binding moieties, a trispecific antibody, or an immune cell-engaging multivalent antibody/fusion protein/construct known in the art that target specifically both PD-1 and ligand PD-L1.

PD-1 (also known as Programmed Death 1, CD279, PDCD1) is a cell surface receptor with a critical role in regulating the balance between stimulatory and inhibitory signals in the immune system and maintaining peripheral tolerance (Ishida, Y et al. 1992 EMBO J. 11 3887; Kier, Mary E et al. 2008 Annu Rev Immunol 26 677-704; Okazaki, Taku et al. 2007 International Immunology 19 813-824). PD-1 is an inhibitory member of the immunoglobulin super-family with homology to CD28. The structure of PD-1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Expression of PD-1 is inducible on T cells, B cells, natural killer (NK) cells and monocytes, for example upon lymphocyte activation via T cell receptor (TCR) or B cell receptor (BCR) signalling (Kier, Mary E et al. 2008 Annu Rev Immunol 26 677-704; Agata, Y et al 1996 Int Immunol 8 765-72). PD-1 is a receptor for the ligands CD80, CD86, PD-L1 (B7-H1, CD274) and PD-L2 (B7-DC, CD273), which are cell surface expressed members of the B7 family (Freeman, Gordon et al. 2000 J Exp Med 192 1027; Latchman, Y et al. 2001 Nat Immunol 2 261). Upon ligand engagement, PD-1 recruits phosphatases such as SHP-1 and SHP-2 to its intracellular tyrosine motifs which subsequently dephosphorylate effector molecules activated by TCR or BCR signalling (Chemnitz, J et al. 2004 J Immunol 173 945-954; Riley, James L 2009 Immunological Reviews 229 114-125) In this way, PD-1 transduces inhibitory signals into T and B cells only when it is engaged simultaneously with the TCR or BCR.

PD-1 has been demonstrated to down-regulate effector T cell responses via both cell-intrinsic and cell-extrinsic functional mechanisms. Inhibitory signaling through PD-1 induces a state of unresponsiveness in T cells, resulting in the cells being unable to clonally expand or produce optimal levels of effector cytokines. PD-1 may also induce apoptosis in T cells via its ability to inhibit survival signals from co-stimulation, which leads to reduced expression of key anti-apoptotic molecules such as Bcl-XL (Kier, Mary E et al. 2008 Annu Rev Immunol 26 677-704). In addition to these direct effects, recent publications have implicated PD-1 as being involved in the suppression of effector cells by promoting the induction and maintenance of regulatory T cells (TREG). For example, PD-L1 expressed on dendritic cells was shown to act in synergy with TGF-β to promote the induction of CD4+FoxP3+ TREG with enhanced suppressor function (Francisco, Loise M et al. 2009 J Exp Med 206 3015-3029).

TIM-3 (also known as T-cell immunoglobulin and mucin-domain containing-3, TIM-3, Hepatitis A virus cellular receptor 2, HAVCR2, HAVcr-2, KIM-3, TIMID-3, TIMID3, Tim-3, and CD366) is a ˜33.4-kDa single-pass type I membrane protein involved in immune responses (Sanchez-Fueyo et al., Tim-3 inhibits T helper type 1-mediated auto- and alloimmune responses and promotes immunological tolerance, Nat. Immunol. 4:1093-1101(2003)).

TIM-3 is selectively expressed on Th1-cells, and phagocytic cells (e.g., macrophages and dendritic cells). The use of siRNA or a blocking antibody to reduce the expression of human resulted in increased secretion of interferon 7 (IFN-7) from CD4 positive T-cells, implicating the inhibitory role of TIM-3 in human T cells. Analysis of clinical samples from autoimmune disease patients showed no expression of TIM-3 in CD4 positive cells. In particular, expression level of TIM-3 is lower and secretion of IFN-γ is higher in T cell clones derived from the cerebrospinal fluid of patients with multiple sclerosis than those in clones derived from normal healthy persons (Koguchi K et al., J Exp Med. 203:1413-8. (2006)).

TIM-3 is the receptor for the ligands Galectin-9, which is a member of galectin family, molecules ubiquitously expressed on a variety of cell types and which binds β-galactoside; Phospatidyl serine (PtdSer) (DeKryff et al., T cell/transmembrane, Ig, and mucin-3 allelic variants differentially recognize phosphatidylserine and mediate phagocytosis of apoptotic cells, J Immunol. 2010 Feb. 15; 184(4):1918-30); High Mobility Group Protein 1 (also known as HMGB1, HMG1, HMG3, SBP-1, HMG-1, and high mobility group box 1) Chiba et al., Tumor-infiltrating DCs suppress nucleic acid-mediated innate immune responses through interactions between the receptor TIM-3 and the alarmin HMGB1, Nat Immunol. 2012 September; 13(9):832-42); and Carcinoembryonic Antigen Related Cell Adhesion Molecule 1 (also known as CEACAM1, BGP, BGP1, BGPI, carcinoembryonic antigen related cell adhesion molecule 1) (Huang et al., CEACAM1 regulates TIM-3-mediated tolerance and exhaustion, Nature. 2015 Jan. 15; 517(7534):386-90).

BTLA (also known as B- and T-lymphocyte attenuator, BTLA1, CD272, and B and T lymphocyte associated) is a ˜27.3-kDa single-pass type I membrane protein involved in lymphocyte inhibition during immune response. BTLA is constitutively expressed in both B and T cells. BTLA interacts with HVEM (herpes virus-entry mediator), a member of the tumor-necrosis factor receptor (TNFR) family (Gonzalez et al., Proc. Natl. Acad. Sci. USA, 2005, 102: 1116-21). The interaction of BTLA, which belongs to the CD28 family of the immunoglobulin superfamily, and HVEM, a costimulatory tumor-necrosis factor (TNF) receptor (TNFR), is unique in that it defines a cross talk between these two families of receptors. BTLA contains a membrane proximal immunoreceptor tyrosine-based inhibitory motif (ITIM) and membrane distal immunoreceptor tyrosine-based switch motif (ITSM). Disruption of either the ITIM or ITSM abrogated the ability of BTLA to recruit either SHP1 or SHP2, suggesting that BTLA recruits SHP1 and SHP2 in a manner distinct from PD-1 and both tyrosine motifs are required to block T cell activation. The BTLA cytoplasmic tail also contains a third conserved tyrosine-containing motif within the cytoplasmic domain, similar in sequence to a Grb-2 recruitment site (YXN). Also, a phosphorylated peptide containing this BTLA N-terminal tyrosine motif can interact with GRB2 and the p85 subunit of PI3K in vitro, although the functional effects of this interaction remain unexplored in vivo (Gavrieli et al., Bioochem. Biophysi Res Commun, 2003, 312, 1236-43). BTLA is the receptor for the ligands PTPN6/SHP-1; PTPN11/SHP-2; TNFRSF14/HVEM; and B7H4.

VISTA (also known as V-domain Ig suppressor of T cell activation VSIR, B7-H5, B7H5, GI24, PP2135, SISP1, DD1alpha, VISTA, C10orf54, chromosome 10 open reading frame 54, PD-1H, and V-set immunoregulatory receptor) is a ˜33.9-kDa single-pass type I membrane protein involved in T-cell inhibitory response, embryonic stem cells differentiation via BMP4 signaling inhibition, and MMP14-mediated MMP2 activation (Yoon et al., Control of signaling-mediated clearance of apoptotic cells by the tumor suppressor p53, Science. 2015 Jul. 31; 349(6247): 1261669). VISTA interacts with the ligand VSIG-3 (Wang et al., VSIG-3 as a ligand of VISTA inhibits human T-cell function, Immunology. 2019 January; 156(1):74-85)

LAG-3 (also known as Lymphocyte-activation gene 3, LAG3, CD223, and lymphocyte activating 3) is a ˜57.4-kDa single-pass type I membrane protein involved in lymphocyte activation that also binds to HLA class-II antigens. LAG-3 is a member of the immunoglobulin supergene family, and is expressed on activated T cells (Huard et al., 1994, Immunogenetics 39:213), NK cells (Triebel et al., 1990, J. Exp. Med. 171:1393-1405), regulatory T cells (Huang et al., 2004, Immunity 21:503-513; Camisaschi et al., 2010, J Immunol. 184:6545-6551; Gagliani et al., 2013, Nat Med 19:739-746), and plasmacytoid dendritic cells (DCs) (Workman et al., 2009, J Immunol 182:1885-1891). LAG-3 is a membrane protein encoded by a gene located on chromosome 12, and is structurally and genetically related to CD4. Similar to CD4, LAG-3 can interact with MHC class II molecules on the cell surface (Baixeras et al., 1992, J. Exp. Med. 176:327-337; Huard et al., 1996, Eur. J. Immunol. 26:1180-1186). It has been suggested that the direct binding of LAG-3 to MHC class II plays a role in down-regulating antigen-dependent stimulation of CD4+ T lymphocytes (Huard et al., 1994, Eur. J. Immunol. 24:3216-3221) and LAG-3 blockade has also been shown to reinvigorate CD8+ lymphocytes in both tumor or self-antigen (Gross et al., 2007, J Clin Invest. 117:3383-3392) and viral models (Blackburn et al., 2009, Nat. Immunol. 10:29-37). Further, the intra-cytoplasmic region of LAG-3 can interact with LAP (LAG-3-associated protein), which is a signal transduction molecule involved in the downregulation of the CD3/TCR activation pathway (Iouzalen et al., 2001, Eur. J. Immunol. 31:2885-2891). Moreover, CD4+CD25+regulatory T cells (Treg) have been shown to express LAG-3 upon activation, which contributes to the suppressor activity of Treg cells (Huang, C. et al., 2004, Immunity 21:503-513). LAG-3 can also negatively regulate T cell homeostasis by Treg cells in both T cell-dependent and independent mechanisms (Workman, C. J. and Vignali, D. A., 2005, J. Immunol. 174:688-695).

LAG-3 has been shown to interact with MHC class II molecules (Huard et al., CD4/major histocompatibility complex class II interaction analyzed with CD4- and lymphocyte activation gene-3 (LAG-3)-Ig fusion proteins, Eur J Immunol. 1995 September; 25(9):2718-21).

Additionally, several kinases are known to be checkpoint inhibitors. For example, CHEK-1, CHEK-2, and A2aR.

CHEK-1 (also known as CHK 1 kinase, CHK1, and checkpoint kinase 1) is a ˜54.4-kDa serine/threonine-protein kinase that is involved with checkpoint-mediated cell cycle arrest, and the activation of DNA repair in response to the DNA damage and/or unreplicated DNA.

CHEK-2 (also known as CHK2 kinase, CDS1, CHK2, HuCds1, LFS2, PP1425, RAD53, hCds1, and checkpoint kinase 2) is a ˜60.9-kDa. serine/threonine-protein kinase involved in checkpoint-mediated cell cycle arrest, DNA-repair activation, and double-strand break-mediated apoptosis.

A2aR (also known as adenosine A2A receptor, ADORA2A, adenosine A2a receptor, A2aR, ADORA2, and RDC8) is a ˜44.7-kDa multi-pass membrane receptor for adenosine and other ligands.

In various embodiments, the immunotherapeutic agent can comprise an antibody or an antigen binding fragment thereof. Within this definition, immune checkpoint inhibitors include bispecific antibodies and immune cell-engaging multivalent antibody/fusion protein/constructs known in the art. In some embodiments, immunotherapeutic agents which comprise bispecific antibodies may include bispecific antibodies that are bivalent and bind either the same epitope of the immune checkpoint molecule, two different epitopes of the same immune checkpoint molecule or different epitopes of two different immune checkpoints.

Persons of ordinary skill in the art can implement several bispecific antibody formats known in the field to target one or more of CTLA4, PD1, PD-L1 TIM-3, LAG-3, various B-7 ligands, B7H3, B7H4, CHK 1 and CHK2 kinases, BTLA, A2aR, OX40, 41BB, LIGHT, CD40, GITR, TGF-beta, SIRP-alpha, TIGIT, VSIG8, SIGLEC7, SIGLEC9, ICOS, FAS, BTNL2 and other for use in the combination described herein.

In various embodiments, the immunotherapeutic agent can include am immune cell-engaging multivalent antibody/fusion protein/construct.

In an embodiment of the disclosure, the checkpoint inhibitor, in combination with a compound of Formula I′, is used to reduce or inhibit metastasis of a primary tumor or cancer to other sites, or the formation or establishment of metastatic tumors or cancers at other sites distal from the primary tumor or cancer thereby inhibiting or reducing tumor or cancer relapse or tumor or cancer progression.

In a further embodiment of the disclosure, there is provided a combination therapy for treating cancer, comprising a compound of Formula I′ and blockade of checkpoint inhibitors with the potential to elicit potent and durable immune responses with enhanced therapeutic benefit and more manageable toxicity.

In a further embodiment of the disclosure, there is provided a combination therapy for treating cancer, comprising a compound of Formula I′ and an immune checkpoint inhibitor. In an embodiment of the disclosure is provided a method for treating cancer and/or preventing the establishment of metastases by employing a checkpoint inhibitor which act synergistically with a compound of Formula I′.

In further embodiments, methods of the disclosure include, one or more of the following: 1) reducing or inhibiting growth, proliferation, mobility or invasiveness of tumor or cancer cells that potentially or do develop metastases, 2) reducing or inhibiting formation or establishment of metastases arising from a primary tumor or cancer to one or more other sites, locations or regions distinct from the primary tumor or cancer; 3) reducing or inhibiting growth or proliferation of a metastasis at one or more other sites, locations or regions distinct from the primary tumor or cancer after a metastasis has formed or has been established, 4) reducing or inhibiting formation or establishment of additional metastasis after the metastasis has been formed or established, 5) prolonged overall survival, 6) prolonged progression free survival, or 7) disease stabilization.

In an embodiment of the disclosure, administration of the immunotherapeutic agent, in combination therapy with a compound of Formula I′, provides a detectable or measurable improvement in a condition of a given subject, such as alleviating or ameliorating one or more adverse (physical) symptoms or consequences associated with the presence of a cell proliferative or cellular hyperproliferative disorder, neoplasia, tumor or cancer, or metastasis, i e., a therapeutic benefit or a beneficial effect.

A therapeutic benefit or beneficial effect is any objective or subjective, transient, temporary, or long-term improvement in the condition or pathology, or a reduction in onset, severity, duration or frequency of adverse symptom associated with or caused by cell proliferation or a cellular hyperproliferative disorder such as a neoplasia, tumor or cancer, or metastasis. It may lead to improved survival. A satisfactory clinical endpoint of a treatment method in accordance with the disclosure is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis. A therapeutic benefit or improvement therefore may be, but is not limited to destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass, size or cell numbers by inhibiting progression or worsening of the tumor or cancer, can reduce mortality and prolong lifespan even if only for a few days, weeks or months, even though a portion or the bulk of the tumor or cancer mass, size or cells remain.

Specific non-limiting examples of therapeutic benefit include a reduction in neoplasia, tumor or cancer, or metastasis volume (size or cell mass) or numbers of cells, inhibiting or preventing an increase in neoplasia, tumor or cancer volume (e.g., stabilizing), slowing or inhibiting neoplasia, tumor or cancer progression, worsening or metastasis, or inhibiting neoplasia, tumor or cancer proliferation, growth or metastasis.

In an embodiment of the disclosure, administration of the immunotherapeutic agent, in combination therapy with a compound of Formula I′, provides a detectable or measurable improvement or overall response according to the irRC (as derived from time-point response assessments and based on tumor burden), including one of more of the following: (i) irCR-complete disappearance of all lesions, whether measurable or not, and no new lesions (confirmation by a repeat, consecutive assessment no less than 4 weeks from the date first documented), (ii) irPR-decrease in tumor burden .gtoreq.50% relative to baseline (confirmed by a consecutive assessment at least 4 weeks after first documentation).

Optionally, any method described herein may not take effect immediately. For example, treatment may be followed by an increase in the neoplasia, tumor or cancer cell numbers or mass, but over time eventual stabilization or reduction in tumor cell mass, size or numbers of cells in a given subject may subsequently occur.

Additional adverse symptoms and complications associated with neoplasia, tumor, cancer and metastasis that can be inhibited, reduced, decreased, delayed or prevented include, for example, nausea, lack of appetite, lethargy, pain and discomfort. Thus, a partial or complete decrease or reduction in the severity, duration or frequency of adverse symptom or complication associated with or caused by a cellular hyperproliferative disorder, an improvement in the subjects quality of life and/or well-being, such as increased energy, appetite, psychological well-being, are all particular non-limiting examples of therapeutic benefit.

A therapeutic benefit or improvement therefore can also include a subjective improvement in the quality of life of a treated subject. In additional embodiment, a method prolongs or extends lifespan (survival) of the subject. In a further embodiment, a method improves the quality of life of the subject.

In one embodiment, administration of the immunotherapeutic agent, in combination therapy with a compound of Formula I′, results in a clinically relevant improvement in one or more markers of disease status and progression selected from one or more of the following: (i): overall survival, (ii): progression-free survival, (iii): overall response rate, (iv): reduction in metastatic disease, (v): circulating levels of tumor antigens such as carbohydrate antigen 19.9 (CA19.9) and carcinembryonic antigen (CEA) or others depending on tumor, (vii) nutritional status (weight, appetite, serum albumin), (viii): pain control or analgesic use, (ix): CRP/albumin ratio.

Treatment with a compound of Formula I′ in combination with an immunotherapeutic agent gives rise to more complex immunity including not only the development of innate immunity and type-1 immunity, but also immunoregulation which more efficiently restores appropriate immune functions.

In various exemplary methods, a checkpoint inhibitor antibody (monoclonal or polyclonal, bispecific, trispecific, or an immune cell-engaging multivalent antibody/fusion protein/construct) directed to a checkpoint molecule of interest (e.g., PD-1) may be sequenced and the polynucleotide sequence may then be cloned into a vector for expression or propagation. The sequence encoding the antibody or antigen-binding fragment thereof of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, methods for producing the recombinant antibodies can include the steps of culturing a host cell containing isolated nucleic acid(s) encoding the antibodies of the present disclosure. Methods for culturing a host cell containing isolated nucleic acid(s) encoding the antibodies of the present disclosure can be done in a variety of ways, depending on the nature of the antibody. In some embodiments, in the case where the antibodies of the disclosure are full length traditional antibodies, for example, a heavy chain variable region and a light chain variable region under conditions such that an antibody is produced and can be isolated.

In general, nucleic acids are provided that encode the antibodies or antigen-binding fragments thereof of the present disclosure. Such polynucleotides encode for both the variable and constant regions of each of the heavy and light chains, although other combinations are also contemplated by the present disclosure. The present disclosure also contemplates oligonucleotide fragments derived from the disclosed polynucleotides and nucleic acid sequences complementary to these polynucleotides.

The polynucleotides can be in the form of RNA, DNA, cDNA, genomic DNA, nucleic acid analogs, and synthetic DNA. The DNA may be double-stranded or single-stranded, and if single stranded, may be the coding (sense) strand or non-coding (anti-sense) strand. The coding sequence that encodes the polypeptide may be identical to the coding sequence or may be a different coding sequence, which sequence, as a result of the redundancy or degeneracy of the genetic code, encodes the same polypeptides.

In some embodiments, nucleic acid(s) encoding the antibodies of the present disclosure are incorporated into expression vectors, which can be extrachromosomal or designed to integrate into the genome of the host cell into which it is introduced. Expression vectors can contain any number of appropriate regulatory sequences (including, but not limited to, transcriptional and translational control sequences, promoters, ribosomal binding sites, enhancers, origins of replication, and the like.) or other components (selection genes, and the like.), all of which are operably linked as is well known in the art. In some cases two nucleic acids are used and each put into a different expression vector (e.g. heavy chain in a first expression vector, light chain in a second expression vector), or alternatively they can be put in the same expression vector. It will be appreciated by those skilled in the art that the design of the expression vector(s), including the selection of regulatory sequences may depend on such factors as the choice of the host cell, the level of expression of protein desired, and the like.

In general, the nucleic acids and/or expression can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g. in the presence of an inducer, in a suitable non-human animal, in suitable culture media supplemented with appropriate salts, growth factors, antibiotics, nutritional supplements, and the like.), whereby the encoded polypeptide(s) are produced. In some cases, the heavy chains are produced in one cell and the light chain in another.

Mammalian cell lines available as hosts for expression are known in the art and include many immortalized cell lines available from the American Type Culture Collection (ATCC), Manassas, VA USA. including but not limited to Chinese hamster ovary (CHO) cells, HEK 293 cells, NSO cells, HeLa cells, baby hamster kidney (BHK) cells, monkey kidney cells (COS), human hepatocellular carcinoma cells (e.g., Hep G2), and a number of other cell lines. Non-mammalian cells including but not limited to bacterial, yeast, insect, and plants can also be used to express recombinant antibodies. In some embodiments, the antibodies can be produced in transgenic animals such as cows or chickens.

Exemplary and illustrative recombinant methods for antibody molecular biology, expression, purification, and screening are described, for example, in Antibody Engineering, edited by Kontermann & Dubel, Springer, Heidelberg, 2001 and 2010 Hayhurst & Georgiou, 2001, Curr. Opin. Chem. Biol. 5:683-689; Maynard & Georgiou, 2000, Annu. Rev. Biomed. Eng. 2:339-76; and Morrison, S. (1985) Science 229:1202, the disclosures of which are incorporated herein by reference in their entireties.

In various embodiments, the polynucleotide sequence encoding the selected variable heavy and light chains may be used for genetic manipulation to humanize the antibody or to improve the affinity, or other characteristics of the antibody. Antibodies may also be customized for use, for example, in dogs, cats, primate, equines and bovines.

In some embodiments, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif) and HuMAb-Mouse® and TC Mouse™ from Medarex, Inc. (Princeton, N.J.).

Immune checkpoint modulator antibodies of the present disclosure can be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or in yeast cells (e.g. Pichia pastoris or Saccharomyces cerevisiae. Methods for expressing antibodies recombinantly in plants or yeast have been disclosed. See, for example, Peeters, et al. Vaccine 19:2756, 2001; Lonberg, N. and D. Huszar Int. Rev. Immunol 13:65, 1995; and Horwitz, A. H. et al., Proc. Natl. Acad. Sci. 85:8678-8682; the disclosures of which are hereby incorporated by reference in their entireties. Methods for making derivatives of antibodies, e.g., domain, single chain, and the like. are known in the art.

Immunoassays and flow cytometry sorting techniques such as fluorescence activated cell sorting (FACS) can also be employed to isolate antibodies that are specific for checkpoint molecules.

In some embodiments, a polynucleotide comprises a sequence encoding the heavy chain and/or the light chain variable regions of the checkpoint inhibitor antibody or antigen-binding fragment thereof of the present disclosure. The sequence encoding the antibody or antigen-binding fragment thereof of interest may be maintained in a vector in a host cell and the host cell can then be expanded and frozen for future use. Vectors (including expression vectors) and host cells are further described herein.

The disclosure includes affinity matured checkpoint modulator antibodies. For example, affinity matured antibodies can be produced by procedures known in the art (Marks et al., 1992, Bio/Technology, 10:779-783; Barbas et al., 1994, Proc Nat. Acad. Sci. USA 91:3809-3813. One way of characterizing a CDR of an antibody and/or altering (such as improving) the binding affinity of a polypeptide, such as an antibody, termed “library scanning mutagenesis”.

An exemplary method for providing affinity matures antibodies and antigen-binding fragments can include replacing one or more amino acid positions in the CDR with two or more (such as 3, 4, 5, 6, 7, 8, 9, 10, 11, 12, 13, 14, 15, 16, 17, 18, 19, or 20) amino acids using art recognized methods. a library of clones are generated, each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased, or no binding are identified. Methods for determining binding affinity are well-known in the art. Binding affinity may be determined using, for example, Biacore™ surface plasmon resonance analysis, which detects differences in binding affinity of about 2-fold or greater, Kinexa® Biosensor, scintillation proximity assays, ELISA, ORIGEN® immunoassay, fluorescence quenching, fluorescence transfer, and/or yeast display. Binding affinity may also be screened using a suitable bioassay. Biacore™ is particularly useful when the starting antibody already binds with a relatively high affinity, for example a KD of about 10 nM or lower. The library of clones can then be recombinantly introduced into a selection construct using any method known in the art for selection, including phage display, yeast display, and ribosome display.

The antibodies may also be modified, e.g., in the variable domains of the heavy and/or light chains, e.g., to alter a binding property of the antibody. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. For example, a mutation may be made in one or more of the CDR regions to increase or decrease the KD of the antibody directed to a checkpoint molecule, to increase or decrease kon or to alter the binding specificity of the antibody. Techniques in site-directed mutagenesis are well-known in the art. See, e.g., Sambrook et al. and Ausubel et al.

Pharmaceutical compositions containing a compound of Formula I′ according to the present disclosure will comprise an effective amount of a compound of Formula I′, an immunotherapeutic agent, and/or both, typically dispersed in a pharmaceutically acceptable carrier. The phrases “pharmaceutically or pharmacologically acceptable” refers to molecular entities and compositions that do not produce adverse, allergic or other untoward reaction when administered to animal, such as, for example, a human, as appropriate. The preparation of an pharmaceutical composition that contains a compound of Formula I′ will be known to those of skill in the art in light of the present disclosure, as exemplified by Remington's Pharmaceutical Sciences, 18th Ed. Mack Printing Company, 1990, Moreover, for animal (e.g., human) administration, it will be understood that preparations should meet sterility, pyrogenicity, general safety and purity standards. A specific example of a pharmacologically acceptable carrier for a combination compositions, containing a compound of Formula I′ in admixture with an immunotherapeutic agent as described herein is borate buffer or sterile saline solution (0.9% NaCl).

Formulations of the an immunotherapeutic agent, for example an immune checkpoint modulator antibody used in accordance with the present disclosure can be prepared for storage by mixing an antibody having the desired degree of purity with optional pharmaceutically acceptable carriers, excipients or stabilizers as amply described and illustrated in Remington's Pharmaceutical Sciences 16th edition, Osol, A. Ed. [1980], in the form of lyophilized formulations or aqueous solutions and/or suspensions. Acceptable carriers, excipients, buffers or stabilizers are nontoxic to recipients at the dosages and concentrations employed, and include suitable aqueous and/or non-aqueous excipients that may be employed in the pharmaceutical compositions of the disclosure, for example, water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants, buffers such as phosphate, citrate, and other organic acids. Antioxidants may be included, for example, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like; preservatives (such as octade-cyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues). Other exemplary pharmaceutically acceptable excipients may include polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In one illustrative embodiment, the pharmaceutical compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. In some embodiments, the checkpoint inhibitor antibodies or antigen-binding fragments thereof of the present disclosure are formulated for and can be lyophilized for storage and reconstituted in a suitable excipient prior to use according to art-known lyophilization and reconstitution techniques. In one exemplary pharmaceutical composition containing one or more checkpoint inhibitor antibodies or antigen-binding fragment thereof, the composition is formulated as a sterile, preservative-free solution of one or more checkpoint inhibitor antibodies or antigen-binding fragment thereof for intravenous or subcutaneous administration. The formulation can be supplied as either a single-use, prefilled pen, as a single-use, for example containing about 1 mL prefilled glass syringe, or as a single-use institutional use vial. Preferably, the pharmaceutical composition containing the checkpoint inhibitor antibody or antigen-binding fragment thereof is clear and colorless, with a pH of about 6.9-5.0, preferably a pH of 6.5-5.0, and even more preferably a pH ranging from about 6.0 to about 5.0. In various embodiments, the formulations comprising the pharmaceutical compositions can contain from about 500 mg to about 10 mg, or from about 400 mg to about 20 mg, or from about 300 mg to about 30 mg or from about 200 mg to about 50 mg of the checkpoint inhibitor antibody or antigen-binding fragment thereof per mL of solution when reconstituted and administered to the subject. Exemplary injection or infusion excipients can include mannitol, citric acid monohydrate, dibasic sodium phosphate dihydrate, monobasic sodium phosphate dihydrate, polysorbate 80, sodium chloride, sodium citrate and water for parenteral administration, for example, intravenously, intramuscularly, intraperitoneally, or subcutaneous administration.

In another exemplary embodiment, one or more immunotherapeutic agents, or an antigen-binding fragment thereof is formulated for intravenous or subcutaneous administration as a sterile aqueous solution containing 1-75 mg/mL, or more preferably, about 5-60 mg/mL, or yet more preferably, about 10-50 mg/mL, or even more preferably, about 10-40 mg/mL of antibody, with sodium acetate, polysorbate 80, and sodium chloride at a pH ranging from about 5 to 6. Preferably, the intravenous or subcutaneous formulation is a sterile aqueous solution containing 5, 10, 15, 20, 25, 30, 35, 40, 45, or 50 mg/mL of the immunotherapeutic agent, for example, an immune checkpoint inhibitor antibody or an antigen-binding fragment thereof, with 20 mM sodium acetate, 0.2 mg/mL polysorbate 80, and 140 mM sodium chloride at pH 5.5. Further, a solution comprising a checkpoint inhibitor antibody or an antigen-binding fragment thereof, can comprise, among many other compounds, histidine, mannitol, sucrose, trehalose, glycine, poly(ethylene)glycol, EDTA, methionine, and any combination thereof, and many other compounds known in the relevant art.

In one embodiment, a pharmaceutical composition of the present disclosure comprises the following components: 5-500 mg of an immunotherapeutic agent or antigen-binding fragment thereof of the present disclosure, 10 mM histidine, 5% sucrose, and 0.01% polysorbate 80 at pH 5.8, with or without a compound of Formula I′. This composition may be provided as a lyophilized powder. When the powder is reconstituted at full volume, the composition retains the same formulation. Alternatively, the powder may be reconstituted at half volume, in which case the composition comprises 10-500 mg of an immunotherapeutic agent or antigen-binding fragment thereof of the present disclosure, 20 mM histidine, 10% sucrose, and 0.02% polysorbate 80 at pH 5.8.

In one embodiment, part of the dose is administered by an intravenous bolus and the rest by infusion of the immunotherapeutic agent formulation. For example, from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg intravenous injection of the immunotherapeutic agent, or antigen-binding fragment thereof, may be given as a bolus, and the rest of the antibody dose may be administered by intravenous injection. A predetermined dose of the immunotherapeutic agent, or antigen-binding fragment thereof, may be administered, for example, over a period of an hour to two hours to five hours.

In a further embodiment, part of the dose is administered by a subcutaneous injection and/or infusion in the form of a bolus and the rest by infusion of the immunotherapeutic agent formulation. In some exemplary doses, the immunotherapeutic agent formulation can be administered subcutaneously in a dose ranging from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg intravenous injection of the immunotherapeutic agent, or antigen-binding fragment thereof. In some embodiments the dose may be given as a bolus, and the rest of the immunotherapeutic agent dose may be administered by subcutaneous or intravenous injection. A predetermined dose of the immunotherapeutic agent, or antigen-binding fragment thereof, may be administered, for example, over a period of an hour to two hours to five hours.

The formulation herein may also contain more than one active compound as necessary for the particular indication being treated, preferably those with complementary activities that do not adversely affect each other. For example, it may be desirable to provide one or more immunotherapeutic agents with other specificities. Alternatively, or in addition, the composition may comprise an anti-inflammatory agent, a chemotherapeutic agent, a cytotoxic agent, a cytokine, a growth inhibitory agent and/or a small molecule antagonist. Such molecules are suitably present in combination in amounts that are effective for the purpose intended.

The formulations to be used for in vivo administration should be sterile, or nearly so. This is readily accomplished by filtration through sterile filtration membranes.

In various embodiments, illustrative formulations of the pharmaceutical compositions described herein can be prepared using methods widely known in the field of pharmaceutical formulations. In general, such preparatory methods can include the step of bringing the active ingredient into association with a carrier or one or more other accessory ingredients, and then, if desirable, packaging the product into a desired single- or multi-dose unit.

In some embodiments, the composition comprising a compound of Formula I′ can be also delivered in a vesicle, and the immunotherapeutic agent can be delivered in the same liposome formulation, or in a separate formulation that is compatible with the liposomal formulation containing the compound of Formula I′, In some illustrative examples, a liposome containing one or more liposomal surface moieties for example, polyethylene glycol, antibodies and antibody fragments thereof that target a desired tumor surface antigen, receptor, growth factor, glycoprotein, glycolipid or neoantigen, which are selectively transported into specific cells or organs, thus enhance targeted drug delivery.

In another embodiment, a compound of Formula I′ can be delivered in a vesicle, in particular a liposome (see Langer, Science 249:1527-1533 (1990); Treat et al., in LIPOSOIES IN THE THERAPY OF INFECTIOUS DISEASE AND CANCER, Lopez-Berestein and Fidler (eds.), Liss, N.Y., pp. 353-365 (1989); Lopez-Berestein, ibid., pp. 317-327; see generally ibid.).

In yet another embodiment, a compound of Formula I′, or the composition containing the combination, or a composition containing the immunotherapeutic agent, can be delivered in a controlled release system. In one embodiment, a pump can be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, controlled release of the compound of Formula I′ can comprise polymeric materials to provide sustained, intermediate, pulsatile, or alternate release (see MEDICAL APPLICATIONS OF CONTROLLED RELEASE, Langer and Wise (eds.), CRC Pres., Boca Raton, Fla. (1974); CONTROLLED DRUG BIOAVAILABILITY, DRUG PRODUCT DESIGN AND PERFORMANCE, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351(1989); Howard et al., J. Neurosurg. 71:105 (1989)). Other controlled-release systems discussed in the review by Langer (Science 249:1527-1533 (1990)) can be used.

The optimum concentration of the active ingredient(s) in the chosen medium can be determined empirically, according to procedures well known to the skilled artisan, and will depend on the ultimate pharmaceutical formulation desired and the use to be employed.

The present disclosure also provides a pharmaceutical pack or kit comprising one or more containers filled with one or more of the ingredients of the pharmaceutical compositions of the disclosure, which at minimum will include a compound of Formula I′ and one or more checkpoint inhibitor antibodies or antigen-binding fragment thereof as described herein. In other embodiments, the kit may contain one or more further containers providing a pharmaceutically acceptable excipient, for example a diluent. In one embodiment a kit may comprise at least one container, wherein the container can include a compound of Formula I′, a checkpoint inhibitor antibody or an antigen-binding fragment thereof of the present disclosure. The kit may also include a set of instructions for preparing and administering the final pharmaceutical composition to the subject in need thereof, for the treatment of a checkpoint molecule-mediated disease or disorder.

Some embodiments of the present disclosure, the immunotherapeutic agent is a population of immune cells, which can be administered in combination with a compound of Formula I′ to treat a subject with cancer. In some embodiments, the immunotherapeutic agent is a population of immune cells, such as leukocytes (nucleated white blood cells), comprising (e.g., expressing) a receptor that binds to an antigen of interest. A leukocyte of the present disclosure may be, for example, a neutrophil, eosinophil, basophil, lymphocyte or a monocyte. In some embodiments, a leukocyte is a lymphocyte. Examples of lymphocytes include T cells, B cells, Natural Killer (NK) cells or NKT cells. In some embodiments, a T-cell is a CD4+ Th (T helper) cell, a CD8+ cytotoxic T cell, a γδT cell or a regulatory (suppressor) T cell. In some embodiments, an immune cell is a dendritic cell.

Immune cells of the present disclosure, in some embodiments, are genetically engineered to express an antigen-binding receptor. A cell is considered “engineered” if it contains an engineered (exogenous) nucleic acid. Engineered nucleic acids of the present disclosure may be introduced into a cell by any known (e.g., conventional) method. For example, an engineered nucleic acid may be introduced into a cell by electroporation (see, e.g., Heiser W. C. Transcription Factor Protocols: Methods in Molecular Biology™ 2000; 130: 117-134), chemical (e.g., calcium phosphate or lipid), transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), microinjection of purified DNA directly into the nucleus of the cell (see, e.g., Capecchi M. R. Cell. 1980 November; 22(2 Pt 2): 479-88), or retrovirus transduction.

Some aspects of the present disclosure provide an “adoptive cell” approach, which involves isolating immune cells (e.g., T-cells) from a subject with cancer, genetically engineering the immune cells (e.g., to express an antigen-binding receptor, such as a chimeric antigen receptor), expanding the cells ex vivo, and then re-introducing the immune cells into the subject. This method results in a greater number of engineered immune cells in the subject relative to what could be achieved by conventional gene delivery and vaccination methods. In some embodiments, immune cells are isolated from a subject, expanded ex vivo without genetic modification, and then re-introduced into the subject.

Immune cells of the present disclosure comprise receptors that bind to antigens, such as an antigen encoded by an exogenously delivered nucleic acid, as provided herein. In some embodiments, a leukocyte is modified (e.g., genetically modified) to express a receptor that binds to an antigen. The receptor may be, in some embodiments, a naturally-occurring antigen receptor (normally expressed on the immune cell), recombinant antigen receptor (not normally expressed on the immune cell) or a chimeric antigen receptor (CAR). Naturally-occurring and recombinant antigen receptors encompassed by the present disclosure include T cell receptors, B cell receptors, NK cell receptors, NKT cell receptors and dendritic cell receptors. A “chimeric antigen receptor” refers to an artificial immune cell receptor that is engineered to recognize and bind to an antigen expressed by tumor cells. Generally, a CAR is designed for a T cell and is a chimera of a signaling domain of the T-cell receptor (TcR) complex and an antigen-recognizing domain (e.g., a single chain fragment (scFv) of an antibody) (Enblad et al., Human Gene Therapy. 2015; 26(8):498-505), the disclosure of which is incorporated herein by reference in its entirety.

In some embodiments, an antigen binding receptor is a chimeric antigen receptor (CAR). A T cell that expressed a CAR is referred to as a “CAR T cell.” A CAR T cell receptor, in some embodiments, comprises a signaling domain of the T-cell receptor (TcR) complex and an antigen-recognizing domain (e.g., a single chain fragment (scFv) of an antibody) (Enblad et al., Human Gene Therapy. 2015; 26(8):498-505) the disclosure of which is incorporated herein by reference in its entirety.

There are four generations of CARs, each of which contains different components. First generation CARs join an antibody-derived scFv to the CD3zeta (zeta. or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TcR CD3-zeta chain. Third-generation costimulatory domains may include, e.g., a combination of CD3z, CD27, CD28, 4-1BB, ICOS, or OX40. CARs, in some embodiments, contain an ectodomain (e.g., CD3), commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood. 2015; 125(26):4017-4023; Kakarla and Gottschalk, Cancer J. 2014; 20(2):151-155) the disclosure of which is incorporated herein by reference in its entirety.

In some embodiments, the chimeric antigen receptor (CAR) is a T-cell redirected for universal cytokine killing (TRUCK), also known as a fourth generation CAR. TRUCKs are CAR-redirected T-cells used as vehicles to produce and release a transgenic cytokine that accumulates in the targeted tissue, e.g., a targeted tumor tissue. The transgenic cytokine is released upon CAR engagement of the target. TRUCK cells may deposit a variety of therapeutic cytokines in the target. This may result in therapeutic concentrations at the targeted site and avoid systemic toxicity.

CARs typically differ in their functional properties. The CD3zeta signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs, but preclinical in vivo studies suggest that 4-1BB CARs may produce superior proliferation and/or persistence. Clinical trials suggest that both of these second-generation CARs are capable of inducing substantial T-cell proliferation in vivo, but CARs containing the 4-1BB costimulatory domain appear to persist longer. Third generation CARs combine multiple signaling domains (costimulatory) to augment potency. Fourth generation CARs are additionally modified with a constitutive or inducible expression cassette for a transgenic cytokine, which is released by the CAR T-cell to modulate the T-cell response. See, for example, Enblad et al., Human Gene Therapy. 2015; 26(8):498-505; Chmielewski and Hinrich, Expert Opinion on Biological Therapy. 2015; 15(8): 1145-1154 the disclosures of which are incorporated herein by reference in their entireties.

In some embodiments, an illustrative immunotherapeutic agent is a first generation chimeric antigen receptor CAR. In some embodiments, a chimeric antigen receptor is a third generation CAR. In some embodiments, a chimeric antigen receptor is a second generation CAR. In some embodiments, a chimeric antigen receptor is a third generation CAR. In some embodiments, the chimeric antigen receptor is a fourth generation CAR or a T-cell redirected for universal cytokine killing (TRUCK).

In some embodiments, a chimeric antigen receptor (CAR) comprises an extracellular domain comprising an antigen binding domain, a transmembrane domain, and a cytoplasmic domain. In some embodiments, a CAR is fully human. In some embodiments, the antigen binding domain of a CAR is specific for one or more antigens. In some embodiments, a “spacer” domain or “hinge” domain is located between an extracellular domain (comprising the antigen binding domain) and a transmembrane domain of a CAR, or between a cytoplasmic domain and a transmembrane domain of the CAR. A “spacer domain” refers to any oligopeptide or polypeptide that functions to link the transmembrane domain to the extracellular domain and/or the cytoplasmic domain in the polypeptide chain. A “hinge domain” refers to any oligopeptide or polypeptide that functions to provide flexibility to the CAR, or domains thereof, or to prevent steric hindrance of the CAR, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of a CAR.

In some embodiments, a CAR of the disclosure comprises an antigen binding domain, such as a single chain Fv (scFv) specific for a tumor antigen. The choice of binding domain depends upon the type and number of ligands that define the surface of a target cell. For example, the antigen binding domain may be chosen to recognize a ligand that acts as a cell surface marker on target cells associated with a particular disease state, such as cancer or an autoimmune disease. Thus, examples of cell surface markers that may act as ligands for the antigen binding domain in the CAR of the present disclosure include those associated with cancer cells and/or other forms of diseased cells. In some embodiments, a CAR is engineered to target a tumor antigen of interest by way of engineering a desired antigen binding domain that specifically binds to an antigen on a tumor cell encoded by an engineered nucleic acid, as provided herein.

An antigen binding domain (e.g., an scFv) that “specifically binds” to a target or an epitope is a term understood in the art, and methods to determine such specific binding are also known in the art. A molecule is said to exhibit “specific binding” if it reacts or associates more frequently, more rapidly, with greater duration and/or with greater affinity with a particular target antigen than it does with alternative targets. An antigen binding domain (e.g., an scFv) that specifically binds to a first target antigen may or may not specifically bind to a second target antigen. As such, “specific binding” does not necessarily require (although it can include) exclusive binding.

In some embodiments, immune cells expressing a CAR are genetically modified to recognize multiple targets or antigens, which permits the recognition of unique target or antigen expression patterns on tumor cells. Examples of CARs that can bind multiple targets include: “split signal CARs,” which limit complete immune cell activation to tumors expressing multiple antigens; “tandem CARs” (TanCARs), which contain ectodomains having two scFvs; and “universal ectodomain CARs,” which incorporate avidin or a fluorescein isothiocyanate (FITC)-specific scFv to recognize tumor cells that have been incubated with tagged monoclonal antibodies (Mabs).

A CAR is considered “bispecific” if it recognizes two distinct antigens (has two distinct antigen recognition domains). In some embodiments, a bispecific CAR is comprised of two distinct antigen recognition domains present in tandem on a single transgenic receptor (referred to as a TanCAR; see, e.g., Grada Z et al. Molecular Therapy Nucleic Acids 2013; 2:e105, incorporated herein by reference in its entirety). Thus, methods, in some embodiments, comprise delivering to a tumor a combination comprising a compound of Formula I′ and an immunotherapeutic agent, wherein the immunotherapeutic agent is an engineered nucleic acid that encodes an antigen, or delivering to a tumor an engineered nucleic acid that induces expression of a self-antigen, and delivering to the tumor an immune cell expressing a bispecific CAR that binds to two antigens, one of which is encoded by the engineered nucleic acid.

In some embodiments, a CAR is an antigen-specific inhibitory CAR (iCAR), which may be used, for example, to avoid off-tumor toxicity (Fedorov, V D et al. Sci. Transl. Med. published online Dec. 11, 2013, incorporated herein by reference in its entirety). iCARs contain an antigen-specific inhibitory receptor, for example, to block nonspecific immunosuppression, which may result from extra tumor target expression. iCARs may be based, for example, on inhibitory molecules CTLA-4 or PD-1. In some embodiments, these iCARs block T cell responses from T cells activated by either their endogenous T cell receptor or an activating CAR. In some embodiments, this inhibiting effect is temporary.

In some embodiments, CARs may be used in adoptive cell transfer, wherein immune cells are removed from a subject and modified so that they express receptors specific to an antigen, e.g., a tumor-specific antigen. The modified immune cells, which may then recognize and kill the cancer cells, are reintroduced into the subject (Pule, et al., Cytotherapy. 2003; 5(3): 211-226; Maude et al., Blood. 2015; 125(26): 4017-4023, each of which is incorporated herein by reference in their entireties).

According to other aspects of the disclosure, the tumor antigenic component in the vaccine of the invention is any natural or synthetic tumor-associated protein or peptide or combination of tumor-associated proteins and/or peptides or glycoproteins or glycopeptides. In still yet other aspects, the antigenic component can be patient-specific or common to many or most patients with a particular type of cancer. According to one aspect, the antigenic component consists of a cell lysate derived from tumor tissue removed from the patient being treated. In another aspect, the lysate can be engineered or synthesized from exosomes derived from tumor tissue. In yet another aspect, the antigenic component consists of a cell lysate derived from tumor tissue extracted from one or more unrelated individuals or from tumor-cell lines.

In various embodiments, an illustrative immunotherapeutic agent comprises one or more cancer vaccines, for use in combination with a compound of Formula I′. The tumor-associated antigen component of the vaccine may be manufactured by any of a variety of well-known techniques. For individual protein components, the antigenic protein is isolated from tumor tissue or a tumor-cell line by standard chromatographic means such as high-pressure liquid chromatography or affinity chromatography or, alternatively, it is synthesized by standard recombinant DNA technology in a suitable expression system, such as E. coli, yeast or plants. The tumor-associated antigenic protein is then purified from the expression system by standard chromatographic means. In the case of peptide antigenic components, these are generally prepared by standard automated synthesis. Proteins and peptides can be modified by addition of amino acids, lipids and other agents to improve their incorporation into the delivery system of the vaccine (such as a multilamellar liposome). For a tumor-associated antigenic component derived from the patient's own tumor, or tumors from other individuals, or cell lines, the tumor tissue, or a single cell suspension derived from the tumor tissue, is typically homogenized in a suitable buffer. The homogenate can also be fractionated, such as by centrifugation, to isolate particular cellular components such as cell membranes or soluble material. The tumor material can be used directly or tumor-associated antigens can be extracted for incorporation in the vaccine using a buffer containing a low concentration of a suitable agent such as a detergent. An example of a suitable detergent for extracting antigenic proteins from tumor tissue, tumor cells, and tumor-cell membranes is diheptanoyl phosphatidylcholine. Exosomes derived from tumor tissue or tumor cells, whether autologous or heterologous to the patient, can be used for the antigenic component for incorporation in the vaccine or as a starting material for extraction of tumor-associated antigens.

In some embodiments of the present disclosure, a cancer vaccine, wherein the cancer vaccine includes at least one tumor-associated antigen, at least one immunostimulant, and optionally, at least one cell-based immunotherapeutic agent. in some embodiments, the immunostimulant component in the cancer vaccine of the disclosure is any Biological Response Modifier (BRM) with the ability to enhance the therapeutic cancer vaccine's effectiveness to induce humoral and cellular immune responses against cancer cells in a patient. According to one aspect, the immunostimulant is a cytokine or combination of cytokines. Examples of such cytokines include the interferons, such as IFN-gamma, the interleukins, such as IL-2, IL-15 and IL-23, the colony stimulating factors, such as M-CSF and GM-CSF, and tumor necrosis factor. According to another aspect, the immunostimulant component of the disclosed cancer vaccine includes one or more adjuvant-type immunostimulatory agents such as APC Toll-like Receptor agonists or costimulatory/cell adhesion membrane proteins, with or without immunostimulatory cytokines. Examples of Toll-like Receptor agonists include lipid A and CpG, and costimulatory/adhesion proteins such as CD80, CD86, and ICAM-1.

In some embodiments, the immunostimulant is selected from the group consisting of IFN-gamma (IFN-γ), IL-2, IL-15, IL-23, M-CSF, GM-CSF, tumor necrosis factor, lipid A, CpG, CD80, CD86, and ICAM-1, or combinations thereof. According to other aspects, the cell-based immunotherapeutic agent is selected from the group consisting of dendritic cells, tumor-infiltrating T lymphocytes, chimeric antigen receptor-modified T effector cells directed to the patient's tumor type, B lymphocytes, natural killer cells, bone marrow cells, and any other cell of a patient's immune system, or combinations thereof. In one aspect, the cancer vaccine immunostimulant includes one or more cytokines, such as interleukin 2 (IL-2), GM-CSF, M-CSF, and interferon-gamma (IFN-γ), one or more Toll-like Receptor agonists and/or adjuvants, such as monophosphoryl lipid A, lipid A, muramyl dipeptide (MDP) lipid conjugate and double stranded RNA, or one or more costimulatory membrane proteins and/or cell adhesion proteins, such CD80, CD86 and ICAM-1, or any combination of the above. In one aspect, the cancer vaccine includes an immunostimulant that is a cytokine selected from the group consisting of interleukin 2 (IL-2), GM-CSF, M-CSF, and interferon-gamma (IFN-γ). In another aspect, the cancer vaccine includes an immunostimulant that is a Toll-like Receptor agonist and/or adjuvant selected from the group consisting of monophosphoryl lipid A, lipid A, and muramyl dipeptide (MDP) lipid conjugate and double stranded RNA. In yet another aspect, the cancer vaccine includes an immunostimulant that is a costimulatory membrane protein and/or cell adhesion protein selected from the group consisting of CD80, CD86, and ICAM-1.

In various embodiments, an immunotherapeutic agent can include a cancer vaccine, wherein the cancer vaccine incorporates any tumor antigen that can be potentially used to construct a fusion protein according to the invention and particularly the following:

(a) cancer-testis antigens including NY-ESO-1, SSX2, SCP1 as well as RAGE, BAGE, GAGE and MAGE family polypeptides, for example, GAGE-1, GAGE-2, MAGE-1 MAGE-2, MAGE-3, MAGE-4, MAGE-5, MAGE-6, and MAGE-12, which can be used, for example, to address melanoma, lung, head and neck, NSCLC, breast, gastrointestinal, and bladder tumors; (b) mutated antigens, including p53, associated with various solid tumors, e.g., colorectal, lung, head and neck cancer; p21/Ras associated with, e.g., melanoma, pancreatic cancer and colorectal cancer; CDK4, associated with, e.g., melanoma; MUM1 associated with, e.g., melanoma; caspase-8 associated with, e.g., head and neck cancer; CIA 0205 associated with, e.g., bladder cancer; HLA-A2-R1701, beta catenin associated with, e.g., melanoma; TCR associated with, e.g., T-cell non-Hodgkin lymphoma; BCR-abl associated with, e.g., chronic myelogenous leukemia; triosephosphate isomerase; KIA 0205; CDC-27, and LDLR-FUT; (c) over-expressed antigens, including, Galectin 4 associated with, e.g., colorectal cancer; Galectin 9 associated with, e.g., Hodgkin's disease; proteinase 3 associated with, e.g., chronic myelogenous leukemia; WT 1 associated with, e.g., various leukemias; carbonic anhydrase associated with, e.g., renal cancer; aldolase A associated with, e.g., lung cancer; PRAME associated with, e.g., melanoma; HER-2/neu associated with, e.g., breast, colon, lung and ovarian cancer; mammaglobin, alpha-fetoprotein associated with, e.g., hepatoma; KSA associated with, e.g., colorectal cancer; gastrin associated with, e.g., pancreatic and gastric cancer; telomerase catalytic protein, MUC-1 associated with, e.g., breast and ovarian cancer; G-250 associated with, e.g., renal cell carcinoma; p53 associated with, e.g., breast, colon cancer; and carcinoembryonic antigen associated with, e.g., breast cancer, lung cancer, and cancers of the gastrointestinal tract such as colorectal cancer; (d) shared antigens, including melanoma-melanocyte differentiation antigens such as MART-1/Melan A; gp100; MC1R; melanocyte-stimulating hormone receptor; tyrosinase; tyrosinase related protein-1/TRP1 and tyrosinase related protein-2/TRP2 associated with, e.g., melanoma; (e) prostate associated antigens including PAP, PSA, PSMA, PSH-P1, PSM-P1, PSM-P2, associated with e.g., prostate cancer; (f) immunoglobulin idiotypes associated with myeloma and B cell lymphomas. In certain embodiments, the one or more TAA can be selected from pi 5, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, pl85erbB2, pl 80erbB-3, c-met, mn-23H1, TAG-72-4, CA 19-9, CA 72-4, CAM 17.1, NuMa, K-ras, pi 6, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS or any combinations thereof.

In some embodiments, cancer vaccines of the present disclosure for use in combination with a compound of Formula I′ can include a tumor antigen comprising the entire amino acid sequence, a portion of it, or specific immunogenic epitopes of one of the following human proteins: TCTN1 (SEQ ID NO. 1; Gene ID: ENSG00000204852), TCTN2 (SEQ ID NO. 2; Gene ID: ENSG00000168778), TCTN3 (SEQ ID NO. 3; Gene ID: ENSG00000119977), HIGD2A (SEQ ID NO. 4; Gene ID: ENSG00000146066), HIGD2B (SEQ ID NO. 5; Gene ID: ENSG00000175202), C40RF32 (SEQ ID NO. 6; Gene ID: ENSG00000174749), FAM62A (E-SYT1, SEQ ID NO. 7; Gene ID: ENSG00000139641), COLEC11 (SEQ ID NO. 8; Gene ID: ENSG00000118004), FSTL5 (SEQ ID NO. 9; Gene ID: ENSG00000168843), FAM82A2 (SEQ ID NO. 10; Gene ID: ENSG00000137824), SCARA5 (SEQ ID NO. 11; Gene ID: ENSG00000168079), VSTM1 (SEQ ID NO. 12; Gene ID: ENSG00000189068), RNF5 (SEQ ID NO. 13; Gene ID: ENSG00000183574), UNQ6126 (SEQ ID NO. 14; Gene ID: gi|169216088), DPY19L3 (SEQ ID NO. 15; Gene ID: ENSG00000178904), SLC39A10 (SEQ ID NO. 16; gene ID: ENSG00000196950), GPR107 (SEQ ID NO. 17; Gene ID: ENSG00000148358), COL20A1 (SEQ ID NO. 18; Gene ID: ENSG00000101203), GLT25D2 (SEQ ID NO. 19; Gene ID: ENSG00000198756), SYTL3 (SEQ ID NO. 20; Gene ID: ENSG00000164674), DENND1B (SEQ ID NO. 21; Gene ID: ENSG00000162701), C6orf98 (SEQ ID NO. 22; Gene ID: EG: 387079), FAM69B (SEQ ID NO. 23; Gene ID: ENSG00000165716), EMID1 (SEQ ID NO. 24; Gene ID: OTTHUMG00000030824), KLRG2 (SEQ ID NO. 25; GENE ID: ENSG00000188883), ERMP1 (SEQ ID NO. 26; GENE ID: ENSG00000099219), VMO1 (SEQ ID NO. 27; Gene ID: ENSG00000182853), C9orf46 (SEQ ID NO. 28; Gene ID: ENSG00000107020), F1137107 (SEQ ID NO. 29; Gene ID: ENSG00000177990), YIPF2 (SEQ ID NO. 30; Gene ID: ENSG00000130733), TRYX3 (SEQ ID NO. 31; PRSS58, ENSG00000258223.2), C14orf135 (SEQ ID NO. 32; Gene ID: ENSG00000126773), ANGPTL7 (SEQ ID NO. 33; Gene ID: ENSG00000171819), TPCN2 (SEQ ID NO. 34; Gene ID: ENSG00000162341), C18orf19 (SEQ ID NO. 35; Gene ID: ENSG00000177150), OLFML1 (SEQ ID NO. 36; Gene ID: ENSG00000183801), LYPD4 (SEQ ID NO. 37; Gene ID: ENSG00000101203), MEGF8 (SEQ ID NO. 38; Gene ID: ENSG00000105429), F1142986 (SEQ ID NO. 39; Gene ID: ENSG00000196460), SLC46A1 (SEQ ID NO. 40; Gene ID: ENSG00000076351), FAM180A (SEQ ID NO. 41; Gene ID: ENSG00000189320), CRISP-3 (SEQ ID NO. 42; GENE ID: ENSG00000096006), or combinations thereof. These tumor antigens are disclosed in WO2010/086162, WO2010/086163, WO2011/051278, WO2011/051276, WO2011/051277, WO2011/051280, WO2011/051271, WO2011/135068, WO2014/198919, the content of which is herein incorporated by reference in their entireties.

In various embodiments, an illustrative immunotherapeutic agent may include an mRNA operable to encode any one or more of the aforementioned cancer antigens useful for synthesizing a cancer vaccine. In some illustrative embodiments, the mRNA based cancer vaccine may have one or more of the following properties: a) the mRNA encoding each cancer antigen is interspersed by cleavage sensitive sites; b) the mRNA encoding each cancer antigen is linked directly to one another without a linker; c) the mRNA encoding each cancer antigen is linked to one another with a single nucleotide linker; d) each cancer antigen comprises a 20-40 amino acids and includes a centrally located SNP mutation; e) at least 40% of the cancer antigens have a highest affinity for class I MHC molecules from the subject; f) at least 40% of the cancer antigens have a highest affinity for class II MHC molecules from the subject; g) at least 40% of the cancer antigens have a predicted binding affinity of IC>500 nM for HLA-A, HLA-B and/or DRB1; h) the mRNA encodes 1 to 15 cancer antigens; i) 10-60% of the cancer antigens have a binding affinity for class I MHIC and 10-60% of the cancer antigens have a binding affinity for class II MHIC; and/or j) the mRNA encoding the cancer antigens is arranged such that the cancer antigens are ordered to minimize pseudo-epitopes.

In various embodiments, the combination comprising a compound of Formula I′ and a cancer vaccine immunotherapeutic agent as disclosed herein can be used to illicit an immune response in a subject against a cancer antigen. The method involves administering to the subject a RNA vaccine comprising at least one RNA polynucleotide having an open reading frame encoding at least one antigenic polypeptide or an immunogenic fragment thereof, thereby inducing in the subject an immune response specific to the antigenic polypeptide or an immunogenic fragment thereof, in combination with administering a compound of Formula I′ either in the same composition or a separate composition, administered at the same time, or sequentially dosed, wherein the anti-antigenic polypeptide antibody titer in the subject is increased following vaccination relative to anti-antigenic polypeptide antibody titer in a subject vaccinated with a prophylactically effective dose of a traditional vaccine against the cancer. An “anti-antigenic polypeptide antibody” is a serum antibody the binds specifically to the antigenic polypeptide.

A prophylactically effective dose is a therapeutically effective dose that prevents advancement of cancer at a clinically acceptable level. In some embodiments the therapeutically effective dose is a dose listed in a package insert for the vaccine. A traditional vaccine, as used herein, refers to a vaccine other than the mRNA vaccines of the invention. For instance, a traditional vaccine includes but is not limited to live microorganism vaccines, killed microorganism vaccines, subunit vaccines, protein antigen vaccines, DNA vaccines, and the like. In exemplary embodiments, a traditional vaccine is a vaccine that has achieved regulatory approval and/or is registered by a national drug regulatory body, for example the Food and Drug Administration (FDA) in the United States or the European Medicines Agency (EMA.)

In some embodiments the anti-antigenic polypeptide antibody titer in the subject is increased 1 log to 10 log following vaccination relative to anti-antigenic polypeptide antibody titer in a subject vaccinated with a prophylactically effective dose of a traditional vaccine against the cancer. In some embodiments the anti-antigenic polypeptide antibody titer in the subject is increased 1 log following vaccination relative to anti-antigenic polypeptide antibody titer in a subject vaccinated with a prophylactically effective dose of a traditional vaccine against the cancer. In some embodiments the anti-antigenic polypeptide antibody titer in the subject is increased 2 log following vaccination relative to anti-antigenic polypeptide antibody titer in a subject vaccinated with a prophylactically effective dose of a traditional vaccine against the cancer.

Aspects of the invention provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer produced by the mRNA vaccines of the invention is a neutralizing antibody titer. In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

In preferred aspects, RNA vaccine immunotherapeutic agents of the present disclosure (e.g., mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, and the like.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary aspects of the invention, antigen-specific antibodies are measured in units of g/ml or are measured in units of IU/L (International Units per liter) or mIU/ml (milli International Units per ml). In exemplary embodiments of the invention, an efficacious vaccine produces >0.5 μg/mL, >0.1 μg/mL, >0.2 μg/mL, >0.35 μg/mL, >0.5 μg/mL, >1 μg/mL, >2 μg/mL, >5 μg/mL or >10 μg/mL. In exemplary embodiments of the invention, an efficacious vaccine produces >10 mIU/mL, >20 mIU/mL, >50 mIU/mL, >100 mIU/mL, >200 mIU/mL, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay. Also provided are nucleic acid vaccines comprising one or more RNA polynucleotides having an open reading frame encoding a first antigenic polypeptide or a concatemeric polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce a neutralizing antibodies within one week of a single administration. In some embodiments, the adjuvant is selected from a cationic peptide and an immunostimulatory nucleic acid. In some embodiments, the cationic peptide is protamine.

Immunotherapeutic agents comprising a nucleic acid vaccine comprising one or more RNA polynucleotides having an open reading frame comprising at least one chemical modification or optionally no nucleotide modification, the open reading frame encoding a first antigenic polypeptide or a concatemeric polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no nucleotide modification, the open reading frame encoding a first antigenic polypeptide or a concatemeric polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Aspects of the invention also provide a unit of use vaccine, comprising between 10 μg and 400 μg of one or more RNA polynucleotides having an open reading frame comprising at least one chemical modification or optionally no nucleotide modification, the open reading frame encoding a first antigenic polypeptide or a concatemeric polypeptide, and a pharmaceutically acceptable carrier or excipient, formulated for delivery to a human subject. In some embodiments, the vaccine further comprises a cationic lipid nanoparticle.

Aspects of the invention provide methods of creating, maintaining or restoring antigenic memory to a tumor in an individual or population of individuals comprising administering to said individual or population an antigenic memory booster nucleic acid vaccine comprising (a) at least one RNA polynucleotide, said polynucleotide comprising at least one chemical modification or optionally no nucleotide modification and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Aspects of the invention provide methods of vaccinating a subject comprising administering to the subject a single dosage of between 25 μg/kg and 400 μg/kg of a nucleic acid vaccine comprising one or more RNA polynucleotides having an open reading frame encoding a first antigenic polypeptide or a concatemeric polypeptide in an effective amount to vaccinate the subject.

Other aspects provide nucleic acid vaccines comprising one or more RNA polynucleotides having an open reading frame comprising at least one chemical modification, the open reading frame encoding a first antigenic polypeptide or a concatemeric polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

In some embodiments, an illustrative immunotherapeutic agent can include one or more interfering RNAs that can be administered in combination with a compound of Formula I′. An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi). Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

An antisense oligonucleotide can be, for example, about 5, 10, 15, 20, 25, 30, 35, 40, 45, or 50 or more nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the present invention are typically administered to a subject or generated in situ such that they hybridize with or bind to cellular mRNA and/or genomic DNA encoding a polypeptide corresponding to a selected marker of the present invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the present invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

Antigens which can be targeted for synthesizing a corresponding antisense RNA molecule can include any antigen that is specific for one or more tumors, for example, antigens exemplified above with reference to cancer vaccines.

In some embodiments, a combination of an immunotherapeutic agent and a compound of Formula I′ can include a bispecific antibody immunotherapeutic agent. The bispecific antibody can include a protein construct having a first antigen binding moiety and a second antigen binding site that binds to a cytotoxic immune cell. The first antigen binding site can bind to a tumor antigen that is specifically being treated with the combination of the present invention.

For example, the first antigen binding moiety may bind to a non-limiting example of tumor antigens selected from: EGFR, HGFR, Her2, Ep-CAM, CD20, CD30, CD33, CD47, CD52, CD133, CEA, gpA33, Mucins, TAG-72, CIX, PSMA, folate-binding protein, GD2, GD3, GM2, VEGF. VEGFR, Integrin αVβ3, Integrin α5β1, MUC1, ERBB2, ERBB3, MET, IGF1R, EPHA3, TRAILR1, TRAILR2, RANKL, FAP and Tenascin among others. In some embodiments, the first antigen binding moiety has specificity to a protein or a peptide that is overexpressed on a tumor cell as compared to a corresponding non-tumor cell. In some embodiments, the first antigen binding moiety has specificity to a protein that is overexpressed on a tumor cell as compared to a corresponding non-tumor cell. A “corresponding non-tumor cell” as used here, refers to a non-tumor cell that is of the same cell type as the origin of the tumor cell. It is noted that such proteins are not necessarily different from tumor antigens. Non-limiting examples include carcinoembryonic antigen (CEA), which is overexpressed in most colon, rectum, breast, lung, pancreas and gastrointestinal tract carcinomas; heregulin receptors (HER-2, neu or c-erbB-2), which is frequently overexpressed in breast, ovarian, colon, lung, prostate and cervical cancers; epidermal growth factor receptor (EGFR), which is highly expressed in a range of solid tumors including those of the breast, head and neck, non-small cell lung and prostate; asialoglycoprotein receptor; transferrin receptor; serpin enzyme complex receptor, which is expressed on hepatocytes; fibroblast growth factor receptor (FGFR), which is overexpressed on pancreatic ductal adenocarcinoma cells; vascular endothelial growth factor receptor (VEGFR), for anti-angiogenesis gene therapy; folate receptor, which is selectively overexpressed in 90% of nonmucinous ovarian carcinomas; cell surface glycocalyx; carbohydrate receptors; and polymeric immunoglobulin receptor.

The second antigen-binding moiety is any molecule that specifically binds to an antigen or protein or polypeptide expressed on the surface of a cytotoxic immune cell (a CIK cell).

Exemplary non-limiting antigens expressed on the surface of the cytotoxic immune cells suitable for use with the present disclosure may include CD2, CD3, CD4, CD5, CD8, CD11a, CD11 b, CD14, CD16a, CD27, CD28, CD45, CD45RA, CD56, CD62L, the Fc receptor, LFA, LFA-1, TCRαβ, CCR7, macrophage inflammatory protein 1a, perforin, PD-1, PD-L1, PD-L2, or CTLA-4, LAG-3, OX40, 41BB, LIGHT, CD40, GITR, TGF-beta, TIM-3, SIRP-alpha, TIGIT, VSIG8, BTLA, SIGLEC7, SIGLEC9, ICOS, B7H3, B7H4, FAS, BTNL2, CD27 and Fas ligand. In some embodiments, the second antigen binding moiety binds to CD3 of the cytotoxic immune cell, e.g., CIK cell. In some embodiments, the second antigen binding moiety binds to CD56 of the cytotoxic immune cell. In some embodiments, the second antigen binding moiety binds to the Fc receptor of the cytotoxic immune cell. In some embodiments, the Fc region of the bispecific antibody binds to the Fc receptor of the cytotoxic immune cell. In some embodiments, a second antigen-binding moiety is any molecule that specifically binds to an antigen expressed on the surface of a cytotoxic immune cell (e.g., a CIK cell). The second antigen binding moiety is specific for an antigen on a cytotoxic immune cell. Exemplary cytotoxic immune cells include, but are not limited to CIK cells, T-cells, CD8+ T cells, activated T-cells, monocytes, natural killer (NK) cells, NK T cells, lymphokine-activated killer (LAK) cells, macrophages, and dendritic cells. The second antigen binding moiety specifically binds to an antigen expressed on the surface of a cytotoxic immune cell. Exemplary non-limiting antigens expressed on the surface of the cytotoxic immune cells suitable for modulation with the present disclosure may include CD2, CD3, CD4, CD5, CD8, CD11a, CD11 b, CD14, CD16a, CD27, CD28, CD45, CD45RA, CD56, CD62L, the Fc receptor, LFA, LFA-1, TCRαβ, CCR7, macrophage inflammatory protein 1a, perforin, PD-1, PD-L1, PD-L2, or CTLA-4, LAG-3, OX40, 41BB, LIGHT, CD40, GITR, TGF-beta, TIM-3, SIRP-alpha, TIGIT, VSIG8, BTLA, SIGLEC7, SIGLEC9, ICOS, B7H3, B7H4, FAS, BTNL2, CD27 and Fas ligand. In other embodiments, the bispecific antibody modulator is an activator of a costimulatory molecule (e.g., an OX40 agonist). In one embodiment, the OX40 agonist is a bispecific antibody molecule to OX40 and another tumor antigen or a costimulatory antigen. The OX40 agonist can be administered alone, or in combination with other immunomodulators, e.g., in combination with an inhibitor (for example an antibody construct) of PD-1, PD-L1, CTLA-4, CEACAM (e.g., CEACAM-1, -3 and/or -5), TIM-3 or LAG-3. In some embodiments, the anti-OX40 antibody molecule is a bispecific antibody that binds to GITR and PD-1, PD-L1, CTLA-4, CEACAM (e.g., CEACAM-1, -3 and/or -5), TIM-3 or LAG-3. In one exemplary embodiment, an OX40 antibody molecule is administered in combination with an anti-PD-1 antibody molecule (e.g., an anti-PD-1 molecule as described herein). The OX40 antibody molecule and the anti-PD-1 antibody molecule may be in the form of separate antibody composition, or as a bispecific antibody molecule. In other embodiments, the OX40 agonist can be administered in combination with other costimulatory molecule, e.g., an agonist of GITR, CD2, CD27, CD28, CDS, ICAM-1, LFA-1 (CD11a/CD18), ICOS (CD278), 4-1BB (CD137), CD30, CD40, BAFFR, HVEM, CD7, LIGHT, NKG2C, SLAMF7, NKp80, CD160, B7-H3, or CD83 ligand. In some embodiments, the second antigen binding moiety binds to the Fc receptor on the cytotoxic immune cell, e.g., CIK cell.

In some embodiments, the bispecific antibody immunotherapeutic agent has specificities for a tumor antigen and a CIK cell, which brings the tumor antigen expressing tumor cell in close proximity of the CIK cell, leading to the elimination of the tumor cell through anti-tumor cytotoxicity of CIK cell. In some embodiments, the bispecific antibody has specificity for a tumor antigen but does not have specificity for a CIK cell, however, the Fc region of the bispecific antibody can bind to the Fc receptor of the CR cell, which in turn brings the tumor cell in close proximity of the CR cell, leading to the elimination of the tumor cell through anti-tumor cytotoxicity of CIK cell. In some embodiments, the bispecific antibody has specificity for a CR cell but does not have specificity for tumor cell, however, the Fc region of the bispecific antibody can bind to the Fc receptor of the tumor cell, which in turn brings the tumor cell in close proximity of the CIK cell, leading to the elimination of the tumor cell through anti-tumor cytotoxicity of CR cell.

In some embodiments, a combination of an immunotherapeutic agent and a compound of Formula I′ can include an immune cell-engaging multivalent antibody/fusion protein/construct immunotherapeutic agent. In various embodiments, an exemplary immunotherapeutic agent can include immune cell-engaging multivalent antibody/fusion protein/construct which may comprise a recombinant structure, for example, all engineered antibodies that do not imitate the original IgG structure. Here, different strategies to multimerize antibody fragments are utilized. For example, shortening the peptide linker between the V domains forces the scFv to self-associate into a dimer (diabody; 55 kDa). Bispecific diabodies are formed by the noncovalent association of two VHA-VLB and VHB-VLA fragments expressed in the same cell. This leads to the formation of heterodimers with two different binding sites. Single-chain diabodies (sc-diabodies) are bispecific molecules where the VHA-VLB and VHB-VLA fragments are linked together by an additional third linker. Tandem-diabodies (Tandabs) are tetravalent bispecific antibodies generated by two scDiabodies.

Also included are the di-diabodies known in the art. This 130-kDa molecule is formed by the fusion of a diabody to the N-terminus of the CH3 domain of an IgG, resulting in an IgG-like structure. Further diabody derivatives are the triabody and the tetra-body, which fold into trimeric and tetrameric fragments by shortening the linker to <5 or 0-2 residues. Also exemplified are (scFv)₂ constructs known as ‘bispecific T cell engager’ (BITE). BITEs are bispecific single-chain antibodies consisting of two scFv antibody fragments, joined via a flexible linker, that are directed against a surface antigen on target cells and CD3 on T cells. Also exemplified are bivalent (Fab)2 and trivalent (Fab)3 antibody formats. Also exemplified are minibodies and trimerbodies generated from scFvs. Exemplary constructs useful to target tumor antigens as can include one or more of: Diabody, Single-chain (sc)-diabody (scFv)2, Miniantibody, Minibody, Barnase-barstar, scFv-Fc, sc(Fab)2, Trimeric antibody constructs, Triabody antibody constructs, Trimerbody antibody constructs, Tribody antibody constructs, Collabody antibody constructs, (scFv-TNFa)3, F(ab)3/DNL. In each of these exemplified constructs, at least one binding moiety may bind to an antigen or protein or polypeptide expressed on the surface of a cytotoxic immune cell, and at least one binding moiety will bind specifically to an antigen on a cytotoxic immune cell. Exemplary cytotoxic immune cells include, but are not limited to CIK cells, T-cells, CD8+ T cells, activated T-cells, monocytes, natural killer (NK) cells, NK T cells, lymphokine-activated killer (LAK) cells, macrophages, and dendritic cells.

In some embodiments, a combination of an immunotherapeutic agent and a compound of Formula I′ can include a radioconjugate immunotherapeutic agent.

In various embodiments, a radioconjugate is a small molecule or large molecule (herein referred to as a “cell targeting agent”), for example and polypeptide, an antibody or an antibody fragment thereof, that is coupled to or otherwise affixed to a radionuclide, or a plurality of radionuclides, such that the binding of the radioconjugate to its target (a protein or molecule on or in a cancer cell), will lead to the death or morbidity of said cancer cell. In various embodiments, the radioconjugate can be a cell targeting agent labelled with a radionuclide, or the cell targeting agent may be coupled or otherwise affixed to a particle, or microparticle, or nanoparticle containing a plurality of radionuclides, wherein the radionuclides are the same or different. Methods for synthesizing radioconjugates are known in the art, and may include the class of immunoglobulin or antigen binding parts thereof, that are conjugated to a toxic radionuclide.

In some embodiments, the molecule that binds to the cancer cell can be known as a “cell targeting agent”. As used herein, an exemplary cell targeting agent can allow the drug-containing nanoparticles or radionuclide to target the specific types of cells of interest. Examples of cell targeting agents include, but are not limited to, small molecules (e.g., folate, adenosine, purine) and large molecule (e.g., peptide or antibody) that bind to or target a tumor associated antigen. Examples of tumor associated antigens include, but are not limited to, adenosine receptors, alpha v beta 3, aminopeptidase P, alpha fetoprotein, cancer antigen 125, carcinoembryonic antigen, cCaveolin-1, chemokine receptors, clusterin, oncofetal antigens, CD20, epithelial tumor antigen, melanoma associated antigen, Ras, p53, Her2/Neu, ErbB2, ErbB3, ErbB4, folate receptor, prostate-specific membrane antigen, prostate specific antigen, purine receptors, radiation-induced cell surface receptor, serpin B3, serpin B4, squamous cell carcinoma antigens, thrombospondin, tumor antigen 4, tumor-associated glycoprotein 72, tyosinase, and tyrosine kinases. In some embodiments, the cell targeting agent is folate or a folate derivative that binds specifically to folate receptors (FRs). In some embodiments, the cell targeting agent is an antibody, a bispecific antibody, a trispecific antibody or an antigen binding construct thereof, that specifically binds to a cancer antigen selected from: EGFR, HGFR, Her2, Ep-CAM, CD20, CD30, CD33, CD47, CD52, CD133, CEA, gpA33, Mucins, TAG-72, CIX, PSMA, folate-binding protein, GD2, GD3, GM2, VEGF. VEGFR, Integrin αVβ3, Integrin α5β1, MUC1, ERBB2, ERBB3, MET, IGF1R, EPHA3, TRAILR1, TRAILR2, RANKL, FAP and Tenascin among others.

The use of folate as a targeting agent in the radioconjugate also allow both tumor cells and regulatory T (Treg) cells to be targeted for destruction. It is well accepted that high numbers of Treg cells suppress tumor immunity. Specifically, Treg cells suppress (foreign and self) reactive T cells without killing them through contact-dependent or cytokine (e.g., IL-10, TGF-.beta., and the like.) secretion. FR4 is selectively upregulated on Treg cells. It has been shown that antibody blockade of FR4 depleted Treg cells and provoked tumor immunity in tumor-bearing mice. Thus, folate-coated PBM nanoparticles carrying a cytotoxic agent would take FR-expressing cells for their destruction, which would both directly (i.e., BrCa cell) and indirectly (i.e., breast tumor associated and peripheral Treg cells) inhibit tumor progression.

In another further embodiment, the targeting agent is an antibody or peptide, or immune cell-engaging multivalent antibody/fusion protein/constructs capable of binding tumor associated antigens consisting of but not limited to: adenosine receptors, alpha v beta 3, aminopeptidase P, alpha fetoprotein, cancer antigen 125, carcinoembryonic antigen, caveolin-1, chemokine receptors, clusterin, oncofetal antigens, CD20, Human Growth Factor Receptor (HGFR), epithelial tumor antigen, melanoma associated antigen, MUC1, Ras, p53, Her2/Neu, ErbB2, ErbB3, ErbB4, folate receptor, prostate-specific membrane antigen, prostate specific antigen, purine receptors, radiation-induced cell surface receptor, serpin B3, serpin B4, squamous cell carcinoma antigens, thrombospondin, tumor antigen 4, tumor-associated glycoprotein 72, tyrosinase, tyrosine kinases, and the like.

In one embodiment, the treatment method includes the co-administration of a compound as disclosed herein or a pharmaceutically acceptable salt thereof and at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

“Chemotherapeutic agents” include chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478; alkylating agents such as thiotepa and CYTOXAN®; cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5 alpha-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma 11 and calicheamicin omega 11 (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Ore.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylomithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX®; and (ix) pharmaceutically acceptable salts, acids and derivatives of any of the above.

Chemotherapeutic agents also include antibodies, as described above, including alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nivolumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-8744695, Wyeth Research and Abbott Laboratories) which is a recombinant exclusively human-sequence, full-length IgG.sub.1.lamda. antibody genetically modified to recognize interleukin-12 p40 protein.

Chemotherapeutic agents also include “tyrosine kinase inhibitors” including the EGFR inhibitors; small molecule HER2 tyrosine kinase inhibitor such as Mubritonib (TAK165, Takeda); CP-724.714, (Axon Medchem BV, an oral selective inhibitor of the ErbB2 receptor tyrosine kinase); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase 1 inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; antisense molecules (e.g. those that bind to HER-encoding nucleic acid); quinoxalines (U.S. Pat. No. 5,804,396); tryphostins (U.S. Pat. No. 5,804,396); Affinitac (ISIS 3521; Isis/Lilly); PKI166 (Novartis); Semaxinib (Pfizer); INC-1C11 (Imclone), rapamycin (sirolimus, RAPAMIUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca). Tyrosine kinase inhibitors also include Erlotinib (Tarceva®), Gefitinib (Iressa®), Dasatinib (Sprycel®), Nilotinib (Tasigna®), Crizotinib (Xalkori®), Ruxolitinib (Jakafi®), Vemurafenib (Zelboraf®), Vandetanib (Caprelsa®), Pazopanib (Votrient®), afatinib, alisertib, amuvatinib, axitinib, bosutinib, brivanib, canertinib, cabozantinib, cediranib, crenolanib, dabrafenib, dacomitinib, danusertib, dovitinib, foretinib, ganetespib, ibrutinib, iniparib, lenvatinib, linifanib, linsitinib, masitinib, momelotinib, motesanib, neratinib, niraparib, oprozomib, olaparib, pictilisib, ponatinib, quizartinib, regorafenib, rigosertib, rucaparib, saracatinib, saridegib, tandutinib, tasocitinib, telatinib, tivantinib, tivozanib, tofacitinib, trametinib, veliparib, vismodegib, volasertib, cobimetinib (Cotellic®), and others.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNF alpha) blockers such as etanercept (Enbrel), infliximab (Remicade), adalimumab (1-Iumira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/132 blockers such as Anti-lymphotoxin alpha (LTa); miscellaneous investigational agents such as thioplatin, PS-341, phenylbutyrate, ET-18-OCH₃, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE) pixantrone; farnesyltransferase inhibitors such as lonafamib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include Poly ADP ribose polymerase (PARP) inhibitors: olaparib (Lynparza®), rucaprib (Rubraca®) niraparib (Zejula®), talzoparib (Talzenna®).

Effective combinations of compounds of Formula I′ or any formulas as described herein with other agents may be identified through preclinical and clinical testing of the combinations, and will depend on many factors, including disease type and stage of development, overall health of the patient, toxicities and side effects of the agents, and the like.

In some embodiments, compounds as disclosed herein may be used in combination therapy with any of the kinase inhibitors disclosed herein for the treatment of diseases such as cancer. Exemplary kinase inhibitors include imatinib, baricitinib gefitinib, erlotinib, sorafenib, dasatinib, sunitinib, lapatinib, nilotinib, pirfenidone, pazopanib, crizotinib, vemurafenib, vandetanib, ruxolitinib, axitinib, bosutinib, regorafenib, tofacitinib, cabozantinib, ponatinib, trametinib, dabrafenib, afatinib, ibrutinib, ceritinib, idelalisib, nintedanib, palbociclib, lenvatinib, cobimetinib, XL-147, XL-765, XL-499, and XL-880. In some embodiments, a compound as described herein can be used in combination with a HSP90 inhibitor (e.g., XL888), liver X receptor (LXR) modulators, retinoid-related orphan receptor gamma (RORy) modulators, a CK1 inhbitor, a CK1-α inhibitor, a Wnt pathway inhibitor (e.g., SST-215), or a mineralocorticoid receptor inhibitor, (e.g., esaxerenone or XL-550) for the treatment of a disease disclosed herein such as cancer.

In some embodiments, for treatment of cancer, compounds as disclosed herein may be used in combination with inhibitors of PD-1 or inhibitors of PD-L1, e.g., an anti-PD-1 monoclonal antibody or an anti-PD-L1 monoclonal antibody, for example, nivolumab (Opdivo), pembrolizumab (Keytruda, MK-3475), atezolizumab, avelumab, AMP-224, AMP-514, PDR001, durvalumab, pidilizumab (CT-011), CK-301, BMS 936559, and MPDL3280A; CTLA-4 inhibitors, e.g., an anti-CTLA-4 antibody, for example, ipilimumab (Yervoy) and tremelimumab; and phosphatidylserine inhibitors, for example, bavituximab (PGN401); antibodies to cytokines (IL-10, TGF-β, and the like.); other anti-cancer agents such as cemiplimab.

In some embodiments, a compound as described herein can be used in combination with a vaccination protocol for the treatment of cancer. In some embodiments, a compound as described herein can be used in combination with vaccines, to stimulate the immune response to pathogens, toxins, and self antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

In some embodiments, compounds as disclosed herein may be used in combination with inhibitors of PARP, for example, olaparib (Lynparza®), rucaprib (Rubraca®) niraparib (Zejula®), talzoparib (Talzenna®).

The amount of both the compound disclosed herein or salt thereof and the additional one or more additional therapeutic agent (in those compositions which comprise an additional therapeutic agent as described above) that may be combined with carrier materials to produce a single dosage form will vary depending upon the host treated and the particular mode of administration. In certain embodiments, compositions of this invention are formulated such that a dosage of between 0.01-100 mg/kg body weight/day of an inventive can be administered.

The additional therapeutic agent and the compound disclosed herein may act synergistically. Therefore, the amount of additional therapeutic agent in such compositions may be less than that required in a monotherapy utilizing only that therapeutic agent, or there may be fewer side effects for the patient given that a lower dose is used. In certain embodiments, in such compositions a dosage of between 0.01-10,000 μg/kg body weight/day of the additional therapeutic agent can be administered.

Labeled Compounds and Assay Methods

Another aspect of the present invention relates to labeled compounds of the invention (radio-labeled, fluorescent-labeled, and the like.) that would be useful not only in imaging techniques but also in assays, both in vitro and in vivo, for localizing and quantitating TAM kinases in tissue samples, including human, and for identifying TAM kinase ligands by inhibition binding of a labeled compound. Accordingly, the present invention includes TAM kinase assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I. The radionuclide that is incorporated in the instant radio-labeled compounds will depend on the specific application of that radio-labeled compound. For example, for in vitro metalloprotease labeling and competition assays, compounds that incorporate ³H, ¹⁴C, ⁸²Br, ¹²⁵I, ¹³¹, or ³⁵S will generally be most useful. For radio-imaging applications ¹¹C, ¹⁸F, ¹²⁵I, ¹²³I, ¹²⁴I, ¹³I, ⁷⁵Br, ⁷⁶Br, or ⁷⁷Br will generally be most useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S, and ⁸²Br.

The present invention can further include synthetic methods for incorporating radio-isotopes into compounds of the invention. Synthetic methods for incorporating radio-isotopes into organic compounds are well known in the art, and a person of ordinary skill in the art will readily recognize the methods applicable for the compounds of invention.

A labeled compound of the invention can be used in a screening assay to identify/evaluate compounds. For example, a newly synthesized or identified compound (i.e., test compound) which is labeled can be evaluated for its ability to bind a TAM by monitoring its concentration variation when contacting with the TAM kinases, through tracking of the labeling. For example, a test compound (labeled) can be evaluated for its ability to reduce binding of another compound which is known to bind to a TAM kinase (i.e., standard compound). Accordingly, the ability of a test compound to compete with the standard compound for binding to the TAM kinase directly correlates to its binding affinity. Conversely, in some other screening assays, the standard compound is labeled, and test compounds are unlabeled. Accordingly, the concentration of the labeled standard compound is monitored in order to evaluate the competition between the standard compound and the test compound, and the relative binding affinity of the test compound is thus ascertained.

Synthesis

Compounds of this invention can be made by the synthetic procedures described below. The starting materials and reagents used in preparing these compounds are either available from commercial suppliers such as Sigma Aldrich Chemical Co. (Milwaukee, Wis.), or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4^{th} Edition); and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography, and the like. Such materials may be characterized using conventional means, including physical constants and spectral data.

Unless specified to the contrary, the reactions described herein take place at atmospheric pressure and over a temperature range from about −78° C. to about 150° C., more preferably from about 0° C. to about 125° C., and most preferably at about room (or ambient) temperature, e.g., about 20° C. Unless otherwise stated (as in the case of a hydrogenation), all reactions are performed under an atmosphere of nitrogen.

The compounds disclosed and claimed herein have asymmetric carbon atoms or quaternized nitrogen atoms in their structure and may be prepared through the syntheses described herein as single stereoisomers, racemates, or mixtures of enantiomers and diastereomers. The compounds may also exist as geometric isomers. All such single stereoisomers, racemates, and geometric isomers, and mixtures thereof are intended to be within the scope of this invention.

Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereomeric salts or complexes which may be separated, for example, by crystallization; via formation of diastereomeric derivatives which may be separated, for example, by crystallization; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomers may be synthesized by asymmetric synthesis using optically active reagents, substrates, catalysts, or solvents, or by converting on enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The methods of the present invention may be carried out as semi-continuous or continuous processes, more preferably as continuous processes.

The present invention as described above unless indicated otherwise may be carried out in the presence of a solvent or a mixture of two or more solvents. In particular the solvent is an aqueous or an organic solvent such as the ether-like solvent (e.g. tetrahydrofuran, methyltetrahydrofuran, diisopropyl ether, t-butylmethyl ether, or dibutyl ether), aliphatic hydrocarbon solvent (e.g. hexane, heptane, or pentane), saturated alicyclic hydrocarbon solvent (e.g. cyclohexane or cyclopentane), or aromatic solvent (e.g. toluene, o-, m-, or p-xylene, or t-butyl-benzene) or mixture thereof.

The starting materials and reagents, which do not have their synthetic route explicitly disclosed herein, are generally available from commercial sources or are readily prepared using methods well known to the person skilled in the art.

Processes

In one aspect, the invention provides a process for making a compound of Formula I.

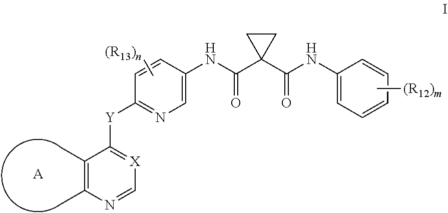

or a pharmaceutically acceptable salt thereof, comprising:
reacting a compound of formula III:

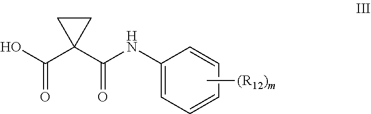

with a compound of formula IV:

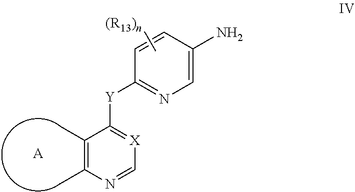

wherein

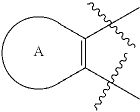

is selected from the group consisting of

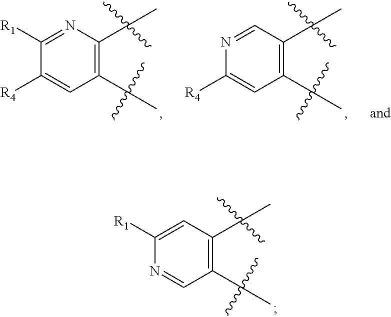

and
X, R₁₃, R₁₂, R₁₈, R₁₉, and Y, are defined herein.

In one aspect, the invention provides a process for making a compound of Formula II:

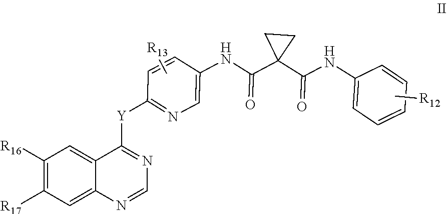

or a pharmaceutically acceptable salt thereof, comprising:
reacting a compound of Formula III:

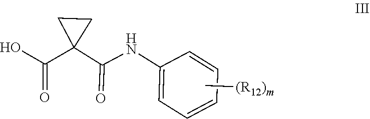

with a compound of Formula V:

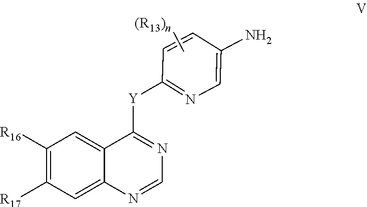

wherein Y, R₁₂, R₁₃, R₁₆, and R₁₇ are defined herein.

In one aspect, the invention provides a process for making a compound of Formula VI

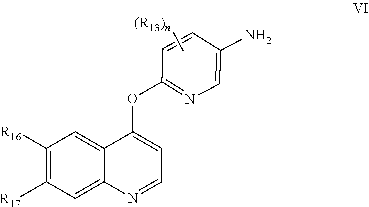

comprising reacting a compound of Formula VII:

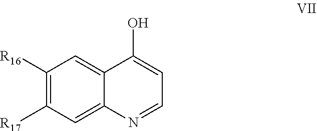

with a compound of Formula VIII:

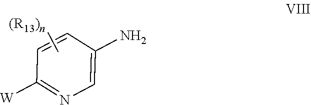

to form a compound of Formula IX:

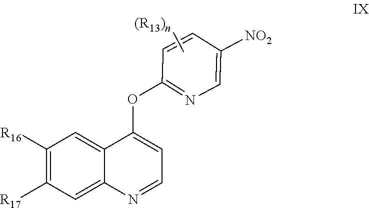

and reducing the compound of Formula IX to provide a compound of Formula VI,
wherein W is a leaving group, such as halogen, and R₁₃, R₁₆, and R₁₇ are defined herein.

The following examples are provided for the purpose of further illustration and are not intended to limit the scope of the claimed invention.

### EXAMPLES

General Experimental Procedures:

The following general procedures are examples of synthesizing compounds of the present invention. One of ordinary skill in the art understands that the general procedures may be adapted to make other compounds of Formula I.

General Procedure A

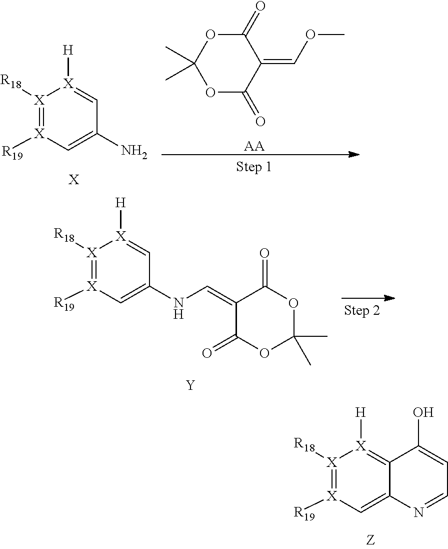

Step 1

A compound of Formula Y can be obtained by reacting a compound of Formula X, wherein the variable X is carbon or nitrogen and wherein R₁₈ and/or R₁₉ is absent when the X variable they are attached to is a nitrogen, with an acetal compound of Formula AA at elevated temperatures in a solvent such as trimethoxymethane or isopropanol. A compound of Formula AA can also be obtained in situ by first reacting 2,2-Dimethyl-1,3-dioxane-4,6-dione in trimethoxymethane prior to adding a compound of Formula X.

Step 2

A compound of Formula Z can be obtained via the intra-cyclization of a compound of Formula Y at elevated temperatures in a high-temperature solvent, such as diphenyl ether or dowtherm.

General Procedure B

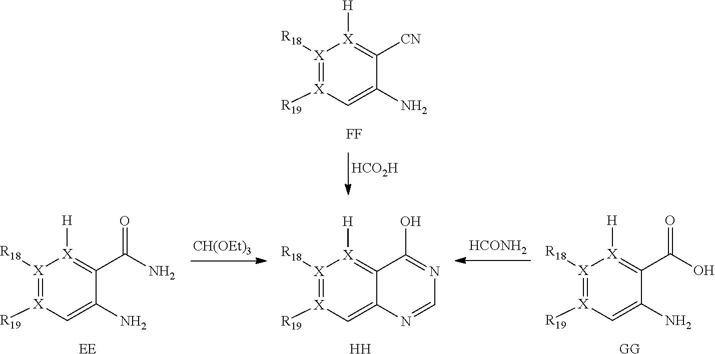

A compound of Formula HH, wherein X is carbon or nitrogen and wherein R₁₈ and/or R₁₉ is absent when the X variable they are attached to is a nitrogen, can be synthesized from a cyano compound of Formula FF, an amide compound of Formula EE, or a carboxylic acid compound of Formula GG. A compound of Formula EE is converted to Formula HH in the presence of triethyl orthoformate under neat conditions at elevated temperatures, optionally under microwave irradiation. A compound of Formula GG is converted to Formula HH in the presence of formamide under neat conditions at elevated temperatures, optionally under microwave irradiation. A compound of Formula FF is converted to Formula HH in the presence of formic acid under neat conditions at elevated temperatures.

General Procedure C

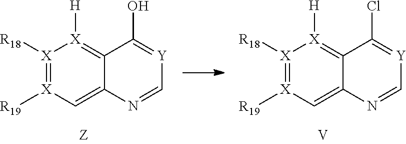

A compound of Formula Z, wherein Y is N or C—H, X is carbon or nitrogen and R₁₈ and/or R₁₉ is absent when the X variable they are attached to is a nitrogen, can be converted to a compound of Formula V by exposure to chloridating reagent such as oxalyl chloride, SOCl₂, or POCl₃. The transformation can be performed in the presence of a solvent or under neat conditions.

General Procedure D

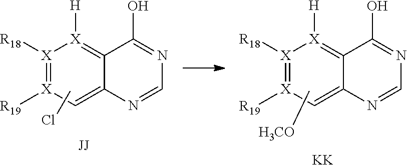

A compound of Formula JJ, wherein X is carbon or nitrogen and wherein R₁₈ and/or R₁₉ is absent when the X variable they are attached to is a nitrogen, can be converted to a compound of Formula KK by reacting with NaOCH₃ in a solvent, preferably anhydrous methanol, at elevated temperature, optionally under microwave irradiation.

General Procedure E

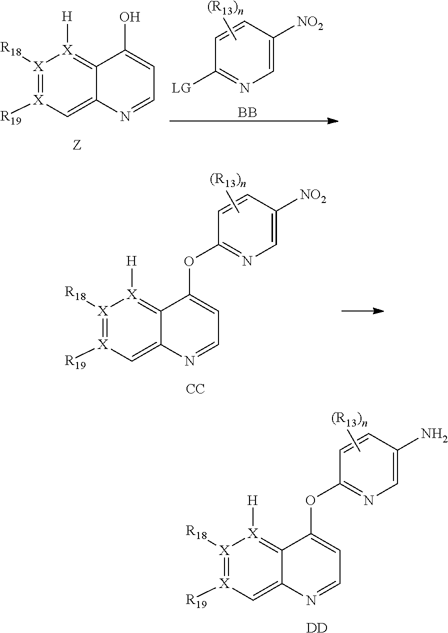

Step 1

A compound of Formula Z, wherein X is carbon or nitrogen and wherein R₁₈ and/or R₁₉ is absent when the X variable they are attached to is a nitrogen, can be converted to a compound of Formula CC by reacting with a compound of Formula BB, wherein “LG” is a leaving group, in the presence of 1) cesium carbonate, or 2) silver oxide, in a solvent such as acetonitrile, DMF, DMSO, or DMA.

Step 2

The nitro moiety of a compound of Formula CC can be reduced to provide a compound of Formula DD using methods known to those skilled in the art, such as hydrogen gas in the presence of Pd/C or nickel metal, or by reduction with iron metal in the presence of NH₄Cl in a solvent such as water, methanol, ethanol, or a combination thereof.

General Procedure F

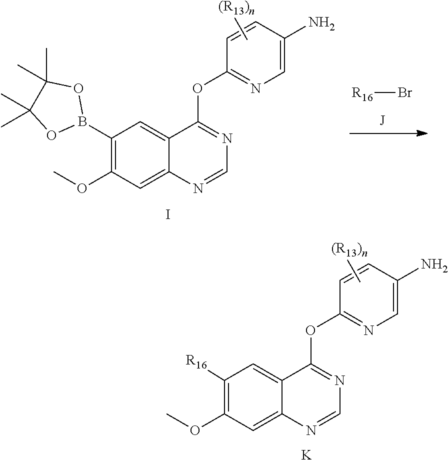

A compound of Formula I can be converted to a compound of Formula K using coupling chemistry, for example a compound of Formula I can be reacted with a compound of Formula J in the presence of a transition metal catalyst, such as bis(di-t-butyl(4-dimethylaminophenyl)phosphine)dichloropalladium(II) in a solvent, such as 1,4-doxane, in the presence of a base, such as sodium carbonate, optionally under microwave irradiation.

General Procedure G

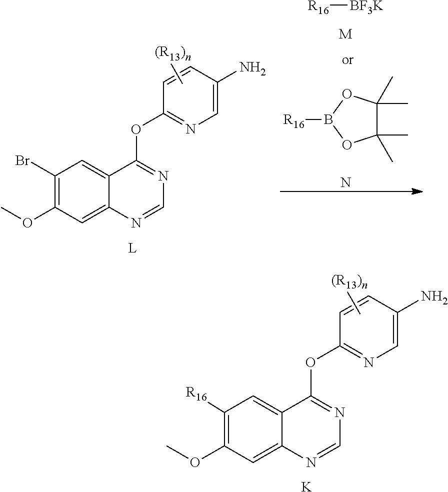

A compound of Formula L can be converted to a compound of Formula K using coupling chemistry, for example a compound of Formula I can be reacted with a compound of Formula M or N in the presence of a transition metal catalyst, such as bis(di-t-butyl(4-dimethylaminophenyl)phosphine)dichloropalladium(II) in a solvent, such as 1,4-doxane, in the presence of a base, such as sodium carbonate, optionally under microwave irradiation.

General Procedure H

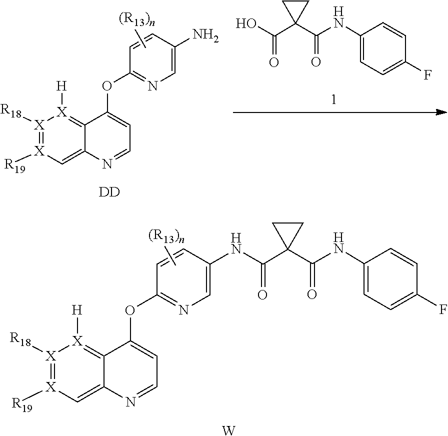

A compound of Formula DD, wherein X is carbon or nitrogen and wherein R₁₈ and/or R₁₉ is absent when the X variable they are attached to is a nitrogen, can be converted to a compound of Formula W by 1) direct coupling with Compound 1 or 2) activation of the carboxylic acid moiety of Compound 1, followed by nucleophilic substitution with a compound of Formula DD. The coupling route can be performed using known coupling reagents, such as EDCI, DCC, HATU, BOP, and the like, in the presence of a base such as, triethylamine, DIEA, pyridine, and the like, and in the presence of a solvent such as DMF, DMA, DCM, THF, and the like. Activation of the carboxylic acid moiety of Compound 1 can be accomplished by first esterification of the carboxylic acid of Compound 1 with a phenolic compound such as pentafluorophenol or para-nitrophenol using means known to one having skill in the art, to form the corresponding phenolate. Second, nucleophilic substitution of the activated Compound 1 with a compound of Formula DD will provide the compound of Formula W.

General Procedure I

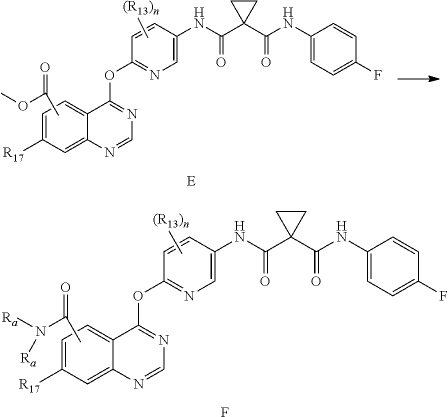

Esters of Formula E can be converted to the corresponding amide compounds of Formula F by first hydrolyzing to the corresponding carboxylic acid and then coupling with ammonia or an amine of the Formula NH(R)₂, wherein each Rₐ can be the same or different, or wherein both Rₐ substituents, together with the nitrogen to which they are attached, form a cyclic structure. The hydrolysis step can be performed with a hydroxide base, such as sodium or lithium hydroxide in a polar solvent such as water, methanol, THF, DMF, DMSO, or any combination thereof. The coupling step can be performed using known coupling reagents, such as EDCI, DCC, HATU, BOP, and the like, in the presence of a base such as, triethylamine, DIEA, pyridine, and the like, and in the presence of a solvent such as DMF, DMA, DCM, THF, and the like.

General Procedure J

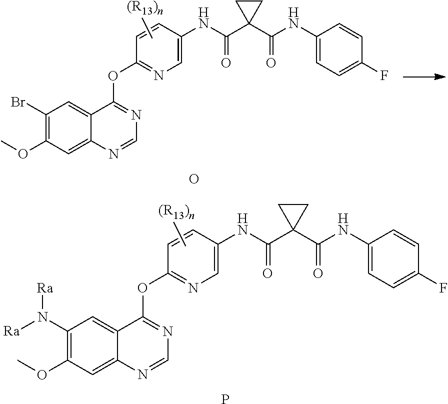

A compound of Formula O can be converted to the corresponding amine compounds of Formula P by coupling with an amine of the Formula NH(Rₐ)₂, wherein each Rₐ can be the same or different, or wherein both Rₐ substituents, together with the nitrogen to which they are attached, form a cyclic structure. The coupling step can be performed using a transition metal catalyst, such as bis(tri-t-butylphosphine)palladium(0) in the presence of a base such as K₃PO₄ in a polar solvent, such as DMF, DMSO, or DMA.

General Procedure K

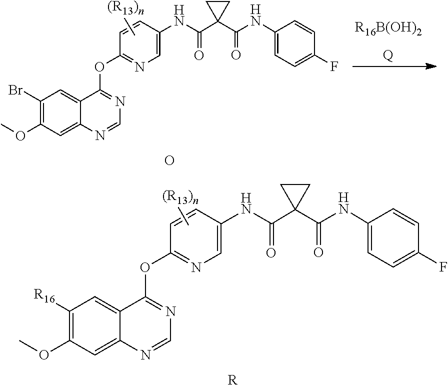

A compound of Formula O can also be converted to a compound of Formula R by coupling with a boronic acid compound of Formula Q in the presence of transition metal catalyst, such as bis(di-t-butyl(4-dimethylaminophenyl)phosphine)dichloropalladium(II), a base, such as sodium carbonate, and a solvent, such as 1,4-dioxane, optionally under microwave irradiation.

Specific Experimental Procedures

### Example 1: Methyl 7-methoxy-4-oxo-1,4-dihydroquinoline-6-carboxylate (4)

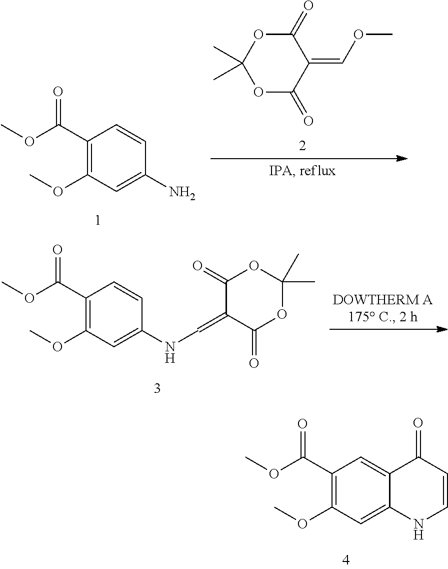

Methyl 4-(((2,2-dimethyl-4,6-dioxo-1,3-dioxan-5-ylidene)methyl)amino)-2-methoxybenzoate (3): To a mixture of Compound 1 (20 g, 110.38 mmol, 1 eq) in IPA (200 mL) was added Compound 2 (21 g, 112.80 mmol, 1.02 eq) in portions at 50° C. The mixture was stirred at 80° C. for 2 h, then cooled down in an ice-water bath for 0.5 h. The resulting mixture was filtered, and the solid was washed with i-PrOH (5 mL) and dried under vacuum to give Compound 3 as a white solid (36 g, 97.3% yield). ¹H NMR (400 MHz, DMSO-d₆) δ 11.26 (br s, 1H), 8.71 (br s, 1H), 7.73 (d, 1H), 7.44 (d, 1H), 7.20 (dd, 1H), 3.87 (s, 3H), 3.77 (s, 3H), 1.68 (s, 6H).

Methyl 7-methoxy-4-oxo-1,4-dihydroquinoline-6-carboxylate (4): After heating biphenylphenoxybenzene (360 mL) to 175° C., Compound 3 (30 g, 89.47 mmol, 1 eq) was added portionwise at 175° C. over 20 min. Stirring was continued at 175° C. for another 2 h. The reaction mixture was cooled to room temperature (16° C.), and petroleum ether (500 mL) was added. The resulting solid was filtered, and the filter cake washed with 100 mL of methyl tertiary butyl ether, dried under vacuum, and purified by flash silica gel chromatography (ISCO®; 120 g SepaFlash® Silica Flash Column, Eluent of 0˜10% CH₃OH/CH₂Cl₂@ 50 mL/min) to give Compound 4 as a yellow solid (6 g, 27.3% yield). ¹H NMR (400 MHz, DMSO-d₆) δ 11.70 (br s, 1H), 8.43 (s, 1H), 7.86 (br d, 1H), 7.02 (s, 1H), 5.99 (d, 1H), 3.89 (s, 3H), 3.81 (s, 3H); MS (EI) for C₁₂H₁₁NO₄, found 233.9 (MH+).

### Example 2: Methyl 7-methoxy-4-((5-nitropyridin-2-yl)oxy)quinoline-6-carboxylate (5)

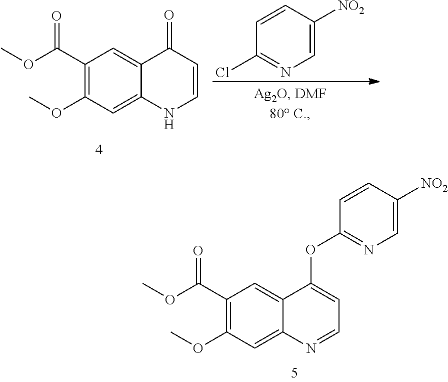

Methyl 7-methoxy-4-((5-nitropyridin-2-yl)oxy)quinoline-6-carboxylate (5): A mixture of Compound 4 (1.0 g, 4.29 mmol, 1 eq), 2-chloro-5-nitro-pyridine (750 mg, 4.73 mmol, 1.1 eq) and Ag₂O (3.00 g, 12.95 mmol, 3.02 eq) in DMF (20 mL) was stirred in the dark at 80° C. for 24 h. The resulting solid was filtered, and the filtrate was diluted with water (100 mL) and extracted with EtOAc (3×80 mL). The combined organic layers were washed with aq saturated NaCl (100 mL), dried over Na₂SO₄, and concentrated. The residue was purified by flash silica gel chromatography (ISCO®; 12 g SepaFlash® Silica Flash Column, Eluent of 0˜4% Methanol/CH₂C₁₂ gradient @ 30 mL/min) to give Compound 5 as a yellow solid (770 mg, 47.99% yield). ¹H NMR (400 MHz, DMSO-d₆) δ 9.06 (d, 1H), 8.95 (d, 1H), 8.76 (dd, 1H), 8.28 (s, 1H), 7.65-7.58 (m, 2H), 7.34 (d, 1H), 3.99 (s, 3H), 3.82 (s, 3H); MS (EI) for C₁₇H₁₃N₃O₆, found 356.1 (MH+).

### Example 3: Methyl 4-((3-chloro-5-nitropyridin-2-yl)oxy)-7-methoxyquinoline-6-carboxylate (6)

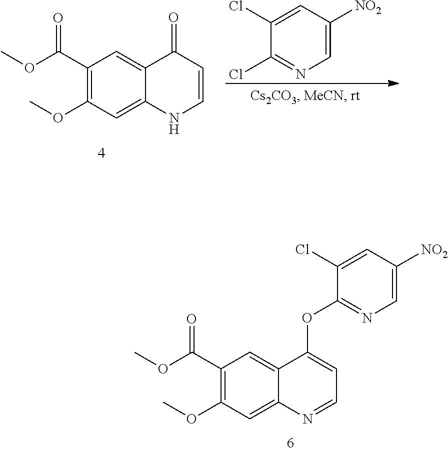

Methyl 4-((3-chloro-5-nitropyridin-2-yl)oxy)-7-methoxyquinoline-6-carboxylate (6): To a solution of Compound 4 (2 g, 8.58 mmol, 1 eq) in CH₃CN (30 mL) was added Cs₂CO₃ (5.59 g, 17.15 mmol, 2 eq) in one portion at 20° C. After stirring at 20° C. for 30 min, 2,3-dichloro-5-nitro-pyridine (1.82 g, 9.43 mmol, 1.1 eq) was added. The mixture was stirred at 20° C. for 36 h. The reaction mixture was filtered and the filter cake was washed with 100 mL of EtOAc. The filter cake diluted with water (100 mL) and extracted with DCM (3×150 mL). The combined DCM extracts were washed with aq saturated NaCl (10 mL), filtered, and concentrated under reduced pressure to give crude Compound 6 as a yellow solid (1.8 g, 53.85% yield). ¹H NMR (400 MHz, DMSO-d₆) δ 9.03 (d, 1H), 8.99-8.95 (m, 2H), 8.26 (s, 1H), 7.64 (s, 1H), 7.42 (d, 1H), 3.99 (s, 3H), 3.82 (s, 3H); MS (EI) for C₁₇H₁₂ClN₃O₆, found 389.9 (MH+).

### Example 4: Methyl 4-((3-fluoro-5-nitropyridin-2-yl)oxy)-7-methoxyquinoline-6-carboxylate (7)

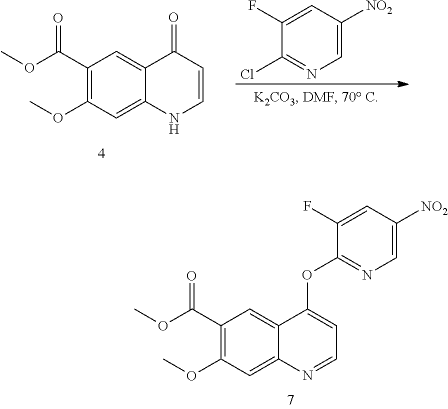 [SMILES: COC(=O)c1cc2c(=O)cc[nH]c2cc1OC ; O=[N+]([O-])c1cnc(Cl)c(F)c1 ; COC(=O)c1cc2c(Oc3ncc([N+](=O)[O-])cc3F)ccnc2cc1OC]

Methyl 4-((3-fluoro-5-nitropyridin-2-yl)oxy)-7-methoxyquinoline-6-carboxylate (7): To a solution of Compound 4 (3 g, 12.22 mmol, 1 eq) and 2-chloro-3-fluoro-5-nitropyridine (2.37 g, 13.44 mmol, 1.1 eq) in DMF (20 mL) was added K₂CO₃ (3.38 g, 24.44 mmol, 2.0 eq) in one portion at 16° C., followed by heating with stirring at 70° C. for 2 h. The reaction mixture was poured into water, and the resulting solids were filtered. The filter cake washed with water (20 mL) and dried under vacuum to give the Compound 7 as a yellow solid (3.5 g, 68.1% yield). ¹H NMR (400 MHz, DMSO-d₆) δ 9.43 (d, 1H), 9.17 (dd, 1H), 8.53 (s, 1H), 8.10 (d, 1H), 6.67 (s, 1H), 6.29 (d, 1H), 3.83 (s, 3H), 3.74 (s, 3H); MS (EI) for C₁₇H₁₂FN₃O₆, found 374.0 (MH+).

### Example 5: Methyl 4-[5-[[1-[(4-fluorophenyl)carbamoyl]cyclopropanecarbonyl]-amino]pyridin-2-yl]oxy-7-methoxyquinoline-6-carboxylate (10)

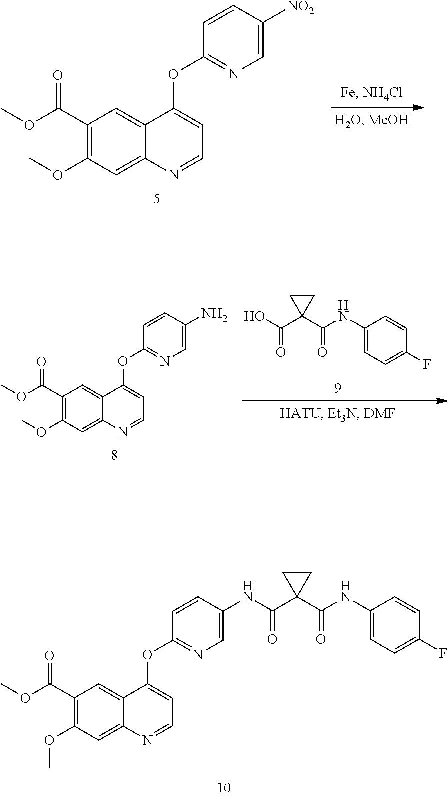 [SMILES: COC(=O)c1cc2c(Oc3ccc([N+](=O)[O-])cn3)ccnc2cc1OC ; COC(=O)c1cc2c(Oc3ccc(N)cn3)ccnc2cc1OC ; O=C(O)C1(C(=O)Nc2ccc(F)cc2)CC1 ; COC(=O)c1cc2c(Oc3ccc(NC(=O)C4(C(=O)Nc5ccc(F)cc5)CC4)cn3)ccnc2cc1OC]

Methyl 4-((5-aminopyridin-2-yl)oxy)-7-methoxyquinoline-6-carboxylate (8): A mixture of Compound 5 (770 mg, 2.17 mmol, 1 eq), Fe (1.21 g, 21.70 mmol, 10 eq) and NH₄Cl (1.16 g, 21.70 mmol, 10 eq) in MeOH (10 mL) and water (2 mL) was heated to reflux at 80° C. for 2.5 h. The mixture was cooled to 20-25° C., and the solid was filtered off through Celite. The filtrate was evaporated, and the residue was diluted with water (30 mL). The resulting precipitate was filtered, and the filter cake was washed with water (2×20 mL) and dried to give Compound 8 as a light yellow solid (500 mg, 1.38 mmol, 63.7% yield). ¹H NMR (400 MHz, CDCl₃) δ 8.78 (s, 1H), 8.68 (d, 1H), 7.84 (d, 1H), 7.49 (s, 1H), 7.18 (d, 1H), 6.98 (d, 1H), 6.69-6.65 (m, 1H), 4.04 (s, 3H), 3.95 (s, 3H), 3.75 (br s, 2H).

Methyl 4-[5-[[1-[(4-fluorophenyl)carbamoyl]cyclopropanecarbonyl]-amino]pyridin-2-yl]oxy-7-methoxyquinoline-6-carboxylate (10): A mixture of Compound 8 (500 mg, 1.54 mmol, 1 eq), Compound 9 (860 mg, 3.85 mmol, 2.5 eq), HATU (1.52 g, 4.00 mmol, 2.6 eq), and triethylamine (470 mg, 4.64 mmol, 3.02 eq) in DMF (10 mL) was stirred at 20° C. for 12 h. The mixture was diluted with water (20 mL) and extracted with EtOAc (3×30 mL). The combined organic layers were washed with aq saturated NaCl (50 mL), dried over Na₂SO₄, and concentrated. The resulting residue was purified by flash silica gel chromatography (ISCO®; 12SepaFlash® Silica Flash Column, Eluent of 0˜100% Ethyl acetate/Petroleum ether gradient @ 25 mL/min) to give Compound 10 as a light yellow solid (550 mg, 67.3% yield). ¹H NMR (400 MHz, CDCl₃) δ 10.15 (s, 1H), 8.75 (d, 1H), 8.71 (s, 1H), 8.41 (d, 1H), 8.22 (dd, 2H), 7.50 (s, 1H), 7.46-7.41 (m, 2H), 7.17 (d, 1H), 7.10-7.03 (m, 2H), 6.85 (d, 1H), 4.04 (s, 3H), 3.95 (s, 3H), 1.87-1.79 (m, 2H), 1.65-1.61 (m, 2H); MS (EI) for C₂₈H₂₃FN₄O₆, found 531.1 (MH+).

The following compounds were prepared in a two-step procedure analogous to that followed for Compound 10 in Example 5:

Methyl 4-[3-chloro-5-[[1-[(4-fluorophenyl)carbamoyl]cyclopropanecarbonyl]-amino]pyridin-2-yl]oxy-7-methoxyquinoline-6-carboxylate (11): Compound 5 in Example 5 was replaced with Compound 6. ¹H NMR (400 MHz, DMSO-d₆) δ 10.52 (s, 1H), 10.05 (s, 1H), 8.80 (d, 1H), 8.51 (d, 1H), 8.46-8.41 (m, 2H), 7.66-7.60 (m, 2H), 7.58 (s, 1H), 7.18-7.12 (m, 2H), 6.91 (d, 1H), 3.99 (s, 3H), 3.85 (s, 3H), 1.51-1.44 (m, 4H); MS (EI) for C₂₈H₂₂ClFN₄O₆, found 565.0 (MH+).

Methyl 4-((3-fluoro-5-(1-((4-fluorophenyl)carbamoyl)cyclopropane-1-carboxamido)pyridin-2-yl)oxy)-7-methoxyquinoline-6-carboxylate (12): Compound 5 in Example 5 was replaced with Compound 7. ¹H NMR (400 MHz, DMSO-d₆) δ 10.89 (br s, 1H), 10.02 (s, 1H), 8.75 (d, 1H), 8.56-8.52 (m, 1H), 8.44 (dd, 1H), 8.03 (d, 1H), 7.64 (dd, 2H), 7.16 (t, 2H), 6.47 (s, 1H), 6.25-6.18 (m, 1H), 3.82 (s, 3H), 3.72-3.69 (m, 3H), 1.58-1.47 (m, 4H); MS (EI) for C₂₈H₂₂F₂N₄O₆, found 549.1 (MH+).

### Example 6: 4-[5-[[1-[(4-Fluorophenyl)carbamoyl]cyclopropanecarbonyl]-amino]pyridin-2-yl]oxy-7-methoxyquinoline-6-carboxylic acid (13)

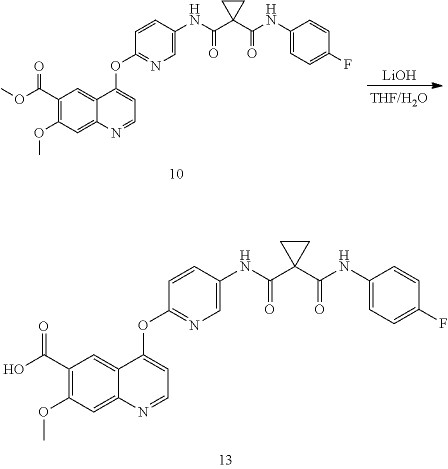

4-[5-[[1-[(4-Fluorophenyl)carbamoyl]cyclopropanecarbonyl]-amino]pyridin-2-yl]oxy-7-methoxyquinoline-6-carboxylic acid (13): A mixture of Compound 10 (300 mg, 565.50 umol, 1 eq) and LiOH·H₂O (71 mg, 1.69 mmol, 2.99 eq) in tetrahydrofuran (3 mL) and water (1 mL) was stirred at 20° C. for 5 h. The mixture was diluted with water (10 mL) and adjusted to pH 5-6 with 1.0 M HCl solution. The resulting precipitate was collected, washed with water (3×20 mL), and lyophilized to give Compound 13 as a white solid (220 mg, 72.3% yield). ¹H NMR (400 MHz, DMSO-d₆) δ 10.32 (s, 1H), 10.11 (s, 1H), 8.72 (d, 1H), 8.48 (d, 1H), 8.25-8.13 (m, 2H), 7.63 (dd, 2H), 7.45 (s, 1H), 7.32 (d, 1H), 7.14 (t, 2H), 6.87 (d, 1H), 3.93 (s, 3H), 1.46 (s, 4H); MS (EI) for C₂₇H₂₁FN₄O₆, found 517.1 (MH+).

The following compounds were prepared in a method analogous to that followed for Compound 13 in Example 6:

4-[3-Chloro-5-[[1-[(4-fluorophenyl)carbamoyl]cyclopropanecarbonyl]amino]pyridin-2-yl]oxy-7-methoxyquinoline-6-carboxylic acid (14): Compound 10 in Example 6 was replaced with Compound 11. ¹H NMR (400 MHz, DMSO-d₆) δ 10.58 (br s, 1H), 10.22 (br s, 1H), 8.67 (d, 1H), 8.50 (br s, 1H), 8.35 (br s, 1H), 7.89 (br s, 1H), 7.64 (br dd, 2H), 7.38 (s, 1H), 7.14 (t, 2H), 6.88 (br d, 1H), 3.89 (s, 3H), 1.45 (br s, 4H); MS (EI) for C₂₇H₂₀ClFN₄O₆, found 551.0 (MH+).

4-[3-Fluoro-5-[[1-[(4-fluorophenyl)carbamoyl]cyclopropanecarbonyl]amino]pyridin-2-yl]oxy-7-methoxyquinoline-6-carboxylic acid (15): Compound 10 in Example 6 was replaced with Compound 12 and NaOH in THF/MeOH/water was used instead of LiOH in THF/water. ¹H NMR (400 MHz, DMSO-d₆) δ 10.16 (br s, 1H), 8.75 (d, 1H), 8.44 (dd, 1H), 8.39 (s, 1H), 7.99 (d, 1H), 7.69-7.61 (m, 2H), 7.20-7.12 (m, 2H), 6.38 (s, 1H), 6.17 (d, 1H), 3.66 (s, 3H), 1.56-1.47 (m, 4H); MS (EI) for C₂₇H₂₀F₂N₄O₆, found 535.1 (MH+).

### Example 7: 1-N′-[6-(6-Carbamoyl-7-methoxyquinolin-4-yl)oxypyridin-3-yl]-1-N-(4-fluorophenyl)cyclopropane-1,1-dicarboxamide (16)

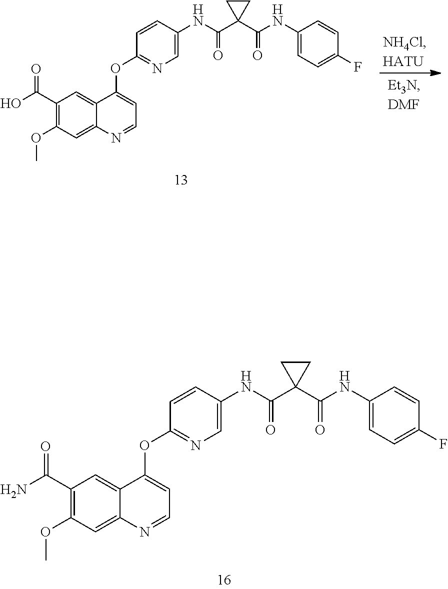 [SMILES: COc1cc2nccc(Oc3ccc(NC(=O)C4(C(=O)Nc5ccc(F)cc5)CC4)cn3)c2cc1C(=O)O ; COc1cc2nccc(Oc3ccc(NC(=O)C4(C(=O)Nc5ccc(F)cc5)CC4)cn3)c2cc1C(N)=O]

1-N′-[6-(6-Carbamoyl-7-methoxyquinolin-4-yl)oxypyridin-3-yl]-1-N-(4-fluorophenyl)-cyclopropane-1,1-dicarboxamide (16): A mixture of Compound 13 (90 mg, 174.26 umol, 1 eq), NH₄Cl (19 mg, 355.20 umol, 2.04 eq), HATU (80 mg, 210.40 umol, 1.21 eq), and TEA (55 mg, 543.53 umol, 3.12 eq) in DMF (2 mL) was stirred at 15° C. for 5 h. The reaction mixture was diluted with water (20 mL) and EtOAc (3 mL). The resulting solids were filtered, and the filter cake was washed with water (2×20 mL), washed with petroleum ether:EtOAc (1:1) (2×20 mL), and lyophilized to give Compound 16 as a white solid (25.0 mg, 27% yield). ¹H NMR (400 MHz, DMSO-d₆) δ 10.33 (br s, 1H), 10.11 (br s, 1H), 8.76 (br d, 1H), 8.50 (br s, 2H), 8.23 (br d, 1H), 7.85 (br s, 1H), 7.73 (br s, 1H), 7.63 (br d, 2H), 7.54 (s, 1H), 7.34 (br d, 1H), 7.14 (br t, 2H), 6.89 (br d, 1H), 4.03 (s, 3H), 1.47 (s, 4H); MS (EI) for C₂₇H₂₂FN₅O₅, found 516.1 (MH+).

The following compound was prepared in a method analogous to that followed for Compound 16 in Example 7:

1-N-(4-Fluorophenyl)-1-N′-[6-[7-methoxy-6-(methylcarbamoyl)quinolin-4-yl]oxypyridin-3-yl]cyclopropane-1,1-dicarboxamide (17): The NH₄Cl in Example 7 was replaced with methylamine hydrochloride. ¹H NMR (400 MHz, DMSO-d₆) δ 10.37 (br s, 1H), 10.14 (br s, 1H), 8.76 (d, 1H), 8.52 (d, 1H), 8.43 (s, 1H), 8.37 (br d, 1H), 8.24 (dd, 1H), 7.65 (dd, 2H), 7.54 (s, 1H), 7.34 (d, 1H), 7.14 (t, 2H), 6.89 (d, 1H), 4.02 (s, 3H), 2.81 (d, 3H), 1.48 (s, 4H); MS (EI) for C₂₈H₂₄FN₅O₅, found 530.1 (MH+).

The following compounds were prepared in a method analogous to that followed for Compound 16 in Example 7, replacing Compound 13 with Compound 14 and using Prep HPLC to purify the final product. DIEA may be used interchangeably with TEA:

1-N′-[6-(6-Carbamoyl-7-methoxyquinolin-4-yl)oxy-5-chloropyridin-3-yl]-1-N-(4-fluorophenyl)cyclopropane-1,1-dicarboxamide (18): ¹H NMR (400 MHz, DMSO-d₆) δ 10.50 (br s, 1H), 10.05 (br s, 1H), 8.78 (d, 1H), 8.51 (s, 1H), 8.48 (s, 1H), 8.40 (s, 1H), 7.86 (br s, 1H), 7.74 (br s, 1H), 7.63 (dd, 2H), 7.56 (s, 1H), 7.15 (t, 2H), 6.93 (br d, 1H), 4.03 (s, 3H), 1.46 (s, 4H); MS (EI) for C₂₇H₂₁ClFN₅O₅, found 550.0 (MH+).

1-N′-[5-Chloro-6-[7-methoxy-6-(methylcarbamoyl)quinolin-4-yl]oxypyridin-3-yl]-1-N-(4-fluorophenyl)cyclopropane-1,1-dicarboxamide (19): The NH₄Cl in Example 7 was replaced with methylamine hydrochloride. ¹H NMR (400 MHz, DMSO-d₆) δ 10.84 (br s, 1H), 10.02 (br s, 1H), 8.87 (d, 1H), 8.64 (d, 1H), 8.53 (s, 1H), 8.19 (br d, 1H), 7.96 (d, 1H), 7.65 (dd, 2H), 7.16 (t, 2H), 6.25-6.13 (m, 2H), 3.71 (s, 3H), 2.80 (d, 3H), 1.52 (br d, 4H); MS (EI) for C₂₈H₂₃ClFN₅O₅, found 564.0 (MH+).

1-N′-[5-Chloro-6-[6-(ethylcarbamoyl)-7-methoxyquinolin-4-yl]oxypyridin-3-yl]-1-N-(4-fluorophenyl)cyclopropane-1,1-dicarboxamide (20): The NH₄Cl in Example 7 was replaced with ethylamine. ¹H NMR (400 MHz, DMSO-d₆) δ 10.50 (br s, 1H), 10.07 (br s, 1H), 8.78 (d, 1H), 8.51 (d, 1H), 8.45-8.36 (m, 3H), 7.63 (dd, 2H), 7.56 (s, 1H), 7.15 (t, 2H), 6.93 (d, 1H), 4.02 (s, 3H), 3.33-3.27 (m, 2H), 1.48-1.46 (m, 4H), 1.13 (t, 3H); MS (EI) for C₂₉H₂₅ClFN₅O₅, found 578.2 (MH+).

The following compounds were prepared in a method analogous to that followed for Compound 16 in Example 7, replacing Compound 13 with Compound 15 and using Prep HPLC to purify the final product:

1-N′-[6-(6-Carbamoyl-7-methoxyquinolin-4-yl)oxy-5-fluoropyridin-3-yl]-1-N-(4-fluorophenyl)cyclopropane-1,1-dicarboxamide (21): ¹H NMR (400 MHz, DMSO-d₆) δ 10.57 (s, 1H), 10.05 (s, 1H), 8.77 (d, 1H), 8.52 (s, 1H), 8.38-8.26 (m, 2H), 7.87 (br s, 1H), 7.75 (br s, 1H), 7.64 (dd, 2H), 7.56 (s, 1H), 7.15 (t, 2H), 6.93 (d, 1H), 4.04 (s, 3H), 1.54-1.43 (m, 4H); MS (EI) for C₂₇H₂₁F₂N₅O₅, found 534.1 (MH+).

1-N′-[5-Fluoro-6-[7-methoxy-6-(methylcarbamoyl)quinolin-4-yl]oxypyridin-3-yl]-1-N-(4-fluorophenyl)cyclopropane-1,1-dicarboxamide (22): The NH₄Cl in Example 7 was replaced with methylamine hydrochloride. ¹H NMR (400 MHz, DMSO-d₆) δ 10.57 (br s, 1H), 10.05 (br s, 1H), 8.77 (d, 1H), 8.46 (s, 1H), 8.42-8.28 (m, 3H), 7.63 (dd, 2H), 7.56 (s, 1H), 7.15 (t, 2H), 6.93 (d, 1H), 4.02 (s, 3H), 2.82 (d, 3H), 1.56-1.41 (m, 4H); MS (EI) for C₂₈H₂₃F₂N₅O₅, found 548.1 (MH+).

1-N′-[6-[6-(Ethylcarbamoyl)-7-methoxyquinolin-4-yl]oxy-5-fluoropyridin-3-yl]-1-N-(4-fluorophenyl)cyclopropane-1,1-dicarboxamide (23): The NH₄Cl in Example 7 was replaced with ethylamine. ¹H NMR (400 MHz, DMSO-d₆) δ 10.57 (s, 1H), 10.05 (s, 1H), 8.76 (d, 1H), 8.47-8.27 (m, 4H), 7.64 (dd, 2H), 7.55 (s, 1H), 7.15 (t, 2H), 6.92 (d, 1H), 4.02 (s, 3H), 3.33-3.27 (m, 2H), 1.54-1.41 (m, 4H), 1.13 (t, 3H); MS (EI) for C₂₉H₂₅F₂N₅O₅, found 562.1 (MH+).

### Example 8: 1-N′-[5-Fluoro-6-[7-methoxy-6-[3-(methoxymethyl)-1,2,4-oxadiazol-5-yl]quinolin-4-yl]oxypyridin-3-yl]-1-N-(4-fluorophenyl)cyclopropane-1,1-dicarboxamide (25)

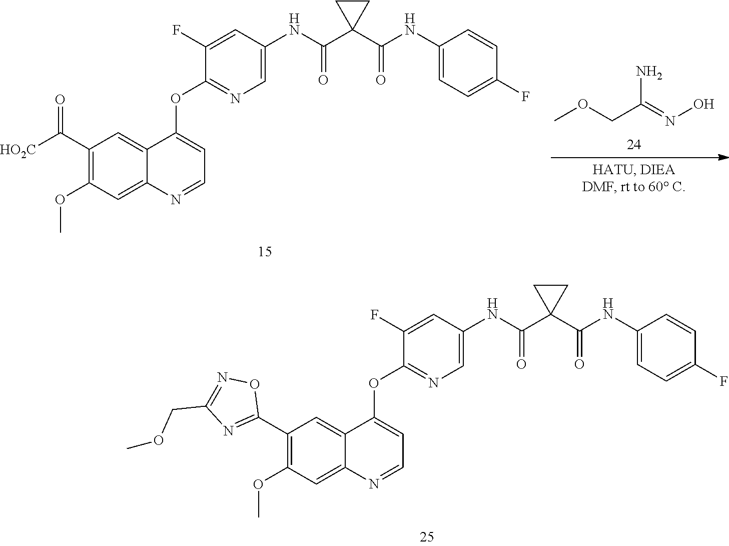

1-N′-[5-Fluoro-6-[7-methoxy-6-[3-(methoxymethyl)-1,2,4-oxadiazol-5-yl]quinolin-4-yl]oxypyridin-3-yl]-1-N-(4-fluorophenyl)cyclopropane-1,1-dicarboxamide hydrochloride (25): To a solution of Compound 15 (50 mg, 0.093 mmol), Compound 24 (10 mg, 0.093 mmol), and DIEA (49 uL, 0.28 mmol) in DMF (0.5 mL) was added HATU (43 mg, 0.112 mmol), and the reaction was stirred at room temperature until the starting material disappeared. The reaction mixture was heated to 60° C. for 2 h. Aq saturated NaHCO₃ was added to the resulting mixture and was extracted with DCM (3×). The combined organic extracts were washed with aq saturated NaCl, dried over Na₂SO₄, and concentrated. The resulting crude residue was purified by prep HPLC (Gemini-NX, 10 uM, 250×30 mm, C₁₈ column, Phenomenex, Torrance, Ca.; eluent: 0.1 to 100% acetonitrile in water, both eluents containing 0.1% trifluoroacetic acid, gradient elution over 15 min). After lyophilizing the purified product, the resulting powder was brought up in 20% MeOH/DCM and passed through an Agilent PL-HCO₃ ion exchange column (or brought up in DCM and washed with saturated sodium bicarbonate) to remove residual acids. After concentrating, to the resulting residue was added HCl (4M in Dioxane, 0.5 ml; or 1M aqueous, 1 mL), and the solvent was removed under reduced pressure and dried under high vacuum to give Compound 25 as the HCl salt (2.1 mg, 4% yield). ¹H NMR (400 MHz, CD₃OD) δ 9.27 (s, 1H), 8.46 (d, 1H), 8.20 (q, 1H), 7.99 (s, 1H), 7.95 (d, 1H), 7.55 (m, 2H), 7.07 (t, 2H), 6.74 (d, 1H), 4.57 (s, 2H), 4.10 (s, 3H), 3.40 (s, 3H), 1.73-1.62 (m, 4H); MS (EI) for C₃₀H₂₄F₂N₆O₆, found 603.1 (MH+).

The following compounds were prepared as the HCl salts in a method analogous to that followed for Compound 25 in Example 8:

1-N′-[5-Fluoro-6-[7-methoxy-6-[3-(2-methoxyethyl)-1,2,4-oxadiazol-5-yl]quinolin-4-yl]oxypyridin-3-yl]-1-N-(4-fluorophenyl)cyclopropane-1,1-dicarboxamide hydrochloride (26): Compound 24 in Example 8 was replaced with (Z)—N′-hydroxy-3-methoxypropanimidamide. ¹H NMR (400 MHz, CD₃OD) δ 9.24 (s, 1H), 8.46 (d, 1H), 8.21 (q, 1H), 7.99 (s, 1H), 7.93 (d, 1H), 7.55 (m, 2H), 7.07 (q, 2H), 6.74 (d, 1H), 4.11 (s, 3H), 3.72 (t, 2H), 3.30 (s, 3H), 2.97 (t, 2H), 1.72-1.58 (m, 4H); MS (EI) for C₃₁H₂₆F₂N₆O₆, found 617.2 (MH+).

1-N′-[6-[6-(3-Ethyl-1,2,4-oxadiazol-5-yl)-7-methoxyquinolin-4-yl]oxy-5-fluoropyridin-3-yl]-1-N-(4-fluorophenyl)cyclopropane-1,1-dicarboxamide hydrochloride (27): Compound 24 in Example 8 was replaced with (Z)—N′-hydroxypropionimidamide. ¹H NMR (400 MHz, CD₃OD) δ 9.25 (s, 1H), 8.57 (d, 1H), 8.20 (q, 1H), 8.06 (s, 1H), 7.94 (d, 1H), 7.56 (q, 2H), 7.07 (q, 2H), 6.85 (d, 1H), 4.16 (s, 3H), 2.75 (q, 2H), 1.75-1.53 (m, 4H), 1.27 (t, 3H); MS (EI) for C₃₀H₂₄F₂N₆O₅, found 587.1 (MH+).

### Example 9: 1-N′-[5-Chloro-6-[7-(2-hydroxyethoxy)-6-(methylcarbamoyl)quinolin-4-yl]oxypyridin-3-yl]-1-N-(4-fluorophenyl)cyclopropane-1,1-dicarboxamide (37)

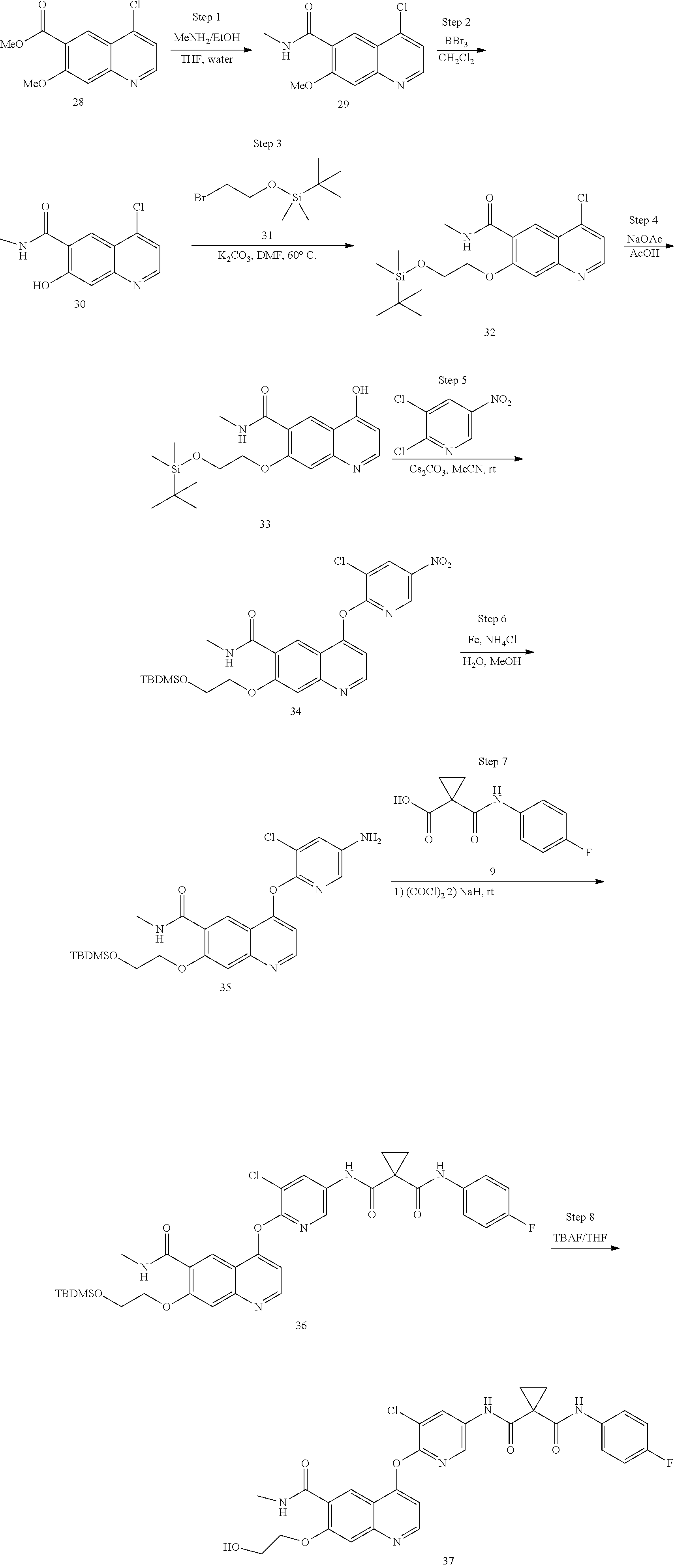

4-Chloro-7-methoxy-N-methylquinoline-6-carboxamide (29): Methylamine (8 M, 50 mL, 10.07 eq) in EtOH was added to a solution of Compound 28 (10 g, 39.74 mmol, 1 eq) in THF (150 mL) at 30° C., and the reaction mixture was stirred at 30° C. for 25 h. The reaction mixture was concentrated under vacuum, and the residue was slurried with warm water (100 mL). The resulting solid was filtered and dried under vacuum to give Compound 29 as a white solid (9 g, 90.4% yield). MS (EI) for C₁₂H₁₁ClN₂O₂, found 251.0 (MH+).

4-Chloro-7-hydroxy-N-methylquinoline-6-carboxamide (30): To a stirred solution of Compound 29 (2 g, 7.98 mmol, 1 eq) in 1, 2-dichloroethane (120 mL) was added BBr₃ (6.00 g, 23.93 mmol, 2.31 mL, 3 eq) dropwise at 0° C. The reaction mixture was stirred at 80° C. for 3 h, after which the mixture was poured into aq saturated NaHCO₃ (300 mL). The resulting precipitate was filtered, and the filter cake was washed with water (75 mL) and dried to give Compound 30 as a yellow solid (1.5 g, 79.45% yield). MS (EI) for C₁₁H₉ClN₂O₂, found 237.0 (MH+).

7-(2-((tert-Butyldimethylsilyl)oxy)ethoxy)-4-chloro-N-methylquinoline-6-carboxamide (32): A mixture of Compound 30 (1.25 g, 5.28 mmol, 1 eq), Compound 31 (2.53 g, 10.56 mmol, 2 eq), and K₂CO₃ (2.19 g, 15.85 mmol, 3 eq) in DMF (30 mL) was stirred at 60° C. for 12 h. Water (250 mL) was added. The resulting solid was filtered, and the filter cake washed with water (50 mL), washed with petroleum ether (80 mL) and dried to give Compound 32 as a yellow solid (1.9 g, 91.1% yield) which was used in subsequent reactions without further purification. ¹H NMR (400 MHz, CDCl₃) δ 9.14 (s, 1H), 8.74 (d, 1H), 8.20 (br s, 1H), 7.51 (s, 1H), 7.41 (d, 1H), 4.37-4.29 (m, 2H), 4.15-4.08 (m, 2H), 3.06 (d, 3H), 0.93 (s, 9H), 0.13 (s, 6H); MS (EI) for C₁₉H₂₇ClN₂O₃Si, found 395.1 (MH+).

7-(2-((tert-Butyldimethylsilyl)oxy)ethoxy)-4-hydroxy-N-methylquinoline-6-carboxamide (33): A mixture of Compound 32 (1.9 g, 4.81 mmol, 1 eq) and NaOAc (789.22 mg, 9.62 mmol, 2 eq) in AcOH (30 mL) was stirred at 120° C. for 1 h. The reaction mixture was concentrated, and water (150 mL) was added at 16° C. The resulting mixture was filtered, and the filter cake was washed with water (50 mL) and EtOAc (15 mL) and dried to give Compound 33 as a yellow solid (1.1 g, 60.73% yield), which was used in subsequent reactions without further purification. ¹H NMR (400 MHz, DMSO-d₆) δ 8.52 (s, 1H), 8.11 (br d, 1H), 7.84 (d, 1H), 7.16 (s, 1H), 5.95 (d, 1H), 4.24-4.15 (m, 2H), 4.07-3.98 (m, 2H), 2.82 (br d, 3H), 0.86 (s, 9H), 0.07 (s, 6H); MS (EI) for C₁₉H₂₈N₂O₄Si, found 377.2 (MH+).

7-(2-((tert-Butyldimethylsilyl)oxy)ethoxy)-4-((3-chloro-5-nitropyridin-2-yl)oxy)-N-methylquinoline-6-carboxamide (34): Compound 34 was synthesized from Compound 33 and 2,3-dichloro-5-nitro-pyridine in the same manner that Compound 6 was synthesized from Compound 4 and 2,3-dichloro-5-nitro-pyridine in Example 3. ¹H NMR (400 MHz, CDCl₃) δ 8.94 (d, 1H), 8.91 (s, 1H), 8.84 (d, 1H), 8.67 (d, 1H), 8.24 (br d, 1H), 7.58 (s, 1H), 7.23 (d, 1H), 4.35 (t, 2H), 4.16-4.09 (m, 2H), 3.03 (d, 3H), 0.95-0.90 (m, 9H), 0.13 (s, 6H); MS (EI) for C₂₄H₂₉ClN₄O₆Si, found 533.2 (MH+).

4-((5-Amino-3-chloropyridin-2-yl)oxy)-7-(2-((tert-butyldimethylsilyl)oxy)ethoxy)-N-methylquinoline-6-carboxamide (35): Compound 35 was synthesized from Compound 34 in the same manner that Compound 8 was synthesized from Compound 5 in the first step of Example 5. MS (EI) for C₂₄H₃₁ClN₄O₄Si, found 503.2 (MH+).

N-(6-((7-(2-((tert-Butyldimethylsilyl)oxy)ethoxy)-6-(methylcarbamoyl)quinolin-4-yl)oxy)-5-chloropyridin-3-yl)-N-(4-fluorophenyl)cyclopropane-1,1-dicarboxamide (36): Compound 9 (532.42 mg, 2.39 mmol, 3 eq) was suspended in anhydrous DCM (25 mL) at 16° C. DMF (5.81 mg, 79.51 umol, 0.1 eq) was added with stirring under nitrogen, followed by (COCl)₂ (605.55 mg, 4.77 mmol, 6 eq). The mixture was stirred at 16° C. for 30 min. Toluene (80 mL) was added, and solvent was removed under reduced pressure. The resulting crude acid chloride of Compound 9 was dissolved in anhydrous THE (8 mL). To a mixture of Compound 35 (400 mg, 795.13 umol, 1 eq) in anhydrous THF (35 mL) was added NaH (127.22 mg, 3.18 mmol, 60% purity, 4 eq) at 16° C., and the mixture was stirred at 16° C. for 15 min. To this was added the THF solution of the acid chloride of Compound 9. The reaction was stirred at 16° C. for 45 min. The reaction mixture was poured into aq saturated NH₄Cl (120 mL) and extracted with DCM (3×75 mL). The combined organic extracts were washed with aq saturated NaCl (20 mL), dried over anhyd Na₂SO₄, and concentrated. The resulting residue was purified by flash silica gel chromatography (ISCO®; 20 g SepaFlash® Silica Flash Column, Eluent of 30˜70% Ethyl acetate/Petroleum ether gradient @ 35 mL/min) to give Compound 36 as a white solid (430 mg, 76.36% yield). ¹H NMR (400 MHz, CDCl₃) δ 10.39 (s, 1H), 9.16 (s, 1H), 9.08 (s, 1H), 8.73 (d, 1H), 8.54 (d, 1H), 8.45 (br d, 1H), 8.39 (d, 1H), 7.49 (s, 1H), 7.47-7.43 (m, 2H), 7.04-6.97 (m, 2H), 6.82 (d, 1H), 4.34 (t, 2H), 4.13-4.09 (m, 2H), 3.09 (d, 3H), 1.75 (t, 2H), 1.69 (t, 2H), 0.94 (s, 9H), 0.14 (s, 6H); MS (EI) for C₃₅H₃₉ClFN₅O₆Si, found 708.1 (MH+).

1-N′-[5-Chloro-6-[7-(2-hydroxyethoxy)-6-(methylcarbamoyl)quinolin-4-yl]oxypyridin-3-yl]-1-N-(4-fluorophenyl)cyclopropane-1,1-dicarboxamide (37): To a solution Compound 36 (250 mg, 352.98 umol, 1 eq) in THE (5 mL) was added TBAF (1 M, 529.47 uL, 1.5 eq) in THF at 16° C. The solution was stirred at 16° C. for 1.5 h, after which the reaction mixture was poured into water (80 mL). The resulting solid was filtered, washed with water (3×50 mL) followed by Petroleum ether (15 mL), and dried to give Compound 37 as a white solid (158.5 mg, 73.1% yield). ¹H NMR (400 MHz, DMSO-d₆) δ 10.51 (s, 1H), 10.06 (br s, 1H), 8.79 (d, 1H), 8.56 (s, 1H), 8.51 (d, 1H), 8.47 (br d, 1H), 8.40 (d, 1H), 7.68-7.57 (m, 3H), 7.15 (t, 2H), 6.94 (d, 1H), 5.17 (t, 1H), 4.33 (t, 2H), 3.89-3.82 (m, 2H), 2.85 (d, 3H), 1.46 (br s, 4H); MS (EI) for C₂₉H₂₅ClFN₅O₆, found 594.2 (MH+).

The following compounds were prepared in a similar multi-step process to that used to generate Compound 37 in Example 9:

1-N′-[6-[6-Carbamoyl-7-(2-hydroxyethoxy)quinolin-4-yl]oxy-5-chloropyridin-3-yl]-1-N-(4-fluorophenyl)cyclopropane-1,1-dicarboxamide (38): A solution of NH₃—H₂O in MeOH was used in place of the methylamine in EtOH in Step 1. ¹H NMR (400 MHz, DMSO-d₆) δ 10.51 (s, 1H), 10.07 (br s, 1H), 8.79 (d, 1H), 8.65 (s, 1H), 8.52 (d, 1H), 8.42 (d, 1H), 7.95 (br s, 1H), 7.85 (br s, 1H), 7.68-7.60 (m, 3H), 7.15 (t, 2H), 6.93 (d, 1H), 5.14 (t, 1H), 4.33 (t, 2H), 3.89-3.82 (m, 2H), 1.47 (s, 4H); MS (EI) for C₂₈H₂₃ClFN₅O₆, found 580.1 (MH+).

1-N′-[6-[6-Carbamoyl-7-(2-hydroxyethoxy)quinolin-4-yl]oxy-5-fluoropyridin-3-yl]-1-N-(4-fluorophenyl)cyclopropane-1,1-dicarboxamide (39): A solution of NH₃—H₂O in MeOH was used in place of the methylamine in EtOH in Step 1 and the 2,3-dichloro-5-nitropyridine in Step 5 was replaced with 2-chloro-3-fluoro-5-nitropyridine. ¹H NMR (400 MHz, DMSO-d₆) δ 10.58 (s, 1H), 10.06 (s, 1H), 8.78 (d, 1H), 8.69 (s, 1H), 8.34 (br s, 2H), 7.96 (br s, 1H), 7.86 (br s, 1H), 7.66-7.60 (m, 3H), 7.18-7.12 (m, 2H), 6.94 (d, 1H), 5.14 (t, 1H), 4.33 (t, 2H), 3.88-3.82 (m, 2H), 1.47 (br s, 4H); MS (EI) for C₂₈H₂₃F₂N₅O₆, found 564.1 (MH+).

### Example 10: 1-N′-[5-Fluoro-6-[7-(2-hydroxyethoxy)-6-(methylcarbamoyl)-quinolin-4-yl]oxypyridin-3-yl]-1-N-(4-fluorophenyl)cyclopropane-1,1-dicarboxamide (44)

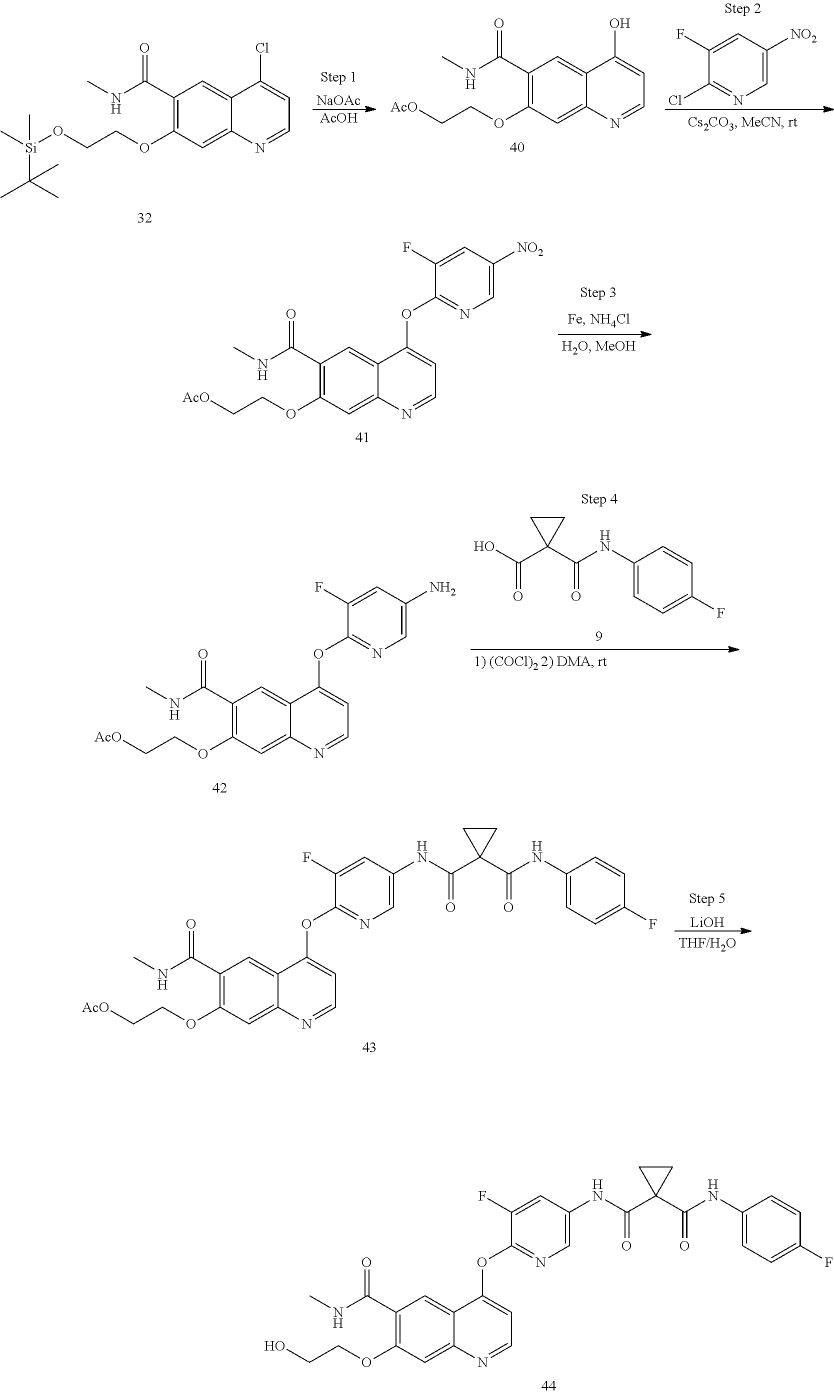

2-((4-Hydroxy-6-(methylcarbamoyl)quinolin-7-yl)oxy)ethyl acetate (40): To a solution of Compound 32 (1.4 g, 3.54 mmol, 1 eq) in AcOH (12 mL) was added NaOAc (581.55 mg, 7.09 mmol, 2 eq), and the mixture was stirred at 90° C. for 15 h to give a brown suspension. After cooling to 20° C., the resulting precipitate was filtered, washed with EtOAc (2×5 mL) and water (2×3 mL), and dried to give Compound 40 as a brown solid (910 mg, 2.81 mmol, 79.31% yield). ¹H NMR (400 MHz, DMSO-d₆) δ 11.65 (br s, 1H), 8.55 (s, 1H), 8.01 (q, 1H), 7.85 (d, 1H), 7.03 (s, 1H), 5.98 (d, 1H), 4.53 (t, 2H), 4.36 (t, 2H), 2.84 (d, 3H), 2.08 (s, 3H); MS (EI) for C₁₅H₁₆N₂O₅, found 305.1 (MH+).

2-((4-((3-Fluoro-5-nitropyridin-2-yl)oxy)-6-(methylcarbamoyl)quinolin-7-yl)oxy)ethyl acetate (41): Compound 41 was synthesized from Compound 40 and 2-chloro-3-fluoro-5-nitro-pyridine in the same manner that Compound 6 was synthesized from Compound 4 and 2,3-dichloro-5-nitro-pyridine in Example 3. ¹H NMR (400 MHz, CDCl₃) δ 8.97-8.93 (m, 2H), 8.78 (d, 1H), 8.40 (dd, 1H), 8.00 (br s, 1H), 7.59 (s, 1H), 7.24 (d, 1H), 4.71-4.67 (m, 2H), 4.49-4.45 (m, 2H), 3.05 (d, 3H), 2.16 (s, 3H); MS (EI) for C₂₀H₁₇FN₄O₇, found 445.1 (MH+).

2-((4-((5-Amino-3-fluoropyridin-2-yl)oxy)-6-(methylcarbamoyl)quinolin-7-yl)oxy)ethyl acetate (42): Compound 42 was synthesized from Compound 41 in the same manner that Compound 8 was synthesized from Compound 5 in the first step of Example 5. MS (EI) for C₂₀H₁₉FN₄O₅, found 437.1 [M+Na]⁺.

2-((4-((3-Fluoro-5-(1-((4-fluorophenyl)carbamoyl)cyclopropane-1-carboxamido)pyridin-2-yl)oxy)-6-(methylcarbamoyl)quinolin-7-yl)oxy)ethyl acetate (43): Using 161.59 mg (723.96 umol) of Compound 9, a THF solution of the acid chloride of Compound 9 was generated in the same manner that it was in Step 7 of Example 9. To a solution of Compound 42 (100 mg, 241.32 umol, 1 eq) in DMA (8 mL) was added the THF solution of the acid chloride of Compound 9 with stirring under nitrogen. The reaction was stirred at 25° C. for 0.5 h. The reaction mixture was poured into aq saturated NH₄Cl (100 mL) and extracted with DCM (3×50 mL). The combined organic extracts were washed with aq saturated NaCl (15 mL), dried over anhyd Na₂SO₄, and concentrated to give Compound 43 as a yellow solid (100 mg, 66.9% yield) which was used into the next step without further purification. MS (EI) for C₃₁H₂₇F₂N₅O₇, found 620.0 (MH+).

1-N′-[5-Fluoro-6-[7-(2-hydroxyethoxy)-6-(methylcarbamoyl)-quinolin-4-yl]oxypyridin-3-yl]-1-N-(4-fluorophenyl)cyclopropane-1,1-dicarboxamide (44): To a mixture of Compound 43 (90 mg, 145.26 umol, 1 eq) in water (5 mL) and THE (2.5 mL) was added LiOH·H₂O (1 M, 1 mL, 6.88 eq) slowly and the mixture was stirred at 25° C. for 0.5 h. Water (15 mL) was added, and the resulting mixture was extracted with DCM (3×20 mL). The combined DCM extracts were washed with aq saturated NaCl (10 mL), dried over anhyd Na₂SO₄, and concentrated. The resulting residue was purified by prep-HPLC (Column: DuraShell 150*25 mm*5 um, gradient: ²⁸-58% of acetonitrile in water (0.05% NH₃·H₂O), flow rate: 30 mL/min) to give Compound 44 as a white solid (73.7 mg, 87.8% yield). ¹H NMR (400 MHz, DMSO-d₆) δ 10.57 (br s, 1H), 10.06 (br s, 1H), 8.77 (br d, 1H), 8.60 (s, 1H), 8.48 (br d, 1H), 8.33 (br s, 2H), 7.73-7.53 (m, 3H), 7.15 (br t, 2H), 6.93 (br d, 1H), 5.19 (br t, 1H), 4.33 (br s, 2H), 3.86 (br s, 2H), 2.85 (br d, 3H), 1.48 (br s, 4H); MS (EI) for C₂₉H₂₅F₂N₅O₆, found 578.1 (MH+).

### Example 11: 1-N′-[5-Chloro-6-[7-(2-hydroxypropoxy)-6-(methylcarbamoyl)-quinolin-4-yl]oxypyridin-3-yl]-1-N-(4-fluorophenyl)cyclopropane-1,1-dicarboxamide (50)

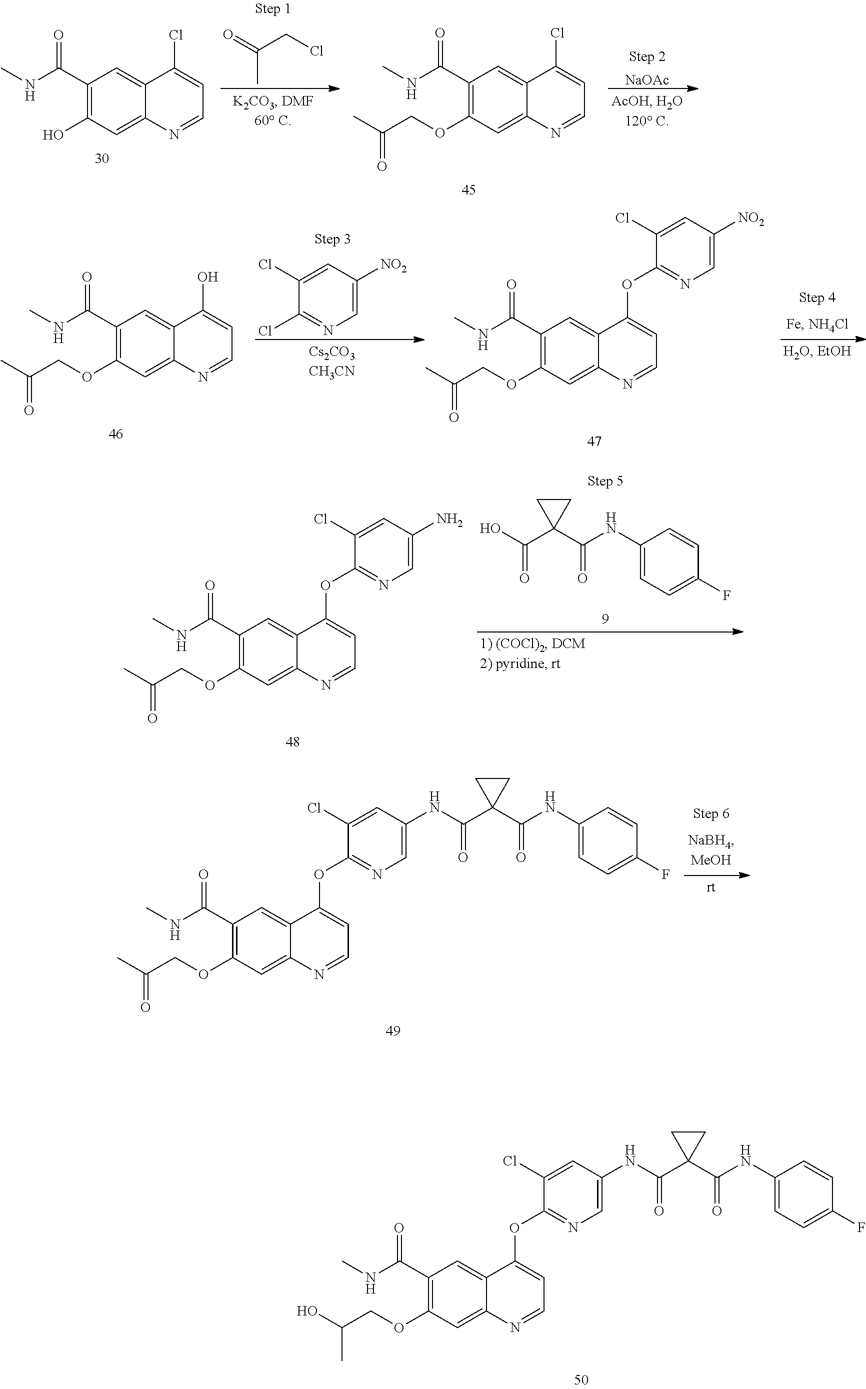

4-Chloro-N-methyl-7-(2-oxopropoxy)quinoline-6-carboxamide (45): A mixture of Compound 30 (1.5 g, 6.34 mmol, 1 eq), 1-chloropropan-2-one (1.78 g, 19.24 mmol, 5.67 mL, 3.04 eq), and K₂C₃(2.63 g, 19.02 mmol, 3 eq) in DMF (80 mL) was stirred at 60° C. for 12 h. Water (200 mL) was added, and the resulting solid was filtered, washed with water (20 mL) followed by petroleum ether (50 mL), and dried to give Compound 45 as a yellow solid (1.3 g, 70.100 yield) which was used into the next step without further purification. ¹H NMR (400 MHz, DMSO-d₆) δ 8.80 (d, 1H), 8.74 (br d, 1H), 8.58 (s, 1H), 7.64 (d, 1H), 7.52 (s, 1H), 5.18 (s, 2H), 2.89 (d, 3H), 2.24 (s, 3H); MS (EI) for C₁₄H₁₃ClN₂O₃, found 292.8 (MH+).

4-Hydroxy-N-methyl-7-(2-oxopropoxy)quinoline-6-carboxamide (46): Compound 46 was synthesized from Compound 45 in the same manner that Compound 33 was synthesized from Compound 32 in the Step 4 of Example 9. MS (EI) for C₁₄1H₁₄N₂O₄, found 274.9 (MH+).

4-((3-Chloro-5-nitropyridin-2-yl)oxy)-N-methyl-7-(2-oxopropoxy)quinoline-6-carboxamide (47): Compound 47 was synthesized from Compound 46 and 2,3-dichloro-5-nitro-pyridine in the same manner that Compound 6 was synthesized from Compound 4 and 2,3-dichloro-5-nitro-pyridine in Example 3. ¹H NMR (400 MHz, DMSO-d₆) δ 9.04 (br s, 1H), 8.96 (br s, 2H), 8.69 (br s, 1H), 8.36 (s, 1H), 7.57 (s, 1H), 7.43 (br d, 1H), 5.19 (s, 2H), 2.84 (br d, 3H), 2.24 (s, 3H); MS (EI) for C₁₉H₁₅ClN₄O₆, found 452.9 [M+Na]⁺.

4-((5-Amino-3-chloropyridin-2-yl)oxy)-N-methyl-7-(2-oxopropoxy)quinoline-6-carboxamide (48): Compound 48 was synthesized from Compound 47 in the same manner that Compound 8 was synthesized from Compound 5 in the first step of Example 5. MS (EI) for C₁₉H₁₇ClN₄O₄, found 423.0 [M+Na]⁺.

N-(5-Chloro-6-((6-(methylcarbamoyl)-7-(2-oxopropoxy)quinolin-4-yl)oxy)pyridin-3-yl)-N-(4-fluorophenyl)cyclopropane-1,1-dicarboxamide (49): Using 618.12 mg (2.77 mmol) of Compound 9, a THF solution of the acid chloride of Compound 9 was generated in the same manner that it was in Step 7 of Example 9. To a mixture of Compound 48 (370 mg, 923.12 umol, 1 eq) in pyridine (30 mL) was added the THF solution of the acid chloride of Compound 9, and the reaction was stirred at 16° C. for 12 h. The reaction mixture was poured into aq saturated NH₄Cl (150 mL) and extracted with DCM (3×80 mL). The combined DCM extracts were washed with aq saturated NaCl (20 mL), dried with anhyd Na₂SO₄, and concentrated. The resulting residue was purified by flash silica gel chromatography (ISCO®; 20 g SepaFlash® Silica Flash Column, Eluent of 0˜5% CH₃OH/CH₂Cl₂ gradient @ 35 mL/min) to give Compound 49 as a yellow solid (110 mg, 19.7% yield). MS (EI) for C₃₀H₂₅ClFN₅O₆, found 606.2 [M+Na]⁺.

1-N′-[5-Chloro-6-[7-(2-hydroxypropoxy)-6-(methylcarbamoyl)-quinolin-4-yl]oxypyridin-3-yl]-1-N-(4-fluorophenyl)cyclopropane-1,1-dicarboxamide (50): To a solution of Compound 49 (110 mg, 181.52 umol, 1 eq) dissolved in MeOH (20 mL) was added solid NaBH₄ (8.24 mg, 217.82 umol, 1.2 eq), and the reaction mixture was stirred at 16° C. for 1 h. Water (30 mL) was added, and the volatile solvents removed under reduced pressure. The resulting solids were filtered and purified by prep-HPLC (Column: Boston Prime C18 150*30 mm 5 um, gradient: 40-60% of acetonitrile in water (0.05% NH₃H₂O), flow rate: 25 mL/min) to give Compound 50 as a white solid (55.8 mg, 50.6% yield). ¹H NMR (400 MHz, DMSO-d₆) δ 10.51 (s, 1H), 10.07 (s, 1H), 8.79 (d, 1H), 8.58-8.48 (m, 3H), 8.40 (d, 1H), 7.68-7.58 (m, 3H), 7.15 (t, 2H), 6.94 (d, 1H), 5.20 (d, 1H), 4.24 (br d, 1H), 4.16-4.05 (m, 2H), 2.84 (d, 3H), 1.46 (br s, 4H), 1.21 (d, 3H); MS (EI) for C₃₀H₂₇ClFN₅O₆, found 608.2 (MH+).

The following compound was prepared in a similar multi-step process to that used to generate Compound 50 in Example 11. Compound 30 was replaced with 4-chloro-7-hydroxyquinoline-6-carboxamide which was made in the same way that Compound 30 was made in 2 steps from Compound 28 in Example 9 using a solution of NH₃—H₂O in MeOH in place of the methylamine in EtOH in Step 1. Step 5 in Example 11 was replaced with the method used in Step 7 of Example 9.

1-N′-[6-[6-Carbamoyl-7-(2-hydroxypropoxy)quinolin-4-yl]oxy-5-chloropyridin-3-yl]-1-N-(4-fluorophenyl)cyclopropane-1,1-dicarboxamide (51): ¹H NMR (400 MHz, DMSO-d₆) δ 10.51 (s, 1H), 10.06 (s, 1H), 8.79 (d, 1H), 8.62 (s, 1H), 8.51 (br d, 1H), 8.42 (d, 1H), 8.01 (br s, 1H), 7.86 (br s, 1H), 7.63 (br dd, 2H), 7.59 (s, 1H), 7.15 (br t, 2H), 6.93 (d, 1H), 5.14 (d, 1H), 4.24 (br d, 1H), 4.17-4.04 (m, 2H), 1.47 (br s, 4H), 1.22 (br d, 3H); MS (EI) for C₂₉H₂₅ClFN₅O₆, found 594.1 (MH+).

### Example 12: 1-N-(4-Fluorophenyl)-1-N′-[6-[7-methoxy-6-(1H-pyrazol-4-yl)quinolin-4-yl]oxypyridin-3-yl]cyclopropane-1,1-dicarboxamide (60)

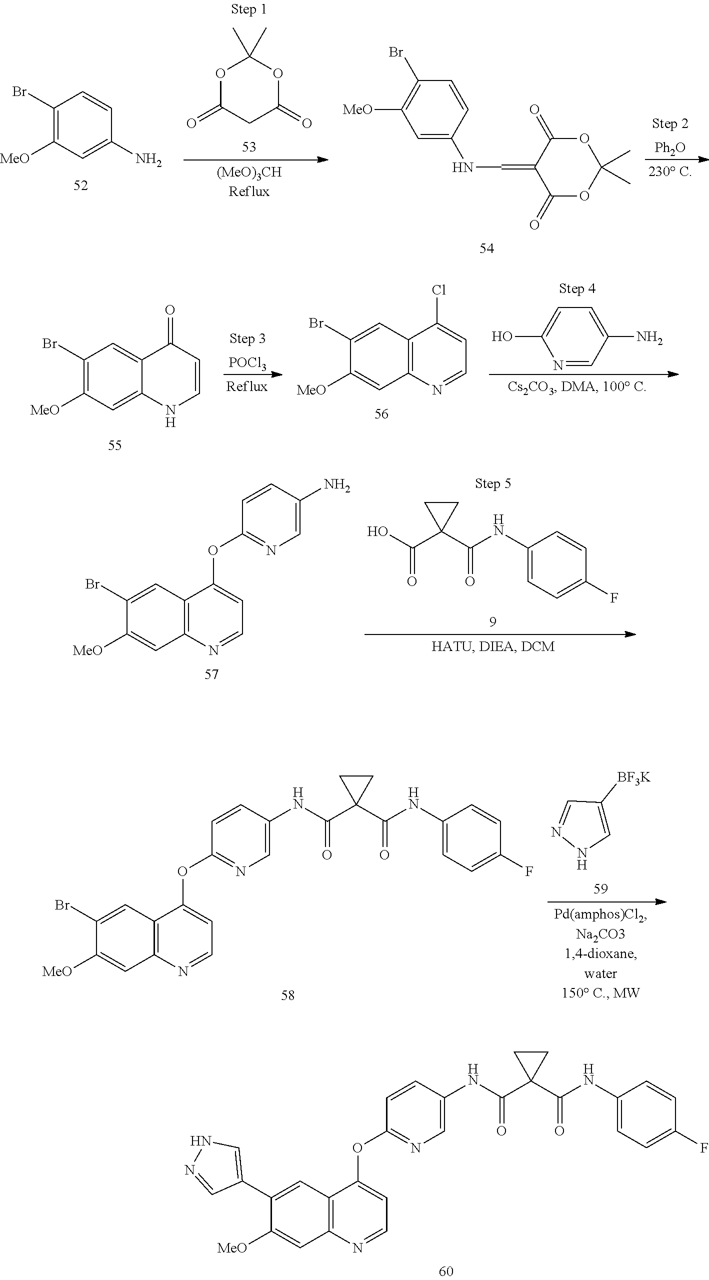

5-(((4-Bromo-3-methoxyphenyl)amino)methylene)-2,2-dimethyl-1,3-dioxane-4,6-dione (54): Compound 53 (4.10 g, 28.46 mmol, 1.15 eq) in trimethoxymethane (25 mL, 228.04 mmol, 9.22 eq) was heated to reflux at 105° C. for 1 h. Compound 52 (5 g, 24.75 mmol, 1 eq) was then added, and the reflux continued at 105° C. for another 1 h. The resulting suspension was filtered, washed with MeOH, and vacuum dried to yield Compound 54 as a white solid (7.7 g, 87.4% yield) which was used in the next step without further purification. ¹H NMR (400 MHz, CDCl₃) δ 11.24-11.20 (d, 1H), 8.62-9.59 (d, 1H), 7.57-7.54 (d, 1H), 6.77-6.74 (m, 2H), 3.94 (s, 3H), 1.76 (s, 3H), 1.74 (s, 3H).

6-Bromo-7-methoxyquinolin-4(1H)-one (55): To Ph₂O (35 mL) at 230° C. was added Compound 54 (7.7 g, 21.62 mmol, 1 eq), and the mixture was stirred for 1 h. After cooling to room temperature, the reaction mixture was poured into hexane (20 mL), and the resulting precipitate was filtered and washed with hexane. The resulting brown solid was dried under vacuum to give Compounds 55 (6.2 g, 75.8% yield, 67.2% purity). ¹H NMR (400 MHz, DMSO-d₆) δ 11.78 (s, 1H), 8.18 (s, 1H), 7.88-7.86 (d, 1H), 7.05 (s, 1H), 6.03-6.01 (d, 1H), 3.92 (s, 3H); MS (EI) for C₁₀H₈BrNO₂, found 254.2 (MH+).

6-Bromo-4-chloro-7-methoxyquinoline (56): Compound 55 (6.2 g, 16.40 mmol, 1 eq) in POCl₃ (15 mL, 161.41 mmol, 9.84 eq) was stirred at 110° C. for 1 h. After cooling, the reaction mixture was cautiously poured into a mixture of aq saturated Na₂CO₃ and ice with stirring. The resulting suspension was filtered, washed with water, and dried under vacuum to give Compound 56 as a dark brown solid (7.78 g, 57.4% purity, 99.9% yield) which was used in subsequent steps without further purification. MS (EI) for C₁₀H₇BrClNO, found 272.2 (MH+).

6-((6-Bromo-7-methoxyquinolin-4-yl)oxy)pyridin-3-amine (57): To a mixture of Compound 56 (548 mg, 2.0 mmol) and 5-aminopyridin-2-ol (330 mg, 3.0 mmol) in DMA (8 mL) was added Cs₂CO₃ (1.3 g, 4 mmol) at room temperature. The mixture was stirred at 100° C. for 12 h. The mixture was then cooled to 20° C., diluted with water, and extracted with EtOAc. The organic phase was concentrated, and the crude residue was purified by flash silica gel chromatography to give Compound 57 as a solid (65% yield). MS (EI) for C₁₅H₁₂BrN₃O₂, found 346 (MH+).

N-(6-((6-Bromo-7-methoxyquinolin-4-yl)oxy)pyridin-3-yl)-N-(4-fluorophenyl)-cyclopropane-1,1-dicarboxamide (58): Compound 58 was synthesized from Compound 57 and Compound 9 in the same manner that Compound 10 was synthesized from Compound 8 and Compound 9 in the second step of Example 5. MS (EI) for C₂₆H₂₀BrFN₄O₄, found 551 (MH+).

1-N-(4-Fluorophenyl)-1-N′-[6-[7-methoxy-6-(1H-pyrazol-4-yl)quinolin-4-yl]oxypyridin-3-yl]cyclopropane-1,1-dicarboxamide (60): Compound 58 (120 mg, 0.22 mmol), Compound 59 (57 mg, 0.33 mmol), Na₂CO₃ (70 mg, 0.66 mmol), bis(di-t-butyl(4-dimethylaminophenyl)phosphine)dichloropalladium(II) (15 mg, 0.02 mmol), 1,4-dioxane (2 mL), and water (0.3 mL) were combined in a microwave reaction tube. The reaction mixture was heated at 150° C. under microwave irradiation for 20 min. After cooling, the mixture was extracted with EtOAc. The organic phase was washed with aq saturated NaCl and concentrated. The crude product was purified by prep HPLC to give Compound 60 (50% yield). MS (EI) for C₂₉H₂₃FN₆O₄, found 539 (MH+).

### Example 13: 1-N′-[5-Chloro-6-[7-methoxy-6-(1,3-oxazol-2-yl)quinolin-4-yl]oxypyridin-3-yl]-1-N-(4-fluorophenyl)cyclopropane-1,1-dicarboxamide (64)

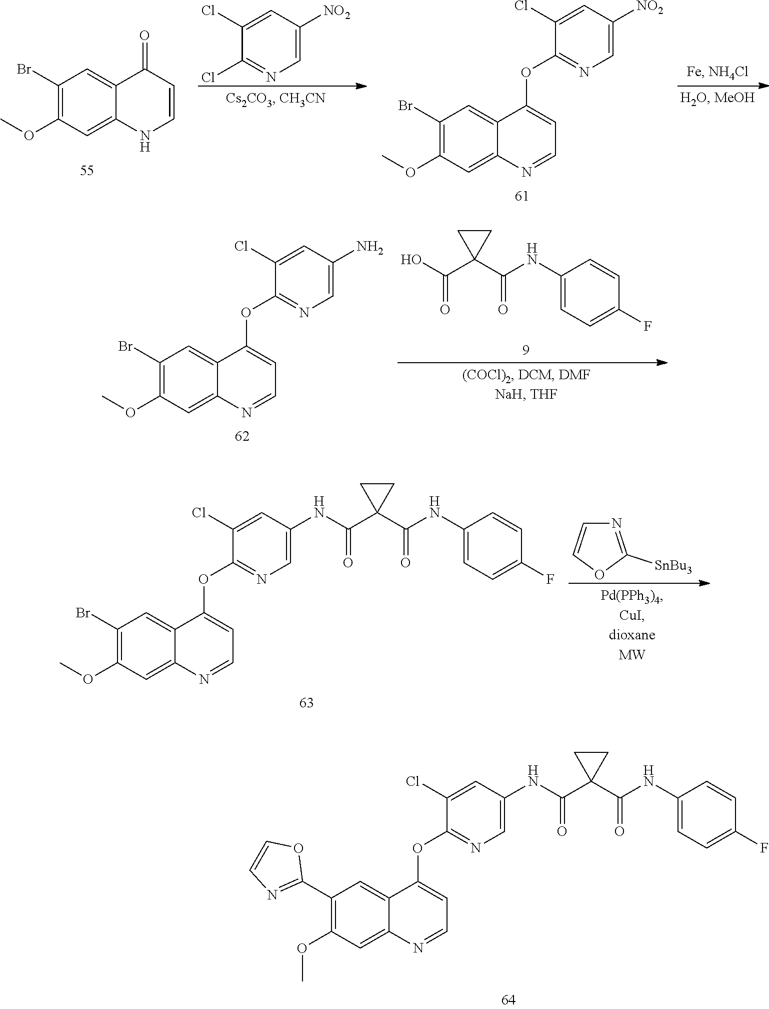

6-Bromo-4-((3-chloro-5-nitropyridin-2-yl)oxy)-7-methoxyquinoline (61): Compound 61 was synthesized from Compound 55 and 2,3-dichloro-5-nitro-pyridine in the same manner that Compound 6 was synthesized from Compound 4 and 2,3-dichloro-5-nitro-pyridine in Example 3. ¹H NMR (400 MHz, CDCl₃) δ 8.90 (d, 1H), 8.88 (d, 1H), 8.69 (d, 1H), 8.20 (s, 1H), 7.53 (s, 1H), 7.22 (d, 1H), 4.07 (s, 3H); MS (EI) for C₁₅H₉BrClN₃O₄, found 411.9 (MH+).

6-((6-Bromo-7-methoxyquinolin-4-yl)oxy)-5-chloropyridin-3-amine (62): Compound 62 was synthesized from Compound 61 in the same manner that Compound 8 was synthesized from Compound 5 in the first step of Example 5. ¹H NMR (400 MHz, DMSO-d₆) δ 8.66 (d, 1H), 8.41 (s, 1H), 7.64 (d, 1H), 7.54 (s, 1H), 7.29 (d, 1H), 6.60 (d, 1H), 5.72 (s, 2H), 4.03 (s, 3H); MS (EI) for C₁₅H₁BrClN₃O₂, found 381.9 (MH+).

N-(6-((6-Bromo-7-methoxyquinolin-4-yl)oxy)-5-chloropyridin-3-yl)-N-(4-fluorophenyl)cyclopropane-1,1-dicarboxamide (63): Compound 63 was synthesized from Compound 62 and Compound 9 in the same manner that Compound 36 was synthesized from Compound 35 and Compound 9 in Step 7 of Example 9. ¹H NMR (400 MHz, CDCl₃) δ 10.60 (s, 1H), 9.71 (s, 1H), 8.73 (br d, 1H), 8.46 (d, 1H), 8.24 (d, 1H), 8.03 (s, 1H), 7.48 (s, 2H), 7.45 (d, 1H), 7.07 (br d, 2H), 6.87 (d, 1H), 4.06 (s, 3H), 1.91-1.85 (m, 2H), 1.66-1.59 (m, 2H); MS (EI) for C₂₆H₁₉BrClFN₄O₄, found 587.0 (MH+).

1-N′-[5-Chloro-6-[7-methoxy-6-(1,3-oxazol-2-yl)quinolin-4-yl]oxypyridin-3-yl]-1-N-(4-fluorophenyl)cyclopropane-1,1-dicarboxamide (64): Compound 63 (200 mg, 341.41 umol, 1 eq), tributyl(oxazol-2-yl)stannane (150.00 mg, 418.87 umol, 1.23 eq), CuI (13.00 mg, 68.26 umol, 0.2 eq), Pd(PPh₃)₄(80.00 mg, 69.23 umol, 2.03e-1 eq), and dioxane (3 mL) were added to a microwave reaction tube. The sealed tube was heated at 100° C. for 2 h under microwave irradiation. Aq saturated KF (50 mL) was added and the mixture was stirred at 20° C. for 1 h. NH₃·H₂O (5 mL) was added, and the resulting mixture was extracted with EtOAc (3×50 mL). The combined organic layers were washed with aq saturated NaCl (30 mL), dried over anhyd Na₂SO₄, and concentrated under reduced pressure. The resulting residue was purified by prep-HPLC (basic condition).column: Xtimate C18 150*25 mm*5 um; mobile phase: [water (0.05% ammonia hydroxide v/v)-ACN]; B %: 50%-80%, 7.8 min to give Compound 64 as a white solid (34.2 mg, 17.4% yield). 1H NMR (400 MHz, DMSO-d6) δ 10.50 (br s, 1H), 10.05 (br s, 1H), 8.80 (d, 1H), 8.62 (s, 1H), 8.50 (s, 1H), 8.42 (s, 1H), 8.29 (s, 1H), 7.67-7.59 (m, 3H), 7.43 (d, 1H), 7.14 (t, 2H), 6.95 (d, 1H), 4.04 (s, 3H), 1.46 (br s, 4H); MS (EI) for C₂₉H₂₁ClFN₅O₅, found 574.0 (MH+).

### Example 14: 1-N′-[5-Fluoro-6-[7-methoxy-6-(1,3,4-oxadiazol-2-yl)quinolin-4-yl]oxypyridin-3-yl]-1-N-(4-fluorophenyl)cyclopropane-1,1-dicarboxamide (67)

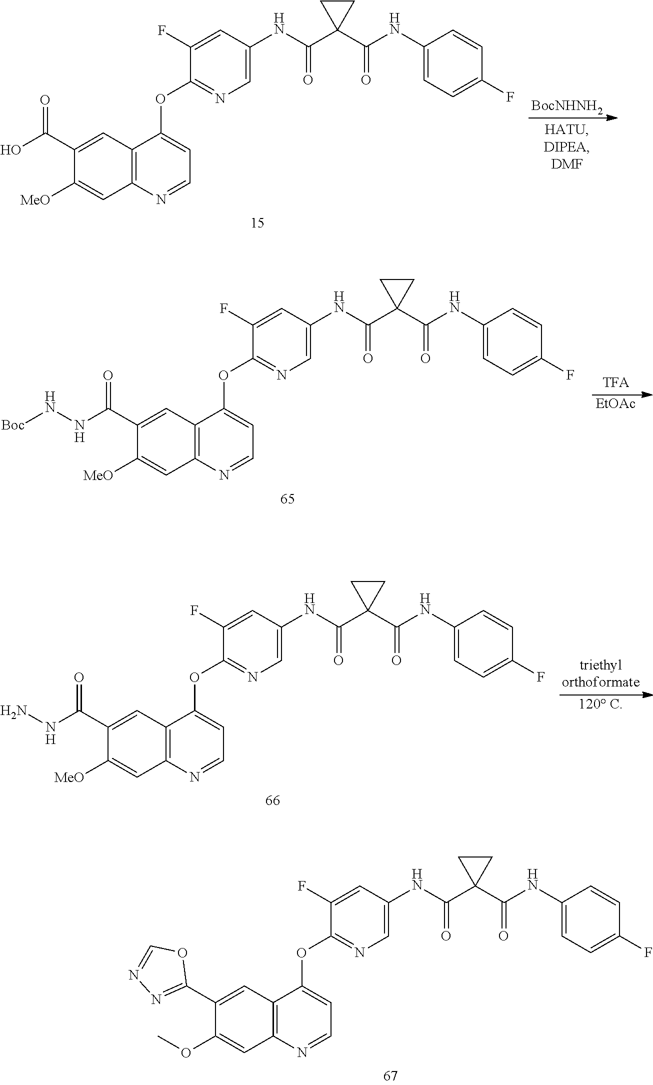

tert-Butyl 2-(4-((3-fluoro-5-(1-((4-fluorophenyl)carbamoyl)cyclopropane-1-carboxamido)pyridin-2-yl)oxy)-7-methoxyquinoline-6-carbonyl)hydrazine-1-carboxylate (65): To a solution of Compound 15 (300 mg, 561.31 umol, 1 eq) in DMF (20 mL) was added HATU (234.77 mg, 617.44 umol, 1.1 eq) and DIEA (217.63 mg, 1.68 mmol, 293.31 uL, 3.0 eq), and the mixture was stirred at 16° C. for 30 min. Tert-butyl N-aminocarbamate (111.27 mg, 841.96 umol, 1.5 eq) was added, and the reaction mixture was stirred at 16° C. for 12 h. The reaction mixture was poured into water (100 mL) and extracted with EtOAc (3×80 mL). The combined organic phases were washed with aq saturated NaCl (20 mL), dried with anhyd Na₂SO₄, and concentrated. The resulting residue was purified by flash silica gel chromatography (ISCO®; 20 g SepaFlash® Silica Flash Column, Eluent of 50˜100% Ethyl acetate/Petroleum ether gradient @ 35 mL/min) to give Compound 65 as a yellow solid (300 mg, 82.40% yield). MS (EI) for C₃₂H₃₀F₂N₆O₇, found 649.2 (MH+).

N-(5-Fluoro-6-((6-(hydrazinecarbonyl)-7-methoxyquinolin-4-yl)oxy)pyridin-3-yl)-N-(4-fluorophenyl)cyclopropane-1,1-dicarboxamide (66): To a solution of Compound 65 (250 mg, 385.44 umol, 1 eq) in EtOAc (15 mL) was added TFA (4.62 g, 40.52 mmol, 3 mL, 105.12 eq), and the reaction mixture was stirred at 16° C. for 12 h. The reaction mixture was diluted with EtOAc (80 mL) and H₂O (80 mL), and K₂CO₃ was added until pH 8 was achieved. The phases were separated, and the aqueous phase was further extracted with EtOAc (3×45 mL). The combined organic phases were washed with aq saturated NaCl (20 mL), dried over anhyd Na₂SO₄, and concentrated to give Compound 66 as a yellow solid (200 mg, 94.6% yield) which was used into the next step without further purification. MS (EI) for C₂₇H₂₂F₂N₆O₅, found 571.1 [M+Na]⁺.

1-N′-[5-Fluoro-6-[7-methoxy-6-(1,3,4-oxadiazol-2-yl)quinolin-4-yl]oxypyridin-3-yl]-1-N-(4-fluorophenyl)cyclopropane-1,1-dicarboxamide (67): A mixture of Compound 66 (200 mg, 364.63 umol, 1 eq) in triethyl orthoformate (8 mL) was stirred at 120° C. for 12 h. The reaction mixture was concentrated to give a residue, which was purified by prep-HPLC (Column: DuraShell 150*25 mm*5 um, gradient: 22-52% of acetonitrile in water (0.1% TFA), flow rate: 25 mL/min). The eluent was evaporated to remove organic solvents, and the pH of the resulting aqueous solution was adjusted to 8 with the addition of K₂CO₃. The resulting mixture was extracted with EtOAc (3×45 mL) and concentrated to give Compound 67 as a yellow solid (45.5 mg, 21.36% yield). 1H NMR (400 MHz, DMSO-d6) δ 10.65 (br s, 1H), 10.12 (br s, 1H), 9.44 (s, 1H), 8.84 (d, 1H), 8.71 (s, 1H), 8.43-8.32 (m, 2H), 7.71 (s, 1H), 7.68-7.62 (m, 2H), 7.15 (br t, 2H), 6.99 (br s, 1H), 4.07 (s, 3H), 1.49 (br s, 4H); MS (EI) for C₂₈H₂₀F₂N₆O₅, found 559.1 (MH+).

The following compound was prepared in a three step procedure analogous to that followed for Compound 67 in Example 14, replacing Compound 15 with Compound 14 in the first step:

1-N′-[5-Chloro-6-[7-methoxy-6-(1,3,4-oxadiazol-2-yl)quinolin-4-yl]oxypyridin-3-yl]-1-N-(4-fluorophenyl)cyclopropane-1,1-dicarboxamide (68): ¹H NMR (400 MHz, DMSO-d₆) δ 10.60 (s, 1H), 10.14 (s, 1H), 9.48 (s, 1H), 8.91 (d, 1H), 8.74 (s, 1H), 8.58 (s, 1H), 8.50 (d, 1H), 7.77 (s, 1H), 7.69 (dd, 2H), 7.21 (t, 2H), 7.05 (d, 1H), 4.13 (s, 3H), 1.54 (s, 4H); MS (EI) for C₂₈H₂₀ClFN₆O₅, found 575.1 (MH+).

### Example 15: 1-N′-[5-Chloro-6-[6-(1H-imidazol-2-yl)-7-methoxyquinolin-4-yl]oxypyridin-3-yl]-1-N-(4-fluorophenyl)cyclopropane-1,1-dicarboxamide (71)

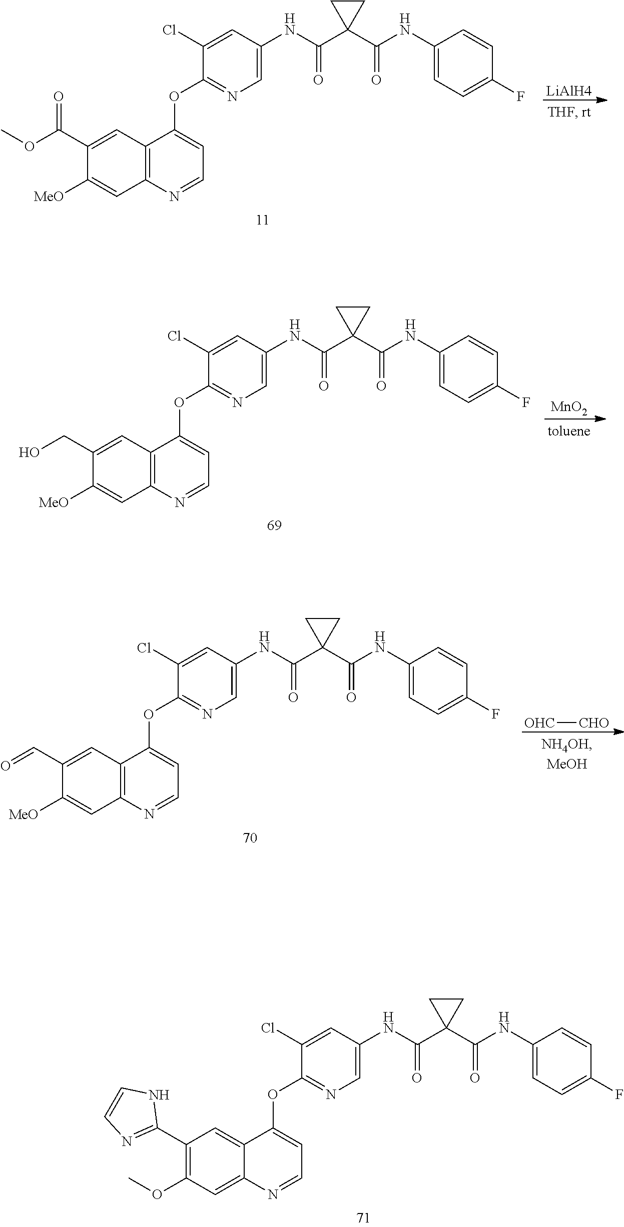

N-(5-Chloro-6-((6-(hydroxymethyl)-7-methoxyquinolin-4-yl)oxy)pyridin-3-yl)-N-(4-fluorophenyl)cyclopropane-1,1-dicarboxamide (69): To a mixture of Compound 11 (300 mg, 531.02 umol, 1 eq) in THF (10 mL) was added dropwise LiAlH₄ (1 M in THF, 1.06 mL, 2 eq) in THF at −78° C. The mixture was stirred at −78° C. for 2 h. The reaction was quenched with Na₂SO₄·10H₂O (0.2 g) and water (0.1 mL) and filtered. The filtrate was concentrated under reduced pressure to Compound 69 as a white solid (230 mg, 77.6% yield) which was used directly without further purification. MS (EI) for C₂₇H₂₂ClFN₄O₅, found 537.1 (MH+).

N-(5-chloro-6-((6-formyl-7-methoxyquinolin-4-yl)oxy)pyridin-3-yl)-N-(4-fluorophenyl)cyclopropane-1,1-dicarboxamide (70): To a mixture of Compound 69 (200 mg, 372.48 umol, 1 eq) in toluene (10 mL) was added MnO₂ (161.92 mg, 1.86 mmol, 5 eq) in one portion at 0° C. The mixture was stirred at 80° C. for 20 h. The mixture was filtered and concentrated to give Compound 70 as a yellow solid (180 mg, 76.8% yield) which was used without further purification. MS (EI) for C₂₇H₂₀ClFN₄O₅, found 535.1(MH+).

1-N′-[5-Chloro-6-[6-(1H-imidazol-2-yl)-7-methoxyquinolin-4-yl]oxypyridin-3-yl]-1-N-(4-fluorophenyl)cyclopropane-1,1-dicarboxamide (71): To a Compound 70 (160 mg, 299.11 umol, 1 eq) in MeOH (1 mL) was added NH₃·H₂O (1.72 g, 12.79 mmol, 1.89 mL, 26-28% purity, 42.75 eq) and GLYOXAL (86.80 mg, 1.50 mmol, 78.20 uL, 5 eq). The mixture was stirred at 10° C. for 16 h. The mixture was concentrated, and the resulting residue was purified by prep-HPLC (column: Waters Xbridge 150*25 5u; mobile phase: [water (0.05% ammonia hydroxide v/v)-ACN]; B %: 45%-75%, 6.5 min) to give Compound 71 as a yellow solid (31.8 mg, 18.4% yield). ¹H NMR (400 MHz, DMSO-d₆) δ 12.10 (s, 1H), 10.50 (s, 1H), 10.07 (s, 1H), 8.94-8.63 (m, 2H), 8.60-8.29 (m, 2H), 7.77-7.50 (m, 3H), 7.27 (s, 1H), 7.17-7.13 (m, 2H), 7.09 (s, 1H), 6.92 (d, 1H), 4.13 (s, 3H), 1.47 (s, 4H); MS (EI) for C₂₉H₂₂ClFN₆O₄, found 573.1 (MH+).

The following compound was prepared in a three step procedure analogous to that followed for Compound 71 in Example 15, replacing Compound 11 with Compound 12 in the first step:

1-N′-[5-Fluoro-6-[6-(1H-imidazol-2-yl)-7-methoxyquinolin-4-yl]oxypyridin-3-yl]-1-N-(4-fluorophenyl)cyclopropane-1,1-dicarboxamide (72): ¹H NMR (400 MHz, DMSO-d₆) δ 12.10 (br s, 1H), 10.57 (s, 1H), 10.06 (s, 1H), 8.87 (s, 1H), 8.71 (d, 1H), 8.38-8.30 (m, 2H), 7.66-7.59 (m, 3H), 7.27 (s, 1H), 7.20-7.12 (m, 2H), 7.09 (s, 1H), 6.92 (d, 1H), 4.12 (s, 3H), 1.50-1.44 (m, 4H); MS (EI) for C₂₉H₂₂F₂N₆O₄, found 557.1 (MH+).

### Example 16: 6,7-Dimethoxy-1,5-naphthyridin-4-ol (77)

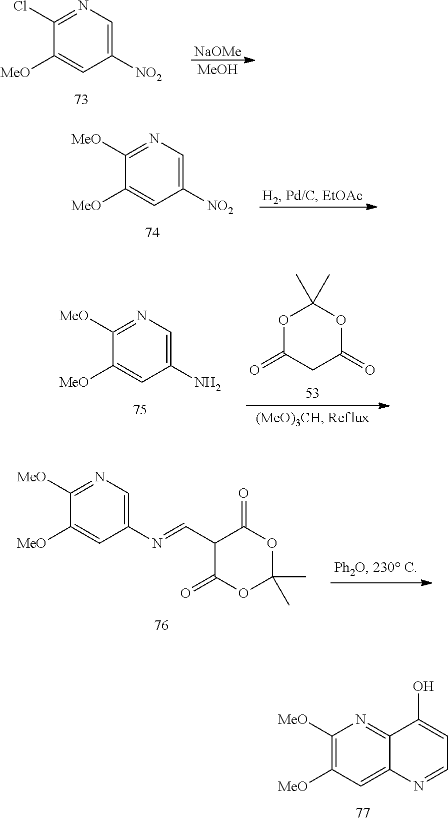

2,3-Dimethoxy-5-nitropyridine (74): Freshly cut sodium (0.6 g, 26 mmol) was added portionwise to MeOH (50 mL), and the mixture was stirred at room temperature until the sodium dissolved. Compound 73 (3.0 g, 15.9 mmol) was added, and the reaction mixture was stirred at room temperature for 1 h. Water (100 mL) was added, and the mixture was filtered. The solids were washed with water and dried to give Compound 74 (2.78 g, 95% yield). MS for C₇H8N₂O₄, found 185 (MH+).

2,3-Dimethoxy-5-nitropyridine (75): To a solution of Compound 74 (2.78 g, 15.1 mmol) in EtOAc (40 mL) under argon was added 10% Pd/C (53% water, 880 mg). The reaction mixture was stirred under one atmosphere of H₂ at room temperature overnight and then filtered through Celite®. The filtrate was concentrated under vacuum to provide crude Compound 75 as brown solid (2.31 g, 100% yield). MS for C₇H₁₀N₂O₂, found 155 (MH+).

5-(((5,6-Dimethoxypyridin-3-yl)imino)methyl)-2,2-dimethyl-1,3-dioxane-4,6-dione (76): Compound 76 was synthesized from Compound 75 and Compound 53 in the same manner that Compound 54 was synthesized from Compound 52 and Compound 53 in Step 1 of Example 12. MS for C₁₄H₁₆N₂O₆, found 309 (MH+).

6,7-Dimethoxy-1,5-naphthyridin-4-ol (77): Compound 77 was synthesized from Compound 76 in the same manner that Compound 55 was synthesized from Compound 54 in Step 2 of Example 12. MS for C₁₀H₁₀N₂O₃, found 207 (MH+).

### Example 17: 1-N′-[5-Chloro-6-[(6,7-dimethoxy-1,5-naphthyridin-4-yl)oxy]pyridin-3-yl]-1-N-(4-fluorophenyl)cyclopropane-1,1-dicarboxamide (80)

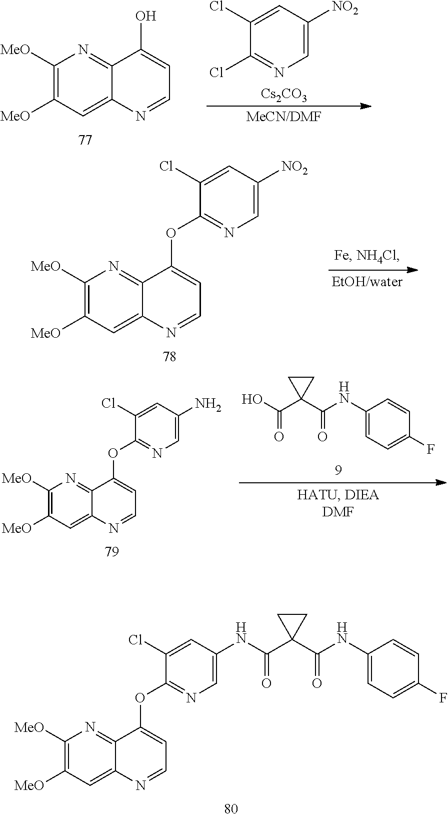

8-((3-Chloro-5-nitropyridin-2-yl)oxy)-2,3-dimethoxy-1,5-naphthyridine (78): Compound 78 was synthesized from Compound 77 and 2,3-dichloro-5-nitro-pyridine in the same manner that Compound 6 was synthesized from Compound 4 and 2,3-dichloro-5-nitro-pyridine in Example 3. MS for C₁₅H₁₁ClN₄O₅, found 363 (MH+).

5-Chloro-6-((6,7-dimethoxy-1,5-naphthyridin-4-yl)oxy)pyridin-3-amine (79): Compound 79 was synthesized from Compound 78 in the same manner that Compound 8 was synthesized from Compound 5 in the first step of Example 5. MS for C₁₅H₁₃ClN₄O₃, found 333 (MH+).

1-N′-[5-Chloro-6-[(6,7-dimethoxy-1,5-naphthyridin-4-yl)oxy]pyridin-3-yl]-1-N-(4-fluorophenyl)cyclopropane-1,1-dicarboxamide (80): Compound 80 was synthesized from Compound 79 and Compound 9 in the same manner that Compound 10 was synthesized from Compound 8 and Compound 9 in the second step of Example 5. MS for C₂₆H₂₁ClFN₅O₅, found 538 (MH+).

The following compounds were prepared from Compound 77 in a three step procedure analogous to that followed for Compound 80 in Example 17. The procedure for the last step was either that followed in producing Compound 80 from Compound 79 and Compound 9 in Example 17 or that followed for producing Compound 43 from Compound 42 and Compound 9 in Step 4 of Example 10:

1-N′-[6-[(6,7-Dimethoxy-1,5-naphthyridin-4-yl)oxy]pyridin-3-yl]-1-N-(4-fluorophenyl)cyclopropane-1,1-dicarboxamide (81): 2-Chloro-5-nitropyridine was used in place of 2,3-dichloro-5-nitro-pyridine. MS (EI) for C₂₆H₂₂FN₅O₅, found 504 (MH+).

1-N′-[6-[(6,7-Dimethoxy-1,5-naphthyridin-4-yl)oxy]-5-fluoropyridin-3-yl]-1-N-(4-fluorophenyl)cyclopropane-1,1-dicarboxamide (82): 2-Chloro-3-fluoro-5-nitropyridine was used in place of 2,3-dichloro-5-nitro-pyridine. MS (EI) for C₂₆H₂₁F₂N₅O₅, found 522 (MH+).

1-N′-[6-[(6,7-Dimethoxy-1,5-naphthyridin-4-yl)oxy]-4-methylpyridin-3-yl]-1-N-(4-fluorophenyl)cyclopropane-1,1-dicarboxamide (83): 2-Chloro-4-methyl-5-nitropyridine was used in place of 2,3-dichloro-5-nitro-pyridine. ¹H NMR (400 MHz, DMSO-d₆) δ 10.27 (s, 1H), 9.81 (br s, 1H), 8.68 (d, 1H), 8.01 (s, 1H), 7.71-7.59 (m, 3H), 7.30 (d, 1H), 7.21-7.11 (m, 3H), 3.94 (s, 3H), 3.59 (s, 3H), 2.25 (s, 3H), 1.50 (s, 4H); MS (EI) for C₂₇H₂₄FN₅O₅, found 518.1 (MH+).

1-N′-[5-Cyano-6-[(6,7-dimethoxy-1,5-naphthyridin-4-yl)oxy]pyridin-3-yl]-1-N-(4-fluorophenyl)cyclopropane-1,1-dicarboxamide (84): 2-Chloro-5-nitronicotinonitrile was used in place of 2,3-dichloro-5-nitro-pyridine. ¹H NMR (400 MHz, DMSO-d₆) δ 10.41 (br s, 1H), 10.09 (br s, 1H), 8.77 (d, 1H), 8.63 (d, 1H), 8.38 (d, 1H), 7.68 (s, 1H), 7.62 (dd, 2H), 7.56 (d, 1H), 7.14 (t, 2H), 3.94 (s, 3H), 3.52 (s, 3H), 1.45 (br d, 4H); MS (EI) for C₂₇H₂₁FN₆O₅, found 529.1 (MH+).

1-N′-[6-[(6,7-Dimethoxy-1,5-naphthyridin-4-yl)oxy]-5-methylpyridin-3-yl]-1-N-(4-fluorophenyl)cyclopropane-1,1-dicarboxamide (85): 2-Fluoro-3-methyl-5-nitropyridine was used in place of 2,3-dichloro-5-nitro-pyridine. ¹H NMR (400 MHz, DMSO-d₆) δ 10.09 (br d, 2H), 8.67 (d, 1H), 8.01 (br d, 2H), 7.70-7.55 (m, 3H), 7.32 (d, 1H), 7.14 (t, 2H), 3.93 (s, 3H), 3.56 (s, 3H), 2.39 (s, 3H), 1.44 (s, 4H); MS (EI) for C₂₇H₂₄FN₅O₅, found 518.1 (MH+).

1-N′-[6-[(6,7-Dimethoxy-1,5-naphthyridin-4-yl)oxy]-2-methoxypyridin-3-yl]-1-N-(4-fluorophenyl)cyclopropane-1,1-dicarboxamide (86): 6-Chloro-2-methoxy-3-nitropyridine was used in place of 2,3-dichloro-5-nitro-pyridine. ¹H NMR (400 MHz, DMSO-d₆) δ 10.60 (s, 1H), 9.77 (s, 1H), 8.65 (d, 1H), 8.37 (d, 1H), 7.64 (s, 1H), 7.56 (dd, 2H), 7.28-7.13 (m, 3H), 6.73 (d, 1H), 3.95 (s, 3H), 3.71 (s, 3H), 3.57 (s, 3H), 1.65-1.59 (m, 2H), 1.59-1.52 (m, 2H); MS (EI) for C₂₇H₂₄FN₅O₆, found 534.1 (MH+).

1-N′-[6-[(6,7-Dimethoxy-1,5-naphthyridin-4-yl)oxy]-2-methylpyridin-3-yl]-1-N-(4-fluorophenyl)cyclopropane-1,1-dicarboxamide (87): 6-Fluoro-2-methyl-3-nitropyridine was used in place of 2,3-dichloro-5-nitro-pyridine. ¹H NMR (400 MHz, DMSO-d₆) δ 10.20 (s, 1H), 9.92 (s, 1H), 8.66 (d, 1H), 7.92 (d, 1H), 7.69-7.58 (m, 3H), 7.25 (d, 1H), 7.15 (t, 2H), 7.05 (d, 1H), 3.94 (s, 3H), 3.62 (s, 3H), 2.15 (s, 3H), 1.51 (s, 4H); MS (EI) for C₂₇H₂₄FN₅O₅, found 518.1 (MH+).

1-N′-[6-[(6,7-Dimethoxy-1,5-naphthyridin-4-yl)oxy]-4-methoxypyridin-3-yl]-1-N-(4-fluorophenyl)cyclopropane-1,1-dicarboxamide (88): 2-Chloro-4-methoxy-5-nitropyridine was used in place of 2,3-dichloro-5-nitro-pyridine. ¹H NMR (400 MHz, DMSO-d₆) δ 10.40 (s, 1H), 9.89 (s, 1H), 8.67 (d, 1H), 8.38 (s, 1H), 7.64 (s, 1H), 7.58 (dd, 2H), 7.28 (d, 1H), 7.18 (t, 2H), 7.06 (s, 1H), 3.95 (s, 6H), 3.65 (s, 3H), 1.64-1.58 (m, 2H), 1.57-1.50 (m, 2H); MS (EI) for C₂₇H₂₄FN₅O₆, found 534.1 (MH+).

1-N′-[6-[(6,7-Dimethoxy-1,5-naphthyridin-4-yl)oxy]-5-(trifluoromethyl)pyridin-3-yl]-1-N-(4-fluorophenyl)cyclopropane-1,1-dicarboxamide (89): 2-Chloro-5-nitro-3-(trifluoromethyl)pyridine was used in place of 2,3-dichloro-5-nitro-pyridine. ¹H NMR (400 MHz, DMSO-d₆) δ 10.37 (s, 1H), 10.09 (s, 1H), 8.74 (d, 1H), 8.60 (s, 1H), 8.36 (s, 1H), 7.66 (s, 1H), 7.62 (m, 2H), 7.49 (s, 1H), 7.14 (t, 2H), 3.93 (s, 3H) 3.45 (s, 3H), 1.44 (s, 4H); MS (EI) for C₂₇H₂₁F4N₅O₅, found 572 (MH+).

1-N′-[5-Bromo-6-[(6,7-dimethoxy-1,5-naphthyridin-4-yl)oxy]-2-methylpyridin-3-yl]-1-N-(4-fluorophenyl)cyclopropane-1,1-dicarboxamide (90): 3-Bromo-2-chloro-6-methyl-5-nitropyridine was used in place of 2,3-dichloro-5-nitro-pyridine. ¹H NMR (400 MHz, CD₃OD) δ 8.67 (d, 1H), 8.40 (s, 1H), 8.35 (s, 1H), 7.55 (t, 3H), 7.37 (s, 1H), 7.08 (t, 2H), 4.00 (s, 3H), 3.66 (s, 3H), 2.17 (s, 3H), 1.90-1.67 (m, 4H); MS (EI) for C₂₇H₂₃BrFN₅O₅, found 596.1 (MH+).

1-N′-[2-Carbamoyl-6-[(6,7-dimethoxy-1,5-naphthyridin-4-yl)oxy]pyridin-3-yl]-1-N-(4-fluorophenyl)cyclopropane-1,1-dicarboxamide (91): 6-Chloro-3-nitropicolinamide was used in place of 2,3-dichloro-5-nitro-pyridine. ¹H NMR (400 MHz, CDCl₃) δ 11.93 (s, 1H), 11.07 (s, 1H), 9.16 (d, 1H), 8.65 (s, 1H), 7.68 (s, 1H), 7.51 (t, 2H), 7.33 (d, 1H), 7.24 (s, 1H), 7.19 (m, 1H), 6.95 (t, 2H), 5.29 (s, 1H), 3.98 (s, 3H), 3.73 (s, 3H), 1.91 (s, 2H), 1.62 (s, 2H); MS (EI) for C₂₇H₂₃FN₆O₆, found 547.1 (MH+).

1-N′-[6-[(6,7-Dimethoxy-1,5-naphthyridin-4-yl)oxy]-5-methoxypyridin-3-yl]-1-N-(4-fluorophenyl)cyclopropane-1,1-dicarboxamide (92): 2-Chloro-3-methoxy-5-nitropyridine was used in place of 2,3-dichloro-5-nitro-pyridine. ¹H NMR (400 MHz, CDCl₃) δ 10.32 (s, 1H), 8.60 (d, 1H), 8.32 (s, 1H), 8.02 (s, 1H), 7.85 (s, 1H), 7.79 (s, 1H), 7.47 (d, 2H), 7.24 (d, 1H), 7.07 (t, 2H), 4.05 (s, 3H), 3.96 (s, 3H), 3.85 (s, 3H), 1.87 (s, 2H), 1.68 (s, 2H); MS (EI) for C₂₇H₂₄FN₅O₆, found 534.1 (MH+).

### Example 18: 1-N′-[6-[(6,7-Dimethoxy-1,5-naphthyridin-4-yl)oxy]-2,5-difluoropyridin-3-yl]-1-N-(4-fluorophenyl)cyclopropane-1,1-dicarboxamide (96)

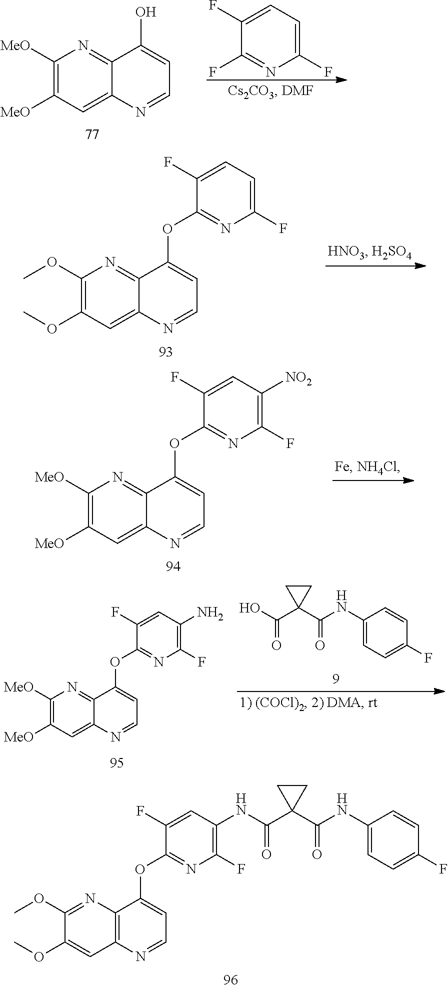

8-((3,6-Difluoropyridin-2-yl)oxy)-2,3-dimethoxy-1,5-naphthyridine (93): To a solution of Compound 77 (400 mg, 1.94 mmol, 1 eq) in DMF (6 mL) was added 2,3,6-trifluoropyridine (387.21 mg, 2.91 mmol, 1.5 eq) and Cs₂CO₃ (1.58 g, 4.85 mmol, 2.5 eq). The mixture was stirred at 25° C. for 0.5 h. The reaction mixture was added to water (20 mL), which was then extracted with EtOAc (3×25 mL). The combined EtOAc extracts were dried over anhyd Na₂SO₄ and concentrated in vacuo. The resulting residue was purified by flash silica gel chromatography (ISCO®; 4 g SepaFlash® Silica Flash Column, Eluent of 0-35% Ethyl acetate/Petroleum ether gradient @ 25 mL/min) to give Compound 93 as a white solid (100 mg, 16.2% yield). ¹H NMR (400 MHz, CDCl₃) δ 7.97-7.86 (m, 1H), 7.57 (dd, 1H), 7.26-7.19 (m, 1H), 6.70 (d, 1H), 6.58 (d, 1H), 4.25 (s, 3H), 3.84 (s, 3H).

8-((3,6-Difluoro-5-nitropyridin-2-yl)oxy)-2,3-dimethoxy-1,5-naphthyridine (94): To a 3-neck, round-bottomed flask was added Compound 93 (120 mg, 375.87 umol, 1 eq) followed by the addition of HNO₃ (592.11 mg, 9.40 mmol, 422.94 uL, 25 eq). H₂SO₄ (552.97 mg, 5.64 mmol, 300.52 uL, 15 eq) was added slowly, maintaining the internal temperature below 40° C. The resulting solution was heated to 60° C. for 30 minutes, then cooled to room temperature, followed by cooling in an ice-water bath. The reaction mixture was quenched with water and extracted with EtOAc (3×20 mL). The combined organic layers were dried over anhyd Na₂SO₄ and concentrated. The resulting residue was purified by prep-TLC (petroleum ether/ethyl acetate=2/1) to give Compound 94 as a yellow solid (50 mg, 29.2% yield). ¹H NMR (400 MHz, CDCl₃) δ 8.81 (d, 1H), 8.47 (s, 1H), 7.58 (s, 1H), 7.38 (d, 1H), 4.08-3.98 (m, 3H), 3.71 (s, 3H).

6-((6,7-Dimethoxy-1,5-naphthyridin-4-yl)oxy)-2,5-difluoropyridin-3-amine (95): Compound 95 was synthesized from Compound 94 in the same manner that Compound 8 was synthesized from Compound 5 in the first step of Example 5. MS for C₁₅H₁₂F₂N₄O₃, found 335.2 (MH+).

1-N′-[6-[(6,7-Dimethoxy-1,5-naphthyridin-4-yl)oxy]-2,5-difluoropyridin-3-yl]-1-N-(4-fluorophenyl)cyclopropane-1,1-dicarboxamide (96): Compound 96 was made from Compound 95 and Compound 9 using a method analogous to that use to make Compound 43 from Compound 42 and Compound 9 in Step 4 of Example 10. ¹H NMR (400 MHz, CDCl₃) δ 10.21 (s, 1H), 8.76-8.65 (m, 2H), 8.02 (s, 1H), 7.53 (s, 1H), 7.48-7.40 (m, 2H), 7.30 (d, 1H), 7.06 (t, 2H), 4.01 (s, 3H), 3.74 (s, 3H), 1.88-1.79 (m, 2H), 1.68-1.62 (m, 2H); MS (EI) for C₂₆H₂₀F₃N₅O₅, found 540.1 (MH+).

### Example 19: 1-N′-[6-(2,3-Dihydro-[1,4]dioxino[2,3-b][1,5]naphthyridin-6-yloxy)pyridin-3-yl]-1-N-(4-fluorophenyl)cyclopropane-1,1-dicarboxamide (103)

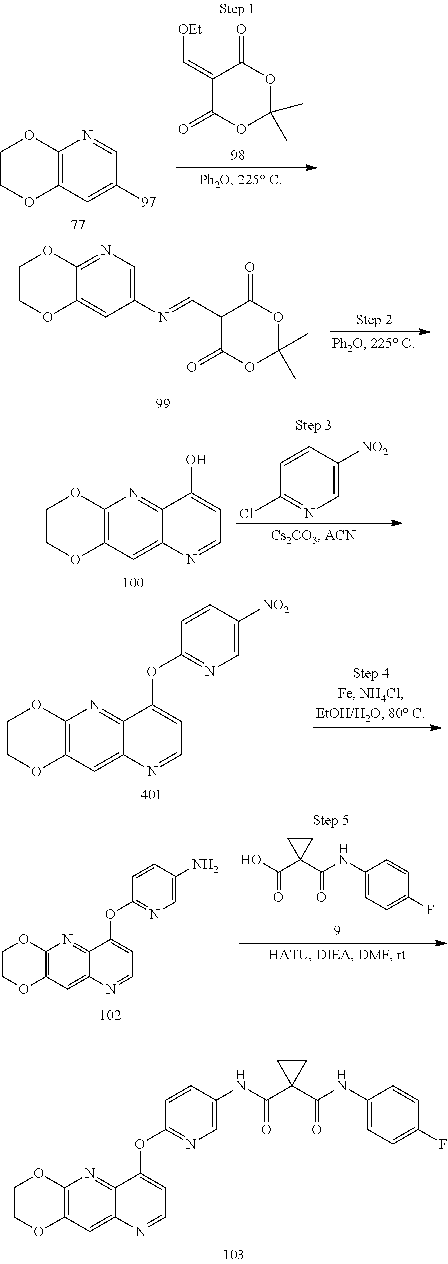

(E)-5-(((2,3-Dihydro-[1,4]dioxino[2,3-b]pyridin-7-yl)imino)methyl)-2,2-dimethyl-1,3-dioxane-4,6-dione (99): Compound 99 was synthesized from Compound 97 in the same manner that Compound 54 was synthesized from Compound 52 in Step 1 of Example 12, substituting triethoxymethane for trimethoxymethane to form Compound 98.

2,3-Dihydro-[1,4]dioxino[2,3-b][1,5]naphthyridin-6-ol (100): Compound 100 was synthesized from Compound 99 in the same manner that Compound 55 was synthesized from Compound 54 in Step 2 of Example 12. MS (EI) for C₁₀H₈N₂O₃, found 199 (MH+).

6-((5-Nitropyridin-2-yl)oxy)-2,3-dihydro-[1,4]dioxino[2,3-b][1,5]naphthyridine (101): Compound 101 was synthesized from Compound 100 and 2-chloro-5-nitropyridine in the same manner that Compound 6 was synthesized from Compound 4 and 2,3-dichloro-5-nitro-pyridine in Example 3.

6-((2,3-Dihydro-[1,4]dioxino[2,3-b][1,5]naphthyridin-6-yl)oxy)pyridin-3-amine (102): Compound 102 was synthesized from Compound 101 in the same manner that Compound 8 was synthesized from Compound 5 in the first step of Example 5.

1-N′-[6-(2,3-Dihydro-[1,4]dioxino[2,3-b][1,5]naphthyridin-6-yloxy)pyridin-3-yl]-1-N-(4-fluorophenyl)cyclopropane-1,1-dicarboxamide (103): Compound 103 was synthesized from Compound 102 and Compound 9 in the same manner that Compound 10 was synthesized from Compound 8 and Compound 9 in the second step of Example 5. MS (EI) for C₂₆H₂₀FN₅O₅, found 502 (MH+).

The following compounds were prepared from Compound 100 in the same manner that Compound 103 was prepared from Compound 100 using Steps 3-5 in Example 19:

1-N′-[5-Chloro-6-(2,3-dihydro-[1,4]dioxino[2,3-b][1,5]naphthyridin-6-yloxy)pyridin-3-yl]-1-N-(4-fluorophenyl)cyclopropane-1,1-dicarboxamide (104): 2,3-Dichloro-5-nitropyridine was used in place of 2-chloro-5-nitropyridine. MS (EI) for C₂₆H₁₉ClFN₅O₅, found 536 (MH+).

1-N′-[6-(2,3-Dihydro-[1,4]dioxino[2,3-b][1,5]naphthyridin-6-yloxy)-5-fluoropyridin-3-yl]-1-N-(4-fluorophenyl)cyclopropane-1,1-dicarboxamide (105): 2-Chloro-3-fluoro-5-nitropyridine was used in place of 2-chloro-5-nitropyridine. MS (EI) for C₂₆H₁₉F₂N₅O₅, found 520 (MH+).

### Example 20: 1-N-(4-Fluorophenyl)-1-N′-[6-[[6-methoxy-7-(2-methoxyethoxy)-1,5-naphthyridin-4-yl]oxy]pyridin-3-yl]cyclopropane-1,1-dicarboxamide (114)

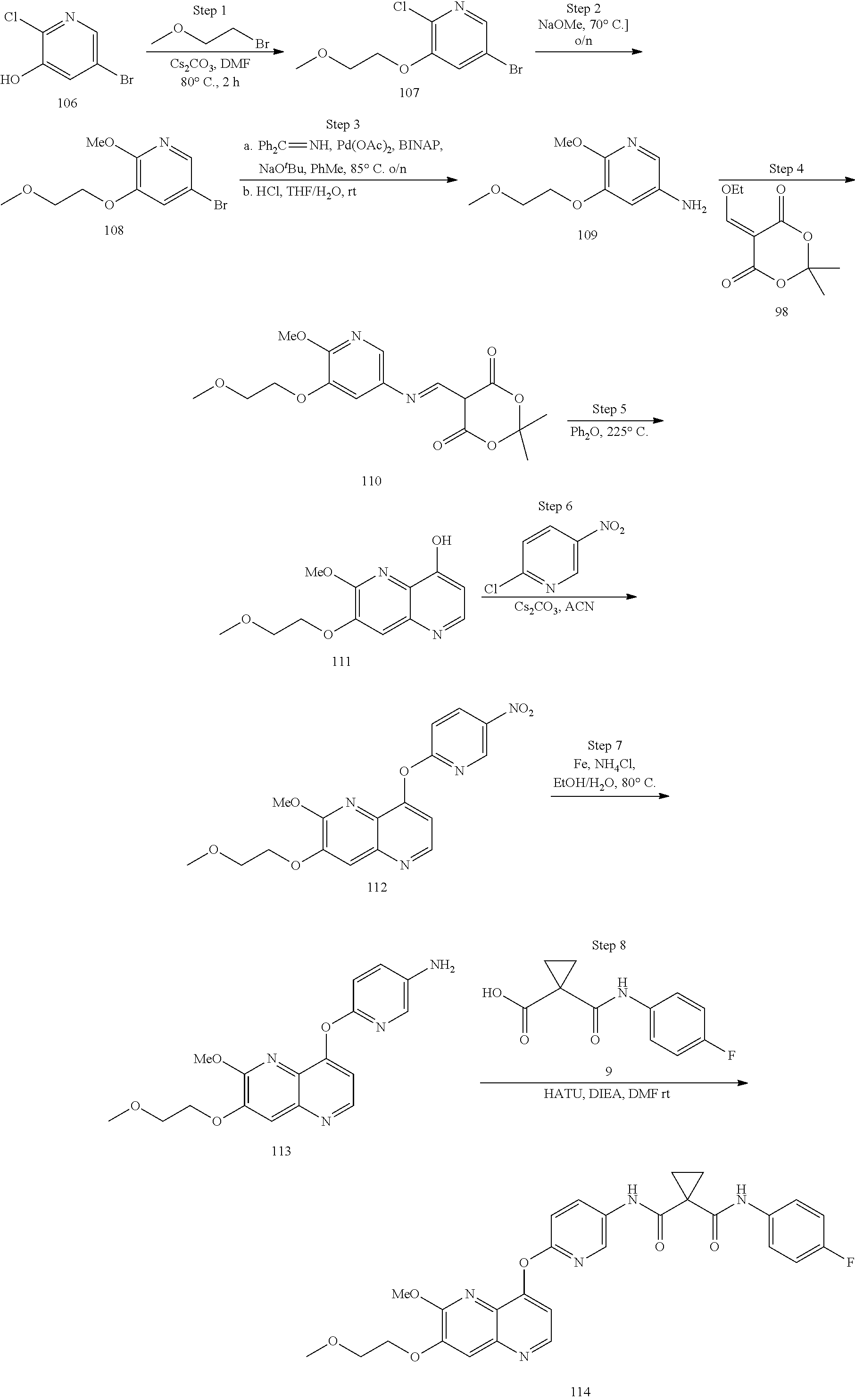

5-Bromo-2-chloro-3-(2-methoxyethoxy)pyridine (107): A mixture of Compound 106 (2.10 g, 10.0 mmol), 1-bromo-2-methoxyethane (1.50 g, 10.8 mmol), and Cs₂CO₃, (6.6 g, 20.2 mmol) in DMF was stirred at 80° C. for 2 h, quenched with water, and extracted with EtOAc (2×), The combined extracts were washed with aq saturated NaCl, dried over Na₂SO₄ and concentrated to give crude Compound 107 as an off-white solid (2.68 g, ˜100% yield). MS for C₈H₉BrClNO₂, found 268 (MH+).

5-Bromo-2-methoxy-3-(2-methoxyethoxy)pyridine (108): Compound 107 (2.68 g, 10.0 mmol) was mixed with NaOMe (3.0 g, 55.5 mmol) in MeOH (40 mL) and heated at 70° C. overnight. The reaction mixture was concentrated to remove MeOH. The residue was partitioned between water and EtOAc. The EtOAc solution was washed with aq saturated NaCl, dried over Na₂SO₄, and evaporated to give crude Compound 108 (3.0 g). MS for C₉H₁₂BrNO₃, found 262/264 (MH+).

6-Methoxy-5-(2-methoxyethoxy)pyridin-3-amine (109): Compound 108 (3.0 g, crude) was mixed with diphenylmethanimine (3.6 g, 20 mmol), Pd(OAc)₂ (360 mg, 1.61 mmol), BINAP (1.3 g, 2.08 mmol), and NaO^{t}Bu (1.6 g, 16.7 mmol) in toluene (60 mL). The resulting mixture was degassed by bubbling argon, stirred at 85° C. overnight, and then partitioned between water and EtOAc. The organic phase was separated and evaporated to dryness. To the residue was added THF (40 mL) and HCl (aq, 2M, 40 mL), and the resulting mixture was stirred at room temperature overnight. The reaction mixture was neutralized to pH 10 with NaHCO₃ and extracted with EtOAc. The extract was concentrated, and the residue was subjected to chromatography on silica gel, eluted with 0-90% EtOAc in hexanes, to give Compound 110 as a brown oil (1.4 g, 71% yield). MS for C₉H₁₄N₂O₃, found 199 (MH+).

(E)-5-(((6-Methoxy-5-(2-methoxyethoxy)pyridin-3-yl)imino)methyl)-2,2-dimethyl-1,3-dioxane-4,6-dione (110): Compound 110 was synthesized from Compound 109 in the same manner that Compound 54 was synthesized from Compound 52 in Step 1 of Example 12, substituting triethoxymethane for trimethoxymethane to form Compound 98.

6-Methoxy-7-(2-methoxyethoxy)-1,5-naphthyridin-4-ol (111): Compound 111 was synthesized from Compound 110 in the same manner that Compound 55 was synthesized from Compound 54 in Step 2 of Example 12. MS for C₁₂H₁₄N₂O₄, found 251 (MH+).

2-Methoxy-3-(2-methoxyethoxy)-8-((5-nitropyridin-2-yl)oxy)-1,5-naphthyridine (112): Compound 112 was synthesized from Compound 111 and 2-chloro-5-nitropyridine in the same manner that Compound 6 was synthesized from Compound 4 and 2,3-dichloro-5-nitro-pyridine in Example 3.

6-((6-Methoxy-7-(2-methoxyethoxy)-1,5-naphthyridin-4-yl)oxy)pyridin-3-amine (113): Compound 113 was synthesized from Compound 112 in the same manner that Compound 8 was synthesized from Compound 5 in the first step of Example 5.

1-N-(4-Fluorophenyl)-1-N′-[6-[[6-methoxy-7-(2-methoxyethoxy)-1,5-naphthyridin-4-yl]oxy]pyridin-3-yl]cyclopropane-1,1-dicarboxamide (114): Compound 114 was synthesized from Compound 113 and Compound 9 in the same manner that Compound 10 was synthesized from Compound 8 and Compound 9 in the second step of Example 5. MS for C₂₈H₂₆FN₅O₆, found 548 (MH+).

The following compounds were prepared from Compound 111 in the same manner that Compound 114 was prepared from Compound 111 using Steps 6-8 in Example 20:

1-N′-[5-Chloro-6-[[6-methoxy-7-(2-methoxyethoxy)-1,5-naphthyridin-4-yl]oxy]pyridin-3-yl]-1-N-(4-fluorophenyl)cyclopropane-1,1-dicarboxamide (115): 2,3-Dichloro-5-nitropyridine was used in place of 2-chloro-5-nitropyridine. MS for C₂₈H₂₅ClFN₅O₆, found 582 (MH+).

1-N′-[5-Fluoro-6-[[6-methoxy-7-(2-methoxyethoxy)-1,5-naphthyridin-4-yl]oxy]pyridin-3-yl]-1-N-(4-fluorophenyl)cyclopropane-1,1-dicarboxamide (116): 2-Chloro-3-fluoro-5-nitropyridine was used in place of 2-chloro-5-nitropyridine. MS for C₂₈H₂₅F₂N₅O₆, found 566 (MH+).

### Example 21: 1-N′-[5-Fluoro-6-[[6-methoxy-7-[(4-methoxyphenyl)methoxy]-1,5-naphthyridin-4-yl]oxy]pyridin-3-yl]-1-N-(4-fluorophenyl)cyclopropane-1,1-dicarboxamide (120)

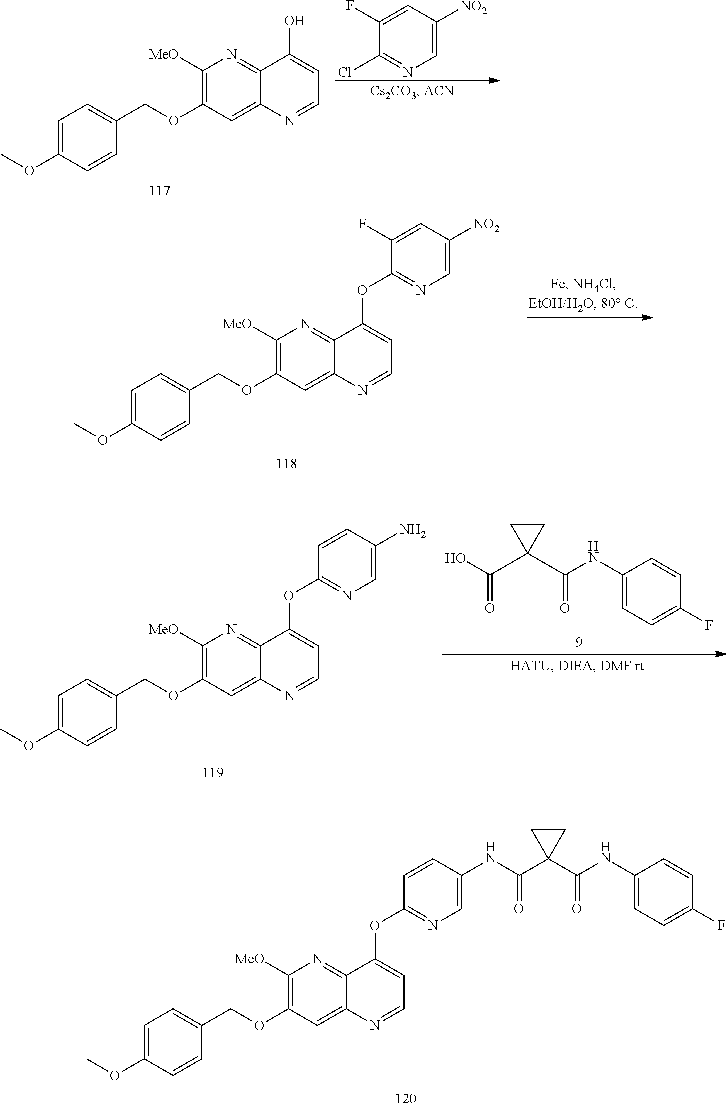

6-Methoxy-7-((4-methoxybenzyl)oxy)-1,5-naphthyridin-4-ol (117): Compound 117 was synthesized from Compound 106 using the same method for the synthesis of Compound 111 in Example 20 (Steps 1-5), replacing 1-bromo-2-methoxyethane with 1-(chloromethyl)-4-methoxybenzene. MS for C₁₇H₁₆N₂O₄, found 313 (MH+).

8-((3-Fluoro-5-nitropyridin-2-yl)oxy)-2-methoxy-3-((4-methoxybenzyl)oxy)-1,5-naphthyridine (118): Compound 118 was synthesized from Compound 117 and 2-chloro-3-fluoro-5-nitropyridine in the same manner that Compound 6 was synthesized from Compound 4 and 2,3-dichloro-5-nitro-pyridine in Example 3.

5-Fluoro-6-((6-methoxy-7-((4-methoxybenzyl)oxy)-1,5-naphthyridin-4-yl)oxy)pyridin-3-amine (119): Compound 119 was synthesized from Compound 118 in the same manner that Compound 8 was synthesized from Compound 5 in the first step of Example 5.

1-N′-[5-Fluoro-6-[[6-methoxy-7-[(4-methoxyphenyl)methoxy]-1,5-naphthyridin-4-yl]oxy]pyridin-3-yl]-1-N-(4-fluorophenyl)cyclopropane-1,1-dicarboxamide (120): Compound 120 was synthesized from Compound 119 and Compound 9 in the same manner that Compound 10 was synthesized from Compound 8 and Compound 9 in the second step of Example 5. MS for C₃₃H₂₇F₂N₅O₆, found 628 (MH+).

### Example 22: 1-N′-[5-Fluoro-6-[(7-hydroxy-6-methoxy-1,5-naphthyridin-4-yl)oxy]pyridin-3-yl]-1-N-(4-fluorophenyl)cyclopropane-1,1-dicarboxamide (121)

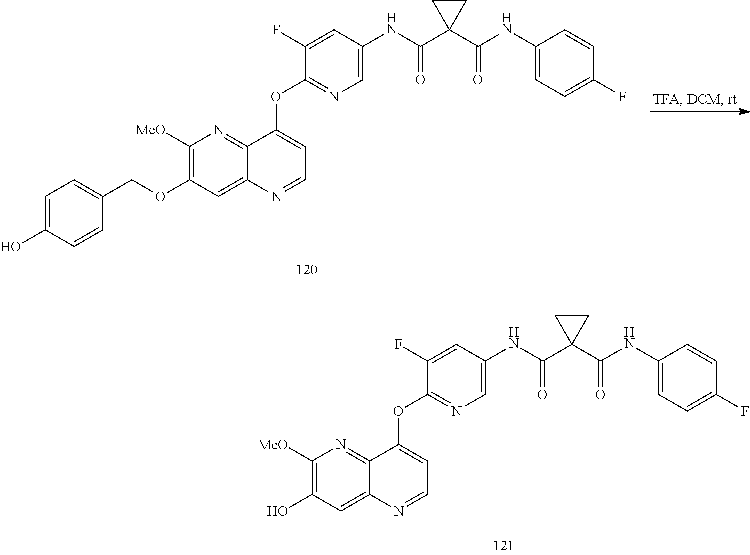 [SMILES: COc1nc2c(Oc3ncc(NC(=O)C4(C(=O)Nc5ccc(F)cc5)CC4)cc3F)ccnc2cc1OCc1ccc(O)cc1 ; COc1nc2c(Oc3ncc(NC(=O)C4(C(=O)Nc5ccc(F)cc5)CC4)cc3F)ccnc2cc1O]

1-N′-[5-Fluoro-6-[(7-hydroxy-6-methoxy-1,5-naphthyridin-4-yl)oxy]pyridin-3-yl]-1-N-(4-fluorophenyl)cyclopropane-1,1-dicarboxamide (121): To a solution of Compound 120 (100 mg, 0.159 mmol) in DCM (6 mL) was added TFA (1 mL), and the solution was stirred at room temperature for 1 h and then concentrated to dryness. The resulting residue was purified by prep-HPLC to give Compound 121. MS for C₂₅H₁₉F₂N₅O₅, found 508 (MH+).

### Example 23: 1-N′-[5-Fluoro-6-[[6-methoxy-7-(3-morpholin-4-ylpropoxy)-1,5-naphthyridin-4-yl]oxy]pyridin-3-yl]-1-N-(4-fluorophenyl)cyclopropane-1,1-dicarboxamide (122)

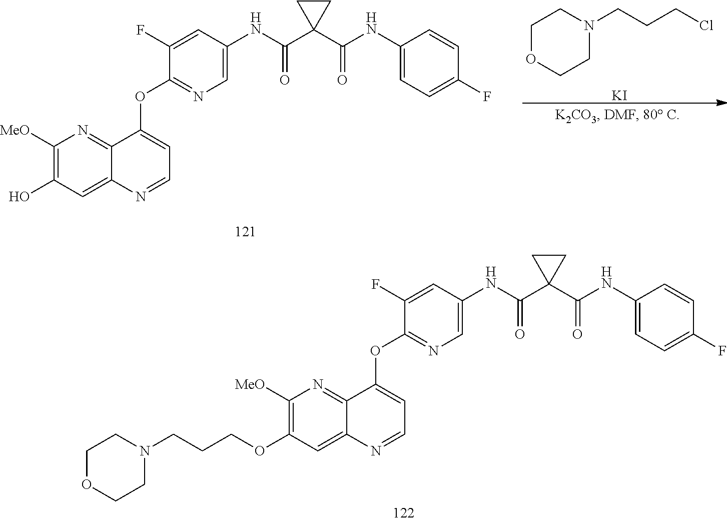

1-N′-[5-Fluoro-6-[[6-methoxy-7-(3-morpholin-4-ylpropoxy)-1,5-naphthyridin-4-yl]oxy]pyridin-3-yl]-1-N-(4-fluorophenyl)cyclopropane-1,1-dicarboxamide (122): A mixture of Compound 121 (20 mg, 0.039 mmol), 4-(3-chloropropyl)morpholine (13 mg, 0.079 mmol), K₂CO₃ (22 mg, 0.16 mmol), and KI (2 mg, 0.012 mmol) in DMF (1 mL) was stirred at 80° C. for 2 h. The reaction was partitioned between water and EtOAc. The EtOAc phase was separated and evaporated and the resulting residue was purified by prep-HPLC to give Compound 122. MS for C₃₂H₃₂F₂N₆O₆, found 635 (MH+).

The following compounds were prepared from Compound 121 in the same manner that Compound 122 was prepared from Compound 111 in Example 23:

1-N′-[5-Fluoro-6-[[6-methoxy-7-(2-morpholin-4-ylethoxy)-1,5-naphthyridin-4-yl]oxy]pyridin-3-yl]-1-N-(4-fluorophenyl)cyclopropane-1,1-dicarboxamide (123): 4-(2-Chloroethyl)morpholine was used in place of 4-(3-chloropropyl)morpholine. MS for C₃₁H₃₀F₂N₆O₆, found 621 (MH+).

1-N′-[6-[[7-(2-Amino-2-oxoethoxy)-6-methoxy-1,5-naphthyridin-4-yl]oxy]-5-fluoropyridin-3-yl]-1-N-(4-fluorophenyl)cyclopropane-1,1-dicarboxamide (124): 2-Chloroacetamide was used in place of 4-(3-chloropropyl)morpholine. MS for C₂₇H₂₂F₂N₆O₆, found 565 (MH+).

1-N′-[6-[[7-[3-(Dimethylamino)propoxy]-6-methoxy-1,5-naphthyridin-4-yl]oxy]-5-fluoropyridin-3-yl]-1-N-(4-fluorophenyl)cyclopropane-1,1-dicarboxamide (125): 3-Chloro-N,N-dimethylpropan-1-amine was used in place of 4-(3-chloropropyl)morpholine. MS for C₃₀H₃₀F₂N₆O₅, found 593 (MH+).

### Example 24: 1-N′-[6-[[7-(2,3-Dihydroxypropoxy)-6-methoxy-1,5-naphthyridin-4-yl]oxy]-5-fluoropyridin-3-yl]-1-N-(4-fluorophenyl)cyclopropane-1,1-dicarboxamide (127)

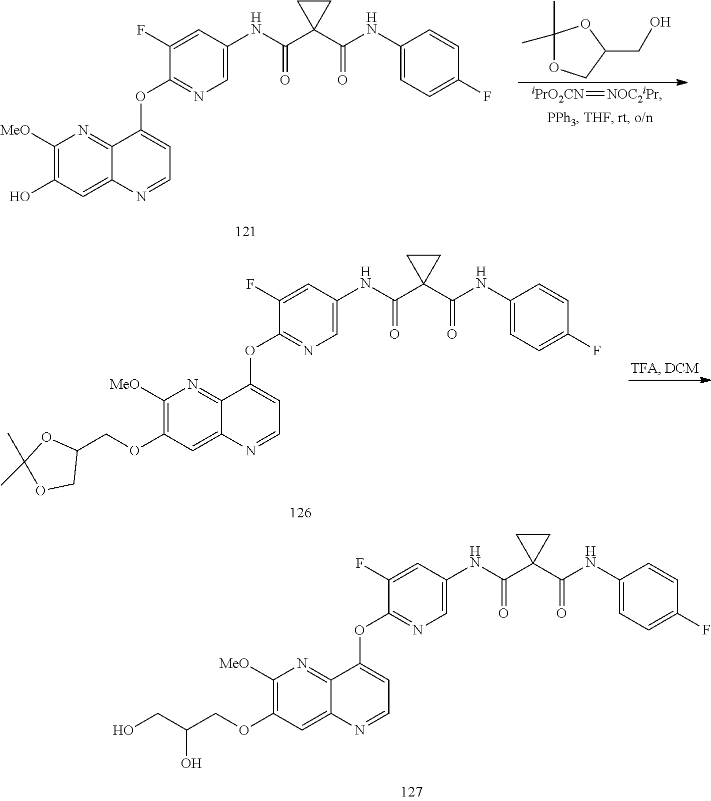

N-(6-((7-((2,2-dimethyl-1,3-dioxolan-4-yl)methoxy)-6-methoxy-1,5-naphthyridin-4-yl)oxy)-5-fluoropyridin-3-yl)-N-(4-fluorophenyl)cyclopropane-1,1-dicarboxamide (126): A mixture of Compound 121 (28 mg, 0.056 mmol), (2,2-dimethyl-1,3-dioxolan-4-yl)methanol (15 mg, 0.11 mmol), diisopropyl azodicarboxylate (24 mg, 0.11 mmol), and PPh₃ (37 mg, 0.14 mmol) in THF (2 mL) was stirred at room temperature overnight. The reaction mixture was subjected to silica gel chromatography, eluted with 0-100% EtOAc in hexanes, to give Compound 126 (26 mg, 76% yield). MS for C₃₁H₂₉F₂N₅O₇, found 622 (MH+).

1-N′-[6-[[7-(2,3-Dihydroxypropoxy)-6-methoxy-1,5-naphthyridin-4-yl]oxy]-5-fluoropyridin-3-yl]-1-N-(4-fluorophenyl)cyclopropane-1,1-dicarboxamide (127): Compound 126 (26 mg, 0.042 mmol) was dissolved in DCM (3 mL) followed by the addition of TFA (0.5 mL). The mixture was stirred at room temperature until complete, then evaporated to dryness.

The resulting residue was purified by prep-HPLC to give Compound 127 as an off-white solid (16 mg, 66% yield). MS for C₂₈H₂₅F₂N₅O₇, found 582 (MH+).

### Example 25: 1-N′-[5-Fluoro-6-[[7-methoxy-6-(2-methoxyethoxy)-1,5-naphthyridin-4-yl]oxy]pyridin-3-yl]-1-N-(4-fluorophenyl)cyclopropane-1,1-dicarboxamide (134)

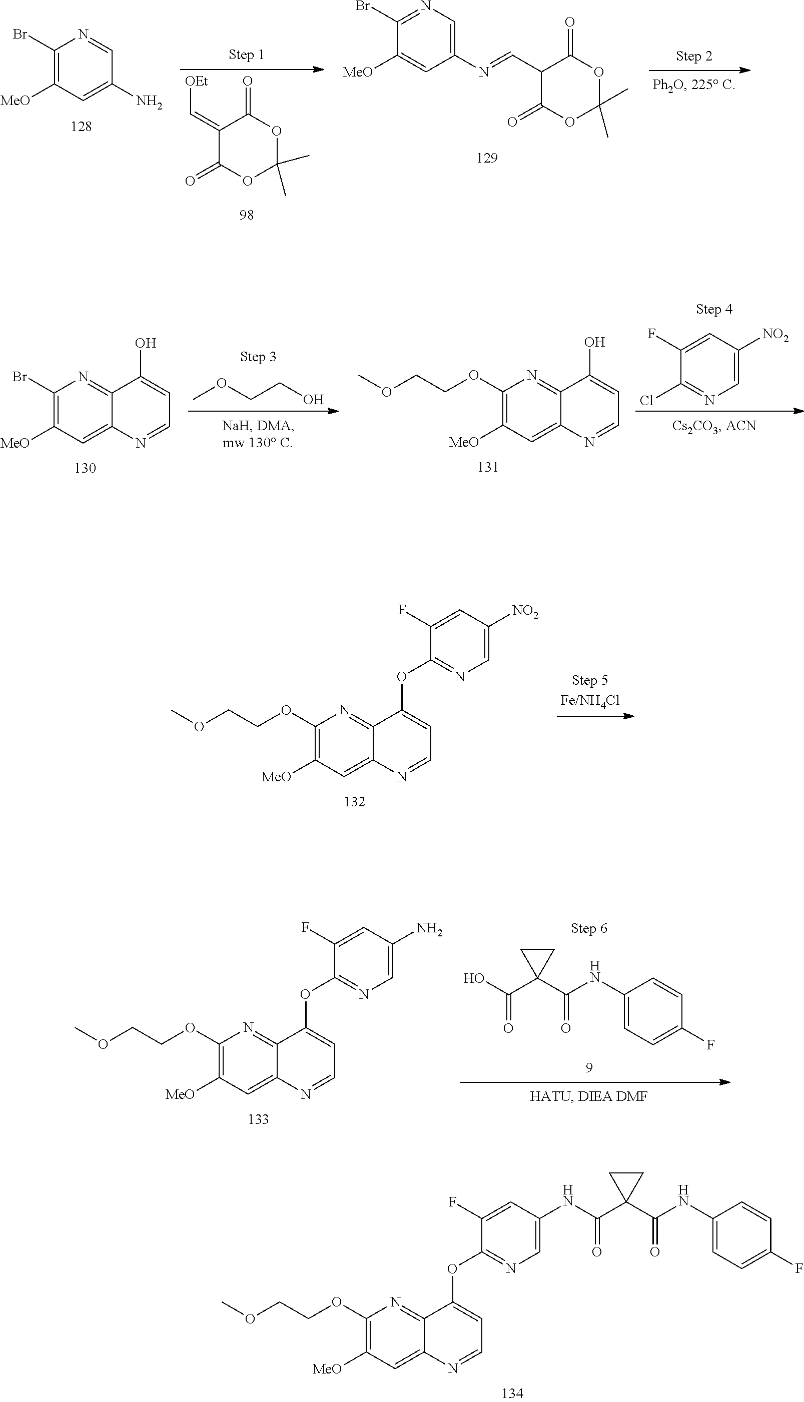

(E)-5-(((6-Bromo-5-methoxypyridin-3-yl)imino)methyl)-2,2-dimethyl-1,3-dioxane-4,6-dione (129): Compound 129 was synthesized from Compound 128 in the same manner that Compound 54 was synthesized from Compound 52 in Step 1 of Example 12, substituting triethoxymethane for trimethoxymethane to form Compound 98.

6-Bromo-7-methoxy-1,5-naphthyridin-4-ol (130): Compound 130 was synthesized from Compound 129 in the same manner that Compound 55 was synthesized from Compound 54 in Step 2 of Example 12. MS for C₉H₇BrN₂O₂, found 255/257 (MH+).

7-Methoxy-6-(2-methoxyethoxy)-1,5-naphthyridin-4-ol (131): To a solution of 2-methoxyethan-1-ol (2.1 mmol) in DMA (4 mL) was slowly added NaH (60% in oil, 2.0 mmol), and the resulting suspension was stirred at room temperature for 15 min, followed by the addition of Compound 130 (100 mg, 0.39 mmol). The mixture was heated under microwave condition at 130° C. for 20 min, cooled to room temperature, and taken into the next step as is.

8-((3-Fluoro-5-nitropyridin-2-yl)oxy)-3-methoxy-2-(2-methoxyethoxy)-1,5-naphthyridine (132): To the reaction mixture of crude Compound 131 above was added 2-chloro-3-fluoro-5-nitropyridine (6 eq, 412 mg, 2.34 mmol) and Cs₂CO₃ (6 eq, 763 mg, 2.34 mmol). The mixture was stirred at room temperature overnight and then partitioned between water and EtOAc. The organic phase was washed with aq saturated NaCl and concentrated. The residue was purified by silica gel column to give Compound 132.

5-Fluoro-6-((7-methoxy-6-(2-methoxyethoxy)-1,5-naphthyridin-4-yl)oxy)pyridin-3-amine (133): Compound 133 was synthesized from Compound 132 in the same manner that Compound 8 was synthesized from Compound 5 in the first step of Example 5.

1-N′-[5-Fluoro-6-[[7-methoxy-6-(2-methoxyethoxy)-1,5-naphthyridin-4-yl]oxy]pyridin-3-yl]-1-N-(4-fluorophenyl)cyclopropane-1,1-dicarboxamide (134): Compound 134 was synthesized from Compound 133 and Compound 9 in the same manner that Compound 10 was synthesized from Compound 8 and Compound 9 in the second step of Example 5. MS (EI) for C₂₈H₂₅F₂N₅O₆, found 566 (MH+).

The following compound was prepared from Compound 130 using the same sequence of steps used to synthesize Compound 134 from Compound 130 in Example 25 (Steps 3-6):

1-N′-[5-Fluoro-6-[[7-methoxy-6-(3-morpholin-4-ylpropoxy)-1,5-naphthyridin-4-yl]oxy]pyridin-3-yl]-1-N-(4-fluorophenyl)cyclopropane-1,1-dicarboxamide (135): The 2-methoxyethan-1-ol in Step 3 of Example 25 was replaced by 3-morpholinopropan-1-ol. MS (EI) for C₃₂H₃₂F₂N₆O₆, found 635 (MH+).

### Example 26: 4-Chloro-6,7-dimethoxypyrido[3,2-d]pyrimidine (143)

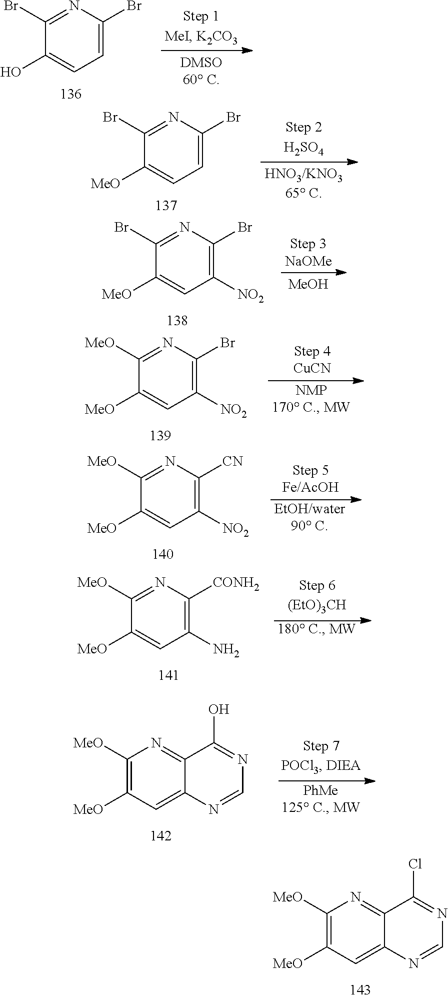

2,6-Dibromo-3-methoxypyridine (137): To a solution of Compound 136 (2.62 g, 10.4 mmol) in DMSO (4.5 mL) were added K₂CO₃ (1.35 g, 9.8 mmol) and methyl iodide (2.2 mL, 35.3 mmol), and the reaction mixture was stirred at 60° C. for 1 h. The mixture was cooled to room temperature and poured into water (50 mL) and filtered. The resulting solids were washed with ice cold water and dried under vacuum to give Compound 137 (2.5 g, 90% yield). MS for C₆H₅Br₂NO, found 268 (MH+).

2,6-Dibromo-3-methoxy-5-nitropyridine (138): To conc H₂SO₄ (15 ml) at 0° C. were added nitric acid (67%, 4.0 mL) and KNO₃ (2.0 g) followed by Compound 137 (2.0 g, 7.5 mmol). The reaction mixture was stirred at 65° C. overnight, after which it was poured into crushed ice and neutralized carefully with solid Na₂CO₃, then extracted with EtOAc (2 times). The combined organic extracts were concentrated, and the resulting residue was purified by flash silica gel chromatography (0-80% of EtOAc in hexanes) to give Compound 138 (732 mg, 31% yield).

2-Bromo-5,6-dimethoxy-3-nitropyridine (139): To a solution of Compound 138 (200 mg, 0.64 mmol) in anhyd MeOH (6 mL) was added NaOMe (46 mg, 0.85 mmol). The reaction mixture was stirred at room temperature for 1 h and then concentrated under vacuum. The resulting residue was washed with water and filtered. The collected solids were washed with ice cold water and dried under vacuum to give Compound 139 (150 mg, 89% yield).

5,6-Dimethoxy-3-nitropicolinonitrile (140): A mixture of Compound 139 (150 mg, 0.57 mmol) and CuCN (170 mg, 1.90 mmol) in NMP (5 mL) was heated at 170° C. under microwave irradiation for 10 min and then cooled to room temperature. The reaction mixture was poured into ice water, and the resulting suspension was filtered, washed with water, and resuspended in hot EtOAc for 30 min. The resulting mixture was filtered through Celite®, and the filtrate was concentrated under vacuum to give Compound 140 which was used without further purification.

3-Amino-5,6-dimethoxypicolinamide (141): Compound 140 was mixed with Fe (130 mg, 2.0 mmol), AcOH (0.4 mL, 6.7 mmol), water (6 mL), and EtOH (14 mL). The mixture was stirred at 90° C. for 20 min and then cooled to room temperature. The pH was adjusted with aq 28% NH₄OH until basic. The resulting mixture was filtered through Celite®. Volatile organics were removed from the filtrate under vacuum, and the resulting mixture was extracted with EtOAc (2 times). The combined EtOAc extracts were concentrated, and the resulting residue purified by flash silica gel chromatography (0-100% EtOAc in hexanes) to give Compound 141 as an off-white solid (49 mg, 44% yield over 2 steps). MS for C₈H₁₁N₃O₃, found 198 (MH+).

6,7-Dimethoxypyrido[3,2-d]pyrimidin-4-ol (142): A suspension of Compound 141 (1 eq) in triethyl orthoformate (2 mL/mmol of Compound 141) was irradiated by microwave at 180° C. for 30 min. After cooling to room temperature, the resulting precipitate was collected by vacuum filtration and washed with hexanes to give Compound 142 (˜ 95% yield). MS for C₉H₉N₃O₃, found 208 (MH+).

6,7-Dimethoxypyrido[3,2-d]pyrimidin-4-ol (143): Compound 143 was made from Compound 142 using standard methods for the conversion of an aromatic alcohol to a chloride using POCl₃. Typically, to a mixture of compound like Compound 142 (1 eq) in toluene (8 mL/1 mmol of Compound 142) was added DIEA (˜3 eq) and phosphorus oxychloride (˜3 eq), and the reaction was stirred at 130° C. under microwave irradiation for 1 h. After cooling to room temperature, the reaction mixture was concentrated, and the resulting residue was subjected to purification by silica gel chromatography. Compound 143 was recovered in such a manner. MS for C₉H₈ClN₃O₂, found 226 (MH+).

### Example 27: N-(4-Fluorophenyl)-N-(6-hydroxypyridin-3-yl)cyclopropane-1,1-dicarboxamide (144)

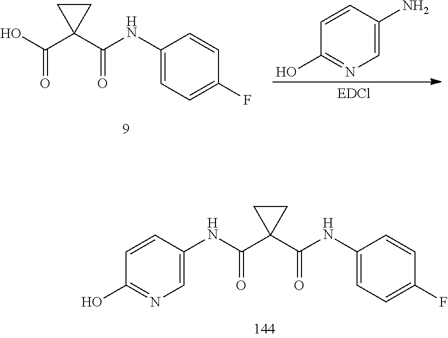 [SMILES: O=C(O)C1(C(=O)Nc2ccc(F)cc2)CC1 ; Nc1ccc(O)nc1 ; O=C(Nc1ccc(F)cc1)C1(C(=O)Nc2ccc(O)nc2)CC1]

N-(4-Fluorophenyl)-N-(6-hydroxypyridin-3-yl)cyclopropane-1,1-dicarboxamide (144): To a mixture of Compound 9 (1.15 g, 5.15 mmol) and 5-aminopyridin-2-ol (530 mg, 4.81 mmol) in DMF (5 mL), was added EDCI·HCl (1.01 g, 5.26 mmol). The reaction mixture was stirred at room temperature overnight and then partitioned between EtOAc and water. The aqueous phase was further extracted twice with EtOAc, and the combined EtOAc extracts were washed once with aq saturated NaCl, dried over Na₂SO₄ and evaporated to dryness. The resulting residue was washed with a mixture of DCM/EtOAc (18/2 mL) to give Compound 144 (780 mg, 51% yield). MS for C₁₆H₁₄FN₃O₃, found 316 (MH+).

### Example 28: 1-N′-[6-(6,7-Dimethoxypyrido[3,2-d]pyrimidin-4-yl)oxypyridin-3-yl]-1-N-(4-fluorophenyl)cyclopropane-1,1-dicarboxamide (145)

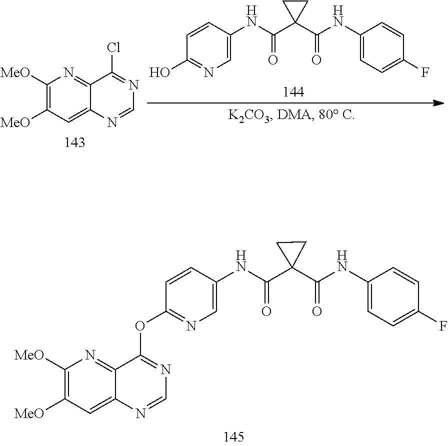

1-N′-[6-(6,7-Dimethoxypyrido[3,2-d]pyrimidin-4-yl)oxypyridin-3-yl]-1-N-(4-fluorophenyl)cyclopropane-1,1-dicarboxamide (145): A mixture of Compound 143 (29 mg, 0.13 mmol), Compound 144 (30 mg, 0.095 mmol), and K₂CO₃ (140 mg, 1.01 mmol) in DMA (1.5 mL) was stirred at 80° C. overnight. The reaction mixture was filtered, and the filtrate was subjected to silica gel chromatography, eluted with 0-100% EtOAc in hexanes, followed by prep-HPLC, to give Compound 145 (9.0 mg, 14% yield). MS for C₂₅H₂₁FN₆O₅, found 505 (MH+).

### Example 29: 4-(3-((4-Chloro-6-methoxypyrido[3,2-d]pyrimidin-7-yl)oxy)propyl)-morpholine (152)

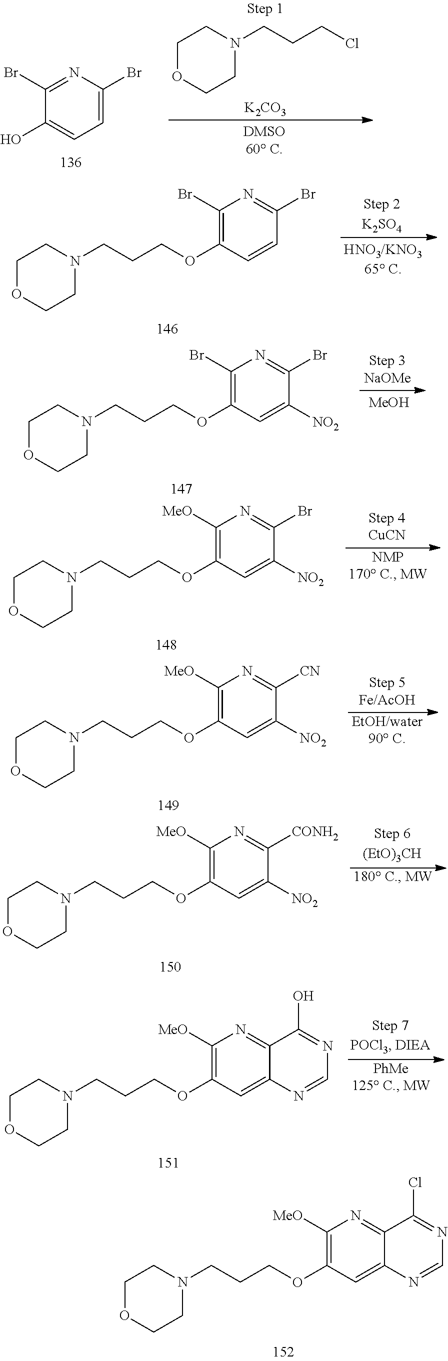

4-(3-((2,6-Dibromopyridin-3-yl)oxy)propyl)morpholine (146): Compound 146 was synthesized from Compound 136 in a manner analogous to that used to convert Compound 136 to Compound 137 in Step 1 of Example 26, replacing the Mel with 4-(3-chloropropyl)morpholine. MS (EI) for C₁₂H₁₆Br₂N₂O₂, found 379 (MH+).

4-(3-((2,6-Dibromo-5-nitropyridin-3-yl)oxy)propyl)morpholine (147): Compound 147 was synthesized from Compound 146 in a manner analogous to that used to convert Compound 137 to Compound 138 in Step 2 of Example 26.

4-(3-((6-Bromo-2-methoxy-5-nitropyridin-3-yl)oxy)propyl)morpholine (148): Compound 148 was synthesized from Compound 147 in a manner analogous to that used to convert Compound 138 to Compound 139 in Step 3 of Example 26.

6-Methoxy-5-(3-morpholinopropoxy)-3-nitropicolinonitrile (149): Compound 149 was synthesized from Compound 148 in a manner analogous to that used to convert Compound 139 to Compound 140 in Step 4 of Example 26.

6-Methoxy-5-(3-morpholinopropoxy)-3-nitropicolinamide (150): Compound 150 was synthesized from Compound 149 in a manner analogous to that used to convert Compound 140 to Compound 141 in Step 5 of Example 26. MS (EI) for C₁₄H₂₂N₄O₄, found 311 (MH+).

6-Methoxy-7-(3-morpholinopropoxy)pyrido[3,2-d]pyrimidin-4-ol (151): Compound 151 was made from Compound 150 in a manner analogous to the preparation of Compound 142 from Compound 141 in Step 6 of Example 26. MS (EI) for C₁₅H₂₀N₄O₄, found 321 (MH+).

4-(3-((4-Chloro-6-methoxypyrido[3,2-d]pyrimidin-7-yl)oxy)propyl)morpholine (152): Compound 152 was made from Compound 151 in a manner analogous to the preparation of Compound 143 from Compound 142 Step 7 of Example 26. MS (EI) for C₁₅H₁₉C₁₄O₃, found 339 (MH+).

The following compounds was prepared from Compound 136 using the same sequence of steps used to synthesize Compound 152 from Compound 136 in Example 29:

6-Methoxy-7-(2-methoxyethoxy)pyrido[3,2-d]pyrimidin-4-ol (153): The 4-(3-chloropropyl)morpholine in Step 1 was replaced with 1-chloro-2-methoxyethane. Steps 1 through 6 were used to produce Compound 153.

4-Chloro-6-methoxy-7-(2-methoxyethoxy)pyrido[3,2-d]pyrimidine (154): Compound 154 was synthesized from Compound 153 using the procedure in Step 7.

### Example 30: 1-N-(4-Fluorophenyl)-1-N′-[6-[6-methoxy-7-(3-morpholin-4-ylpropoxy)pyrido[3,2-d]pyrimidin-4-yl]oxypyridin-3-yl]cyclopropane-1,1-dicarboxamide (155)

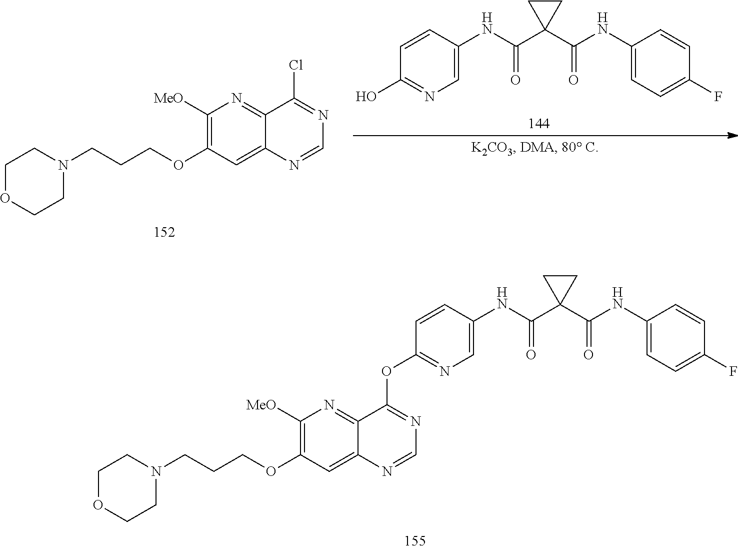 [SMILES: COc1nc2c(Cl)ncnc2cc1OCCCN1CCOCC1 ; O=C(Nc1ccc(F)cc1)C1(C(=O)Nc2ccc(O)nc2)CC1 ; COc1nc2c(Oc3ccc(NC(=O)C4(C(=O)Nc5ccc(F)cc5)CC4)cn3)ncnc2cc1OCCCN1CCOCC1]

1-N-(4-Fluorophenyl)-1-N′-[6-[6-methoxy-7-(3-morpholin-4-ylpropoxy)pyrido[3,2-d]pyrimidin-4-yl]oxypyridin-3-yl]cyclopropane-1,1-dicarboxamide (155): Compound 155 was made from Compound 152 and Compound 144 in the same manner that Compound 145 was made from Compound 143 and Compound 144 in Example 28. MS for C₃₁H₃₂FN₇O₆, found 618 (MH+).

The following compound was prepared from Compound 154 using the same method used to synthesize Compound 155 from Compound 152 in Example 30:

1-N-(4-Fluorophenyl)-1-N′-[6-[6-methoxy-7-(2-methoxyethoxy)pyrido[3,2-d]pyrimidin-4-yl]oxypyridin-3-yl]cyclopropane-1,1-dicarboxamide (156): MS for C₂₇H₂₅FN₆O₆, found 549 (MH+).

### Example 31: 1-N′-[5-Fluoro-6-[6-methoxy-7-(2-methoxyethoxy)pyrido[3,2-d]pyrimidin-4-yl]oxypyridin-3-yl]-1-N-(4-fluorophenyl)cyclopropane-1,1-dicarboxamide (159)

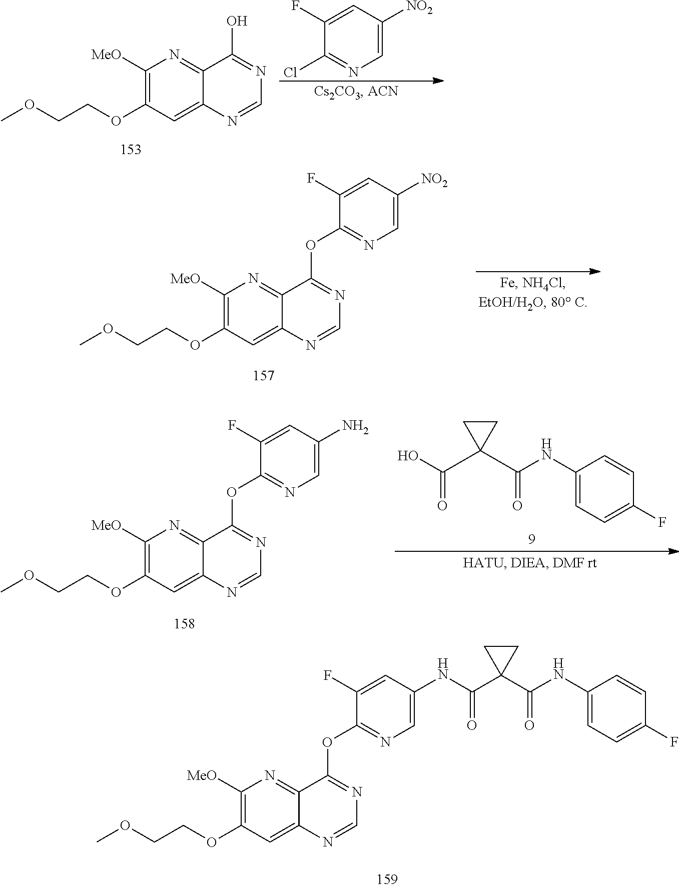

4-((3-Fluoro-5-nitropyridin-2-yl)oxy)-6-methoxy-7-(2-methoxyethoxy)pyrido[3,2-d]pyrimidine (157): Compound 157 was made from Compound 153 in the same manner that Compound 132 was made from Compound 131 in Step 4 of Example 25.

5-Fluoro-6-((6-methoxy-7-(2-methoxyethoxy)pyrido[3,2-d]pyrimidin-4-yl)oxy)pyridin-3-amine (158): Compound 158 was made from Compound 157 in the same manner that Compound 133 was made from Compound 132 in Step 5 of Example 25. MS for C₁₆H₁₆FN₅O₄, found 362 (MH+)

1-N′-[5-Fluoro-6-[6-methoxy-7-(2-methoxyethoxy)pyrido[3,2-d]pyrimidin-4-yl]oxypyridin-3-yl]-1-N-(4-fluorophenyl)cyclopropane-1,1-dicarboxamide (159): Compound 159 was synthesized from Compound 158 and Compound 9 in the same manner that Compound 10 was synthesized from Compound 8 and Compound 9 in the second step of Example 5. MS for C₂₇H₂₄F₂N₆O₆, found 567 (MH+).

### Biological Examples

Kinase Assays

Kinase activity and compound inhibition were investigated using the ³³P-Phosphoryl transfer radiometric kinase assay, performed using the KinaseProfiler™ service of Eurofins Pharma Discovery Services UK Limited. Dose-response experiments were performed using nine compound concentrations in a 96-well microtiter plate. For each assay, all compounds were prepared to a 50× final assay concentration (50 μM) in 100% DMSO, then diluted in a half-log series, with the final top concentration at 1 μM. This working stock of the compound was added to the assay well as the first component in the reaction, followed by the remaining components as detailed in the following assay protocols below. The positive control wells (100% kinase activity) contain all components of the reaction including 2% DMSO (control for solvent effects), except the compound of interest. Blank wells contain all components of the reaction, with the reference inhibitor, staurosporine. This reference compound was used to abolish kinase activity and generated the 0% kinase activity base-line. IC₅₀ values were calculated by nonlinear regression analysis using the sigmoidal dose-response (variable slope) curve fit on XLFit version 5.3 (ID Business Solutions).

### Example A: Human AXL Kinase Assay

Human Axl (residues H473-A894 with Q764R, 161 nM) was incubated with 8 mM MOPS pH 7.0, 0.2 mM EDTA, 250 μM KKSRGDYMTMQIG (SEQ ID NO. 43), 10 mM magnesium acetate, and 10 μM [γ-³³P-ATP]. The reaction was initiated by the addition of the Mg/ATP mix. After incubation for 40 minutes at room temperature, the reaction was stopped by the addition of phosphoric acid to a concentration of 0.5%. A reaction aliquot of 10 μL was then spotted onto a P30 filtermat and washed four times for 4 minutes in 0.425% phosphoric acid and once in methanol prior to drying and scintillation counting. Incorporated ³³P was measured using the Wallac Microbeta scintillation counter (Perkin Elmer).

### Example B: Human KDR Kinase Assay

Human KDR (residues K790-V1356, 55 nM) was incubated with 8 mM MOPS pH 7.0, 0.2 mM EDTA, 0.33 mg/mL myelin basic protein, 10 mM magnesium acetate, and 10 μM [7-³³P-ATP]. The reaction was initiated by the addition of the Mg/ATP mix. After incubation for 40 minutes at room temperature, the reaction was stopped by the addition of phosphoric acid to a concentration of 0.5%. A reaction aliquot of 10 μL was then spotted onto a P30 filtermat and washed four times for 4 minutes in 0.425% phosphoric acid and once in methanol prior to drying and scintillation counting. Incorporated ³³P was measured using the Wallac Microbeta scintillation counter (Perkin Elmer).

### Example C: Human Mer Kinase Assay

Human Mer (residues R557-E882 with H628Q and R794A, 0.7 nM) was incubated with 8 mM MOPS pH 7.0, 0.2 mM EDTA, 30 mM NaCl, 250 μM GGMEDIYFEFMGGKKK (SEQ ID NO. 44), 10 mM magnesium acetate, and 10 μM [γ-³³P-ATP]. The reaction was initiated by the addition of the Mg/ATP mix. After incubation for 40 minutes at room temperature, the reaction was stopped by the addition of phosphoric acid to a concentration of 0.5%. A reaction aliquot of 10 μL was then spotted onto a P30 filtermat and washed four times for 4 minutes in 0.425% phosphoric acid and once in methanol prior to drying and scintillation counting. Incorporated ³³P was measured using the Wallac Microbeta scintillation counter (Perkin Elmer).

### Example D: Human Met Kinase Assay

Human Met (residues R974-S1390 with A1209G and V1290L, 3.4 nM) was incubated with 8 mM MOPS pH 7.0, 0.2 mM EDTA, 250 μM KKKGQEEEYVFIE (SEQ ID NO. 45), 1 mM sodium orthovanadate, 5 mM sodium-6-glycerophosphate, 10 mM magnesium acetate, and 10 μM [7-33P-ATP]. The reaction was initiated by the addition of the Mg/ATP mix. After incubation for 40 minutes at room temperature, the reaction was stopped by the addition of phosphoric acid to a concentration of 0.5%. A reaction aliquot of 10 μL was then spotted onto a P30 filtermat and washed four times for 4 minutes in 0.425% phosphoric acid and once in methanol prior to drying and scintillation counting. Incorporated 33P was measured using the Wallac Microbeta scintillation counter (Perkin Elmer).

Activity data obtained for the Example compounds using the kinase assays in Examples A, C, and D is provided in Table 4 (A: IC₅₀≤10 nM; B: 10 nM<IC50≤100 nM; C: 100 nM<IC₅₀≤1000 nM; D: IC₅₀>1000 nM).

TABLE 4
Biological Activities of Selected Compounds

| Com- | | Axl | Mer | c-Met |
|---|---|---|---|---|
| pound | | IC₅₀ | IC₅₀ | IC₅₀ |
| No. | IUPAC Name | (nM) | (nM) | (nM) |
| 10 | methyl 4-[5-[[1-[(4- fluorophenyl)carbamoyl]cyclopropanecar bonyl]amino]pyridin-2-yl]oxy-7- methoxyquinoline-6-carboxylate | A | A | A |
| 11 | methyl 4-[3-chloro-5-[[1-[(4- fluorophenyl)carbamoyl]cyclopropanecar bonyl]amino]pyridin-2-yl]oxy-7- methoxyquinoline-6-carboxylate | A | A | A |
| 13 | 4-[5-[[1-[(4- fluorophenyl)carbamoyl]cyclopropanecar bonyl]amino]pyridin-2-yl]oxy-7- methoxyquinoline-6-carboxylic acid | C | C | C |
| 14 | 4-[3-chloro-5-[[1-[(4- fluorophenyl)carbamoyl]cyclopropanecar bonyl]amino]pyridin-2-yl]oxy-7- methoxyquinoline-6-carboxylic acid | C | C | B |
| 16 | 1-N′-[6-(6-carbamoyl-7-methoxyquinolin- 4-yl)oxypyridin-3-yl]-1-N-(4- fluorophenyl)cyclopropane-1,1- dicarboxamide | B | A | A |
| 17 | 1-N-(4-fluorophenyl)-1-N′-[6-[7-methoxy- 6-(methylcarbamoyl)quinolin-4- yl]oxypyridin-3-yl]cyclopropane-1,1- dicarboxamide | B | B | B |
| 18 | 1-N′-[6-(6-carbamoyl-7-methoxyquinolin- 4-yl)oxy-5-chloropyridin-3-yl]-1-N-(4- fluorophenyl)cyclopropane-1,1- dicarboxamide | A | A | A |
| 60 | 1-N-(4-fluorophenyl)-1-N′-[6-[7-methoxy- 6-(1H-pyrazol-4-yl)quinolin-4- yl]oxypyridin-3-yl]cyclopropane-1,1- dicarboxamide | A | A | A |
| 80 | 1-N′-[5-chloro-6-[(6,7-dimethoxy-1,5- naphthyridin-4-yl)oxy]pyridin-3-yl]-1-N- (4-fluorophenyl)cyclopropane-1,1- dicarboxamide | B | B | B |
| 81 | 1-N′-[6-[(6,7-dimethoxy-1,5-naphthyridin- 4-yl)oxy]pyridin-3-yl]-1-N-(4- fluorophenyl)cyclopropane-1,1- dicarboxamide | B | B | B |
| 82 | 1-N′-[6-[(6,7-dimethoxy-1,5-naphthyridin- 4-yl)oxy]-5-fluoropyridin-3-yl]-1-N-(4- fluorophenyl)cyclopropane-1,1- dicarboxamide | A | A | A |

### Example E: AXL Auto Phosphorylation ELISA in A-172 Cells

A-172 glioblastoma cells (ATCC #CRL-1620) were seeded at 2.5×10⁵ cells/well onto 24-well plates (Greiner #662165), in DiVEM (Thermo Fisher #11995-040) containing 10% FBS (Thermo Fisher #26140-079), 1% MEM NEAA (Thermo Fisher #11140-050), 1% GlutaMax (Thermo Fisher #35050-061), and 1% Penicillin Streptomycin (Thermo Fisher #15140-122). A-172 cells were incubated at 37° C., 5% CO₂ for 24 h and then starved for 24 h in serum-free medium. Test compounds were serially diluted to produce an 8-point dose curve in fresh serum-free medium to a final concentration of 0.3% DMSO (vehicle) and added to the cells and incubated for 1 h. Cells were then stimulated with 1 μg/mL recombinant human Gas6 (R&D Systems #885-GSB-500) for 15 min, washed with cold PBS, and immediately lysed with 150 μL of cold 1× lysis buffer [20 mM Tris, 137 mM sodium chloride, 2 mM EDTA, 10% glycerol, 1% NP-40 alternative, 1 mM activated sodium orthovanadate, 1 mM PefaBloc SC (Sigma-Aldrich #11429868001), protease/phosphatase inhibitor tablet (Thermo Fisher #A32959)]. Lysates were collected and 100 μL/well added into the human phospho-AXL DuoSet IC ELISA (R&D Systems #DYC2228-2). Assay was performed according to manufacturer's instructions and sample phospho-AXL concentrations were extrapolated using human phospho-AXL control (R&D Systems #841645) as a standard. Positive control wells (100% activity) contained Gas6-stimulated, DMSO-treated cell lysates. Negative control wells (0% activity) contained Gas6-stimulated, reference inhibitor-treated cell lysates. IC₅₀ values were calculated by nonlinear regression analysis using a 4-parameter logistic curve fit in ActivityBase XE (IDBS).

### Example F: Met Autophosphorylation ELISA in PC-3 Cells

PC-3 prostate cancer cells (ATCC #CRL-1435) were seeded at 4×10⁴ cells/well onto 24-well plates (Greiner #662165), in DMEM (Thermo Fisher #11995-040) containing 10% FBS (Thermo Fisher #26140-079), 1% MEM NEAA (Thermo Fisher #11140-050), 1% GlutaMax (Thermo Fisher #35050-061), and 1% Penicillin Streptomycin (Thermo Fisher #15140-122). PC-3 cells were incubated at 37° C., 5% CO₂ for 24 h and then starved for 3 h in serum-free medium. Test compounds were serially diluted to produce an 8-point dose curve in fresh serum-free medium to a final concentration of 0.3% DMSO (vehicle) and added to the cells and incubated for 1 h. Cells were then stimulated with 100 ng/mL recombinant human HGF (R&D Systems #294-HG-250) for 10 min, washed with cold PBS, and immediately lysed with 130 μL of cold 1× lysis buffer [20 mM Tris, 137 mM sodium chloride, 2 mM EDTA, 10% glycerol, 1% NP-40 alternative, 1 mM activated sodium orthovanadate, 1 mM PefaBloc SC (Sigma-Aldrich #11429868001), and protease/phosphatase inhibitor tablet (Thermo Fisher #A32959)]. Lysates were clarified by centrifugation and 100 μL/well added into the PathScan phospho-Met (panTyr) Sandwich ELISA (Cell Signaling Technology #7333). Assay was performed according to manufacturer's instructions. Positive control wells (100% activity) contained HGF-stimulated, DMSO-treated cell lysates. Negative control wells (0% activity) contained HGF-stimulated, reference inhibitor-treated cell lysates. IC₅₀ values were calculated by nonlinear regression analysis using a 4-parameter logistic curve fit in ActivityBase XE (IDBS).

### Example G: KDR Autophosphorylation ELISA in HUVEC Cells

Human umbilical vein endothelial cells or HUVEC (Lonza #C2519A) were seeded at 2×10⁴ cells/well onto 96-well plates (Corning #3904), in EGM-2 growth medium (Lonza #CC-3162) containing 1% Penicillin Streptomycin (Thermo Fisher #15140-122). HUVEC cells were incubated at 37° C., 5% CO₂ for 24 h and then starved for 24 h in serum-free EBM-2 basal medium (Lonza #CC-3156) containing 1% Penicillin Streptomycin. Test compounds were serially diluted to produce an 8-point dose curve in fresh serum-free medium to a final concentration of 0.3% DMSO (vehicle) and added to the cells and incubated for 1 h. Cells were then stimulated with 100 ng/mL recombinant human VEGF165 (R&D Systems #293-VE-500) for 5 min, washed with cold PBS, and immediately lysed with 130 μL of cold 1× lysis buffer [20 mM Tris, 137 mM sodium chloride, 2 mM EDTA, 10% glycerol, 1% NP-40 alternative, 1 mM activated sodium orthovanadate, 1 mM PefaBloc SC (Sigma-Aldrich #11429868001), protease/phosphatase inhibitor tablet (Thermo Fisher #A32959)]. Lysates were collected and 100 μL/well added into the human phospho-KDR DuoSet IC ELISA (R&D Systems #DYC1766-2). Assay was performed according to manufacturer's instructions and sample phospho-KDR concentrations were extrapolated using human phospho-KDR control (R&D Systems #841421) as a standard. Positive control wells (100% activity) contained VEGF165-stimulated, DMSO-treated cell lysates. Negative control wells (0% activity) contained non-stimulated cell lysates. IC₅₀ values were calculated by nonlinear regression analysis using a 4-parameter logistic curve fit in ActivityBase XE (IDBS).

### Example H: Mer Autophosphorylation ELISA in Transient Transfected 293A Cells

293A cells (Thermo Fisher #R₇₀₅₀₇) were seeded at 1.5×10⁶ cells/well onto 100 mm dish (Greiner #664169), in DMEM (Thermo Fisher #11995-040) containing 10% FBS (Thermo Fisher #26140-079), 1% MEM NEAA (Thermo Fisher #11140-050), 1% GlutaMax (Thermo Fisher #35050-061), and 1% Penicillin Streptomycin (Thermo Fisher #15140-122). 293A cells were incubated at 37° C., 5% CO₂ for 24 h and then transfected with 6 μg MERTK DNA (Genecopoeia #EX-Z8208-M02) using TransIT LT1 transfection reagent (Mirus-Bio #MIR2305). After 24 h incubation, the transfected 293A cells were seeded at 1×10⁵ cells/well onto 96-well plates (Corning #3904) in DMEM growth medium overnight. Test compounds were serially diluted to produce an 8-point dose curve in fresh serum-free medium to a final concentration of 0.3% DMSO (vehicle) and added to the cells and incubated for 1 h. Cells were then immediately lysed with 150 μL of cold 1× lysis buffer [20 mM Tris, 137 mM sodium chloride, 2 mM EDTA, 10% glycerol, 1% NP-40 alternative, 1 mM activated sodium orthovanadate, 1 mM PefaBloc SC (Sigma-Aldrich #11429868001), protease/phosphatase inhibitor tablet (Thermo Fisher #A32959)]. Lysates were clarified by centrifugation and 50 μL/well added into the human phospho-Mer DuoSet IC ELISA (R&D Systems #DYC2579-2). Assay was performed according to manufacturer's instructions and sample phospho-Mer concentrations were extrapolated using human phospho-Mer control (R&D Systems #841793) as a standard. Positive control wells (100% activity) contained DMSO-treated cell lysates. Negative control wells (0% activity) contained reference inhibitor-treated cell lysates. IC₅₀ values were calculated by nonlinear regression analysis using a 4-parameter logistic curve fit in ActivityBase XE (IDBS).

Compounds of the present disclosure, as exemplified herein, showed IC₅₀ values in the following ranges: A: IC₅₀≤10 nM; B: 10 nM<IC₅₀≤100 nM; C: 100 nM<IC₅₀≤300 nM; D: IC₅₀>300 nM. “NT” means not tested.

Activity data obtained for the Example compounds using cell based kinase assays in Examples F, G, H and I is provided in Table 5.

TABLE 5
Cellular Activities of Selected Compounds

| Compound | Axl | Mer | c-Met | KDR |
|---|---|---|---|---|
| No. | IC₅₀ (nM) | IC₅₀ (nM) | IC₅₀ (nM) | IC₅₀ (nM) |
| 10 | B | NT | C | B |
| 11 | B | B | B | D |
| 13 | NT | NT | NT | NT |
| 14 | D | NT | NT | D |
| 15 | D | NT | D | D |
| 16 | B | NT | C | B |
| 17 | B | NT | C | B |
| 18 | A | B | B | D |
| 19 | A | B | B | D |
| 20 | B | B | B | D |
| 21 | A | B | B | D |
| 22 | A | B | B | D |
| 23 | B | B | B | D |
| 25 | D | NT | D | D |
| 26 | D | NT | D | D |
| 27 | D | NT | D | D |
| 37 | B | C | B | D |
| 38 | B | B | B | D |
| 39 | B | C | B | D |
| 44 | B | NT | B | D |
| 50 | B | B | B | D |
| 51 | B | B | B | D |
| 60 | B | B | B | C |
| 64 | A | B | B | D |
| 67 | B | B | B | D |
| 68 | B | B | B | D |
| 71 | B | B | B | D |
| 72 | A | B | B | D |
| 80 | C | C | B | D |
| 81 | C | NT | C | D |
| 82 | B | B | B | D |
| 83 | D | NT | D | D |
| 84 | D | NT | D | D |
| 85 | D | NT | D | D |
| 86 | B | B | C | D |
| 87 | B | D | D | D |
| 88 | C | NT | D | D |
| 89 | D | NT | D | D |
| 90 | D | NT | D | D |
| 91 | D | NT | D | D |
| 92 | D | NT | D | D |
| 96 | A | B | B | D |
| 103 | D | NT | D | D |
| 104 | D | NT | D | D |
| 105 | D | NT | D | D |
| 114 | C | NT | C | D |
| 115 | C | NT | B | D |
| 116 | B | B | C | D |
| 120 | C | D | C | D |
| 121 | D | NT | D | D |
| 122 | B | B | B | D |
| 123 | B | B | B | D |
| 124 | C | C | C | D |
| 125 | B | B | B | D |
| 127 | B | C | B | D |
| 134 | D | NT | D | D |
| 135 | D | NT | D | D |
| 145 | C | NT | D | D |
| 155 | B | C | C | D |
| 156 | B | NT | D | D |
| 159 | D | NT | D | D |

### Example I: Pharmacokinetic Studies

Pharmacokinetic properties of select compounds as described herein were assessed in male Sprague-Dawley rats. The non-GLP study was designed to investigate the pharmacokinetics of chosen compounds in plasma following an intravenous or oral dose administration to male Sprague Dawley rats. Two groups of male Sprague-Dawley rats (three animals per group) received either an intravenous or oral (gavage) dose of compound at target dose levels of 3 mg/kg. Animals were observed for any clinically relevant abnormalities during dosing and at each sample collection period.

Animals in the PO group were fasted overnight prior to dose administration. Food was returned following the collection of the 4-hour blood sample. Water was not withheld.

Immediately prior to dosing, the body weight of each animal was recorded. Doses (rounded to the nearest 0.001 mL) were calculated based on the pretreatment body weight (kg) and a dose volume of 2.5 mL/kg for intravenous administration and 5 mL/kg for oral administration. Intravenous formulations were administered via a jugular vein cannula. Immediately after dosing, the cannula was flushed with saline and the line was tied off. The oral dose was administered via a ball-tipped feeding needle. Dosing syringe volumes for administration were second-person verified prior to dosing and that volume along with the results for the concentration verification analysis were used to calculate the actual dose administered. Dosing syringes were weighed immediately prior to and immediately after dosing each animal as a gravimetric check.

Serial blood samples (approximately 200 μL per sample) were collected from each animal at 0.083 (IV dosing only), 0.25, 0.5, 1, 2, 4, 6 (PO dosing only), 8, 24, 32, 48, and 72 hours after dosing. Blood samples were collected into tubes containing K₂EDTA via the non-dosing jugular-vein cannula (JVC), which was flushed with an approximately equal volume of saline following each collection.

Blood samples were stored on wet ice until processed to plasma by centrifugation (3500 rpm at 5° C. for 10 minutes) within 1 hour of collection. Plasma samples were transferred into matrix tubes and then stored in a −80° C. freezer.

Plasma samples and dose formulation samples were analyzed for the compounds of interest using liquid chromatography-tandem mass spectrometry (LC-MS/MS) methods. Pharmacokinetic parameter estimates were calculated from the individual animal plasma concentration-time data using the actual dose based on the analysis of the dosing formulations, nominal sampling times (all collections were within an acceptable range of target), and non-compartmental methods. The concentration-time data were analyzed to fit either an intravenous—bolus (IV) plasma analysis model (201) or extra-vascular (PO) dosing plasma analysis model (200) using the software WinNonlin Phoenix version 6.3 (Pharsight). The single-dose pharmacokinetic parameters assessed include, as appropriate: Cₘₐₓ (observed peak or maximum concentration); Tₘₐₓ (observed time of peak concentration); T_{1/2} (terminal half-life); V_{z} (volume of distribution based on the terminal phase); Vₛₛ (volume of distribution at steady state); AUC_{INF} (area under the concentration-time curve computed from time zero to infinity); AUCₗₐₛₜ (area under the concentration-time curve computed from time zero to the time of the last quantifiable concentration); C₀ (back-extrapolated concentration at time zero); CL (total body clearance); Vz/F (volume of distribution for extravascular administration based on the terminal phase); CL/F (total body clearance for extravascular administration); F % (bioavailability); and MRTₗₐₛₜ (mean residence time).

Areas-under-the-plasma concentration-time curves (AUC) were estimated using the linear-log trapezoidal rule. The area through the time (Tₗₐₛₜ) of the last observable concentration (Cₗₐₛₜ) is reported as AUCₗₐₛₜ. AUC extrapolated to infinity, (AUC_{INF}) was estimated by adding AUCₗₐₛₜ and the ratio of Cₗₐₛₜ/λ_{z}, where λ_{z} is the terminal rate constant. Apparent terminal half-life (T_{1/2}) was calculated as ln(2)/λ_{z} and determined using the slope of the log-linear terminal phase of the concentration-time curve, defined by a minimum of three plasma concentration-time points. Half-lives are reported if the correlation for the regression line, as measured by r squared, is ≥0.9 when rounded. After IV administration, volume of distribution (Vz) was calculated as Dose/λ_{z}*AUC_{INF-obs}, clearance (CL) was calculated as Dose/AUC_{INF-obs} and volume of distribution at steady state (Vₛₛ) was estimated as MRT_{INF}*CL. Mean residence time (MRT) from the time of dosing to the time of the last measurable concentration was calculated as AUMCₗₐₛₜ/AUCₗₐₛₜ. For model 200 the bioavailability (i.e. fraction of total dose that reaches the systemic circulation) cannot be calculated. Consequently, volume and clearance for this model is Vz/F or CL/F, respectively; where F is defined as bioavailability (i.e. fraction of total dose that reaches the systemic circulation; (Average AUCₗₐₛₜ₋ₚₒ/Average AUCₗₐₛₜ₋ᵢᵥ)*[Dose_{IV}/Dose_{PO}]*100).

### OTHER EMBODIMENTS

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1. A compound of Formula I′: 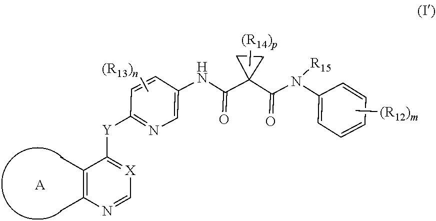
or a pharmaceutically acceptable salt thereof, wherein:
X is N or CH;
Y is selected from O, S, SO, SO₂, NH, and —N(C₁₋₆ alkyl)-;
ring A is 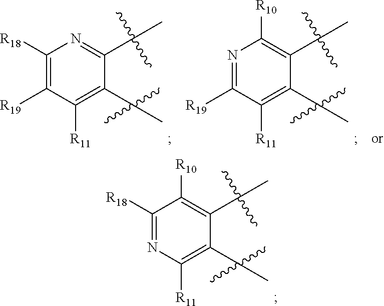
R₁₈ and R₁₉ are each independently selected from —H; halo; (C₁-C₆) alkyl; (C₂-C₆) alkenyl; (C₂-C₆) alkynyl; (C₁-C₆) haloalkyl; (C₁-C₆) haloalkoxy; (C₆-C₁₀) aryl; (C₃-C₁₀) cycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; (4-14 membered heterocycloalkyl)-(C₁-C₄) alkylene-; —CN; —NO₂; —OR^{a}; —SR^{a}; —NHOR^{a}; —C(O)R^{a}; —C(O)NR^{a}R^{a}; —C(O)NHOR^{a}; —C(O)OR^{a}; —C(O)NR^{a}S(O)₂R^{a}; —OC(O)R^{a}; —OC(O)NR^{a}R^{a}; —NHR^{a}; —NR^{a}R^{a}; —NR^{a}C(O)R^{a}; —NR^{a}C(═NR^{a})R^{a}; —NR^{a}C(O)OR^{a}; —NR^{a}C(O)NR^{a}R^{a}; —C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —NR^{a}C(═NR^{a})NR^{a}R^{a}; —NR^{a}S(O)R^{a}; —NR^{a}S(O)₂R^{a}; —NR^{a}S(O)₂NR^{a}R^{a}; —S(O)R^{a}; —S(O)NR^{a}R^{a}; —S(O)₂R^{a}; —S(O)₂NR^{a}C(O)R^{a}; —P(O)R^{a}R^{a}; —P(O)(OR^{a})(OR^{a}); —B(OH)₂; —B(OR^{a})₂; and —S(O)₂NR^{a}R^{a}; wherein the (C₁-C₆) alkyl; (C₂-C₆) alkenyl; (C₂-C₆) alkynyl; (C₆-C₁₀) aryl; (C₃-C₁₀) cycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R₁₈ or R₁₉ are each optionally substituted with 1, 2, 3, 4, or 5 independently selected R^{b} substituents; or
R₁₈ and R₁₉ taken together with the atoms to which they are attached form a fused C₃₋₇ cycloalkyl ring or a fused 4- to 10-membered heterocycloalkyl ring; wherein the fused C₃₋₇ cycloalkyl ring and fused 4- to 10-membered heterocycloalkyl ring are each optionally substituted with 1, 2, or 3 independently selected R^{b} substituents;
R₁₀ and R₁₁ are each independently selected from the group consisting of —H; halo; (C₁-C₆) alkyl; (C₁-C₆) haloalkyl; (C₁-C₆) haloalkoxy; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; (4-14 membered heterocycloalkyl)-(C₁-C₄) alkylene-; —CN; —NO₂; —OR^{a}; —SR^{a}; —NHOR^{a}; —C(O)R^{a}; —C(O)NR^{a}R^{a}; —C(O)OR^{a}; —C(O)NR^{a}S(O)₂R^{a}; —OC(O)R^{a}; —OC(O)NR^{a}R^{a}; —NHR^{a}; —NR^{a}R^{a}; —NR^{a}C(O)R^{a}; —NR^{a}C(═NR^{a})R^{a}; —NR^{a}C(O)OR^{a}; —NR^{a}C(O)NR^{a}R^{a}; —C(═NR^{a})R^{a}; —C(═NOH)R^{a}; —C(═NOH)NR^{a}; —C(═NCN)NR^{a}R^{a}; —NR^{a}C(═NCN)NR^{a}R^{a}; —C(═NR^{a})NR^{a}R^{a}; —NR^{a}C(═NR^{a})NR^{a}R^{a}; —NR^{a}S(O)R^{a}; —NR^{a}S(O)₂R^{a}; —NR^{a}S(O)₂NR^{a}R^{a}; —S(O)R^{a}; —S(O)NR^{a}R^{a}; —S(O)₂R^{a}; —S(O)₂NR^{a}C(O)R^{a}; —P(O)R^{a}R^{a}; —P(O)(OR^{a})(OR^{a}); —B(OH)₂; —B(OR^{a})₂; and S(O)₂NR^{a}R^{a}; wherein the (C₁-C₆) alkyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R₁ or R₂ are each optionally substituted with 1, 2, 3, 4, or 5 independently selected R^{b} substituents;
each R₁₃ is independently selected from the group consisting of —H; halo; —OH; —CN; optionally substituted (C₁-C₆) alkyl; (C₁-C₆) alkoxy; (C₁-C₆) haloalkoxy; —NH₂; —NH(C₁-C₆)alkyl; —N(C₁-C₆ alkyl)₂; and (C₃-C₆) cycloalkyl; wherein the (C₁-C₆) alkoxy; —NH(C₁-C₆)alkyl; —N(C₁-C₆ alkyl)₂; and (C₃-C₆) cycloalkyl of R₃ are each optionally substituted with 1, 2, or 3 independently selected R^{g} substituents;
each R₁₄ is independently selected from the group consisting of halo; —OH; —NH₂; —CN; (C₁-C₆) alkyl; (C₁-C₆) alkoxy; (C₁-C₆) haloalkyl; (C₁-C₆) haloalkoxy; —COOH; —NH(C₁-C₆)alkyl; —N(C₁-C₆ alkyl)₂; phenyl; phenyl-(C₁-C₂) alkylene; (C₃-C₆) cycloalkyl; (C₃-C₆) cycloalkyl-(C₁-C₄) alkylene-; 4- to 6-membered heterocycloalkyl; (4- to 6-membered heterocycloalkyl)-(C₁-C₄) alkylene-; 5- to 6-membered heteroaryl; (5- to 6-membered heteroaryl)-(C₁-C₄) alkylene-; and —OR^{e}; wherein the (C₁-C₆) alkyl; phenyl; phenyl-(C₁-C₂) alkylene; (C₃-C₆) cycloalkyl; (C₃-C₆) cycloalkyl-(C₁-C₄) alkylene-; 4- to 6-membered heterocycloalkyl; (4- to 6-membered heterocycloalkyl)-(C₁-C₄) alkylene-; 5- to 6-membered heteroaryl; and (5- to 6-membered heteroaryl)-(C₁-C₄) alkylene- of R₁₄ are each optionally substituted with 1, 2, or 3 independently selected R^{g} substituents;
R₁₅ is H;
each R₁₂ is independently selected from the group consisting of —H; halo; —OH; —COOR^{e}; —CONR^{e}R^{e}; —CN; —NH₂; —NH((C₁-C₆) alkyl); —N((C₁-C₆) alkyl)₂; (C₁-C₆) alkyl; (C₁-C₆) alkoxy; (C₁-C₆) haloalkyl; (C₁-C₆) haloalkoxy; —CONR^{a}R^{a}; —NR^{a}COR^{a}; —NR^{a}CONR^{a}R^{a}; —SO₂R^{a}; —NR^{a}S(O)₂R^{a}; —NR^{a}S(O)₂NR^{a}R^{a}; (C₃-C₆) cycloalkyl; 4- to 6-membered heterocycloalkyl; phenyl; 5- or 6-membered heteroaryl; (C₃-C₆) cycloalkyl-(C₁-C₄) alkylene-; (4- to 6-membered heterocycloalkyl)-(C₁-C₄) alkylene-; phenyl-(C₁-C₂) alkylene; and (5- or 6-membered heteroaryl)-(C₁-C₄) alkylene-; wherein the (C₁-C₆) alkyl; (C₃-C₆) cycloalkyl; 4- to 6-membered heterocycloalkyl; phenyl; 5- or 6-membered heteroaryl; (C₃-C₆) cycloalkyl-(C₁-C₄) alkylene-; (4- to 6-membered heterocycloalkyl)-(C₁-C₄) alkylene-; phenyl-(C₁-C₂) alkylene; and (5- or 6-membered heteroaryl)-(C₁-C₄) alkylene- of R₁₂ are each optionally substituted with 1, 2, or 3 independently selected R^{f} substituents;
each R^{a} is independently selected from the group consisting of —H; —CN; (C₁-C₆) alkyl; (C₁-C₆) haloalkyl; (C₂-C₆) alkenyl; (C₂-C₆) alkynyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene-; wherein the (C₁-C₆) alkyl; (C₁-C₆) haloalkyl; (C₂-C₆) alkenyl; (C₂-C₆) alkynyl; (C₆-C₁₀) aryl; (C₃-C₁₀) cycloalkyl; 5-14 membered heteroaryl; 4-14 membered heterocycloalkyl; (C₆-C₁₀) aryl-(C₁-C₄) alkylene-; (C₃-C₁₀) cycloalkyl-(C₁-C₄) alkylene-; (5-14 membered heteroaryl)-(C₁-C₄) alkylene-; and (4-14 membered heterocycloalkyl)-(C₁-C₄) alkylene- of R^{a} are each optionally substituted with 1, 2, 3, 4, or 5 independently selected R^{d} substituents;
each R^{b} is independently selected from the group consisting of halo; (C₁-C₆) alkyl; (C₂-C₆) alkenyl; (C₂-C₆) alkynyl; (C₁-C₆) haloalkyl; (C₁-C₆) haloalkoxy; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; (4-10 membered heterocycloalkyl)-(C₁-C₄) alkylene-; —CN; —OH; —NH₂; —NO₂; —NHOR^{c}; —OR^{c}; —SR^{c}; —C(O)R^{c}; —C(O)NR^{c}R^{c}; —C(O)OR^{c}; —C(O)NR^{c}S(O)₂R^{c}; —OC(O)R^{c}; —OC(O)NR^{c}R^{c}; —C(═NOH)R^{c}; —C(═NOH)NR^{c}; —C(═NCN)NR^{c}R^{c}; —NR^{c}C(═NCN)NR^{c}R^{c}; —C(═NR^{c})NR^{c}R^{c}; —NR^{c}C(═NR^{c})NR^{c}R^{c}; —NHR^{c}; —NR^{c}R^{c}; —NR^{c}C(O)R^{c}; —NR^{c}C(═NR^{c})R^{c}; —NR^{c}C(O)OR^{c}; —NR^{c}C(O)NR^{c}R^{c}; —NR^{c}S(O)R^{c}; —NR^{c}S(O)₂R^{c}; —NR^{c}S(O)₂NR^{c}R^{c}; —S(O)R^{c}; —S(O)NR^{c}R^{c}; —S(O)₂R^{c}; —S(O)₂NR^{c}C(O)R^{c}; —Si(R^{c})₃; —P(O)R^{c}R^{c}; —P(O)(OR^{c})(OR^{c}); —B(OH)₂; —B(OR)₂; and —S(O)₂NR^{c}R^{c}; wherein the (C₁-C₆) alkyl; (C₁-C₆) haloalkyl; (C₁-C₆) haloalkoxy; (C₂-C₆) alkenyl; (C₂-C₆) alkynyl; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalky-(C₁-C₄) alkylene-; (5-10 membered heteroaryl)-(C₁-C₄) alkylene-; and (4-10 membered heterocycloalkyl)-(C₁-C₄) alkylene- of R^{b} are each further optionally substituted with 1, 2, or 3 independently selected R^{d} substituents;
each R^{c} is independently selected from the group consisting of —H; (C₁-C₆) alkyl; (C₁-C₆) haloalkyl; (C₂-C₆) alkenyl; (C₂-C₆) alkynyl; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; and (4-10 membered heterocycloalkyl)-(C₁-C₄) alkylene-; wherein the (C₁-C₆) alkyl; (C₂-C₆) alkenyl; (C₂-C₆) alkynyl; (C₆-C₁₀) aryl; (C₃-C₁₀) cycloalkyl; 5-10 membered heteroaryl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; and (4-10 membered heterocycloalkyl)-(C₁-C₄) alkylene- of R^{c} are each optionally substituted with 1, 2, 3, 4, or 5 independently selected R^{f} substituents;
each R^{d} is independently selected from the group consisting of (C₁-C₆) alkyl; (C₁-C₆) haloalkyl; halo; (C₆-C₁₀) aryl; 5-10 membered heteroaryl; (C₃-C₁₀) cycloalkyl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; (4-10 membered heterocycloalkyl)-(C₁-C₄) alkylene-; —CN; —NH₂; —NHOR^{e}; —OR^{e}; —SR^{e}; —C(O)R^{e}; —C(O)NR^{e}R^{e}; —C(O)OR^{e}; —OC(O)R^{e}; —OC(O)NR^{e}R^{e}; —NHR^{e}; —NR^{e}R^{e}; —NR^{e}C(O)R^{e}; —NR^{e}C(O)NR^{e}R^{e}; —NR^{e}C(O)OR^{e}; —C(═NR^{e})NR^{e}R^{e}; —NR^{e}C(═NR^{e})NR^{e}R^{e}; —NR^{e}C(═NOH)NR^{e}R^{e}; —NR^{e}C(═NCN)NR^{e}R^{e}; —S(O)R^{e}; —S(O)NR^{e}R^{e}; —S(O)₂R^{e}; —NR^{e}S(O)₂R^{e}; —NR^{e}S(O)₂NR^{e}R^{e}; and —S(O)₂NR^{e}R^{e}; wherein the (C₁-C₆) alkyl; (C₁-C₆)haloalkyl; (C₆-C₁₀) aryl; 5-10 membered heteroaryl; (C₃-C₁₀) cycloalkyl; 4-10 membered heterocycloalkyl; (C₆-C₁₀) aryl-(C₁-C₄) alkylene-; (C₃-C₁₀) cycloalkyl-(C₁-C₄) alkylene-; (5-10 membered heteroaryl)-(C₁-C₄) alkylene-; and (4-10 membered heterocycloalkyl)-(C₁-C₄) alkylene- of R^{d} are each optionally substituted with 1, 2, or 3 independently selected R^{f} substituents;
each R^{e} is independently selected from the group consisting of —H; (C₁-C₆) alkyl; (C₃-C₆) cycloalkyl; (C₃-C₆) cycloalkyl-(C₁-C₄) alkylene-; (C₆-C₁₀) aryl; (C₆-C₁₀) aryl-(C₁-C₄) alkylene-; 5- or 6-membered heteroaryl; (5- or 6-membered heteroaryl)-(C₁-C₄) alkylene-; 4-7-membered heterocycloalkyl; (4-7-membered heterocycloalkyl)-(C₁-C₄) alkylene-; (C₁-C₆) haloalkyl; (C₁-C₆) haloalkoxy; (C₂-C₄) alkenyl; and (C₂-C₄) alkynyl; wherein the (C₁-C₄) alkyl; (C₃-C₆) cycloalkyl; (C₆-C₁₀) aryl; 5 or 6-membered heteroaryl; 4-7-membered heterocycloalkyl; (C₆-C₁₀) aryl-(C₁-C₄) alkylene-; (5- or 6-membered heteroaryl)-(C₁-C₄) alkylene-; (4-7-membered heterocycloalkyl)-(C₁-C₄) alkylene-; (C₂-C₄) alkenyl; and (C₂-C₄) alkynyl of R^{e} are each optionally substituted with 1, 2, or 3 R^{f} substituents;
or any two R^{a} substituents together with the nitrogen atom to which they are attached form 4-, 5-, 6-, 7-, 8-, 9-, or 10-membered heterocycloalkyl, each of which is optionally substituted with 1, 2, or 3 independently selected R^{f} substituents;
or any two R^{c} substituents together with the nitrogen atom to which they are attached form 4-, 5-, 6-, 7-, 8-, 9-, or 10-membered heterocycloalkyl, each of which is optionally substituted with 1, 2, or 3 independently selected R^{f} substituents;
or any two R^{e} substituents together with the nitrogen atom to which they are attached form 4-, 5-, 6-, 7-, 8-, 9-, or 10-membered heterocycloalkyl, each of which is optionally substituted with 1, 2, or 3 independently selected R^{f} substituents;
each R^{f} is independently selected from the group consisting of halo; —OH; —CN; —COOH; —NH₂; —NH—(C₁-C₆) alkyl; —N((C₁-C₆) alky)₂; (C₁-C₆) alkyl; (C₁-C₆) alkoxy; (C₁-C₆) alkylthio; (C₁-C₆) haloalkyl; (C₁-C₆)haloalkoxy; phenyl; 5-6 membered heteroaryl; 4-6 membered heterocycloalkyl; and (C₃-C₆) cycloalkyl; wherein the (C₁-C₆) alkyl; phenyl; (C₃-C₆) cycloalkyl; 4-6 membered heterocycloalkyl; and 5-6 membered heteroaryl of R^{f} are each optionally substituted with 1, 2, or 3 substituents selected from halo; —OH; —CN; —COOH; —NH₂; (C₁-C₄) alkyl; (C₁-C₄) alkoxy; (C₁-C₄) haloalkyl; (C₁-C₄) haloalkoxy; phenyl; (C₃-C₁₀) cycloalkyl; 5-6 membered heteroaryl; and 4-6 membered heterocycloalkyl;
each R^{g} is independently selected from the group consisting of halo; —OH; —CN; —COOH; —COO—(C₁-C₄) alkyl; —NH₂; —NH—(C₁-C₆) alkyl; —N((C₁-C₆) alky)₂; (C₁-C₆) alkyl; (C₁-C₆) alkoxy; (C₁-C₆) alkylthio; (C₁-C₆)haloalkyl; (C₁-C₆)haloalkoxy; phenyl; 5-6 membered heteroaryl; 4-6 membered heterocycloalkyl; and (C₃-C₆) cycloalkyl;
the ring nitrogen atom on the quinoline moiety in Formula A is optionally oxidized;
the subscript n is an integer of 1, 2, 3, or 4;
the subscript m is an integer of 1, 2, 3, 4, or 5; and
the subscript p is an integer of 0, 1, 2, 3, or 4.

2. (canceled)

3. The compound of claim 1, having Formula I′b, I′c or I′d:

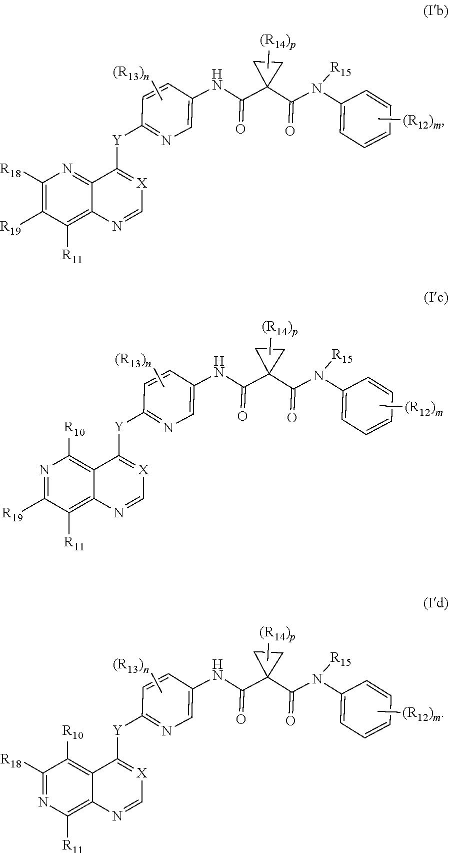

4. (canceled)

5. The compound of claim 1, having Formula (I′b-1), (I′b-2), (I′c-1), (I′c-2), (I′d-1), or (I′d-2):

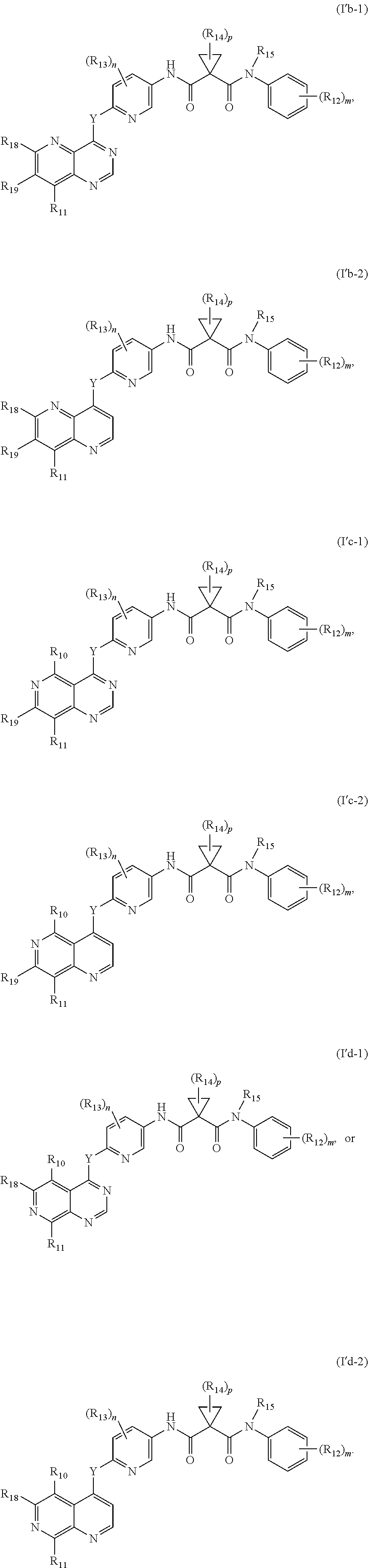

6-12. (canceled)

13. The compound of claim 1, wherein R₁₈ and R₁₉ are each independently selected from —H, (C₁-C₆) alkyl; (C₂-C₆) alkenyl, (C₂-C₆) alkynyl, —C(═NO—(C₁-C₆) alkyl)R^{a}; halo, —CN, OR^{a}, —C(O)OR^{a}; —C(O)NR^{a}R^{a}, —C(O)NHOR^{a}, —S(O)₂NR^{a}R^{a}, phenyl, 5- to 6-membered heteroaryl, (C₃-C₆) cycloalkyl, and 4- to 6-membered heterocycloalkyl, wherein the (C₁-C₆) alkyl, (C₂-C₆) alkenyl, (C₂-C₆) alkynyl, phenyl, 5- to 6-membered heteroaryl, (C₃-C₆) cycloalkyl, and 4- to 6-membered heterocycloalkyl of R₁₆ are each optionally substituted with 1, 2 or 3 R^{b} substituents.

14. (canceled)

15. The compound of claim 1, wherein R₁₈ and R₁₉ are each independently selected from H, halo, NH₂, NH(C₁₋₆ alkyl), N(C₁₋₆ alkyl), methoxy, methyl, CN, 3-morphlinopropoxy, 2-methoxyethoxy, (oxetan-3-yloxy)carbamoyl, cyclopropylcarbamoyl, carbamoyl, 2-(pyrrolidin-1-yl)ethylcarbamoyl, 1-(t-butoxycarbonylpyrrolidin-2-yl)methylcarbamoyl, 1-(pyrrolidin-2-yl)methylcarbamoyl, 2-methoxyethylamino; azetidin-1-yl; dimethylcarbamoyl, methylamino; 3-morpholinopropoxy; 2-methoxyethoxy; 2-hydroxyethoxy; propoxy; 2-hydroxypropoxy; methoxycarbonyl; carboxy; methylcarbamoyl; 2-oxazolyl; pyrazol-3-yl; pyrazol-4-yl; 4-isoxazolyl; 3,5-dimethylisoxazol-4-yl; 1-methyl-pyrazol-4-yl; 2-methyl-pyrazol-3-yl; 2-ethyl-pyrazol-3-yl; 2-(2-hydroxyethyl)-pyrazol-3-yl; 2-(2,2,2-trifluoroethyl)-pyrazol-3-yl; 2-(2-fluoroethyl)-pyrazol-3-yl; 2-(2,2-difluoroethyl)-pyrazol-3-yl; 2-trifluoromethyl-pyrazol-3-yl; 2-difluoromethyl-pyrazol-3-yl; 1-methyl-imidazol-4-yl; 1-methyl-imidazol-2-yl; 1H-imidazol-2-yl; (2-hydroxyethoxy)carbamoyl; (2,2-dihydroxyethoxy)carbamoyl; (oxetan-3-yloxy)carbamoyl; methoxycarbamoyl; 2-trimethylsilylethynyl; ethynyl; 1,3,4-oxadiazol-3-yl; 1H-1,2,3-triazol-5-yl; sulfamoyl; acetyl, —OH, 2-morpholinoethoxy, carbamoylmethyloxy, —OCH₂C(O)NH₂, 3-dimethylaminopropyloxy, 2,3-dihydroxypropoxy, and —C(═NOCH₃)CH₃.

16. (canceled)

17. The compound of claim 5, wherein:
1) R₁₈ and R₁₉ are each independently H, halo, CN, R^{a}NHC(O)—, —OR^{a} or 5- or 6-membered heteroaryl optionally substituted with 1-3 independently selected R^{b} substituents;
2) R₁₈ is H and R₁₉ is —OR^{a}; or
3) R₁₉ is H and R₁₈ is —OR^{a}; or
4) R₁₈ and R₁₉ are each independently —OR^{a}; or
5) R₁₈ is 5- or 6-membered heteroaryl optionally substituted with 1-3 independently selected R^{b} substituents and R₁₉ is H or —OR^{a}; or
6) R₁₈ is H or —OR^{a} and R₁₉ is 5- or 6-membered heteroaryl optionally substituted with 1-3 independently selected R^{b} substituents; or
7) R₁₈ is R^{a}NHC(O)— and R₁₉ is H or —OR^{a}; or
8) R₁₉ is R^{a}NHC(O)— and R₁₈ is H or —OR^{a}.

18. The compound of claim 1, wherein R₁₀ and R₁₁ are each H.

19. The compound of claim 1, wherein the subscript m is 1, n is 1, and p is 0 or 1.

20-21. (canceled)

22. The compound of claim 1, wherein R₁₃ is H, F, Cl, Br, CH₃, CH₃O, CN, —C(O)NH₂, or CF₃ and the subscript n is 1 or 2.

23. (canceled)

24. The compound of claim 1, having Formula I: 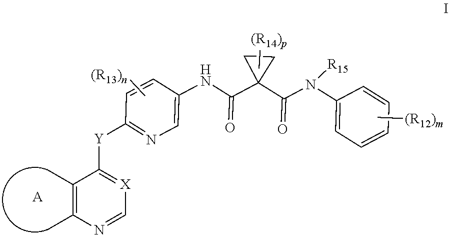
wherein:
X is selected from N and C—H;
Y is O, S, SO, SO₂, NH, or N—(C₁-C₆ alkyl);
R₁₃ is selected from —H, halo, —CN, —C(O)NH₂, and optionally substituted C₁₋₆ alkyl;
R₁₂ is —H or halo; 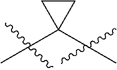 [SMILES: *C1(*)CC1]
is optionally substituted with one, two, three, or four groups independently selected from the group consisting of halo, and C₁-C₆ alkyl, wherein “ [symbol] ” indicate points of attachment;
wherein R₁₈ and R₁₉ are selected from the group consisting of H, halo, —CN, optionally substituted C₁-C₆ alkyl, C(O)NR₅R₆, and optionally substituted C₁-C₆ alkoxy; or
when 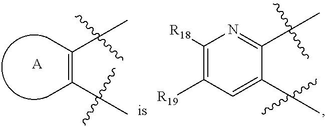
R₁₈ and R₁₉ can be joined together to form a 5 or 6-membered optionally substituted cycloalkyl or optionally substituted heterocycloalkyl;
R₅ and R₆ are selected from the group consisting of H, optionally substituted C₁₋₆ alkyl, or R₅ and R₆ taken together with the nitrogen to which they are attached to form a 5- or 6-membered optionally substituted heterocycle; and
n and m are each independently 1 or 2;
wherein when 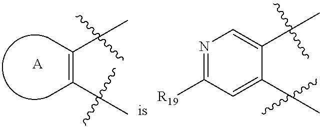
and X is C—H, R₁₉ is not optionally substituted C₁-C₆ alkyl, halo, or optionally substituted C₁-C₆ alkoxy.

25. The compound of claim 24, wherein R₁₉ is selected from the group consisting of optionally substituted C₁-C₆ alkoxy and —CN.

26. The compound of claim 1, wherein

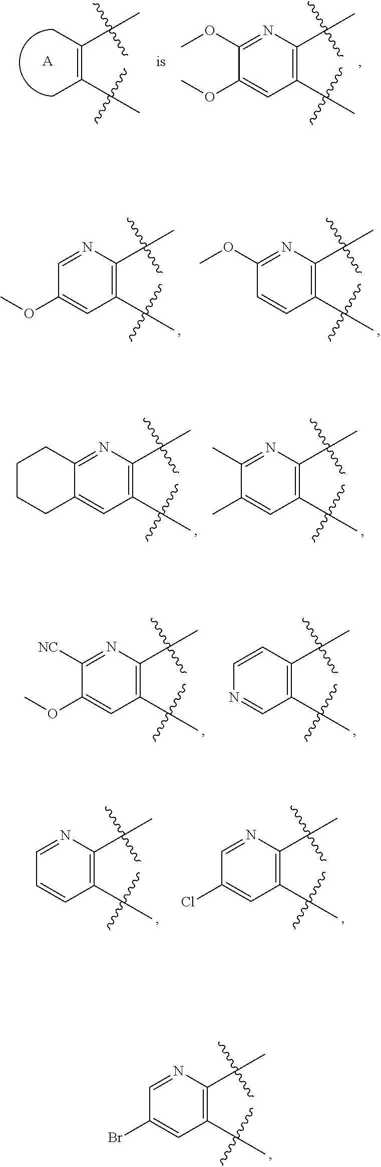

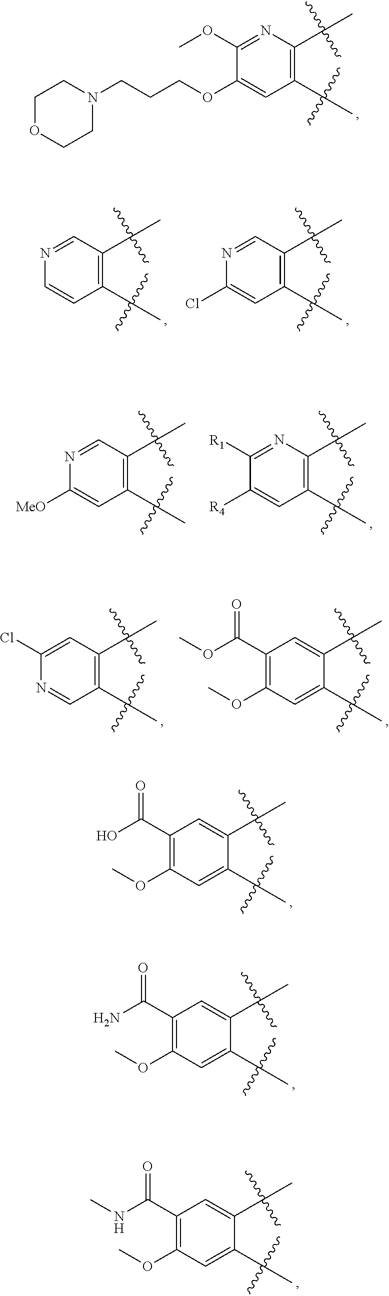

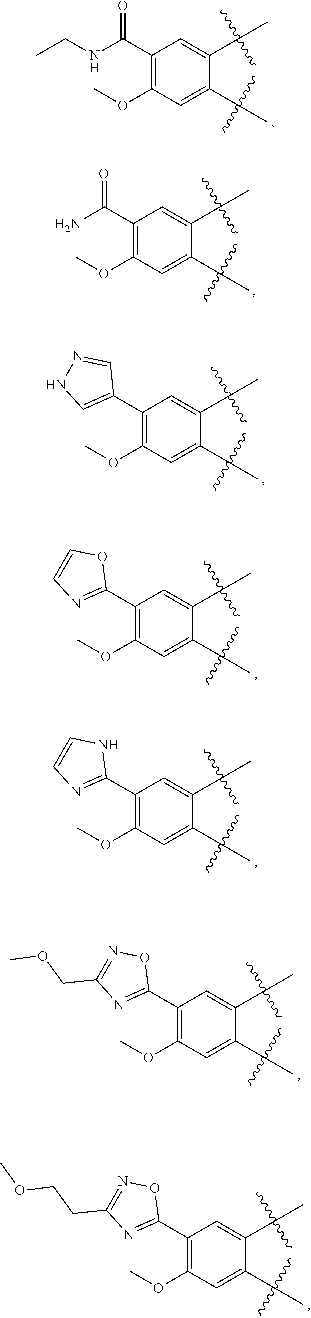

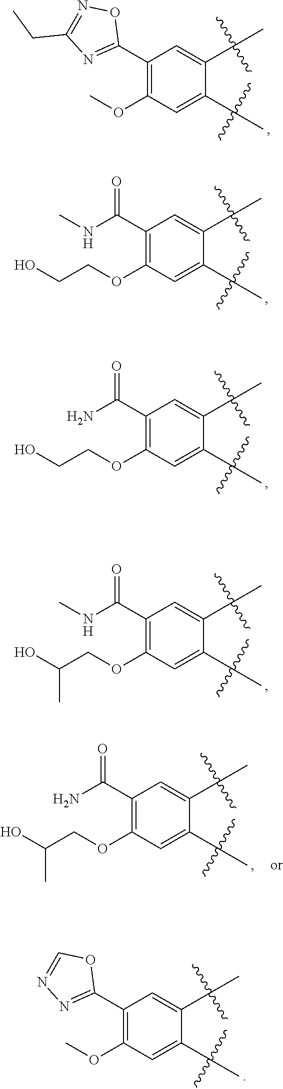 [SMILES: *c1cc(OC)c(-c2nc(CC)no2)cc1* ; *c1cc(OCCO)c(C(=O)NC)cc1* ; *c1cc(OCCO)c(C(N)=O)cc1* ; *c1cc(OCC(C)O)c(C(=O)NC)cc1* ; *c1cc(OCC(C)O)c(C(N)=O)cc1* ; *c1cc(OC)c(-c2nnco2)cc1*]

27. The compound of claim 1, wherein X is N and Y is O.

28. (canceled)

29. The compound of claim 1, wherein the compound is selected from the compounds listed in the table below
TABLE 2
Compounds of Formula I

| # | Structure | IUPAC Name |
|---|---|---|
| 80 | 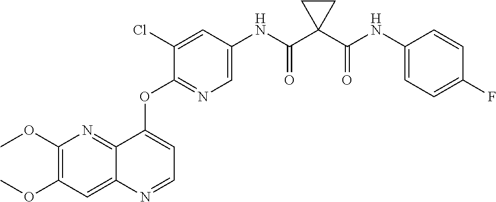 [SMILES: COc1cc2nccc(Oc3ncc(NC(=O)C4(C(=O)Nc5ccc(F)cc5)CC4)cc3Cl)c2nc1OC] | 1-N′-[5-chloro-6-[(6,7-dimethoxy- 1,5-naphthyridin-4-yl)oxy]pyridin- 3-yl]-1-N-(4- fluorophenyl)cyclopropane-1,1- dicarboxamide |
| 81 | 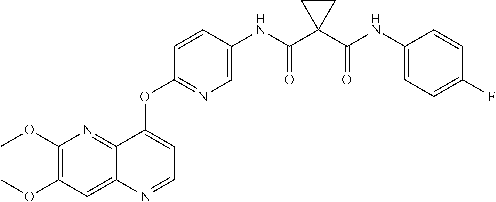 [SMILES: COc1cc2nccc(Oc3ccc(NC(=O)C4(C(=O)Nc5ccc(F)cc5)CC4)cn3)c2nc1OC] | 1-N′-[6-[(6,7-dimethoxy-1,5- naphthyridin-4-yl)oxy]pyridin-3- yl]-1-N-(4- fluorophenyl)cyclopropane-1,1- dicarboxamide |
| 82 | 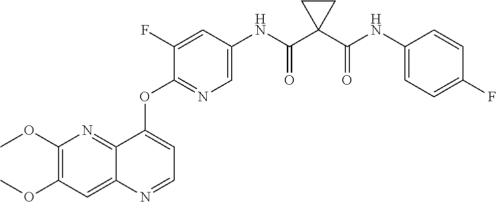 [SMILES: COc1cc2nccc(Oc3ncc(NC(=O)C4(C(=O)Nc5ccc(F)cc5)CC4)cc3F)c2nc1OC] | 1-N′-[6-[(6,7-dimethoxy-1,5- naphthyridin-4-yl)oxy]-5- fluoropyridin-3-yl]-1-N-(4- fluorophenyl)cyclopropane-1,1- dicarboxamide |
| 83 | 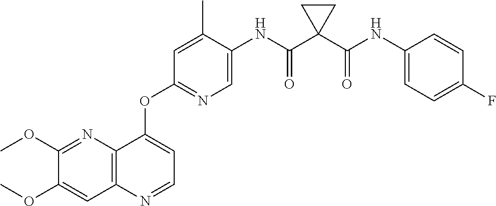 [SMILES: COc1cc2nccc(Oc3cc(C)c(NC(=O)C4(C(=O)Nc5ccc(F)cc5)CC4)cn3)c2nc1OC] | 1-N′-[6-[(6,7-dimethoxy-1,5- naphthyridin-4-yl)oxy]-4- methylpyridin-3-yl]-1-N-(4- fluorophenyl)cyclopropane-1,1- dicarboxamide |
| 84 | 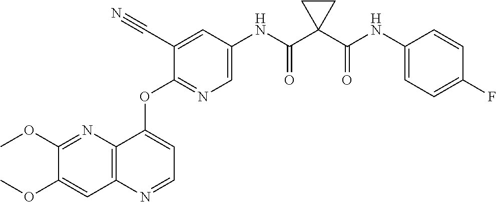 [SMILES: COc1cc2nccc(Oc3ncc(NC(=O)C4(C(=O)Nc5ccc(F)cc5)CC4)cc3C#N)c2nc1OC] | 1-N′-[5-cyano-6-[(6,7-dimethoxy- 1,5-naphthyridin-4-yl)oxy]pyridin- 3-yl]-1-N-(4- fluorophenyl)cyclopropane-1,1- dicarboxamide |
| 85 | 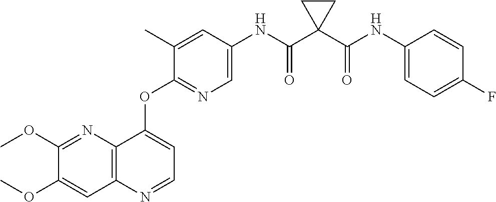 [SMILES: COc1cc2nccc(Oc3ncc(NC(=O)C4(C(=O)Nc5ccc(F)cc5)CC4)cc3C)c2nc1OC] | 1-N′-[6-[(6,7-dimethoxy-1,5- naphthyridin-4-yl)oxy]-5- methylpyridin-3-yl]-1-N-(4- fluorophenyl)cyclopropane-1,1 dicarboxamide |
| 86 | 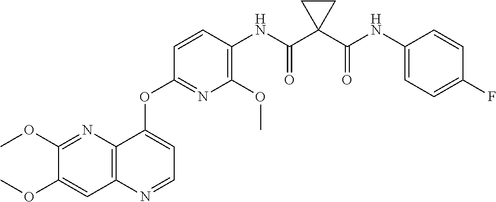 [SMILES: COc1cc2nccc(Oc3ccc(NC(=O)C4(C(=O)Nc5ccc(F)cc5)CC4)c(OC)n3)c2nc1OC] | 1-N′-[6-[(6,7-dimethoxy-1,5- naphthyridin-4-yl)oxy]-2- methoxypyridin-3-yl]-1-N-(4- fluorophenyl)cyclopropane-1,1- dicarboxamide |
| 87 | 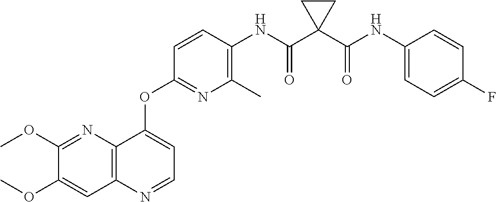 [SMILES: COc1cc2nccc(Oc3ccc(NC(=O)C4(C(=O)Nc5ccc(F)cc5)CC4)c(C)n3)c2nc1OC] | 1-N'-[6-[(6,7-dimethoxy-1,5- naphthyridin-4-yl)oxy]-2- methylpyridin-3-yl]-1-N-(4- fluorophenyl)cyclopropane-1,1- dicarboxamide |
| 88 | 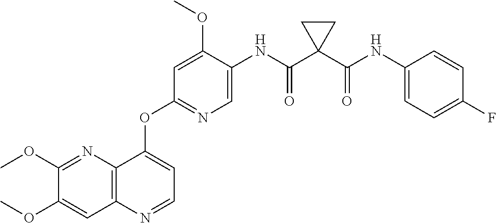 [SMILES: COc1cc(Oc2ccnc3cc(OC)c(OC)nc23)ncc1NC(=O)C1(C(=O)Nc2ccc(F)cc2)CC1] | 1 -N'-[6-[(6,7-dimethoxy-1,5- naphthyridin-4-yl)oxy]-4- methoxypyridin-3-yl]-1-N-(4- fluorophenyl)cyclopropane-1,1- dicarboxamide |
| 89 | 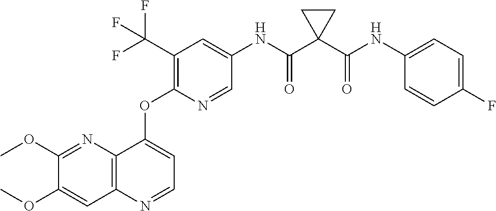 [SMILES: COc1cc2nccc(Oc3ncc(NC(=O)C4(C(=O)Nc5ccc(F)cc5)CC4)cc3C(F)(F)F)c2nc1OC] | 1-N′-[6-[(6,7-dimethoxy-1,5- naphthyridin-4-yl)oxy]-5- (trifluoromethyl)pyridin-3-yl]-1- N-(4-fluorophenyl)cyclopropane- 1,1-dicarboxamide |
| 90 | 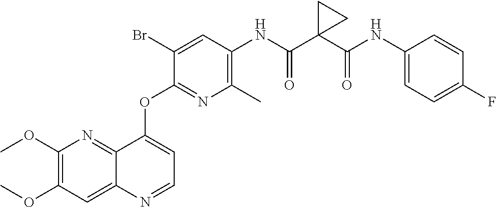 [SMILES: COc1cc2nccc(Oc3nc(C)c(NC(=O)C4(C(=O)Nc5ccc(F)cc5)CC4)cc3Br)c2nc1OC] | 1-N′-[5-bromo-6-[(6,7-dimethoxy- 1,5-naphthyridin-4-yl)oxy]-2- methylpyridin-3-yl]-1-N-(4- fluorophenyl)cyclopropane-1,1- dicarboxamide |
| 91 | 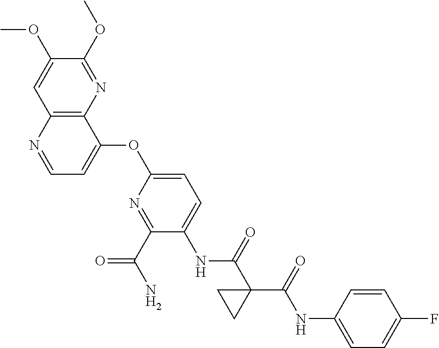 [SMILES: COc1cc2nccc(Oc3ccc(NC(=O)C4(C(=O)Nc5ccc(F)cc5)CC4)c(C(N)=O)n3)c2nc1OC] | 1-N′-[2-carbamoyl-6-[(6,7- dimethoxy-1,5-naphthyridin-4- yl)oxy]pyridin-3-yl]-1-N-(4- fluorophenyl)cyclopropane-1,1- dicarboxamide |
| 92 | 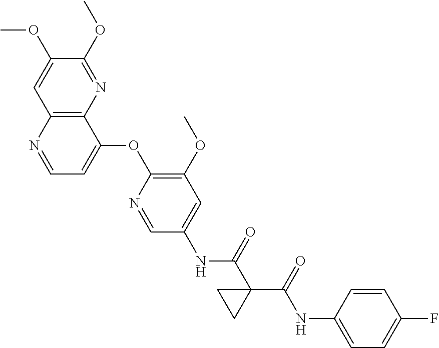 [SMILES: COc1cc2nccc(Oc3ncc(NC(=O)C4(C(=O)Nc5ccc(F)cc5)CC4)cc3OC)c2nc1OC] | 1-N′-[6-[(6,7-dimethoxy-1,5- naphthyridin-4-yl)oxy]-5- methoxypyridin-3-yl]-1-N-(4- fluorophenyl)cyclopropane-1,1- dicarboxamide |
| 96 | 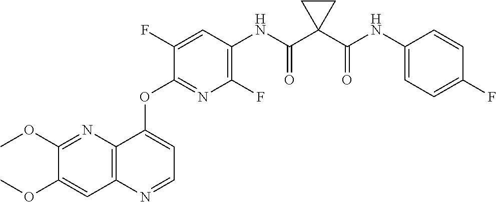 [SMILES: COc1cc2nccc(Oc3nc(F)c(NC(=O)C4(C(=O)Nc5ccc(F)cc5)CC4)cc3F)c2nc1OC] | 1-N′-[6-[(6,7-dimethoxy-1,5- naphthyridin-4-yl)oxy]-2,5- difluoropyridin-3-yl]-1-N-(4- fluorophenyl)cyclopropane-1,1- dicarboxamide |
| 103 | 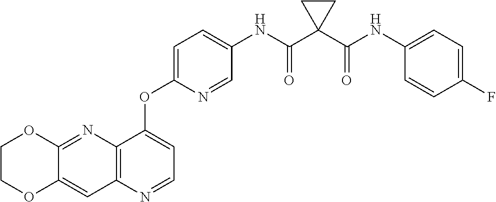 [SMILES: O=C(Nc1ccc(F)cc1)C1(C(=O)Nc2ccc(Oc3ccnc4cc5c(nc34)OCCO5)nc2)CC1] | 1-N′-[6-(2,3-dihydro- [1,4]dioxino[2,3- b][1,5]naphthyridin-6- yloxy)pyridin-3-yl]-1-N-(4- fIuorophenyl)cyclopropane-1,1- dicarboxamide |
| 104 | 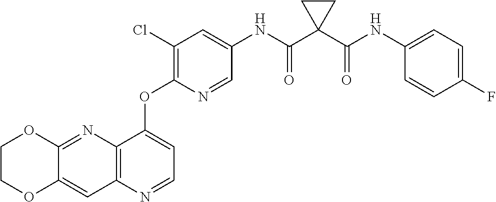 [SMILES: O=C(Nc1ccc(F)cc1)C1(C(=O)Nc2cnc(Oc3ccnc4cc5c(nc34)OCCO5)c(Cl)c2)CC1] | 1-N′-[5-chloro-6-(2,3-dihydro- [1,4]dioxino[2,3- b][1,5]naphthyridin-6- yloxy)pyridin-3-yl]-1-N-(4- fluorophenyl)cyclopropane-1,1- dicarboxamide |
| 105 | 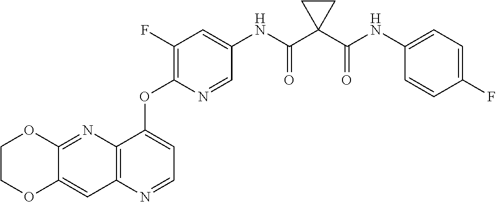 [SMILES: O=C(Nc1ccc(F)cc1)C1(C(=O)Nc2cnc(Oc3ccnc4cc5c(nc34)OCCO5)c(F)c2)CC1] | 1-N′-[6-(2,3-dihydro- [1,4]dioxino[2,3- b][1,5]naphthyridin-6-yloxy)-5 fluoropyridin-3-yl]-1-N-(4- fluorophenyl)cyclopropane-1,1 dicarboxamide |
| 114 | 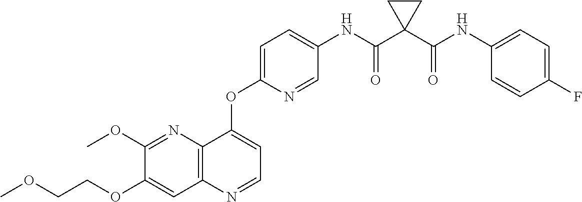 [SMILES: COCCOc1cc2nccc(Oc3ccc(NC(=O)C4(C(=O)Nc5ccc(F)cc5)CC4)cn3)c2nc1OC] | 1-N-(4-fluorophenyl)-1-N′-[6-[[6- methoxy-7-(2-methoxyethoxy)- 1,5-naphthyridin-4-yl]oxy]pyridin- 3-yl]cyclopropane-1,1- dicarboxamide |
| 115 | 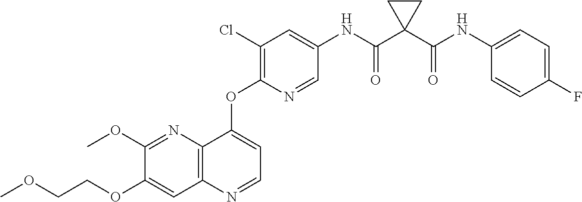 [SMILES: COCCOc1cc2nccc(Oc3ncc(NC(=O)C4(C(=O)Nc5ccc(F)cc5)CC4)cc3Cl)c2nc1OC] | 1-N′-[5-chloro-6-[[6-methoxy-7- (2-methoxyethoxy)-1,5- naphthyridin-4-yl]oxy]pyridin-3 - yl]-1-N-(4- fluorophenyl)cyclopropane-1,1- dicarboxamide |
| 116 | 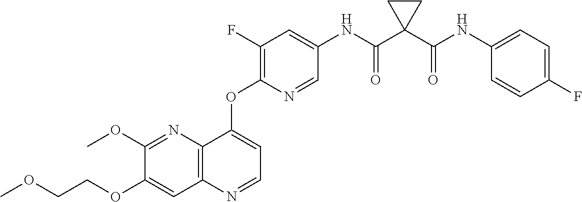 [SMILES: COCCOc1cc2nccc(Oc3ncc(NC(=O)C4(C(=O)Nc5ccc(F)cc5)CC4)cc3F)c2nc1OC] | 1-N′-[5-fluoro-6-[[6-methoxy-7- (2-methoxyethoxy)-1,5- naphthyridin-4-yl]oxy]pyridin-3- yl]-1-N-(4- fluorophenyl)cyclopropane-1,1- dicarboxamide |
| 120 | 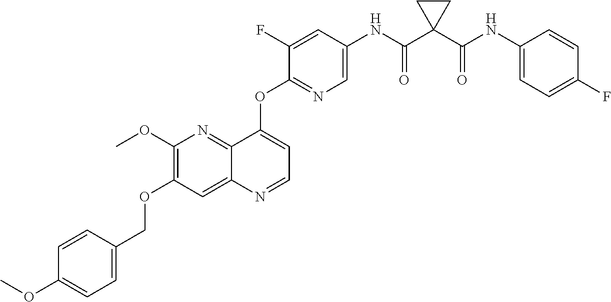 [SMILES: COc1ccc(COc2cc3nccc(Oc4ncc(NC(=O)C5(C(=O)Nc6ccc(F)cc6)CC5)cc4F)c3nc2OC)cc1] | 1-N′-[5-fluoro-6-[[6-methoxy-7- [(4-methoxyphenyl)methoxy]-1,5- naphthyridin-4-yl]oxy]pyridin-3- yl]-1-N-(4- fluorophenyl)cyclopropane-1,1- dicarboxamide |
| 121 | 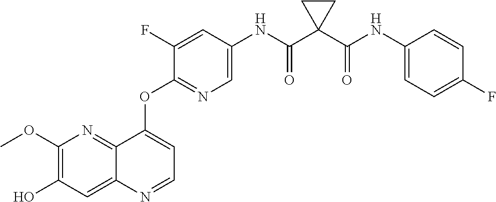 [SMILES: COc1nc2c(Oc3ncc(NC(=O)C4(C(=O)Nc5ccc(F)cc5)CC4)cc3F)ccnc2cc1O] | 1-N′-[5-fluoro-6-[(7-hydroxy-6- methoxy-1,5-naphthyridin-4- yl)oxy]pyridin-3-yl]-1-N-(4- fluorophenyl)cyclopropane-1,1- dicarboxamide |
| 122 | 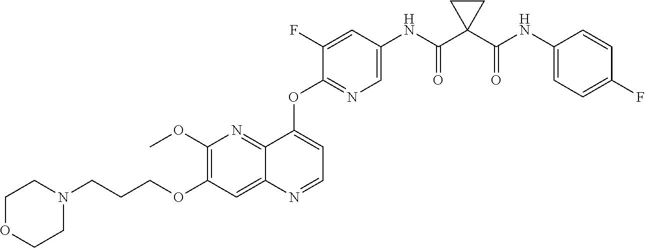 [SMILES: COc1nc2c(Oc3ncc(NC(=O)C4(C(=O)Nc5ccc(F)cc5)CC4)cc3F)ccnc2cc1OCCCN1CCOCC1] | 1-N′-[5-fluoro-6-[[6-methoxy-7- (3-morpholin-4-ylpropoxy)-1,5- naphthyridin-4-yl]oxy]pyridin-3- yl]-1-N-(4- fluorophenyl)cyclopropane-1,1- dicarboxamide |
| 123 | 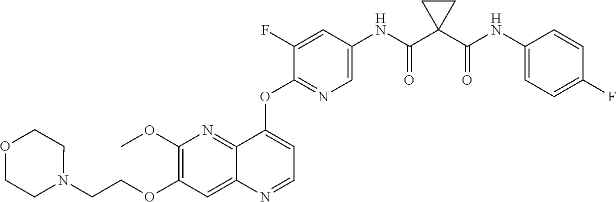 [SMILES: COc1nc2c(Oc3ncc(NC(=O)C4(C(=O)Nc5ccc(F)cc5)CC4)cc3F)ccnc2cc1OCCN1CCOCC1] | 1-N′-[5-fluoro-6-[[6-methoxy-7- (2-morpholin-4-ylethoxy)-1,5- naphthyridin-4-yl]oxy]pyridin-3- yl]-1-N-(4- fluorophenyl)cyclopropane-1,1- dicarboxamide |
| 124 | 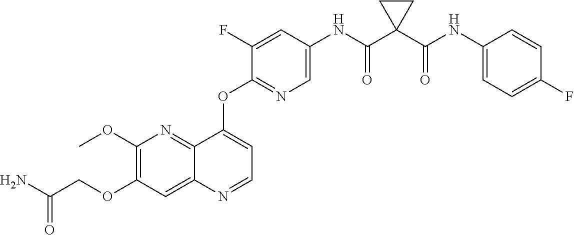 [SMILES: COc1nc2c(Oc3ncc(NC(=O)C4(C(=O)Nc5ccc(F)cc5)CC4)cc3F)ccnc2cc1OCC(N)=O] | 1-N′-[6-[[7-(2-amino-2- oxoethoxy)-6-methoxy-1,5- naphthyridin-4-yl]oxy]-5- fluoropyridin-3-yl]-1-N-(4- fluorophenyl)cyclopropane-1,1- dicarboxamide |
| 125 | 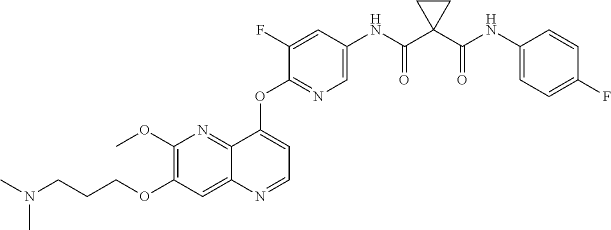 [SMILES: COc1nc2c(Oc3ncc(NC(=O)C4(C(=O)Nc5ccc(F)cc5)CC4)cc3F)ccnc2cc1OCCCN(C)C] | 1-N′-[6-[[7-[3- (dimethylamino)propoxy]-6- methoxy-1,5-naphthyridin-4- yl]oxy]-5-fluoropyridin-3-yl]-1-N- (4-fluorophenyl)cyclopropane-1,1- dicarboxamide |
| 127 | 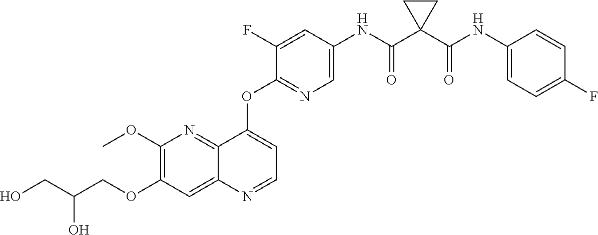 [SMILES: COc1nc2c(Oc3ncc(NC(=O)C4(C(=O)Nc5ccc(F)cc5)CC4)cc3F)ccnc2cc1OCC(O)CO] | 1-N′-[6-[[7-(2,3- dihydroxypropoxy)-6-methoxy- 1,5-naphthyridin-4-yl]oxy]-5- fluoropyridin-3-yl]-l-N-(4- fluorophenyl)cyclopropane-1,1- dicarboxamide |
| 134 | 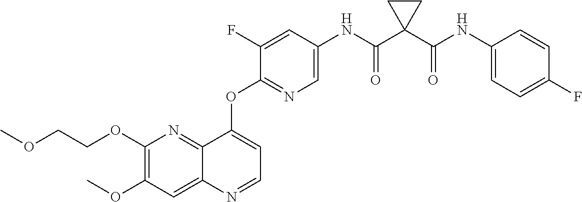 [SMILES: COCCOc1nc2c(Oc3ncc(NC(=O)C4(C(=O)Nc5ccc(F)cc5)CC4)cc3F)ccnc2cc1OC] | 1-N′-[5-fluoro-6-[[7-methoxy-6- (2-methoxyethoxy)-l ,5- naphthyridin-4-yl]oxy]pyridin-3- yl]-1-N-(4- fluorophenyl)cyclopropane-1,1- dicarboxamide |
| 135 | 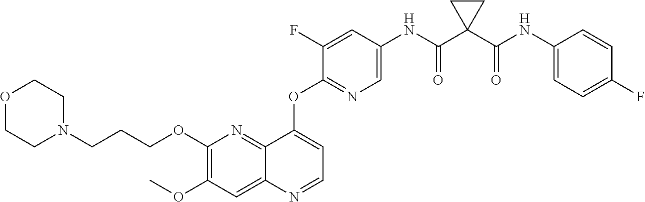 [SMILES: COc1cc2nccc(Oc3ncc(NC(=O)C4(C(=O)Nc5ccc(F)cc5)CC4)cc3F)c2nc1OCCCN1CCOCC1] | 1-N′-[5-fluoro-6-[[7-methoxy-6- (3-morpholin-4-ylpropoxy)-1,5- naphthyridin-4-yl]oxy]pyridin-3- yl]-1-N-(4- fluorophenyl)cyclopropane-1,1- dicarboxamide |
| 145 | 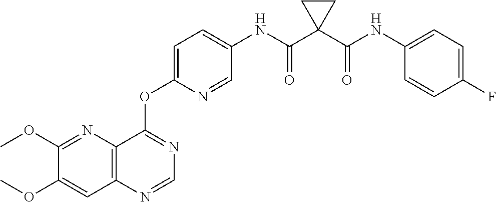 [SMILES: COc1cc2ncnc(Oc3ccc(NC(=O)C4(C(=O)Nc5ccc(F)cc5)CC4)cn3)c2nc1OC] | 1-N′-[6-(6,7-dimethoxypyrido[3,2- d]pyrimidin-4-yl)oxypyridin-3-yl]- 1-N-(4- fluorophenyl)cyclopropane-1,1- dicarboxamide |
| 155 | 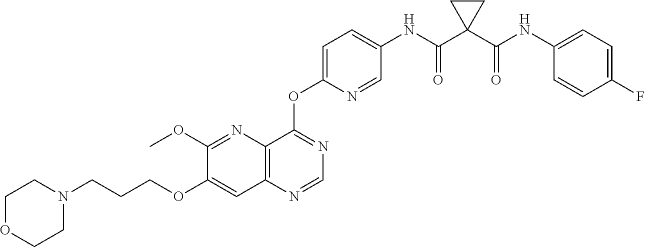 [SMILES: COc1nc2c(Oc3ccc(NC(=O)C4(C(=O)Nc5ccc(F)cc5)CC4)cn3)ncnc2cc1OCCCN1CCOCC1] | 1-N-(4-fluorophenyl)-l-N′-[6-[6- methoxy-7-(3-morpholin-4- ylpropoxy)pyrido[3,2-d]pyrimidin- 4-yl]oxypyridin-3- yl]cydopropane-1,1- dicarboxamide |
| 156 | 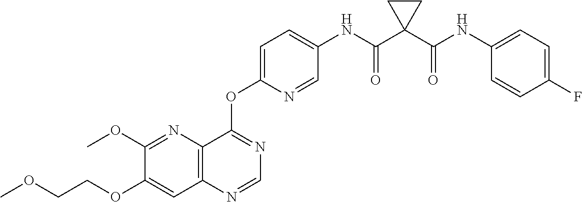 [SMILES: COCCOc1cc2ncnc(Oc3ccc(NC(=O)C4(C(=O)Nc5ccc(F)cc5)CC4)cn3)c2nc1OC] | 1-N-(4-fluorophenyl)-1-N′-[6-[6- methoxy-7-(2- methoxyethoxy)pyrido[3,2- d]pyrimidin-4-yl]oxypyridin-3- yl]cyclopropane-1,1- dicarboxamide |
| 159 | 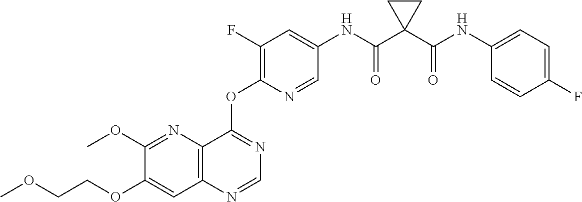 [SMILES: COCCOc1cc2ncnc(Oc3ncc(NC(=O)C4(C(=O)Nc5ccc(F)cc5)CC4)cc3F)c2nc1OC] | 1-N′-[5-fluoro-6-[6-methoxy-7-(2- methoxyethoxy)pyrido[3,2- d]pyrimidin-4-yl]oxypyridin-3-yl]- 1-N-(4- fluorophenyl)cyclopropane-1,1- dicarboxamide |

30-32. (canceled)

33. The compound of claim 24, wherein R₁₂ is halo.

34-39. (canceled)

40. A pharmaceutical composition comprising a compound of claim 1, and a pharmaceutically acceptable carrier or excipient.

41. A method of treating a disease, disorder, or syndrome mediated at least in part by modulating in vivo activity of a protein kinase, comprising administering to a subject in need thereof a therapeutically effective amount of a compound of claim 1.


SEQUENCE LISTING (45 sequences)

SEQ ID NO: 1 - protein, 587 aa, Homo sapiens
MRPRGLPPLLVVLLGCWASVSAQTDATPAVTTEGLNSTEAALATFGTFPSTRPPGTPRAPGPSSGPRPTPVTDVAVLCVCDLSPAQCDINCCCDPDCSSVDFSVFSACSVPVVTGDSQFCSQKAVIYSLNFTANPPQRVFELVDQINPSIFCIHITNYKPALSFINPEVPDENNFDTLMKTSDGFTLNAESYVSFTTKLDIPTAAKYEYGVPLQTSDSFLRFPSSLTSSLCTDNNPAAFLVNQAVKCTRKINLEQCEEIEALSMAFYSSPEILRVPDSRKKVPITVQSIVIQSLNKTLTRREDTDVLQPTLVNAGHFSLCVNVVLEVKYSLTYTDAGEVTKADLSFVLGTVSSVVVPLQQKFEIHFLQENTQPVPLSGNPGYVVGLPLAAGFQPHKGSGIIQTTNRYGQLTILHSTTEQDCLALEGVRTPVLFGYTMQSGCKLRLTGALPCQLVAQKVKSLLWGQGFPDYVAPFGNSQAQDMLDWVPIHFITQSFNRKDSCQLPGALVIEVKWTKYGSLLNPQAKIVNVTANLISSSFPEANSGNERTILISTAVTFVDVSAPAEAGFRAPPAINARLPFNFFFPFV

SEQ ID NO: 2 - protein, 697 aa, Homo sapiens
MGFQPPAALLLRLFLLQGILRLLWGDLAFIPPFIRMSGPAVSASLVGDTEGVTVSLAVLQDEAGILPIPTCGVLNNETEDWSVTVIPGAKVLEVTVRWKRGLDWCSSNETDSFSESPCILQTLLVSASHNSSCSAHLLIQVEIYANSSLTHNASENVTVIPNQVYQPLGPCPCNLTAGACDVRCCCDQECSSNLTTLFRRSCFTGVFGGDVNPPFDQLCSAGTTTRGVPDWFPFLCVQSPLANTPFLGYFYHGAVSPKQDSSFEVYVDTDAKDFADFGYKQGDPIMTVKKAYFTIPQVSLAGQCMQNAPVAFLHNFDVKCVTNLELYQERDGIINAKIKNVALGGIVTPKVIYEEATDLDKFITNTETPLNNGSTPRIVNVEEHYIFKWNNNTISEINVKIFRAEINAHQKGIMTQRFVVKFLSYNSGNEEELSGNPGYQLGKPVRALNINRMNNVTTLHLWQSAGRGLCTSATFKPILFGENVLSGCLLEVGINENCTQLRENAVERLDSLIQATHVAMRGNSDYADLSDGWLEIIRVDAPDPGADPLASSVNGMCLDIPAHLSIRILISDAGAVEGITQQEILGVETRFSSVNWQYQCGLTCEHKADLLPISASVQFIKIPAQLPHPLTRFQINYTEYDCNRNEVCWPQLLYPWTQYYQGELHSQCVAKGLLLLLFLTLALFLSNPWTRICKAYS

SEQ ID NO: 3 - protein, 607 aa, Homo sapiens
MRTPQLALLQVFFLVFPDGVRPQPSSSPSGAVPTSLELQRGTDGGTLQSPSEATATRPAVPGLPTVVPTLVTPSAPGNRTVDLFPVLPICVCDLTPGACDINCCCDRDCYLLHPRTVFSFCLPGSVRSSSWVCVDNSVIFRSNSPFPSRVFMDSNGIRQFCVHVNNSNLNYFQKLQKVNATNFQALAAEFGGESFTSTFQTQSPPSFYRAGDPILTYFPKWSVISLLRQPAGVGAGGLCAESNPAGFLESKSTTCTRFFKNLASSCTLDSALNAASYYNFTVLKVPRSMTDPQNMEFQVPVILTSQANAPLLAGNTCQNVVSQVTYEIETNGTFGIQKVSVSLGQTNLTVEPGASLQQHFILRFRAFQQSTAASLTSPRSGNPGYIVGKPLLALTDDISYSMTLLQSQGNGSCSVKRHEVQFGVNAISGCKLRLKKADCSHLQQEIYQTLHGRPRPEYVAIFGNADPAQKGGWTRILNRHCSISAINCTSCCLIPVSLEIQVLWAYVGLLSNPQAHVSGVRFLYQCQSIQDSQQVTEVSLTTLVNFVDITQKPQPPRGQPKMDWKWPFDFFPFKVAFSRGVFSQKCSVSPILILCLLLLGVLNLETM

SEQ ID NO: 4 - protein, 106 aa, Homo sapiens
MATPGPVIPEVPFEPSKPPVIEGLSPTVYRNPESFKEKFVRKTRENPVVPIGCLATAAALTYGLYSFHRGNSQRSQLMMRTRIAAQGFTVAAILLGLAVTAMKSRP

SEQ ID NO: 5 - protein, 106 aa, Homo sapiens
MATLGFVTPEAPFESSKPPIFEGLSPTVYSNPEGFKEKFLRKTRENPVVPIGFLCTAAVLTNGLYCFHQGNSQCSRLMMHTQIAAQGFTIAAILLGLAATAMKSPP

SEQ ID NO: 6 - protein, 132 aa, Homo sapiens
MCSAGELLRGGDGGERDEDGDALAEREAAGTGWDPGASPRRRGQRPKESEQDVEDSQNHTGEPVGDDYKKMGTLFGELNKNLINMGFTRMYFGERIVEPVIVIFFWVMLWFLGLQALGLVAVLCLVIIYVQQ

SEQ ID NO: 7 - protein, 1104 aa, Homo sapiens
MERSPGEGPSPSPMDQPSAPSDPTDQPPAAHAKPDPGSGGQPAGPGAAGEALAVLTSFGRRLLVLIPVYLAGAVGLSVGFVLFGLALYLGWRRVRDEKERSLRAARQLLDDEEQLTAKTLYMSHRELPAWVSFPDVEKAEWLNKIVAQVWPFLGQYMEKLLAETVAPAVRGSNPHLQTFTFTRVELGEKPLRIIGVKVHPGQRKEQILLDLNISYVGDVQIDVEVKKYFCKAGVKGMQLHGVLRVILEPLIGDLPFVGAVSMFFIRRPTLDINWTGMTNLLDIPGLSSLSDTMIMDSIAAFLVLPNRLLVPLVPDLQDVAQLRSPLPRGIIRIHLLAARGLSSKDKYVKGLIEGKSDPYALVRLGTQTFCSRVIDEELNPQWGETYEVMVHEVPGQEIEVEVFDKDPDKDDFLGRMKLDVGKVLQASVLDDWFPLQGGQGQVHLRLEWLSLLSDAEKLEQVLQWNWGVSSRPDPPSAAILVVYLDRAQDLPLKKGNKEPNPMVQLSIQDVTQESKAVYSTNCPVWEEAFRFFLQDPQSQELDVQVKDDSRALTLGALTLPLARLLTAPELILDQWFQLSSSGPNSRLYMKLVMRILYLDSSEICFPTVPGCPGAWDVDSENPQRGSSVDAPPRPCHTTPDSQFGTEHVLRIHVLEAQDLIAKDRFLGGLVKGKSDPYVKLKLAGRSFRSHVVREDLNPRWNEVFEVIVTSVPGQELEVEVFDKDLDKDDFLGRCKVRLTTVLNSGFLDEWLTLEDVPSGRLHLRLERLTPRPTAAELEEVLQVNSLIQTQKSAELAAALLSIYMERAEDLPLRKGTKHLSPYATLTVGDSSHKTKTISQTSAPVWDESASFLIRKPHTESLELQVRGEGTGVLGSLSLPLSELLVADQLCLDRWFTLSSGQGQVLLRAQLGILVSQHSGVEAHSHSYSHSSSSLSEEPELSGGPPHITSSAPELRQRLTHVDSPLEAPAGPLGQVKLTLWYYSEERKLVSIVHGCRSLRQNGRDPPDPYVSLLLLPDKNRGTKRRTSQKKRTLSPEFNERFEWELPLDEAQRRKLDVSVKSNSSFMSRERELLGKVQLDLAETDLSQGVARWYDLMDNKDKGSS

SEQ ID NO: 8 - protein, 271 aa, Homo sapiens
MRGNLALVGVLISLAFLSLLPSGHPQPAGDDACSVQILVPGLKGDAGEKGDKGAPGRPGRVGPTGEKGDMGDKGQKGSVGRHGKIGPIGSKGEKGDSGDIGPPGPNGEPGLPCECSQLRKAIGEMDNQVSQLTSELKFIKNAVAGVRETESKIYLLVKEEKRYADAQLSCQGRGGTLSMPKDEAANGLMAAYLAQAGLARVFIGINDLEKEGAFVYSDHSPMRTFNKWRSGEPNNAYDEEDCVEMVASGGWNDVACHTTMYFMCEFDKENM

SEQ ID NO: 9 - protein, 847 aa, Homo sapiens
MFKCWSVVLVLGFIFLESEGRPTKEGGYGLKSYQPLMRLRHKQEKNQESSRVKGFMIQDGPFGSCENKYCGLGRHCVTSRETGQAECACMDLCKRHYKPVCGSDGEFYENHCEVHRAACLKKQKITIVHNEDCFFKGDKCKTTEYSKMKNMLLDLQNQKYIMQENENPNGDDISRKKLLVDQMFKYFDADSNGLVDINELTQVIKQEELGKDLFDCTLYVLLKYDDFNADKHLALEEFYRAFQVIQLSLPEDQKLSITAATVGQSAVLSCAIQGTLRPPIIWKRNNIILNNLDLEDINDFGDDGSLYITKVTTTHVGNYTCYADGYEQVYQTHIFQVNVPPVIRVYPESQAREPGVTASLRCHAEGIPKPQLGWLKNGIDITPKLSKQLTLQANGSEVHISNVRYEDTGAYTCIAKNEAGVDEDISSLFVEDSARKTLANILWREEGLGIGNMFYVFYEDGIKVIQPIECEFQRHIKPSEKLLGFQDEVCPKAEGDEVQRCVWASAVNVKDKFIYVAQPTLDRVLIVDVQSQKVVQAVSTDPVPVKLHYDKSHDQVWVLSWGTLEKTSPTLQVITLASGNVPHHTIHTQPVGKQFDRVDDFFIPTTTLIITHMRFGFILHKDEAALQKIDLETMSYIKTINLKDYKCVPQSLAYTHLGGYYFIGCKPDSTGAVSPQVMVDGVTDSVIGFNSDVTGTPYVSPDGHYLVSINDVKGLVRVQYITIRGEIQEAFDIYTNLHISDLAFQPSFTEAHQYNIYGSSSTQTDVLFVELSSGKVKMIKSLKEPLKAEEWPWNRKNRQIQDSGLFGQYLMTPSKDSLFILDGRLNKLNCEITEVEKGNTVIWVGDA

SEQ ID NO: 10 - protein, 470 aa, Homo sapiens
MSRLGALGGARAGLGLLLGTAAGLGFLCLLYSQRWKRTQRHGRSQSLPNSLDYTQTSDPGRHVMLLRAVPGGAGDASVLPSLPREGQEKVLDRLDFVLTSLVALRREVEELRSSLRGLAGEIVGEVRCHMEENQRVARRRRFPFVRERSDSTGSSSVYFTASSGATFTDAESEGGYTTANAESDNERDSDKESEDGEDEVSCETVKMGRKDSLDLEEEAASGASSALEAGGSSGLEDVLPLLQQADELHRGDEQGKREGFQLLLNNKLVYGSRQDFLWRLARAYSDMCELTEEVSEKKSYALDGKEEAEAALEKGDESADCHLWYAVLCGQLAEHESIQRRIQSGFSFKEHVDKAIALQPENPMAHFLLGRWCYQVSHLSWLEKKTATALLESPLSATVEDALQSFLKAEELQPGFSKAGRVYISKCYRELGKNSEARWWMKLALELPDVTKEDLAIQKDLEELEVILRD

SEQ ID NO: 11 - protein, 495 aa, Homo sapiens
MENKAMYLHTVSDCDTSSICEDSFDGRSLSKLNLCEDGPCHKRRASICCTQLGSLSALKHAVLGLYLLVFLILVGIFILAVSRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASGVEAPMMIRLVNGSGPHEGRVEVYHDRRWGTVCDDGWDKKDGDVVCRMLGFRGVEEVYRTARFGQGTGRIWMDDVACKGTEETIFRCSFSKWGVTNCGHAEDASVTCNRH

SEQ ID NO: 12 - protein, 236 aa, Homo sapiens
MTAEFLSLLCLGLCLGYEDEKKNEKPPKPSLHAWPSSVVEAESNVTLKCQAHSQNVTFVLRKVNDSGYKQEQSSAENEAEFPFTDLKPKDAGRYFCAYKTTASHEWSESSEHLQLVVTDKHDELEAPSMKTDTRTIFVAIFSCISILLLFLSVFIIYRCSQHSSSSEESTKRTSHSKLPEQEAAEADLSNMERVSLSTADPQGVTYAELSTSALSEAASDTTQEPPGSHEYAALKV

SEQ ID NO: 13 - protein, 180 aa, Homo sapiens
MAAAEEEDGGPEGPNRERGGAGATFECNICLETAREAVVSVCGHLYCWPCLHQWLETRPERQECPVCKAGISREKVVPLYGRGSQKPQDPRLKTPPRPQGQRPAPESRGGFQPFGDTGGFHFSFGVGAFPFGFFTTVFNAHEPFRRGTGVDLGQGHPASSWQDSLFLFLAIFFFFWLLSI

SEQ ID NO: 14 - protein, 157 aa, Homo sapiens
MLPEQGPQPSTMPLWCLLAACTSLPRQAATMLEEAASPNEAVHASTSGSGALTDQTFTDLSAAEASSEEVPDFMEVPHSVHHKINCFFYLEKQLCQLPSPLCLSSLLTLKLKTTVPAPGRWWSFQPHKAFPLLVGTPGSWQSTIDPAWAAPSQPSPG

SEQ ID NO: 15 - protein, 716 aa, Homo sapiens
MMSIRQRREIRATEVSEDFPAQEENVKLENKLPSGCTSRRLWKILSLTIGGTIALCIGLLTSVYLATLHENDLWFSNIKEVEREISFRTECGLYYSYYKQMLQAPTLVQGFHGLIYDNKTESMKTINLLQRMNIYQEVFLSILYRVLPIQKYLEPVYFYIYTLFGLQAIYVTALYITSWLLSGTWLSGLLAAFWYVTNRIDTTRVEFTIPLRENWALPFFAIQIAAITYFLRPNLQPLSERLTLLAIFISTFLFSLTWQFNQFMMLMQALVLFTLDSLDMLPAVKATWLYGIQITSLLLVCILQFFNSMILGSLLISFNLSVFIARKLQKNLKTGSFLNRLGKLLLHLFMVLCLTLFLNNIIKKILNLKSDEHIFKFLKAKFGLGATRDFDANLYLCEEAFGLLPFNTFGRLSDTLLFYAYIFVLSITVIVAFVVAFHNLSDSTNQQSVGKMEKGTVDLKPETAYNLIHTILFGFLALSTMRMKYLWTSHMCVFASFGLCSPEIWELLLKSVHLYNPKRICIMRYSVPILILLYLCYKFWPGMMDELSELREFYDPDTVELMNWINSNTPRKAVFAGSMQLLAGVKLCTGRTLTNHPHYEDSSLRERTRAVYQIYAKRAPEEVHALLRSFGTDYVILEDSICYERRHRRGCRLRDLLDIANGHMMDGPGENDPDLKPADHPRFCEEIKRNLPPYVAYFTRVFQNKTFHVYKLSRNK

SEQ ID NO: 16 - protein, 831 aa, Homo sapiens
MKVHMHTKFCLICLLTFIFHHCNHCHEEHDHGPEALHRQHRGMTELEPSKFSKQAAENEKKYYIEKLFERYGENGRLSFFGLEKLLTNLGLGERKVVEINHEDLGHDHVSHLDILAVQEGKHFHSHNHQHSHNHLNSENQTVTSVSTKRNHKCDPEKETVEVSVKSDDKHMHDHNHRLRHHHRLHHHLDHNNTHHFHNDSITPSERGEPSNEPSTETNKTQEQSDVKLPKGKRKKKGRKSNENSEVITPGFPPNHDQGEQYEHNRVHKPDRVHNPGHSHVHLPERNGHDPGRGHQDLDPDNEGELRHTRKREAPHVKNNAIISLRKDLNEDDHHHECLNVTQLLKYYGHGANSPISTDLFTYLCPALLYQIDSRLCIEHFDKLLVEDINKDKNLVPEDEANIGASAWICGIISITVISLLSLLGVILVPIINQGCFKFLLTFLVALAVGTMSGDALLHLLPHSQGGHDHSHQHAHGHGHSHGHESNKFLEEYDAVLKGLVALGGIYLLFIIEHCIRMFKHYKQQRGKQKWFMKQNTEESTIGRKLSDHKLNNTPDSDWLQLKPLAGTDDSVVSEDRLNETELTDLEGQQESPPKNYLCIEEEKIIDHSHSDGLHTIHEHDLHAAAHNHHGENKTVLRKHNHQWHHKHSHHSHGPCHSGSDLKETGIANIAWMVIMGDGIHNFSDGLAIGAAFSAGLTGGISTSIAVFCHELPHELGDFAVLLKAGMTVKQAIVYNLLSAMMAYIGMLIGTAVGQYANNITLWIFAVTAGMFLYVALVDMLPEMLHGDGDNEEHGFCPVGQFILQNLGLLFGFAIMLVIALYEDKIVFDIQF

SEQ ID NO: 17 - protein, 600 aa, Homo sapiens
MAALAPVGSPASRGPRLAAGLRLLPMLGLLQLLAEPGLGRVHHLALKDDVRHKVHLNTFGFFKDGYMVVNVSSLSLNEPEDKDVTIGFSLDRTKNDGFSSYLDEDVNYCILKKQSVSVTLLILDISRSEVRVKSPPEAGTQLPKIIFSRDEKVLGQSQEPNVNPASAGNQTQKTQDGGKSKRSTVDSKAMGEKSFSVHNNGGAVSFQFFFNISTDDQEGLYSLYFHKCLGKELPSDKFTFSLDIEITEKNPDSYLSAGEIPLPKLYISMAFFFFLSGTIWIHILRKRRNDVFKIHWLMAALPFTKSLSLVFHAIDYHYISSQGFPIEGWAVVYYITHLLKGALLFITIALIGTGWAFIKHILSDKDKKIFMIVIPLQVLANVAYIIIESTEEGTTEYGLWKDSLFLVDLLCCGAILFPVVWSIRHLQEASATDGKGDSMGPLQQRANLRAGSRIESHHFAQADLELLASSCPPASVSQRAGITAAINLAKLKLFRHYYVLIVCYIYFTRIIAFLLKLAVPFQWKWLYQLLDETATLVFFVLTGYKFRPASDNPYLQLSQEEEDLEMESVVTTSGVMESMKKVKKVTNGSVEPQGEWEGAV

SEQ ID NO: 18 - protein, 1284 aa, Homo sapiens
MSSGDPAHLGLCLWLWLGATLGREQVQASGLLRLAVLPEDRLQMKWRESEGSGLGYLVQVKPMAGDSEQEVILTTKTPKATVGGLSPSKGYTLQIFELTGSGRFLLARREFVIEDLKSSSLDRSSQRPLGSGAPEPTPSHTGSPDPEQASEPQVAFTPSQDPRTPAGPQFRCLPPVPADMVFLVDGSWSIGHSHFQQVKDFLASVIAPFEIGPDKVQVGLTQYSGDAQTEWDLNSLSTKEQVLAAVRRLRYKGGNTFTGLALTHVLGQNLQPAAGLRPEAAKVVILVTDGKSQDDVHTAARVLKDLGVNVFAVGVKNADEAELRLLASPPRDITVHSVLDFLQLGALAGLLSRLICQRLQGGSPRQGPAAAPALDTLPAPTSLVLSQVTSSSIRLSWTPAPRHPLKYLIVWRASRGGTPREVVVEGPAASTELHNLASRTEYLVSVFPIYEGGVGEGLRGLVTTAPLPPPRALTLAAVTPRTVHLTWQPSAGATHYLVRCSPASPKGEEEEREVQVGRPEVLLDGLEPGRDYEVSVQSLRGPEGSEARGIRARTPTLAPPRHLGFSDVSHDAARVFWEGAPRPVRLVRVTYVSSEGGHSGQTEAPGNATSATLGPLSSSTTYTVRVTCLYPGGGSSTLTGRVTTKKAPSPSQLSMTELPGDAVQLAWVAAAPSGVLVYQITWTPLGEGKAHEISVPGNLGTAVLPGLGRHTEYDVTILAYYRDGARSDPVSLRYTPSTVSRSPPSNLALASETPDSLQVSWTPPLGRVLHYWLTYAPASGLGPEKSVSVPGARSHVTLPDLQAATKYRVLVSAIYAAGRSEAVSATGQTACPALRPDGSLPGFDLMVAFSLVEKAYASIRGVAMEPSAFGGTPTFTLFKDAQLTRRVSDVYPAPLPPEHTIVFLVRLLPETPREAFALWQMTAEDFQPLLGVLLDAGKKSLTYFHRDPRAALQEATFDPQEVRKIFFGSFHKVHVAVGRSKVRLYVDCRKVAERPLGEMGSPPAAGFVTLGRLAKARGPRSSSAAFQLQMLQIVCSDTWADEDRCCELPASRDGETCPAFVSACSCSSETPGPPGPQGPPGLPGRNGTPGEQGFPGPRGPPGVKGEKGDHGLPGLQGHPGHQGIPGRVGLQGPKGMRGLEGTAGLPGPPGPRGFQGMAGARGTSGERGPPGTVGPTGLPGPKGERGEKGEPQSLATLYQLVSQASHVSKFDSFHENTRPPMPILEQKLEPGTEPLGSPGTRSKALVPGEWGRGGRHLEGRGEPGAVGQMGSPGQQGASTQGLWE

SEQ ID NO: 19 - protein, 626 aa, Homo sapiens
MAARPAATLAWSLLLLSSALLREGCRARFVAERDSEDDGEEPVVFPESPLQSPTVLVAVLARNAAHTLPHFLGCLERLDYPKSRMAIWAATDHNVDNTTEIFREWLKNVQRLYHYVEWRPMDEPESYPDEIGPKHWPTSRFAHVMKLRQAALRTAREKWSDYILFIDVDNFLTNPQTLNLLIAENKTIVAPMLESRGLYSNFWCGITPKGFYKRTPDYVQIREWKRTGCFPVPMVHSTFLIDLRKEASDKLTFYPPHQDYTWTFDDIIVFAFSSRQAGIQMYLCNREHYGYLPIPLKPHQTLQEDIENLIHVQIEAMIDRPPMEPSQYVSVVPKYPDKMGFDEIFMINLKRRKDRRDRMLRTLYEQEIEVKIVEAVDGKALNTSQLKALNIEMLPGYRDPYSSRPLTRGEIGCFLSHYSVWKEVIDRELEKTLVIEDDVRFEHQFKKKLMKLMDNIDQAQLDWELIYIGRKRMQVKEPEKAVPNVANLVEADYSYWTLGYVISLEGAQKLVGANPFGKMLPVDEFLPVMYNKHPVAEYKEYYESRDLKAFSAEPLLIYPTHYTGQPGYLSDTETSTIWDNETVATDWDRTHAWKSRKQSRIYSNAKNTEALPPPTSLDTVPSRDEL

SEQ ID NO: 20 - protein, 610 aa, Homo sapiens
MAQEIDLSALKELEREAILQVLYRDQAVQNTEEERTRKLKTHLQHLRWKGAKNTDWEHKEKCCARCQQVLGFLLHRGAVCRGCSHRVCAQCRVFLRGTHAWKCTVCFEDRNVKIKTGEWFYEERAKKFPTGGKHETVGGQLLQSYQKLSKISVVPPTPPPVSESQCSRSPGRLQEFGQFRGFNKSVENLFLSLATHVKKLSKSQNDMTSEKHLLATGPRQCVGQTERRSQSDTAVNVTTRKVSAPDILKPLNQEDPKCSTNPILKQQNLPSSPAPSTIFSGGFRHGSLISIDSTCTEMGNFDNANVTGEIEFAIHYCFKTHSLEICIKACKNLAYGEEKKKKCNPYVKTYLLPDRSSQGKRKTGVQRNTVDPTFQETLKYQVAPAQLVTRQLQVSVWHLGTLARRVFLGEVIIPLATWDFEDSTTQSFRWHPLRAKAEKYEDSVPQSNGELTVRAKLVLPSRPRKLQEAQEGTDQPSLHGQLCLVVLGAKNLPVRPDGTLNSFVKGCLTLPDQQKLRLKSPVLRKQACPQWKHSFVFSGVTPAQLRQSSLELTVWDQALFGMNDRLLGGTRLGSKGDTAVGGDACSLSKLQWQKVLSSPNLWTDMTLVLH

SEQ ID NO: 21 - protein, 775 aa, Homo sapiens
MDCRTKANPDRTFDLVLKVKCHASENEDPVVLWKFPEDFGDQEILQSVPKFCFPFDVERVSQNQVGQHFTFVLTDIESKQRFGFCRLTSGGTICLCILSYLPWFEVYYKLLNTLADYLAKELENDLNETLRSLYNHPVPKANTPVNLSVNQEIFIACEQVLKDQPALVPHSYFIAPDVTGLPTIPESRNLTEYFVAVDVNNMLQLYASMLHERRIVIISSKLSTLTACIHGSAALLYPMYWQHIYIPVLPPHLLDYCCAPMPYLIGIHSSLIERVKNKSLEDVVMLNVDTNTLESPFSDLNNLPSDVVSALKNKLKKQSTATGDGVARAFLRAQAALFGSYRDALRYKPGEPITFCEESFVKHRSSVMKQFLETAINLQLFKQFIDGRLAKLNAGRGFSDVFEEEITSGGFCGGNPRSYQQWVHTVKKGGALFNTAMTKATPAVRTAYKFAKNHAKLGLKEVKSKLKHKENEEDYGTCSSSVQYTPVYKLHNEKGGNSEKRKLAQARLKRPLKSLDGALYDDEDDDDIERASKLSSEDGEEASAYLYESDDSVETRVKTPYSGEMDLLGEILDTLSTHSSDQGKLAAAKSLDFFRSMDDIDYKPTNKSNAPSENNLAFLCGGSGDQAEWNLGQDDSALHGKHLPPSPRKRVSSSGLTDSLFILKEENSNKHLGADNVSDPTSGLDFQLTSPEVSQTDKGKTEKRETLSQISDDLLIPGLGRHSSTFVPWEKEGKEAKETSEDIGLLHEVVSLCHMTSDFQQSLNISDKNTNGNQT

SEQ ID NO: 22 - protein, 8797 aa, Homo sapiens
MATSRGASRCPRDIANVMQRLQDEQEIVQKRTFTKWINSHLAKRKPPMVVDDLFEDMKDGVKLLALLEVLSGQKLPCEQGRRMKRIHAVANIGTALKFLEGRKIKLVNINSTDIADGRPSIVLGLMWTIILYFQIEELTSNLPQLQSLSSSASSVDSIVSSETPSPPSKRKVTTKIQGNAKKALLKWVQYTAGKQTGIEVKDFGKSWRSGVAFHSVIHAIRPELVDLETVKGRSNRENLEDAFTIAETELGIPRLLDPEDVDVDKPDEKSIMTYVAQFLKHYPDIHNASTDGQEDDEILPGFPSFANSVQNFKREDRVIFKEMKVWIEQFERDLTRAQMVESNLQDKYQSFKHFRVQYEMKRKQIEHLIQPLHRDGKLSLDQALVKQSWDRVTSRLFDWHIQLDKSLPAPLGTIGAWLYRAEVALREEITVQQVHEETANTIQRKLEQHKDLLQNTDAHKRAFHEIYRTRSVNGIPVPPDQLEDMAERFHFVSSTSELHLMKMEFLELKYRLLSLLVLAESKLKSWIIKYGRRESVEQLLQNYVSFIENSKFFEQYEVTYQILKQTAEMYVKADGSVEEAENVMKFMNETTAQWRNLSVEVRSVRSMLEEVISNWDRYGNTVASLQAWLEDAEKMLNQSENAKKDFFRNLPHWIQQHTAMNDAGNFLIETCDEMVSRDLKQQLLLLNGRWRELFMEVKQYAQADEMDRMKKEYTDCVVTLSAFATEAHKKLSEPLEVSFMNVKLLIQDLEDIEQRVPVMDAQYKIITKTAHLITKESPQEEGKEMFATMSKLKEQLTKVKECYSPLLYESQQLLIPLEELEKQMTSFYDSLGKINEIITVLEREAQSSALFKQKHQELLACQENCKKTLTLIEKGSQSVQKFVTLSNVLKHFDQTRLQRQIADIHVAFQSMVKKTGDWKKHVETNSRLMKKFEESRAELEKVLRIAQEGLEEKGDPEELLRRHTEFFSQLDQRVLNAFLKACDELTDILPEQEQQGLQEAVRKLHKQWKDLQGEAPYHLLHLKIDVEKNRFLASVEECRTELDRETKLMPQEGSEKIIKEHRVFFSDKGPHHLCEKRLQLIEELCVKLPVRDPVRDTPGTCHVTLKELRAAIDSTYRKLMEDPDKWKDYTSRFSEFSSWISTNETQLKGIKGEAIDTANHGEVKRAVEEIRNGVTKRGETLSWLKSRLKVLTEVSSENEAQKQGDELAKLSSSFKALVTLLSEVEKMLSNFGDCVQYKEIVKNSLEELISGSKEVQEQAEKILDTENLFEAQQLLLHHQQKTKRISAKKRDVQQQIAQAQQGEGGLPDRGHEELRKLESTLDGLERSRERQERRIQVTLRKWERFETNKETVVRYLFQTGSSHERFLSFSSLESLSSELEQTKEFSKRTESIAVQAENLVKEASEIPLGPQNKQLLQQQAKSIKEQVKKLEDTLEEDIKTMEMVKTKWDHFGSNFETLSVWITEKEKELNALETSSSAMDMQISQIKVTIQEIESKLSSIVGLEEEAQSFAQFVTTGESARIKAKLTQIRRYGEELREHAQCLEGTILGHLSQQQKFEENLRKIQQSVSEFEDKLAVPIKICSSATETYKVLQEHMDLCQALESLSSAITAFSASARKVVNRDSCVQEAAALQQQYEDILRRAKERQTALENLLAHWQRLEKELSSFLTWLERGEAKASSPEMDISADRVKVEGELQLIQALQNEVVSQASFYSKLLQLKESLFSVASKDDVKMMKLHLEQLDERWRDLPQIINKRINFLQSVVAEHQQFDELLLSFSVWIKLFLSELQTTSEISIMDHQVALTRHKDHAAEVESKKGELQSLQGHLAKLGSLGRAEDLHLLQGKAEDCFQLFEEASQVVERRQLALSHLAEFLQSHASLSGILRQLRQTVEATNSMNKNESDLIEKDLNDALQNAKALESAAVSLDGILSKAQYHLKIGSSEQRTSCRATADQLCGEVERIQNLLGTKQSEADALAVLKKAFQDQKEELLKSIEDIEERTDKERLKEPTRQALQQRLRVFNQLEDELNSHEHELCWLKDKAKQIAQKDVAFAPEVDREINRLEVTWDDTKRLIHENQGQCCGLIDLMREYQNLKSAVSKVLENASSVIVTRTTIKDQEDLKWAFSKHETAKNKMNYKQKDLDNFTSKGKHLLSELKKIHSSDFSLVKTDMESTVDKWLDVSEKLEENMDRLRVSLSIWDDVLSTRDEIEGWSNNCVPQMAENISNLDNHLRAEELLKEFESEVKNKALRLEELHSKVNDLKELTKNLETPPDLQFIEADLMQKLEHAKEITEVAKGTLKDFTAQSTQVEKFINDITTWFTKVEESLMNCAQNETCEALKKVKDIQKELQSQQSNISSTQENLNSLCRKYHSAELESLGRAMTGLIKKHEAVSQLCSKTQASLQESLEKHFSESMQEFQEWFLGAKAAAKESSDRTGDSKVLEAKLHDLQNILDSVSDGQSKLDAVTQEGQTLYAHLSKQIVSSIQEQITKANEEFQAFLKQCLKDKQALQDCASELGSFEDQHRKLNLWIHEMEERFNTENLGESKQHIPEKKNEVHKVEMFLGELLAARESLDKLSQRGQLLSEEGHGAGQEGRLCSQLLTSHQNLLRMTKEKLRSCQVALQEHEALEEALQSMWFWVKAIQDRLACAESTLGSKDTLEKRLSQIQDILLMKGEGEVKLNMAIGKGEQALRSSNKEGQRVIQTQLETLKEVWADIMSSSVHAQSTLESVISQWNDYVERKNQLEQWMESVDQKIEHPLQPQPGLKEKFVLLDHLQSILSEAEDHTRALHRLIAKSRELYEKTEDESFKDTAQEELKTQFNDIMTVAKEKMRKVEEIVKDHLMYLDAVHEFTDWLHSAKEELHRWSDMSGDSSATQKKLSKIKELIDSREIGASRLSRVESLAPEVKQNTTASGCELMHTEMQALRADWKQWEDSVFQTQSCLENLVSQMALSEQEFSGQVAQLEQALEQFSALLKTWAQQLTLLEGKNTDEEIVECWHKGQEILDALQKAEPRTEDLKSQLNELCRFSRDLSTYSGKVSGLIKEYNCLCLQASKGCQNKEQILQQRFRKAFRDFQQWLVNAKITTAKCFDIPQNISEVSTSLQKIQEFLSESENGQHKLNMMLSKGELLSTLLTKEKAKGIQAKVTAAKEDWKNFHSNLHQKESALENLKIQMKDFEVSAEPIQDWLSKTEKMVHESSNRLYDLPAKRREQQKLQSVLEEIHCYEPQLNRLKEKAQQLWEGQAASKSFRHRVSQLSSQYLALSNLTKEKVSRLDRIVAEHNQFSLGIKELQDWMTDAIHMLDSYCHPTSDKSVLDSRTLKLEALLSVKQEKEIQMKMIVTRGESVLQNTSPEGIPTIQQQLQSVKDMWASLLSAGIRCKSQLEGALSKWTSYQDGVRQFSGWMDSMEANLNESERQHAELRDKTTMLGKAKLLNEEVLSYSSLLETIEVKGAGMTEHYVTQLELQDLQERYRAIQERAKEAVTKSEKLVRLHQEYQRDLKAFEVWLGQEQEKLDQYSVLEGDAHTHETTLRDLQELQVHCAEGQALLNSVLHTREDVIPSGIPQAEDRALESLRQDWQAYQHRLSETRTQFNNVVNKLRLMEQKFQQVDEWLKTAEEKVSPRTRRQSNRATKEIQLHQMKKWHEEVTAYRDEVEEVGARAQEILDESHVNSRMGCQATQLTSRYQALLLQVLEQIKFLEEEIQSLEESESSLSSYSDWYGSTHKNFKNVATKIDKVDTVMMGKKLKTLEVLLKDMEKGHSLLKSAREKGERAVKYLEEGEAERLRKEIHDHMEQLKELTSTVRKEHMTLEKGLHLAKEFSDKCKALTQWIAEYQEILHVPEEPKMELYEKKAQLSKYKSLQQTVLSHEPSVKSVREKGEALLELVQDVTLKDKIDQLQSDYQDLCSIGKEHVFSLEAKVKDHEDYNSELQEVEKWLLQMSGRLVAPDLLETSSLETITQQLAHHKAMMEEIAGFEDRLNNLQMKGDTLIGQCADHLQAKLKQNVHAHLQGTKDSYSAICSTAQRMYQSLEHELQKHVSRQDTLQQCQAWLSAVQPDLEPSPQPPLSRAEAIKQVKHFRALQEQARTYLDLLCSMCDLSNASVKTTAKDIQQTEQTIEQKLVQAQNLTQGWEEIKHLKSELWIYLQDADQQLQNMKRRHSELELNIAQNMVSQVKDFVKKLQSKQASVNTIIEKVNKLTKKEESPEHKEINHLNDQWLDLCRQSNNLCLQREEDLQRTRDYHDCMNVVEVFLEKFTTEWDNLARSDAESTAVHLEALKKLALALQERKYAIEDLKDQKQKMIEHLNLDDKELVKEQTSHLEQRWFQLEDLIKRKIQVSVTNLEELNVVQSRFQELMEWAEEQQPNIAEALKQSPPPDMAQNLLMDHLAICSELEAKQMLLKSLIKDADRVMADLGLNERQVIQKALSDAQSHVNCLSDLVGQRRKYLNKALSEKTQFLMAVFQATSQIQQHERKIMFREHICLLPDDVSKQVKTCKSAQASLKTYQNEVTGLWAQGRELMKEVTEQEKSEVLGKLQELQSVYDSVLQKCSHRLQELEKNLVSRKHFKEDFDKACHWLKQADIVTFPEINLMNESSELHTQLAKYQNILEQSPEYENLLLTLQRTGQTILPSLNEVDHSYLSEKLNALPRQFNVIVALAKDKFYKVQEAILARKEYASLIELTTQSLSELEAQFLRMSKVPTDLAVEEALSLQDGCRAILDEVAGLGEAVDELNQKKEGFRSTGQPWQPDKMLHLVTLYHRLKRQTEQRVSLLEDTTSAYQEHEKMCQQLERQLKSVKEEQSKVNEETLPAEEKLKMYHSLAGSLQDSGIVLKRVTIHLEDLAPHLDPLAYEKARHQIQSWQGELKLLTSAIGETVTECESRMVQSIDFQTEMSRSLDWLRRVKAELSGPVYLDLNLQDIQEEIRKIQIHQEEVQSSLRIMNALSHKEKEKFTKAKELISADLEHSLAELSELDGDIQEALRTRQATLTEIYSQCQRYYQVFQAANDWLEDAQELLQLAGNGLDVESAEENLKSHMEFFSTEDQFHSNLEELHSLVATLDPLIKPTGKEDLEQKVASLELRSQRMSRDSGAQVDLLQRCTAQWHDYQKAREEVIELMNDTEKKLSEFSLLKTSSSHEAEEKLSEHKALVSVVNSFHEKIVALEEKASQLEKTGNDASKATLSRSMTTVWQRWTRLRAVAQDQEKILEDAVDEWTGFNNKVKKATEMIDQLQDKLPGSSAEKASKAELLTLLEYHDTFVLELEQQQSALGMLRQQTLSMLQDGAAPTPGEEPPLMQEITAMQDRCLNMQEKVKTNGKLVKQELKDREMVETQINSVKCWVQETKEYLGNPTIEIDAQLEELQILLTEATNHRQNIEKMAEEQKEKYLGLYTILPSELSLQLAEVALDLKIRDQIQDKIKEVEQSKATSQELSRQIQKLAKDLTTILTKLKAKTDNVVQAKTDQKVLGEELDGCNSKLMELDAAVQKFLEQNGQLGKPLAKKIGKLTELHQQTIRQAENRLSKLNQAASHLEEYNEMLELILKWIEKAKVLAHGTIAWNSASQLREQYILHQTLLEESKEIDSELEAMTEKLQYLTSVYCTEKMSQQVAELGRETEELRQMIKIRLQNLQDAAKDMKKFEAELKKLQAALEQAQATLTSPEVGRLSLKEQLSHRQHLLSEMESLKPKVQAVQLCQSALRIPEDVVASLPLCHAALRLQEEASRLQHTAIQQCNIMQEAVVQYEQYEQEMKHLQQLIEGAHREIEDKPVATSNIQELQAQISRHEELAQKIKGYQEQIASLNSKCKMLTMKAKHATMLLTVTEVEGLAEGTEDLDGELLPTPSAHPSVVMMTAGRCHTLLSPVTEESGEEGTNSEISSPPACRSPSPVANTDASVNQDIAYYQALSAERLQTDAAKIHPSTSASQEFYEPGLEPSATAKLGDLQRSWETLKNVISEKQRTLYEALERQQKYQDSLQSISTKMEAIELKLSESPEPGRSPESQMAEHQALMDEILMLQDEINELQSSLAEELVSESCEADPAEQLALQSTLTVLAERMSTIRMKASGKRQLLEEKLNDQLEEQRQEQALQRYRCEADELDSWLLSTKATLDTALSPPKEPMDMEAQLMDCQNMLVEIEQKVVALSELSVHNENLLLEGKAHTKDEAEQLAGKLRRLKGSLLELQRALHDKQLNMQGTAQEKEESDVDLTATQSPGVQEWLAQARTTWTQQRQSSLQQQKELEQELAEQKSLLRSVASRGEEILIQHSAAETSGDAGEKPDVLSQELGMEGEKSSAEDQMRMKWESLHQEFSTKQKLLQNVLEQEQEQVLYSRPNRLLSGVPLYKGDVPTQDKSAVTSLLDGLNQAFEEVSSQSGGAKRQSIHLEQKLYDGVSATSTWLDDVEERLFVATALLPEETETCLFNQEILAKDIKEMSEEMDKNKNLFSQAFPENGDNRDVIEDTLGCLLGRLSLLDSVVNQRCHQMKERLQQILNFQNDLKVLFTSLADNKYIILQKLANVFEQPVAEQIEAIQQAEDGLKEFDAGIIELKRRGDKLQVEQPSMQELSKLQDMYDELMMIIGSRRSGLNQNLTLKSQYERALQDLADLLETGQEKMAGDQKIIVSSKEEIQQLLDKHKEYFQGLESHMILTETLFRKIISFAVQKETQFHTELMAQASAVLKRAHKRGVELEYILETWSHLDEDQQELSRQLEVVESSIPSVGLVEENEDRLIDRITLYQHLKSSLNEYQPKLYQVLDDGKRLLISISCSDLESQLNQLGECWLSNTNKMSKELHRLETILKHWTRYQSESADLIHWLQSAKDRLEFWTQQSVTVPQELEMVRDHLNAFLEFSKEVDAQSSLKSSVLSTGNQLLRLKKVDTATLRSELSRIDSQWTDLLTNIPAVQEKLHQLQMDKLPSRHAISEVMSWISLMENVIQKDEDNIKNSIGYKAIHEYLQKYKGFKIDINCKQLTVDFVNQSVLQISSQDVESKRSDKTDFAEQLGAMNKSWQILQGLVTEKIQLLEGLLESWSEYENNVQCLKTWFETQEKRLKQQHRIGDQASVQNALKDCQDLEDLIKAKEKEVEKIEQNGLALIQNKKEDVSSIVMSTLRELGQTWANLDHMVGQLKILLKSVLDQWSSHKVAFDKINSYLMEARYSLSRFRLLTGSLEAVQVQVDNLQNLQDDLEKQERSLQKFGSITNQLLKECHPPVTETLTNTLKEVNMRWNNLLEEIAEQLQSSKALLQLWQRYKDYSKQCASTVQQQEDRTNELLKAATNKDIADDEVATWIQDCNDLLKGLGTVKDSLFFLHELGEQLKQQVDASAASAIQSDQLSLSQHLCALEQALCKQQTSLQAGVLDYETFAKSLEALEAWIVEAEEILQGQDPSHSSDLSTIQERMEELKGQMLKFSSMAPDLDRLNELGYRLPLNDKEIKRMQNLNRHWSLISSQTTERFSKLQSFLLQHQTFLEKCETWMEFLVQTEQKLAVEISGNYQHLLEQQRAHELFQAEMFSRQQILHSIIIDGQRLLEQGQVDDRDEFNLKLTLLSNQWQGVIRRAQQRRGIIDSQIRQWQRYREMAEKLRKWLVEVSYLPMSGLGSVPIPLQQARTLFDEVQFKEKVFLRQQGSYILTVEAGKQLLLSADSGAEAALQAELAEIQEKWKSASMRLEEQKKKLAFLLKDWEKCEKGIADSLEKLRTFKKKLSQSLPDHHEELHAEQMRCKELENAVGSWTDDLTQLSLLKDTLSAYISADDISILNERVELLQRQWEELCHQLSLRRQQIGERLNEWAVFSEKNKELCEWLTQMESKVSQNGDILIEEMIEKLKKDYQEEIAIAQENKIQLQQMGERLAKASHESKASEIEYKLGKVNDRWQHLLDLIAARVKKLKETLVAVQQLDKNMSSLRTWLAHIESELAKPIVYDSCNSEEIQRKLNEQQELQRDIEKHSTGVASVLNLCEVLLHDCDACATDAECDSIQQATRNLDRRWRNICAMSMERRLKIEETWRLWQKFLDDYSRFEDWLKSSERTAAFPSSSGVIYTVAKEELKKFEAFQRQVHECLTQLELINKQYRRLARENRTDSACSLKQMVHEGNQRWDNLQKRVTSILRRLKHFIGQREEFETARDSILVWLTEMDLQLTNIEHFSECDVQAKIKQLKAFQQEISLNHNKIEQIIAQGEQLIEKSEPLDAAIIEEELDELRRYCQEVFGRVERYHKKLIRLPLPDDEHDLSDRELELEDSAALSDLHWHDRSADSLLSPQPSSNLSLSLAQPLRSERSGRDTPASVDSIPLEWDHDYDLSRDLESAMSRALPSEDEEGQDDKDFYLRGAVGLSGDHSALESQIRQLGKALDDSRFQIQQTENIIRSKTPTGPELDTSYKGYMKLLGECSSSIDSVKRLEHKLKEEEESLPGFVNLHSTETQTAGVIDRWELLQAQALSKELRMKQNLQKWQQFNSDLNSIWAWLGDTEEELEQLQRLELSTDIQTIELQIKKLKELQKAVDHRKAIILSINLCSPEFTQADSKESRDLQDRLSQMNGRWDRVCSLLEEWRGLLQDALMQCQGFHEMSHGLLLMLENIDRRKNEIVPIDSNLDAEILQDHHKQLMQIKHELLESQLRVASLQDMSCQLLVNAEGTDCLEAKEKVHVIGNRLKLLLKEVSRHIKELEKLLDVSSSQQDLSSWSSADELDTSGSVSPTSGRSTPNRQKTPRGKCSLSQPGPSVSSPHSRSTKGGSDSSLSEPGPGRSGRGFLFRVLRAALPLQLLLLLLIGLACLVPMSEEDYSCALSNNFARSFHPMLRYTNGPPPL

SEQ ID NO: 23 - protein, 431 aa, Homo sapiens
MRRLRRLAHLVLFCPFSKRLQGRLPGLRVRCIFLAWLGVFAGSWLVYVHYSSYSERCRGHVCQVVICDQYRKGIISGSVCQDLCELHMVEWRTCLSVAPGQQVYSGLWRDKDVTIKCGIEETLDSKARSDAAPRRELVLFDKPTRGTSIKEFREMTLSFLKANLGDLPSLPALVGQVLLMADFNKDNRVSLAEAKSVWALLQRNEFLLLLSLQEKEHASRLLGYCGDLYLTEGVPHGAWHAAALPPLLRPLLPPALQGALQQWLGPAWPWRAKIAIGLLEFVEELFHGSYGTFYMCETTLANVGYTATYDFKMADLQQVAPEATVRRFLQGRRCEHSTDCTYGRDCRAPCDRLMRQCKGDLIQPNLAKVCALLRGYLLPGAPADLREELGTQLRTCTTLSGLASQVEAHHSLVLSHLKTLLWKKISNTKYS

SEQ ID NO: 24 - protein, 441 aa, Homo sapiens
MGGPRAWALLCLGLLLPGGGAAWSIGAAPFSGRRNWCSYVVTRTISCHVQNGTYLQRVLQNCPWPMSCPGSSYRTVVRPTYKVMYKIVTAREWRCCPGHSGVSCEEASSASLEPMWSGSTMRRMALRPTAFSGCLNCSKVSELTERLKVLEAKMTMLTVIEQPVPPTPATPEDPAPLWGPPPAQGSPGDGGLQDQVGAWGLPGPTGPKGDAGSRGPMGMRGPPGPQGPPGSPGRAGAVGTPGERGPPGPPGPPGPPGPPAPVGPPHARISQHGDPLLSNTFTETNNHWPQGPTGPPGPPGPMGPPGPPGPTGVPGSPGHIGPPGPTGPKGISGHPGEKGERGLRGEPGPQGSAGQRGEPGPKGDPGEKSHWGEGLHQLREALKILAERVLILETMIGLYEPELGSGAGPAGTGTPSLLRGKRGGHATNYRIVAPRSRDERG

SEQ ID NO: 25 - protein, 409 aa, Homo sapiens
MEESWEAAPGGQAGAELPMEPVGSLVPTLEQPQVPAKVRQPEGPESSPSPAGAVEKAAGAGLEPSSKKKPPSPRPGSPRVPPLSLGYGVCPEPPSPGPALVKLPRNGEAPGAEPAPSAWAPMELQVDVRVKPVGAAGGSSTPSPRPSTRFLKVPVPESPAFSRHADPAHQLLLRAPSQGGTWGRRSPLAAARTESGCDAEGRASPAEGSAGSPGSPTCCRCKELGLEKEDAALLPRAGLDGDEKLPRAVTLTGLPMYVKSLYWALAFMAVLLAVSGVVIVVLASRAGARCQQCPPGWVLSEEHCYYFSAEAQAWEASQAFCSAYHATLPLLSHTQDFLGRYPVSRHSWVGAWRGPQGWHWIDEAPLPPQLLPEDGEDNLDINCGALEEGTLVAANCSTPRPWVCAKGTQ

SEQ ID NO: 26 - protein, 904 aa, Homo sapiens
MEWGSESAAVRRHRVGVERREGAAAAPPPEREARAQEPLVDGCSGGGRTRKRSPGGSGGASRGAGTGLSEVRAALGLALYLIALRTLVQLSLQQLVLRGAAGHRGEFDALQARDYLEHITSIGPRTTGSPENEILTVHYLLEQIKLIEVQSNSLHKISVDVQRPTGSFSIDFLGGFTSYYDNITNVVVKLEPRDGAQHAVLANCHFDSVANSPGASDDAVSCSVMLEVLRVLSTSSEALHHAVIFLFNGAEENVLQASHGFITQHPWASLIRAFINLEAAGVGGKELVFQTGPENPWLVQAYVSAAKHPFASVVAQEVFQSGIIPSDTDFRIYRDFGNIPGIDLAFIENGYIYHTKYDTADRILTDSIQRAGDNILAVLKHLATSDMLAAASKYRHGNMVFFDVLGLFVIAYPSRIGSIINYMVVMGVVLYLGKKFLQPKHKTGNYKKDFLCGLGITLISWFTSLVTVLIIAVFISLIGQSLSWYNHFYVSVCLYGTATVAKIILIHTLAKRFYYMNASAQYLGEVFFDISLFVHCCFLVTLTYQGLCSAFISAVWVAFPLLTKLCVHKDFKQHGAQGKFIAFYLLGMFIPYLYALYLIWAVFEMFTPILGRSGSEIPPDVVLASILAGCTMILSSYFINFIYLAKSTKKTMLTLTLVCAITFLLVCSGTFFPYSSNPANPKPKRVFLQHMTRTFHDLEGNAVKRDSGIWINGFDYTGISHITPHIPEINDSIRAHCEENAPLCGFPWYLPVHFLIRKNWYLPAPEVSPRNPPHFRLISKEQTPWDSIKLTFEATGPSHMSFYVRAHKGSTLSQWSLGNGTPVTSKGGDYFVFYSHGLQASAWQFWIEVQVSEEHPEGMVTVAIAAHYLSGEDKRSPQLDALKEKFPDWTFPSAWVCTYDLFVF

SEQ ID NO: 27 - protein, 202 aa, Homo sapiens
MERGAGAKLLPLLLLLRATGFTCAQTDGRNGYTAVIEVTSGGPWGDWAWPEMCPDGFFASGFSLKVEPPQGIPGDDTALNGIRLHCARGNVLGNTHVVESQSGSWGEWSEPLWCRGGAYLVAFSLRVEAPTTLGDNTAANNVRFRCSDGEELQGPGLSWGDFGDWSDHCPKGACGLQTKIQGPRGLGDDTALNDARLFCCRS

SEQ ID NO: 28 - protein, 147 aa, Homo sapiens
MGFIFSKSMNESMKNQKEFMLMNARLQLERQLIMQSEMRERQMAMQIAWSREFLKYFGTFFGLAAISLTAGAIKKKKPAFLVPIVPLSFILTYQYDLGYGTLLERMKGEAEDILETEKSKLQLPRGMITFESIEKARKEQSRFFIDK

SEQ ID NO: 29 - protein, 758 aa, Homo sapiens
MRKQGVSSKRLQSSGRSQSKGRRGASLAREPEVEEEMEKSALGGGKLPRGSWRSSPGRIQSLKERKGLELEVVAKTFLLGPFQFVRNSLAQLREKVQELQARRFSSRTTLGIAVFVAILHWLHLVTLFENDRHFSHLSSLEREMTFRTEMGLYYSYFKTIIEAPSFLEGLWMIMNDRLTEYPLIINAIKRFHLYPEVIIASWYCTFMGIMNLFGLETKTCWNVTRIEPLNEVQSCEGLGDPACFYVGVIFILNGLMMGLFFMYGAYLSGTQLGGLITVLCFFFNHGEATRVMWTPPLRESFSYPFLVLQMCILTLILRTSSNDRRPFIALCLSNVAFMLPWQFAQFILFTQIASLFPMYVVGYIEPSKFQKIIYMNMISVTLSFILMFGNSMYLSSYYSSSLLMTWAIILKRNEIQKLGVSKLNFWLIQGSAWWCGTIILKFLTSKILGVSDHIRLSDLIAARILRYTDFDTLIYTCAPEFDFMEKATPLRYTKTLLLPVVMVITCFIFKKTVRDISYVLATNIYLRKQLLEHSELAFHTLQLLVFTALAILIMRLKMFLTPHMCVMASLICSRQLFGWLFRRVRFEKVIFGILTVMSIQGYANLRNQWSIIGEFNNLPQEELLQWIKYSTTSDAVFAGAMPTMASIKLSTLHPIVNHPHYEDADLRARTKIVYSTYSRKSAKEVRDKLLELHVNYYVLEEAWCVVRTKPGCSMLEIWDVEDPSNAANPPLCSVLLEDARPYFTTVFQNSVYRVLKVN

SEQ ID NO: 30 - protein, 316 aa, Homo sapiens
MASADELTFHEFEEATNLLADTPDAATTSRSDQLTPQGHVAVAVGSGGSYGAEDEVEEESDKAALLQEQQQQQQPGFWTFSYYQSFFDVDTSQVLDRIKGSLLPRPGHNFVRHHLRNRPDLYGPFWICATLAFVLAVTGNLTLVLAQRRDPSIHYSPQFHKVTVAGISIYCYAWLVPLALWGFLRWRKGVQERMGPYTFLETVCIYGYSLFVFIPMVVLWLIPVPWLQWLFGALALGLSAAGLVFTLWPVVREDTRLVATVLLSVVVLLHALLAMGCKLYFFQSLPPENVAPPPQITSLPSNIALSPTLPQSLAPS

SEQ ID NO: 31 - protein, 241 aa, Homo sapiens
MKFILLWALLNLTVALAFNPDYTVSSTPPYLVYLKSDYLPCAGVLIHPLWVITAAHCNLPKLRVILGVTIPADSNEKHLQVIGYEKMIHHPHFSVTSIDHDIMLIKLKTEAELNDYVKLANLPYQTISENTMCSVSTWSYNVCDIYKEPDSLQTVNISVISKPQCRDAYKTYNITENMLCVGIVPGRRQPCKEVSAAPAICNGMLQGILSFADGCVLRADVGIYAKIFYYIPWIENVIQNN

SEQ ID NO: 32 - protein, 1172 aa, Homo sapiens
MSPDVPLLNDYKQDFFLKRFPQTVLGGPRFKLGYCAPPYIYVNQIILFLMPWVWGGVGTLLYQLGILKDYYTAALSGGLMLFTAFVIQFTSLYAKNKSTTVERILTTDILAEEDEHEFTSCTGAETVKFLIPGKKYVANTVFHSILAGLACGLGTWYLLPNRITLLYGSTGGTALLFFFGWMTLCIAEYSLIVNTATETATFQTQDTYEIIPLMRPLYIFFFVSVDLAHRFVVNMPALEHMNQILHILFVFLPFLWALGTLPPPDALLLWAMEQVLEFGLGGSSMSTHLRLLVMFIMSAGTAIASYFIPSTVGVVLFMTGFGFLLSLNLSDMGHKIGTKSKDLPSGPEKHFSWKECLFYIIILVLALLETSLLHHFAGFSQISKSNSQAIVGYGLMILLIILWILREIQSVYIIGIFRNPFYPKDVQTVTVFFEKQTRLMKIGIVRRILLTLVSPFAMIAFLSLDSSLQGLHSVSVCIGFTRAFRMVWQNTENALLETVIVSTVHLISSTDIWWNRSLDTGLRLLLVGIIRDRLIQFISKLQFAVTVLLTSWTEKKQRRKTTATLCILNIVFSPFVLVIIVFSTLLSSPLLPLFTLPVFLVGFPRPIQSWPGAAGTTACVCADTVYYYQMVPRLTAVLQTAMAAGSLGLLLPGSHYLGRFQDRLMWIMILECGYTYCSINIKGLELQETSCHTAEARRVDEVFEDAFEQEYTRVCSLNEHFGNVLTPCTVLPVKLYSDARNVLSGIIDSHENLKEFKGDLIKVLVWILVQYCSKRPGMKENVHNTENKGKAPLMLPALNTLPPPKSPEDIDSLNSETFNDWSDDNIFDDEPTIKKVIEEKHQLKDLPGTNLFIPGSVESQRVGDHSTGTVPENDLYKAVLLGYPAVDKGKQEDMPYIPLMEFSCSHSHLVCLPAEWRTSCMPSSKMKEMSSLFPEDWYQFVLRQLECYHSEEKASNVLEEIAKDKVLKDFYVHTVMTCYFSLFGIDNMAPSPGHILRVYGGVLPWSVALDWLTEKPELFQLALKAFRYTLKLMIDKASLGPIEDFRELIKYLEEYERDWYIGLVSDEKWKEAILQEKPYLFSLGYDSNMGIYTGRVLSLQELLIQVGKLNPEAVRGQWANLSWELLYATNDDEERYSIQAHPLLLRNLTVQAAEPPLGYPIYSSKPLHIHLY

SEQ ID NO: 33 - protein, 346 aa, Homo sapiens
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 34 - protein, 752 aa, Homo sapiens
MAEPQAESEPLLGGARGGGGDWPAGLTTYRSIQVGPGAAARWDLCIDQAVVFIEDAIQYRSINHRVDASSMWLYRRYYSNVCQRTLSFTIFLILFLAFIETPSSLTSTADVRYRAAPWEPPCGLTESVEVLCLLVFAADLSVKGYLFGWAHFQKNLWLLGYLVVLVVSLVDWTVSLSLVCHEPLRIRRLLRPFFLLQNSSMMKKTLKCIRWSLPEMASVGLLLAIHLCLFTMFGMLLFAGGKQDDGQDRERLTYFQNLPESLTSLLVLLTTANNPDVMIPAYSKNRAYAIFFIVFTVIGSLFLMNLLTAIIYSQFRGYLMKSLQTSLFRRRLGTRAAFEVLSSMVGEGGAFPQAVGVKPQNLLQVLQKVQLDSSHKQAMMEKVRSYGSVLLSAEEFQKLFNELDRSVVKEHPPRPEYQSPFLQSAQFLFGHYYFDYLGNLIALANLVSICVFLVLDADVLPAERDDFILGILNCVFIVYYLLEMLLKVFALGLRGYLSYPSNVFDGLLTVVLLVLEISTLAVYRLPHPGWRPEMVGLLSLWDMTRMLNMLIVFRFLRIIPSMKLMAVVASTVLGLVQNMRAFGGILVVVYYVFAIIGINLFRGVIVALPGNSSLAPANGSAPCGSFEQLEYWANNFDDFAAALVTLWNLMVVNNWQVFLDAYRRYSGPWSKIYFVLWWLVSSVIWVNLFLALILENFLHKWDPRSHLQPLAGTPEATYQMTVELLFRDILEEPGEDELTERLSQHPHLWLCR

SEQ ID NO: 35 - protein, 272 aa, Homo sapiens
MQWNVPRTVSRLARRTCLEPHNAGLFGHCQNVKGPLLLYNAESKVVLVQGPQKQWLHLSAAQCVAKERRPLDAHPPQPGVLRHKQGKQHVSFRRVFSSSATAQGTPEKKEEPDPLQDKSISLYQRFKKTFRQYGKVLIPVHLITSGVWFGTFYYAALKGVNVVPFLELIGLPDSVVSILKNSQSGNALTAYALFKIATPARYTVTLGGTSVTVKYLRSHGYMSTPPPVKEYLQDRMEETKELITEKMEETKDRLTEKLQETKEKVSFKKKVE

SEQ ID NO: 36 - protein, 402 aa, Homo sapiens
MMVALRGASALLVLFLAAFLPPPQCTQDPAMVHYIYQRFRVLEQGLEKCTQATRAYIQEFQEFSKNISVMLGRCQTYTSEYKSAVGNLALRVERAQREIDYIQYLREADECIESEDKTLAEMLLQEAEEEKKIRTLLNASCDNMLMGIKSLKIVKKMMDTHGSWMKDAVYNSPKVYLLIGSRNNTVWEFANIRAFMEDNTKPAPRKQILTLSWQGTGQVIYKGFLFFHNQATSNEIIKYNLQKRTVEDRMLLPGGVGRALVYQHSPSTYIDLAVDEHGLWAIHSGPGTHSHLVLTKIEPGTLGVEHSWDTPCRSQDAEASFLLCGVLYVVYSTGGQGPHRITCIYDPLGTISEEDLPNLFFPKRPRSHSMIHYNPRDKQLYAWNEGNQIIYKLQTKRKLPLK

SEQ ID NO: 37 - protein, 246 aa, Homo sapiens
MGPQHLRLVQLFCLLGAISTLPRAGALLCYEATASRFRAVAFHNWKWLLMRNMVCKLQEGCEETLVFIETGTARGVVGFKGCSSSSSYPAQISYLVSPPGVSIASYSRVCRSYLCNNLTNLEPFVKLKASTPKSITSASCSCPTCVGEHMKDCLPNFVTTNSCPLAASTCYSSTLKFQAGFLNTTFLLMGCAREHNQLLADFHHIGSIKVTEVLNILEKSQIVGAASSRQDPAWGVVLGLLFAFRD

SEQ ID NO: 38 - protein, 2845 aa, Homo sapiens
MALGKVLAMALVLALAVLGSLSPGARAGDCKGQRQVLREAPGFVTDGAGNYSVNGNCEWLIEAPSPQHRILLDFLFLDTECTYDYLFVYDGDSPRGPLLASLSGSTRPPPIEASSGKMLLHLFSDANYNLLGFNASFRFSLCPGGCQSHGQCQPPGVCACEPGWGGPDCGLQECSAYCGSHGTCASPLGPCRCEPGFLGRACDLHLWENQGAGWWHNVSARDPAFSARIGAAGAFLSPPGLLAVFGGQDLNNALGDLVLYNFSANTWESWDLSPAPAARHSHVAVAWAGSLVLMGGELADGSLTNDVWAFSPLGRGHWELLAPPASSSSGPPGLAGHAAALVDDVWLYVSGGRTPHDLFSSGLFRFRLDSTSGGYWEQVIPAGGRPPAATGHSMVFHAPSRALLVHGGHRPSTARFSVRVNSTELFHVDRHVWTTLKGRDGLQGPRERAFHTASVLGNYMVVYGGNVHTHYQEEKCYEDGIFFYHLGCHQWVSGAELAPPGTPEGRAAPPSGRYSHVAAVLGGSVLLVAGGYSGRPRGDLMAYKVPPFVFQAPAPDYHLDYCSMYTDHSVCSRDPECSWCQGACQAAPPPGTPLGACPAASCLGLGRLLGDCQACLAFSSPTAPPRGPGTLGWCVHNESCLPRPEQARCRGEQISGTVGWWGPAPVFVTSLEACVTQSFLPGLHLLTFQQPPNTSQPDKVSIVRSTTITLTPSAETDVSLVYRGFIYPMLPGGPGGPGAEDVAVWTRAQRLHVLARMARGPDTENMEEVGRWVAHQEKETRRLQRPGSARLFPLPGRDHKYAVEIQGQLNGSAGPGHSELTLLWDRTGVPGGSEISFFFLEPYRSSSCTSYSSCLGCLADQGCGWCLTSATCHLRQGGAHCGDDGAGGSLLVLVPTLCPLCEEHRDCHACTQDPFCEWHQSTSRKGDAACSRRGRGRGALKSPEECPPLCSQRLTCEDCLANSSQCAWCQSTHTCFLFAAYLARYPHGGCRGWDDSVHSEPRCRSCDGFLTCHECLQSHECGWCGNEDNPTLGRCLQGDFSGPLGGGNCSLWVGEGLGLPVALPARWAYARCPDVDECRLGLARCHPRATCLNTPLSYECHCQRGYQGDGISHCNRTCLEDCGHGVCSGPPDFTCVCDLGWTSDLPPPTPAPGPPAPRCSRDCGCSFHSHCRKRGPGFCDECQDWTWGEHCERCRPGSFGNATGSRGCRPCQCNGHGDPRRGHCDNLSGLCFCQDHTEGAHCQLCSPGYYGDPRAGGSCFRECGGRALLTNVSSVALGSRRVGGLLPPGGGAARAGPGLSYCVWVVSATEELQPCAPGTLCPPLTLTFSPDSSTPCTLSYVLAFDGFPRFLDTGVVQSDRSLIAAFCGQRRDRPLTVQALSGLLVLHWEANGSSSWGFNASVGSARCGSGGPGSCPVPQECVPQDGAAGAGLCRCPQGWAGPHCRMALCPENCNAHTGAGTCNQSLGVCICAEGFGGPDCATKLDGGQLVWETLMDSRLSADTASRFLHRLGHTMVDGPDATLWMFGGLGLPQGLLGNLYRYSVSERRWTQMLAGAEDGGPGPSPRSFHAAAYVPAGRGAMYLLGGLTAGGVTRDFWVLNLTTLQWRQEKAPQTVELPAVAGHTLTARRGLSLLLVGGYSPENGFNQQLLEYQLATGTWVSGAQSGTPPTGLYGHSAVYHEATDSLYVFGGFRFHVELAAPSPELYSLHCPDRTWSLLAPSQGAKRDRMRNVRGSSRGLGQVPGEQPGSWGFREVRKKMALWAALAGTGGFLEEISPHLKEPRPRLFHASALLGDTMVVLGGRSDPDEFSSDVLLYQVNCNAWLLPDLTRSASVGPPMEESVAHAVAAVGSRLYISGGFGGVALGRLLALTLPPDPCRLLSSPEACNQSGACTWCHGACLSGDQAHRLGCGGSPCSPMPRSPEECRRLRTCSECLARHPRTLQPGDGEASTPRCKWCTNCPEGACIGRNGSCTSENDCRINQREVFWAGNCSEAACGAADCEQCTREGKCMWTRQFKRTGETRRILSVQPTYDWTCFSHSLLNVSPMPVESSPPLPCPTPCHLLPNCTSCLDSKGADGGWQHCVWSSSLQQCLSPSYLPLRCMAGGCGRLLRGPESCSLGCAQATQCALCLRRPHCGWCAWGGQDGGGRCMEGGLSGPRDGLTCGRPGASWAFLSCPPEDECANGHHDCNETQNCHDQPHGYECSCKTGYTMDNMTGLCRPVCAQGCVNGSCVEPDHCRCHFGFVGRNCSTECRCNRHSECAGVGARDHCLLCRNHTKGSHCEQCLPLFVGSAVGGGTCRPCHAFCRGNSHICISRKELQMSKGEPKKYSLDPEEIENWVTEGPSEDEAVCVNCQNNSYGEKCESCLQGYFLLDGKCTKCQCNGHADTCNEQDGTGCPCQNNTETGTCQGSSPSDRRDCYKYQCAKCRESFHGSPLGGQQCYRLISVEQECCLDPTSQTNCFHEPKRRALGPGRTVLFGVQPKFTNVDIRLTLDVTFGAVDLYVSTSYDTFVVRVAPDTGVHTVHIQPPPAPPPPPPPADGGPRGAGDPGGAGASSGPGAPAEPRVREVWPRGLITYVTVTEPSAVLVVRGVRDRLVITYPHEHHALKSSRFYLLLLGVGDPSGPGANGSADSQGLLFFRQDQAHIDLFVFFSVFFSCFFLFLSLCVLLWKAKQALDQRQEQRRHLQEMTKMASRPFAKVTVCFPPDPTAPASAWKPAGLPPPAFRRSEPFLAPLLLTGAGGPWGPMGGGCCPPAIPATTAGLRAGPITLEPTEDGMAGVATLLLQLPGGPHAPNGACLGSALVTLRHRLHEYCGGGGGAGGSGHGTGAGRKGLLSQDNLTSMSL

SEQ ID NO: 39 - protein, 586 aa, Homo sapiens
MAEGVPASPSSGEGSRGPHSGVIQWLVDNFCICEECSVPRCLMYEIYVETCGQNTENQVNPATFGKLVRLVFPDLGTRRLGTRGSARYHYDGICIKKSSFFYAQYCYLIGEKRYHSGDAIAFEKSTNYNSIIQQEATCEDHSPMKTDPVGSPLSEFRRCPFLEQEQAKKYSCNMMAFLADEYCNYCRDILRNVEDLLTSFWKSLQQDTVMLMSLPDVCQLFKCYDVQLYKGIEDVLLHDFLEDVSIQYLKSVQLFSKKFKLWLLNALEGVPALLQISKLKEVTLFVKRLRRKTYLSNMAKTMRMVLKSKRRVSVLKSDLQAIINQGTLATSKKALASDRSGADELENNPEMKCLRNLISLLGTSTDLRVFLSCLSSHLQAFVFQTSRSKEEFTKLAASFQLRWNLLLTAVSKAMTLCHRDSFGSWHLFHLLLLEYMIHILQSCLEEEEEEEDMGTVKEMLPDDPTLGQPDQALFHSLNSSLSQACASPSMEPLGVMPTHMGQGRYPVGVSNMVLRILGFLVDTAMGNKLIQVLLEDETTESAVKLSLPMGQEALITLKDGQQFVIQISDVPQSSEDIYFRENNANV

SEQ ID NO: 40 - protein, 459 aa, Homo sapiens
MEGSASPPEKPRARPAAAVLCRGPVEPLVFLANFALVLQGPLTTQYLWHRFSADLGYNGTRQRGGCSNRSADPTMQEVETLTSHWTLYMNVGGFLVGLFSSTLLGAWSDSVGRRPLLVLASLGLLLQALVSVFVVQLQLHVGYFVLGRILCALLGDFGGLLAASFASVADVSSSRSRTFRMALLEASIGVAGMLASLLGGHWLRAQGYANPFWLALALLIAMTLYAAFCFGETLKEPKSTRLFTFRHHRSIVQLYVAPAPEKSRKHLALYSLAIFVVITVHFGAQDILTLYELSTPLCWDSKLIGYGSAAQHLPYLTSLLALKLLQYCLADAWVAEIGLAFNILGMVVFAFATITPLMFTGYGLLFLSLVITPVIRAKLSKLVRETEQGALFSAVACVNSLAMLTASGIFNSLYPATLNFMKGFPFLLGAGLLLIPAVLIGMLEKADPHLEFQQFPQSP

SEQ ID NO: 41 - protein, 173 aa, Homo sapiens
MHWKMLLLLLLYYNAEASMCHRWSRAVLFPAAHRPKRSSSLPLNPVLQTSLEEVELLYEFLLAELEISPDLQISIKDEELASLRKASDFRTVCNNVIPKSIPDIRRLSASLSSHPGILKKEDFERTVLTLAYTAYRTALSHGHQKDIWAQSLVSLFQALRHDLMRSSQPGVPP

SEQ ID NO: 42 - protein, 245 aa, Homo sapiens
MTLFPVLLFLVAGLLPSFPANEDKDPAFTALLTTQTQVQREIVNKHNELRRAVSPPARNMLKMEWNKEAAANAQKWANQCNYRHSNPKDRMTSLKCGENLYMSSASSSWSQAIQSWFDEYNDFDFGVGPKTPNAVVGHYTQVVWYSSYLVGCGNAYCPNQKVLKYYYVCQYCPAGNWANRLYVPYEQGAPCASCPDNCDDGLCTNGCKYEDLYSNCKSLKLTLTCKHQLVRDSCKASCNCSNSIY

SEQ ID NO: 43 - protein, 13 aa, synthetic construct
  REGION 1..13: note = Synthetically generated peptide
KKSRGDYMTMQIG

SEQ ID NO: 44 - protein, 16 aa, synthetic construct
  REGION 1..16: note = Synthetically generated peptide
GGMEDIYFEFMGGKKK

SEQ ID NO: 45 - protein, 13 aa, synthetic construct
  REGION 1..13: note = Synthetically generated peptide
KKKGQEEEYVFIE